(19) United States
(12) Patent Application Publication
(10) Pub. No.: US 2017/0172871 A1
(43) Pub. Date: Jun. 22, 2017

(54) PROPELLANT-INCLUDING DEODORANTS AND/OR ANTIPERSPIRANTS COMPRISING AT LEAST TWO DIFFERENT PRESERVATIVES

(71) Applicant: Henkel AG & Co. KGaA, Duesseldorf (DE)

(72) Inventors: Bernhard Banowski, Duesseldorf (DE); Rainer Simmering, Grevenbroich (DE); Thomas Doering, Dormagen (DE); Melanie Rauschenberg, Kamen (DE)

(73) Assignee: Henkel AG & Co. KGaA, Duesseldorf (DE)

(21) Appl. No.: 15/377,621

(22) Filed: Dec. 13, 2016

(30) Foreign Application Priority Data
Dec. 17, 2015 (DE) 10 2015 225 686.3

(51) Int. Cl. A61K 8/26; A61K 8/23; A61K 8/41; A61K 8/34; A61K 8/36; A61K 8/44; A61Q 15/00; A61K 8/49

(52) U.S. Cl. CPC A61K 8/26; A61Q 15/00; A61K 8/23; A61K 8/4953; A61K 8/34; A61K 8/361; A61K 8/4926; A61K 8/44; A61K 8/36; A61K 8/416; A61K 8/347; A61K 8/41; A61K 2800/592; A61K 2800/524

(57) ABSTRACT

The present invention relates to propellant-containing deodorant and/or antiperspirant compositions comprising at least two different preservatives. The present invention furthermore relates to a method for preventing and/or reducing perspiration of the body and/or for preventing and/or reducing body odor, using the cosmetic agent according to the invention.

### FIELD OF THE INVENTION

The present invention generally relates to propellant-containing cosmetic agents, comprising at least one active ingredient, selected from deodorant active ingredients and/or antiperspirant aluminum salts and at least two different preservatives. The aforementioned compositions exhibit an outstanding deodorant and/or antiperspirant performance, while offering good preservation.

The present invention furthermore relates to a non-therapeutic cosmetic method for preventing and/or reducing body odor, in which a cosmetic agent according to the invention is applied to the skin, and in particular to the skin of the axilla region, and remains on the skin for at least 1 hour, especially for at least 2 hours, preferably for at least 4 hours, and in particular for at least 6 hours.

### BACKGROUND OF THE INVENTION

Due to the composition thereof, cosmetic agents can serve as a culture medium for microorganisms, such as bacteria, yeasts and fungi. These microorganisms can cause cosmetic products to be rendered unusable, for example as a result of mold formation. Moreover, a microbial contamination of the consumer may also occur. Additionally, these are capable of changing ingredients of the cosmetics, forming substances that have undesirable effects such as sensitivity or skin irritation. To prevent these undesirable consequences and ensure a certain minimum shelf life of the cosmetics, these need be preserved. Since preservatives, in turn, have irritating potential, use of the same in cosmetics is strictly regulated and governed by statutory requirements.

The skin microflora can have a decisive influence on different cosmetic parameters. Pathogenic bacteria such as Staphylococcus aureus, for example, play a crucial role in the development of blemished skin. Recent studies also indicate that an unbalanced skin microflora can have an influence on skin aging, since undesirable bacteria cause an increased immune defense of the skin, which in turn results in increased inflammatory responses during the course of which skin aging markers are stimulated.

A continued need therefore exists for preservative compositions that, on the one hand, prevent the colonization of the product by bacteria that are undesirable for the skin and, on the other hand, do not, or not significantly, impair the natural skin flora.

It is known, in essence, to mix different antimicrobial substances to increase the antimicrobial activity. WO 03/043593 A1, for example, proposes to combine conventional antibacterial substances such as triclosan, phenoxyethanol or hexetidine with ethyl lauroyl arginate to enhance the antibacterial action. WO 2007/014580 A1 proposes preservative mixtures that, in addition to ethyl lauroyl arginate, comprise salts of organic or inorganic acids, in particular sodium citrate, sodium acetate, sodium glutamate, sodium fumarate, sodium malate, sodium gluconate, sodium laurate, sodium lactate, sodium hexametaphosphate, sodium tertiary butyl hydroquinonate, sodium propylparabenate, or glucosamine hydrochloride or ethanolamine hydrochloride. Cosmetic compositions comprising a preservative mixture of ethyl lauroyl arginate and parabens, imidazolyl urea, phenoxyethanol, DMDM hydantoin, 2-methyl-5-chloro-3,4-isothiazolinone/2-methyl-3,4-isothiazolinone and Quaternium-15 are disclosed in EP 1414394 B1.

As a result, there is a continued need to provide antimicrobial compositions that are highly effective in small amounts.

It was therefore the object of the present invention to provide propellant-containing deodorant and/or antiperspirant compositions that exhibit an outstanding deodorant and/or antiperspirant performance, while offering good preservation. In particular, synergistic preservative mixtures were to be found, which are highly effective in low concentrations and, due to the lower overall usage amounts, enable deodorant and/or antiperspirant compositions that cause little irritation and sensitivity.

Surprisingly, it was now found that the use of certain mixtures of preservative mixtures in propellant-containing deodorant and/or antiperspirant compositions results in a synergistic effect with respect to the preserving action. As a result, the amount of preservatives used can be reduced, without adversely affecting the preserving action. Due to the reduced amount of preservatives, the deodorant and/or antiperspirant compositions according to the invention cause little irritation and sensitivity. Moreover, the addition of the preservative mixtures does not negatively influence the deodorant and/or antiperspirant performance.

Furthermore, other desirable features and characteristics of the present invention will become apparent from the subsequent detailed description of the invention and the appended claims, taken in conjunction with this background of the invention.

### BRIEF SUMMARY OF THE INVENTION

A cosmetic agent, comprising in at least one cosmetically compatible carrier 0.0005 to 35 wt. %, based on the total weight of the propellant-free cosmetic agent, of at least one active ingredient, selected from the group consisting of deodorant active ingredients, antiperspirant aluminum salts and the mixtures thereof; 2.0 to 95 wt. %, based on the total weight of the propellant-containing cosmetic agent, of at least one propellant; at least one first preservative (A), selected from the group consisting of sulfite(s), hexetidine, benzyl alcohol(s), undecylenic acid, phenoxyisopropanol, piroctone olamine, ethyl lauroyl arginate, formic acid, quaternary ammonium compounds, o-cymen-5-ol and hexamidine and the salts thereof; and at least one second preservative (B), selected from the group consisting of benzoic acid and the salts thereof, propionic acid and the salts thereof, salicylic acid and the salts thereof, sorbic acid and the salts thereof, zinc salts, paraben(s), polyaminopropyl biguanide, phenoxyethanol, climbazole, chlorhexidine and the salts thereof, glutaral, chlorphenesin, dimethyl oxazolidine, diazolidinyl urea, and mixtures of these preservatives.

### DETAILED DESCRIPTION OF THE INVENTION

The following detailed description of the invention is merely exemplary in nature and is not intended to limit the invention or the application and uses of the invention. Furthermore, there is no intention to be bound by any theory presented in the preceding background of the invention or the following detailed description of the invention.

The present invention relates to a cosmetic agent, comprising, in a cosmetically compatible carrier,
a) 0.0005 to 35 wt. %, based on the total weight of the propellant-free cosmetic agent, of at least one active ingredient, selected from the group consisting of deodorant active ingredients, antiperspirant aluminum salts and the mixtures thereof;
b) 2.0 to 95 wt. %, based on the total weight of the propellant-containing cosmetic agent, of at least one propellant;
c) at least one first preservative (A), selected from the group consisting of sulfite(s), hexetidine, benzyl alcohol(s), undecylenic acid, phenoxyisopropanol, piroctone olamine, ethyl lauroyl arginate, formic acid, quaternary ammonium compounds, o-cymen-5-ol and hexamidine and the salts thereof; and
d) at least one second preservative (B), selected from the group consisting of benzoic acid and the salts thereof, propionic acid and the salts thereof, salicylic acid and the salts thereof, sorbic acid and the salts thereof, zinc salts, paraben(s), polyaminopropyl biguanide, phenoxyethanol, climbazole, chlorhexidine and the salts thereof, glutaral, chlorphenesin, dimethyl oxazolidine, diazolidinyl urea, and mixtures of these preservatives.

The term “deodorant active ingredient” according to the invention shall be understood to mean an active ingredient that reduces and/or prevents the development of body odor as a result of the bacterial decomposition of perspiration, and in particular of axillary perspiration. However, this does not cover the above-described preservatives (A) and (B), or ethanol, which is possibly used as a component of the cosmetic carrier.

The information in % by weight in the present invention refers to the total weight of the propellant-free cosmetic agent, unless indicated otherwise.

The cosmetic agent includes components a) to d) in a cosmetically compatible carrier. This preferably comprises at least one component, selected from water, a C₁-C₄ alcohol, a cosmetic oil liquid under normal conditions, and mixtures thereof. The cosmetic oils liquid under normal conditions are not miscible with water and are neither odorants nor essential oils. “Normal conditions” within the meaning of the present invention refer to a temperature of 20° C. and a pressure of 1,013 hPa.

Suitable cosmetic carriers are aqueous or aqueous-alcoholic carriers, for example. This shall be understood to mean carriers that comprise more than 5.0 wt. % water, or more than 5 wt. % water and at least one C₁-C₄ alcohol, in each case based on the total weight of the propellant-free cosmetic agent. Aqueous carriers preferably comprise free water in a total amount of 10 to 96 wt. %, especially 15 to 80 wt. %, preferably 30 to 70 wt. %, and in particular 40 to 60 wt. %, based on the total weight of the propellant-free cosmetic agent. In aqueous-alcoholic carriers, ethanol is preferably present in a total amount of 0.1 to 60 wt. %, especially 0.5 to 30 wt. %, preferably 1.0 to 10 wt. %, and in particular 1.0 to 9.0 wt. %, based on the total weight of the propellant-free cosmetic agent.

The above-mentioned aqueous and aqueous-alcoholic carriers can moreover comprise at least one (C₂-C₆) alkyl alcohol having 2 or 3 hydroxy groups, in particular 1,2-propylene glycol, 1,3-propylene glycol, glycerol and 1,3-butylene glycol.

In this context, it is furthermore particularly preferred to use liquid cosmetic oils as cosmetic carriers. These cosmetic oils can be selected from the group consisting of (i) liquid non-silicone oils, in particular liquid paraffin oils and isoparaffin oils, such as isodecane, isoundecane, isododecane, isotridecane, isotetradecane, isopentadecane, isohexadecane and isoeicosane; (ii) non-volatile non-silicone oils, in particular the esters of linear or branched saturated or unsaturated C₂₋₃₀ fatty alcohols with linear or branched saturated or unsaturated C₂₋₃₀ fatty acids, which may be hydroxylated, the C₈ to C₂₂ fatty alcohol esters of monovalent or polyvalent C₂ to C₇ hydroxycarboxylic acids, the triethyl citrates, the dicarboxylic acid esters of linear or branched C₂ to C₁₀ alkanols, the addition products of ethylene oxide and/or propylene oxide to monohydric or polyhydric C₃₋₂₂ alkanols, which may optionally be esterified, the symmetrical, asymmetrical or cyclic esters of carbonic acid with fatty alcohols, the esters of dimers of unsaturated C₁₂₋₂₂ fatty acids with monohydric, linear, branched and cyclic C₂₋₁₈ alkanols or C₂₋₆ alkanols, the benzoic acid esters of linear or branched C₈₋₂₂ alkanols, such as benzoic acid-C₁₂₋₁₅-alkyl esters and benzoic acid isostearyl esters and benzoic acid octyldodecyl esters, the synthetic hydrocarbons, such as polyisobutene and polydecene, the alicyclic hydrocarbons; and (iii) mixtures thereof.

The term “volatile cosmetic oil” according to the invention refers to cosmetic oils that, at 20° C. and an ambient pressure of 1,013 hPa, have a vapor pressure of 2.66 Pa to 40,000 Pa (0.02 to 300 mm Hg), especially of 10 to 12,000 Pa (0.1 to 90 mm Hg), more preferably of 13 to 3,000 Pa (0.1 to 23 mm Hg), and in particular of 15 to 500 Pa (0.1 to 4 mm Hg). Moreover, the term “non-volatile cosmetic oils” within the meaning of the present invention shall be understood to mean cosmetic oils that, at 20° C. and an ambient pressure of 1,013 hPa, have a vapor pressure of less than 2.66 Pa (0.02 mm Hg).

Furthermore preferred according to the invention is the use of mixtures of the above-mentioned cosmetic oils, in particular of non-volatile and volatile cosmetic oils, since in this way parameters such as skin sensation, visibility of the residue, and stability of the cosmetic agent according to the invention can be set, and the agent can thus be better adapted to the needs of the consumers.

It is preferred within the scope of the present invention if the cosmetic oil that is liquid at 20° C. and 1,013 hPa is used in a total amount of 1.0 to 75 wt. %, especially of 2.0 to 95 wt. %, preferably of 5.0 to 70 wt. %, more preferably of 10 to 60 wt. %, and in particular of 15 to 50 wt. %, based on the total weight of the propellant-free cosmetic agent.

In addition, O/W emulsions, W/O emulsions and multiple emulsions, in particular W/O/W and O/W/O emulsions, are preferred cosmetic carriers.

As a first essential component a), the cosmetic agent according to the invention comprises at least one deodorant active ingredient and/or at least one antiperspirant aluminum salt.

Preferred cosmetic agents comprise certain deodorant active ingredients. It is therefore advantageous within the scope of the present invention if the cosmetic agent comprises at least one deodorant active ingredient, selected from the group consisting of (i) silver salts; (ii) alkanediols having 5 to 12 carbon atoms, in particular 3-(2-ethylhexyloxy)-1,2-propanediol, 1,2-hexanediol and 1,2-octanediol; (iii) triethyl citrates; (iv) active ingredients against exoesterases, in particular against arylsulfatase, lipase, beta-glucuronidase and cystathionine β-lyase; (v) cationic phospholipids; (vii) odor absorbers, in particular silicates, such as montmorillonite, kaolinite, illite, beidellite, nontronite, saponite, hectorite, bentonite, smectite, and talcum, zeolites, zinc ricinoleate, cyclodextrins; (vii) deodorizingly acting ion exchangers; (viii) antimicrobial substances; (ix) prebiotically active components; and (x) the mixtures thereof.

Preferred silver salts and/or silver complexes are, for example, silver lactate, silver nitrate, silver acetate, silver sulfate and silver citrate, and the combinations thereof with phyllosilicates and/or talcum. The alkanediol having 5 to 12 carbon atoms used is preferably 1,2-pentanediol, 1,2-hexanediol, 1,2-octanediol, 1,2-decanediol and the mixtures thereof. A mixture of 1,2-hexanediol and 1,2-octanediol in a weight ratio of 5:1 to 1:5 is particularly preferred. It may furthermore be preferred to use a combination of at least one alkanediol with tropolone.

Silicates serve as odor absorbers, which at the same time can also advantageously support the rheological properties of the cosmetic agents according to the invention. The particularly advantageous silicates according to the invention include in particular phyllosilicates, and among these in particular montmorillonite, kaolinite, illite, beidellite, nontronite, saponite, hectorite, bentonite, smectite, and talcum. Further advantageous odor absorbers are, for example, zeolites, zinc ricinoleate, cyclodextrins, certain metal oxides, such as aluminum oxide, and chlorophyll.

Moreover, the deodorant active ingredient can be selected from the group of the terpene alcohols, such as farnesol, chlorophyllin copper complexes, α-monoalkylglycerol ethers having a branched or linear, saturated or unsaturated, optionally hydroxylated, C₆-C₂₂ alkyl group, particularly preferably a-(2-ethylhexyl)glycerol ether, commercially available as Sensiva® SC 50 (ex Schülke & Mayr), carboxylic acid esters, in particular carboxylic acid monoesters of monoglycerol, diglycerol and triglycerol (in particular glycerol monolaurate, diglycerol monocaprinate, diglycerol monolaurate, triglycerol monolaurate and triglycerol monomyristate), and plant extracts (such as green tea and components of lime blossom oil).

Further preferred deodorant active ingredients are selected from what are known as prebiotically acting components, which according to the invention shall be understood to mean such components which inhibit solely, or at least predominantly, the odor-causing bacteria of the skin microflora, but not the desired, which is to say not odor-causing, microbes. The active ingredients disclosed as being prebiotically active in unexamined patent applications DE 10333245 and DE 102004011968 are explicitly included herein; among these are coniferous tree extracts, in particular from the group of the Pinaceae, and plant extracts from the group of the Sapindaceae, Araliaceae, Lamiaceae and Saxifragaceae, in particular extracts of Picea spp., Paullinia sp., Panax sp., Lamium album or Ribes nigrum, and mixtures of these substances.

Further preferred deodorant active ingredients are selected from the antimicrobially acting perfume oils and the Deosafe perfume oils, which are available from Symrise, formerly Haarmann and Reimer.

The enzyme inhibitors include substances that inhibit the enzymes responsible for the decomposition of sweat, in particular the arylsulfatase, β-glucuronidase, aminoacylase, the ester-cleaving lipases and lipoxigenase, such as trialkyl citrate, in particular triethyl citrate, or zinc glycinate.

The deodorant active ingredients can be used either individually or in mixtures. Particularly preferred are α-(2-ethylhexyl) glycerol ether, diglycerol monocaprinate, 2-methyl-4-phenylbutan-2-ol, mixtures of phenoxyethanol and α-(2-ethylhexyl)glycerol ether and mixtures of aryl alcohols, in particular phenoxyethanol, with α-(2-ethylhexyl) glycerol ether and diglycerol monocaprinate.

The at least one deodorant active ingredient is preferably used in certain quantity ranges in the cosmetic agents according to the invention. Preferred cosmetic agents according to the present invention are thus characterized by comprising, based on the total weight of the propellant-free cosmetic agents, 0.0005 to 20 wt. %, especially 0.1 to 12 wt. %, preferably 0.1 to 10 wt. %, and in particular 0.1 to 2.0 wt. % of at least one deodorant active ingredient. If a mixture of deodorant active ingredients is used, the above-described quantity information refers to the mixture of these active ingredients. The use of the aforementioned amounts ensures sufficient deodorant performance. Furthermore, these amounts have no negative influence on the preserving action of the mixture made of at least two preservatives.

In addition to, or in combination with, the at least one deodorant active ingredient, the cosmetic agents according to the invention can additionally comprise at least one antiperspirant aluminum salt. It is therefore preferred within the scope of the present invention if the cosmetic agent comprises at least one antiperspirant aluminum salt, selected from the group consisting of (i) water-soluble astringent inorganic salts of aluminum, in particular aluminum chlorohydrate, aluminum sesquichlorohydrate, aluminum dichlorohydrate, aluminum hydroxide, potassium aluminum sulfate, aluminum bromohydrate, aluminum chloride, aluminum sulfate; (ii) water-soluble astringent organic salts of aluminum, in particular aluminum chlorohydrex propylene glycol, aluminum chlorohydrex polyethylene glycol, aluminum propylene glycol complexes, aluminum sesquichlorohydrex propylene glycol, aluminum sesquichlorohydrex polyethylene glycol, aluminum propylene glycol dichlorohydrex, aluminum polyethylene glycol dichlorohydrex, aluminum undecylenoyl collagen amino acid, sodium aluminum lactate, sodium aluminum chlorohydroxy lactate, aluminum lipoamino acids, aluminum lactate, aluminum chlorohydroxy allantoinate, sodium aluminum chlorohydroxy lactate; and (iii) the mixtures thereof.

Particularly preferred inorganic aluminum salts are selected from aluminum chlorohydrate, in particular aluminum chlorohydrate of the general formula [Al₂(OH)₅Cl.1-6H₂O]ₙ, preferably [Al₂(OH)₅Cl.2-3H₂O]ₙ, which may be present in non-activated (polymerized) or in activated (depolymerized) form, and aluminum chlorohydrate of the general formula [Al₂(OH)₄Cl₂.1-6H₂O]ₙ, preferably [Al₂(OH)₄Cl₂.2-3H₂O]ₙ, which may be present in non-activated (polymerized) or in activated (depolymerized) form. The production of such antiperspirant aluminum salts is disclosed in documents U.S. Pat. No. 3,887,692 A, U.S. Pat. No. 3,904,741 A, U.S. Pat. No. 4,359,456 A, GB 2048229 A, and GB 1347950 A, for example.

Particularly preferred antiperspirant aluminum salts according to the invention are selected from what are known as “activated” aluminum salts, which are also referred to as “enhanced-activity” antiperspirant active ingredients. Such active ingredients are known from the prior art and are also commercially available. Production of these is disclosed in documents GB 2048229 A, U.S. Pat. No. 4,775,528 A, and U.S. Pat. No. 6,010,688 A. Activated aluminum salts are generally produced by heat treating a dilute solution of the corresponding salt (such as a solution containing 10 wt. % salt), so as to increase the HPLC peak 4 to peak 3 area ratio of the same. The activated salt can subsequently be dried to obtain a powder, in particular spray-dried. In addition to spray drying, drum drying also suited, for example. Activated aluminum salts typically have an HPLC peak 4 to peak 3 area ratio of at least 0.4, especially of at least 0.7, and in particular of at least 0.9, wherein at least 70% of the aluminum can be assigned to these HPLC peaks.

It is likewise possible according to the invention to use antiperspirant aluminum salts as non-aqueous solutions or solubilizates of an activated antiperspirant aluminum salt, for example in accordance with document U.S. Pat. No. 6,010,688 A. As a result of the addition of an effective amount of a polyhydric alcohol comprising 3 to 6 carbon atoms and 3 to 6 hydroxyl groups, preferably propylene glycol, sorbitol and pentaerythritol, such aluminum salts are stabilized against the loss of the activation of the salt.

Particularly preferred are also complexes of activated antiperspirant aluminum salts with a polyhydric alcohol, which include 20 to 50 wt. %, especially 20 to 42 wt. %, activated antiperspirant aluminum salt and 2 to 16 wt. % molecularly bound water, wherein the remainder to make up 100 wt. % is at least one polyhydric alcohol comprising 3 to 6 carbon atoms and 3 to 6 hydroxyl groups. Propylene glycol, propylene glycol/sorbitol mixtures, and propylene glycol/pentaerythritol mixtures are preferred such alcohols. Such preferred complexes according to the invention of an activated antiperspirant aluminum salt with a polyhydric alcohol are disclosed in documents U.S. Pat. No. 5,643,558 A and U.S. Pat. No. 6,245,325 A for example.

It is likewise possible within the scope of the present invention to use alkaline calcium-aluminum salts, as they are disclosed in document U.S. Pat. No. 2,571,030 A, for example, as antiperspirant aluminum salts. These salts can be obtained by reacting calcium carbonate with aluminum chlorhydroxide or aluminum chloride and aluminum powder, or by adding calcium chloride dihydrate to aluminum chlorhydroxide. However, it is likewise possible to use aluminum-zirconium complexes that are buffered with salts of amino acids, in particular with alkali and alkaline earth glycinates, as they are disclosed in document U.S. Pat. No. 4,017,599 A, for example.

The aluminum salts listed in the documents U.S. Pat. No. 6,245,325 A, U.S. Pat. No. 6,042,816 A, U.S. Pat. No. 6,245,325 A, U.S. Pat. No. 6,042,816 A, U.S. Pat. No. 6,245,325 A, U.S. Pat. No. 6,042,816 A, U.S. Pat. No. 6,245,325 A, U.S. Pat. No. 6,042,816 A or U.S. Pat. No. 7,105,691 A, which are preferably stabilized by amino acids, in particular glycine, hydroxyalkanoic acids, in particular glycolic acid and lactic acid, or betaines, can also be used as preferred antiperspirant activated aluminum salts according to the invention.

Further preferred activated aluminum salts are those of the general formula Al₂(OH)₆₋ₐXa, where X is Cl, Br, I or NO₃, and “a” is a number from 0.3 to 5, especially from 0.8 to 2.5, and in particular from 1 to 2, so that the molar ratio of Al:X is 0.9:1 to 2.1:1. Such activated antiperspirant aluminum salts are disclosed in document U.S. Pat. No. 6,074,632 A, for example. Aluminum chlorohydrate (which is to say X is Cl in the aforementioned formula) is particularly preferred, and specifically 5/6 basic aluminum chlorohydrate, where “a” is 1, so that the molar ratio of aluminum to chlorine is 1.9:1 to 1.9:1.

Antiperspirant aluminum salts that are particularly preferred according to the invention have a metal-to-chloride molar ratio of 1.9 to 2.1. The metal-to-chloride ratio of likewise particularly preferred aluminum sesquichlorohydrates within the scope of the invention is 1.5:1 to 1.8:1.

The at least one antiperspirant aluminum salt is preferably used in certain quantity ranges in the cosmetic agents according to the invention. Preferred cosmetic agents according to the present invention are thus characterized by comprising, based on the total weight thereof, 0.1 to 35 wt. %, especially 0.5 to 25 wt. %, preferably 1 to 15 wt. %, more preferably 1.5 to 10 wt. %, and in particular 2.0 to 8.0 wt. % of at least one antiperspirant aluminum salt and/or aluminum-zirconium salt. If a mixture of different antiperspirant aluminum salts is used, the above-described quantity information refers to the mixture of these salts. The use of the aforementioned amounts ensures sufficient deodorant and antiperspirant performance. As a result of the above-described special preservative combination, an outstanding preserving performance can be ensured even if the above-described amounts of the antiperspirant aluminum salt are used.

As a second essential component b), the cosmetic agents according to the invention comprise at least one propellant. Cosmetic agents according to the invention are thus formulated as a spray application. The spray application is carried out using a spraying device, which in a container includes a filling composed of the antiperspirant cosmetic agent according to the invention in liquid, viscous-flowable, suspension or powder form and is pressurized by a propellant (pressurized cans, pressurized containers, aerosol dispensers). The containers comprise a withdrawal device, preferably in the form of valves, allowing the content to be withdrawn in the form of a mist, smoke, foam, powder, paste or fluid jet. Especially cylindrical vessels made of metal (aluminum, tinplate, volume preferably no more than 1,000 mol), protected or shatterproof glass or plastic (volume preferably no more than 220 ml) or shattering glass or plastic (volume preferably 50 to 400 ml) can be used as containers for the spraying devices. The packaging for the agents according to the invention can be opaque, but may also be transparent or translucent. \

Suitable propellants (propellant gases) within the scope of the invention are, for example, propane, propene, n-butane, iso-butane, iso-butene, n-pentane, pentene, iso-pentane, iso-pentene, methane, ethane, dimethylether, nitrogen, air, oxygen, nitrous oxide, 1, 1,1,3-tetrafluoroethane, heptafluoro-n-propane, perfluoroethane, monochlorodifluoromethane, 1, 1-difluoroethane, tetrafluoropropene, and more particularly both individually and in the mixtures thereof. It is also possible to advantageously use hydrophilic propellants, such as carbon dioxide, within the meaning of the present invention if the proportion of hydrophilic gases is selected to be low, and lipophilic propellant (such as propane/butane) is present in excess. Preferred cosmetic agents according to the invention are thus characterized by comprising at least one propellant, selected from the group consisting of propane, propene, n-butane, iso-butane, iso-butene, n-pentane, pentene, iso-pentane, iso-pentene, methane, ethane, dimethyl ether, nitrogen, air, oxygen, nitrous oxide, 1,1,1,3-tetrafluoroethane, heptafluoro-n-propane, perfluoroethane, monochlorodifluoromethane, 1,1-difluoroethane, tetrafluoropropene, carbon dioxide, and the mixtures thereof.

According to the invention, the at least one propellant is preferably used in certain total amounts. Preferred cosmetic agents according to the present invention are thus characterized by comprising, based on the total weight of the propellant-containing cosmetic agents, 2.0 to 90 wt. %, especially 30 to 85 wt. %, preferably 35 to 80 wt. %, and in particular 40 to 75 wt. % of at least one propellant. If a mixture of propellants is used, the above-described quantity information refers to the mixture of these propellants. The use of the above-described amounts ensures adequate sprayability and complete emptying of the aerosol can. The even application of the cosmetic agents using the above-described propellants and propellant amounts ensures an outstanding deodorant and/or antiperspirant performance.

As a third essential component b), the cosmetic agents according to the invention comprise at least one first preservative (A), selected from the group consisting of sulfite(s), hexetidine, benzyl alcohol(s), undecylenic acid, phenoxyisopropanol, piroctone olamine, ethyl lauroyl arginate, formic acid, quaternary ammonium compounds, o-cymen-5-ol, and hexamidine and the salts thereof. These compounds have the following chemical names and CAS numbers.

| Preservative | Chemical name | CAS number |
|---|---|---|
| Hexetidine | 1,3-Bis(2-ethylhexyl)hexahydro-5- | 141-94-6 |
| | methyl-5-pyrimidiamine | |
| Benzyl alcohol | Benzyl alcohol | 100-51-6 |
| Undecylenic acid | Undec-10-enoic acid | 112-38-9 |
| Phenoxyisopropanol | 1-Phenoxypropan-2-ol | 770-35-4 |
| Piroctone olamine | 1-hydroxy-4-methyl-6-(2,4,4- | 68890-66-4 |
| | trimethylpentyl)-2-pyridone | |
| Ethyl lauroyl | Ethyl-N-alpha-dodecanoyl-L-arginate | 60372-77-2 |
| arginate*HCl | hydrochloride | |
| o-cymen-5-ol | 4-isopropyl-m-cresol | 2/2/3228 |
| Hexamidine | 4,4′-[1,6-hexanediylbis(oxy)]-bis- | 3811-75-4 |
| | [benzenecaroxamidine] | |
| Hexamidine | 2-hydroxyethanesulfonic acid, | 659-40-5 |
| diisethionate | compd. with 4,4′-[hexane-1,6- | |
| | diylbis(oxy)]-bis- | |
| | benzenecaroxamidine] (2:1) | |
| Hexamidine paraben | p-hydroxbenzoic acid, compd. with | 93841-83-9 |
| | 4,4′-[hexane-1,6-diylbis(oxy)]- | |
| | bis-[benzenecaroxamidine] (2:1) | |

In conjunction with the second preservative (B), these preservatives have a synergistic effect with respect to the antimicrobial action and thus result in a particularly effective preservation of the cosmetic agents according to the invention. Furthermore, due to the synergistic effect, the amount used can be reduced, whereby cosmetic agents that cause little irritation and sensitivity are obtained.

The preserving action can be further enhanced if the cosmetic agents comprise more than two of the above-described preservative. Preferred cosmetic agents are thus characterized by comprising at least two preservatives (A), selected from the group consisting of sulfite(s), hexetidine, benzyl alcohol(s), undecylenic acid, phenoxyisopropanol, piroctone olamine, ethyl lauroyl arginate, formic acid, quaternary ammonium compounds, o-cymen-5-ol and hexamidine and the salts thereof.

Further preferred cosmetic agents according to the invention are characterized by comprising at least three preservatives (A), selected from the group consisting of sulfite(s), hexetidine, benzyl alcohol(s), undecylenic acid, phenoxyisopropanol, piroctone olamine, ethyl lauroyl arginate, formic acid, quaternary ammonium compounds, o-cymen-5-ol and hexamidine and the salts thereof.

In addition, cosmetic agents according to the invention that comprise at least four preservatives (A), selected from the group consisting of sulfite(s), hexetidine, benzyl alcohol(s), undecylenic acid, phenoxyisopropanol, piroctone olamine, ethyl lauroyl arginate, formic acid, quaternary ammonium compounds, o-cymen-5-ol and hexamidine and the salts thereof, are advantageous.

In addition, within the scope of the present invention, preferred cosmetic agents are those that comprise at least five preservatives (A), selected from the group consisting of sulfite(s), hexetidine, benzyl alcohol(s), undecylenic acid, phenoxyisopropanol, piroctone olamine, ethyl lauroyl arginate, formic acid, quaternary ammonium compounds, o-cymen-5-ol and hexamidine and the salts thereof.

Finally, cosmetic agents according to the invention that comprise a mixture of sulfite(s), hexetidine, benzyl alcohol(s), undecylenic acid, phenoxyisopropanol, piroctone olamine, ethyl lauroyl arginate, formic acid, quaternary ammonium compounds, o-cymen-5-ol, and hexamidine and the salts thereof, as preservative (A) are preferred.

The cosmetic agents according to the invention can comprise at least one quaternary ammonium compound as the preservative. It has been found to be advantageous within the scope of the present invention to use certain quaternary ammonium compounds. It is thus preferred within the scope of the present invention if the cosmetic agent comprises at least one compound, selected from the group consisting of behentrimonium chloride, cetrimonium bromide, cetrimonium chloride, laurtrimonium bromide, laurtrimonium chloride, steartrimonium bromide, steartrimonium chloride, benzethonium chloride, benzalkonium chloride, and the mixtures thereof, as quaternary ammonium compounds. The above-described compounds are C₁₂-C₂₂ alkyltrimethylammonium bromides and chlorides, wherein the respective designation denotes the length of the alkyl chain in the particular compound. Behentrimonium chloride is thus C₂₂ alkyltrimethylammonium chloride, cetrimonium chloride denotes C₁₆ alkyltrimethylammonium chloride, laurtrimonium bromide is C₁₂ alkyltrimethylammonium bromide, and steartrimonium bromide denotes C₁₈ alkyltrimethylammonium bromide. The use of such quaternary ammonium compounds, in combination with at least one further claimed preservative, has proven to be particularly advantageous with respect to the synergistic increase in the preservative performance of this mixture.

The cosmetic agents according to the invention preferably comprise the first preservative (A) in certain quantity ranges. Preferred cosmetic agents according to the invention are thus characterized by comprising, based on the total weight thereof, 0.001 to 10 wt. %, especially 0.005 to 7.0 wt. %, preferably 0.01 to 4.0 wt. %, and in particular 0.05 to 2.0 wt. % of at least one first preservative (A). If more than one preservative (A) is used, the above-mentioned total amounts refer to the mixture of these preservatives. The use of such amounts of the first preservative (A) results in an outstanding preservation of the cosmetic agents according to the invention. Moreover, the synergistic effect of the combination of preservative (A) with preservative (B) allows the amount of preservatives used to be reduced, without adversely influencing the preserving performance. The cosmetic agents according to the invention thus cause particularly little irritation and sensitivity.

As a fourth essential component d), the cosmetic agents according to the invention comprise at least one second preservative (B), selected from the group consisting of benzoic acid and the salts thereof, propionic acid and the salts thereof, salicylic acid and the salts thereof, sorbic acid and the salts thereof, zinc salts, paraben(s), polyaminopropyl biguanide, phenoxyethanol, climbazole, chlorhexidine and the salts thereof, glutaral, chlorphenesin, dimethyl oxazolidine, diazolidinyl urea, and mixtures of these preservatives. These compounds have the following chemical names and CAS numbers.

| Preservative | Chemical name | CAS number |
|---|---|---|
| Zinc pyrithione | Pyridine-2-thiol-1-oxide | 13463-41-7 |
| Polyaminopropyl | Poly(1-hexamethylene biguanide | 133029-32-0 |
| biguanide | hydrochloride) | |
| Phenoxyethanol | 2-phenoxy-1-ethanol | 122-99-6 |
| Climbazole | 1-(4-chlorophenoxy)-1-(imidazole- | 38083-17-9 |
| | l-yl)-3,3-dimethylbutan-2-one | |
| Chlorhexidine | N,N′-bis(4-chlorophenyl)-3,12- | 55-56-1 |
| | diimino-2,4,11,13- | |
| | tetraazatetradecanediamidine | |
| Glutaral | Glutaraldehyde | 111-30-8 |
| Chlorphenesin | 3-(4-chlorophenoxy)-1,2- | 104-29-0 |
| | propanediol | |
| Dimethyloxazolidine | 4,4-dimethyl-1,3-oxazolidine | 51200-87-4 |
| Diazolidinyl urea | 1-[3,4-bis(hydroxymethyl)-2,5- | 78491-02-8 |
| | dioxoimidazolidin-4-yl]-1,3- | |
| | bis(hydroxymethyl)urea | |

The addition of the second preservative (B), in conjunction with the above-described preservatives (A), results in a synergistic increase in the preserving performance.

The cosmetic agents according to the invention particularly preferably comprises the preservatives cited in Table 1 below, or mixtures of these preservatives ZK1 to ZK361 (be=sodium benzoate, pr=propionic acid, sa=sodium salicylate, zn=zinc pyrithione, pb=paraben, pg=propylaminopropyl biguanide, pe=phenoxyethanol, cl=climbazole, chi=chlorhexidine, ch=chlorphenesin, gl=glutaral, dx=dimethyloxazolidine, dh=diazolidinyl urea) as the second preservative (B).

TABLE 1
Second preservative (B) or mixtures of these preservatives

| ZK1 | ZK2 | ZK3 | ZK4 | ZK5 | ZK6 | ZK7 | ZK8 |
|---|---|---|---|---|---|---|---|
| be | pr | sa | Zn | pb¹⁾ | pg | pe | cl |
| ZK9 | ZK10 | ZK11 | ZK12 | ZK13 | ZK14 | ZK15 | ZK16 |
| chi | ch | gl | Dx | be + pr | be + pr + sa | be + pr + zn | be + pr + pb |
| ZK17 | ZK18 | ZK19 | ZK20 | ZK21 | ZK22 | ZK23 | ZK24 |
| be + pr + pg | be + pr + pe | be + pr + cl | be + pr + chi | be + pr + ch | be + pr + gl | be + pr + dx | be + sa + zn |
| ZK25 | ZK26 | ZK27 | ZK28 | ZK29 | ZK30 | ZK31 | ZK32 |
| be + sa + | be + sa + pg | be + sa + pe | be + sa + cl | be + sa + | be + sa + ch | be + sa + gl | be + sa + dx |
| pb¹⁾ | | | | chi | | | |
| ZK33 | ZK34 | ZK35 | ZK36 | ZK37 | ZK38 | ZK39 | ZK40 |
| be + zn + | be + zn + pg | be + zn + pe | be + zn + cl | be + zn + | be + zn + ch | be + zn + gl | be + zn + dx |
| pb¹⁾ | | | | chi | | | |
| ZK41 | ZK42 | ZK43 | ZK44 | ZK45 | ZK46 | ZK47 | ZK48 |
| be + pb¹⁾ + | be + pb¹⁾ + | be + pb + cl | be + pb¹⁾ + | be + pb¹⁾ + | be + pb¹⁾ + | be + pb¹⁾ + | be + pg + pe |
| pg | pe | | chi | ch | gl | dx | |
| ZK49 | ZK50 | ZK51 | ZK52 | ZK53 | ZK54 | ZK55 | ZK56 |
| be + pg + cl | be + pg + | be + pg + ch | be + pg + gl | be + pg + dx | be + pe + cl | be + pe + | be + pe + ch |
| | chi | | | | | chi | |
| ZK57 | ZK58 | ZK59 | ZK60 | ZK61 | ZK62 | ZK63 | ZK64 |
| be + pe + gl | be + pe + dx | be + cl + chi | be + cl + ch | be + cl + gl | be + cl + dx | be + chi + | be + chi + gl |
| | | | | | | ch | |
| ZK65 | ZK66 | ZK67 | ZK68 | ZK69 | ZK70 | ZK71 | ZK72 |
| be + chi + dx | pr + sa | pr + sa + zn | pr + sa + | pr + sa + pg | pr + sa + pe | pr + sa + cl | pr + sa + chi |
| | | | pb¹⁾ | | | | |
| ZK73 | ZK74 | ZK75 | ZK76 | ZK77 | ZK78 | ZK79 | ZK80 |
| pr + sa + gl | pr + sa + dx | pr + zn + | pr + zn + pg | pr + za + pe | pr + za + cl | pr + za + chi | pr + za + gl |
| | | pb¹⁾ | | | | | |
| ZK81 | ZK82 | ZK83 | ZK84 | ZK85 | ZK86 | ZK87 | ZK88 |
| pr + za + dx | pr + pb¹⁾ + | pr + pb¹⁾ + | pr + pb¹⁾ + | pr + pb¹⁾ + | pr + pb¹⁾ + | pr + pb¹⁾ + | pr + pg + pe |
| | pg | pe | cl | chi | gl | dx | |
| ZK89 | ZK90 | ZK91 | ZK92 | ZK93 | ZK94 | ZK95 | ZK96 |
| pr + pg + cl | pr + pg + chi | pr + pg + gl | pr + pg + dx | pr + pe + cl | pr + pe + chi | pr + pe + ch | pr + pe + gl |
| ZK97 | ZK98 | ZK99 | ZK100 | ZK101 | ZK102 | ZK103 | ZK104 |
| pr + pe + dx | pr + cl + chi | pr + cl + gl | pr + cl + dx | pr + chi + gl | pr + chi + dx | pr + sa + ch | pr + za + ch |
| ZK105 | ZK106 | ZK107 | ZK108 | ZK109 | ZK110 | ZK111 | ZK112 |
| pr + pb¹⁾ + | pr + pg + ch | pr + cl + ch | pr + chi + ch | pr + ch + gl | pr + ch + dx | pr + gl + dx | be + sa |
| ch | | | | | | | |
| ZK113 | ZK114 | ZK115 | ZK116 | ZK117 | ZK118 | ZK119 | ZK120 |
| be + zn | pr + pg | pr + pg | pr + pe | pr + cl | pr + chi | pr + ch | pr + gl |
| ZK121 | ZK122 | ZK123 | ZK124 | ZK125 | ZK126 | ZK127 | ZK128 |
| be + pb¹⁾ | be + pg | be + pe | be + cl | be + chi | be + ch | be + gl | be + dx |
| ZK129 | ZK130 | ZK131 | ZK132 | ZK133 | ZK134 | ZK135 | ZK136 |
| pr + zn | pr + pb¹⁾ | pr + pg | pr + pe | pr + cl | pr + chi | pr + ch | pr + gl |
| ZK137 | ZK138 | ZK139 | ZK140 | ZK141 | ZK142 | ZK143 | ZK144 |
| pr + dx | sa + zn | sa + pb¹⁾ | sa + pg | sa + pe | sa + cl | sa + chi | sa + ch |
| ZK145 | ZK146 | ZK147 | ZK148 | ZK149 | ZK150 | ZK151 | ZK152 |
| sa + gl | sa + dx | zn + pb¹⁾ | zn + pg | zn + pe | zn + cl | zn + chi | zn + ch |
| ZK153 | ZK154 | ZK155 | ZK156 | ZK157 | ZK158 | ZK159 | ZK160 |
| zn + gl | zn + dx | pb¹⁾ + pg | pb¹⁾ + pe | pb¹⁾ + cl | pb¹⁾ + chi | pb¹⁾ + ch | pb¹⁾ + gl |
| ZK161 | ZK162 | ZK163 | ZK164 | ZK165 | ZK166 | ZK167 | ZK168 |
| pb¹⁾ + dx | pg + pe | pg + cl | pg + chi | pg + ch | pg + gl | pg + dx | pe + cl |
| ZK169 | ZK170 | ZK171 | ZK172 | ZK173 | ZK174 | ZK175 | ZK176 |
| pe + chi | pe + ch | pe + gl | pe + dx | cl + chi | cl + ch | cl + gl | cl + dx |
| ZK177 | ZK178 | ZK179 | ZK180 | ZK181 | ZK182 | ZK183 | ZK184 |
| chi + ch | chi + gl | chi + dx | ch + gl | ch + dx | gl + dx | sa + pb¹⁾ + | sa + pb¹⁾ + |
| | | | | | | pg | pe |
| ZK185 | ZK186 | ZK187 | ZK188 | ZK189 | ZK190 | ZK191 | ZK192 |
| sa + pb¹⁾ + | sa + pb¹⁾ + | sa + pb¹⁾ + | sa + pb¹⁾ + | sa + pb¹⁾ + | sa + zn + | sa + zn + pg | sa + zn + pe |
| cl | chi | ch | gl | dx | pb¹⁾ | | |
| ZK193 | ZK194 | ZK195 | ZK196 | ZK197 | ZK198 | ZK199 | ZK200 |
| sa + zn + cl | sa + zn + chi | sa + zn + ch | sa + zn + gl | sa + zn + dx | sa + pg + pe | sa + pg + cl | sa + pg + |
| | | | | | | | chi |
| ZK201 | ZK202 | ZK203 | ZK204 | ZK205 | ZK206 | ZK207 | ZK208 |
| sa + pg + ch | sa + pg + gl | sa + pg + dx | sa + pe + cl | sa + pe + | sa + pe + ch | sa + pe + gl | sa + pe + dx |
| | | | | chi | | | |
| ZK209 | ZK210 | ZK211 | ZK212 | ZK213 | ZK214 | ZK215 | ZK216 |
| sa + cl + chi | sa + cl + ch | sa + cl + gl | sa + cl + dx | sa + chi + ch | sa + chi + gl | sa + chi + dx | sa + ch + gl |
| ZK217 | ZK218 | ZK219 | ZK220 | ZK221 | ZK222 | ZK223 | ZK224 |
| sa + ch + dx | sa + gl + dx | zn + pb¹⁾ + | zn + pb¹⁾ + | zn + pb¹⁾ + | zn + pb¹⁾ + | zn + pb¹⁾ + | zn + pb¹⁾ + |
| | | pg | pe | cl | chi | ch | gl |
| ZK225 | ZK226 | ZK227 | ZK228 | ZK229 | ZK230 | ZK231 | ZK232 |
| zn + pb¹⁾ + | zn + pg + pe | zn + pg + cl | zn + pg + | zn + pg + ch | zn + pg + gl | zn + pg + dx | zn + pe + cl |
| dx | | | chi | | | | |
| ZK233 | ZK234 | ZK235 | ZK236 | ZK237 | ZK238 | ZK239 | ZK240 |
| zn + pe + | zn + pe + ch | zn + pe + gl | zn + pe + dx | zn + cl + chi | zn + cl + ch | zn + cl + gl | zn + cl + dx |
| chi | | | | | | | |
| ZK241 | ZK242 | ZK243 | ZK244 | ZK245 | ZK246 | ZK247 | ZK248 |
| zn + chi + ch | zn + chi + gl | zn + chi + | zn + ch + gl | zn + ch + dx | zn + gl + dx | pb¹⁾ + | pb¹⁾ + |
| | | dx | | | | pe + cl | pe + chi |
| ZK249 | ZK250 | ZK251 | ZK252 | ZK253 | ZK254 | ZK255 | ZK256 |
| pb¹⁾ + | pb¹⁾ + | pb¹⁾ + | pb¹⁾ + | pb¹⁾ + | pb¹⁾ + | pb¹⁾ + | pb¹⁾ + |
| pe + ch | pe + gl | pe + dx | cl + chi | cl + ch | cl + gl | cl + dx | chi + ch |
| ZK257 | ZK258 | ZK259 | ZK260 | ZK261 | ZK262 | ZK263 | ZK264 |
| pb¹⁾ + | pb¹⁾ + | pb¹⁾ + | pb¹⁾ + | pb¹⁾ + | pg + pe + cl | pg + pe + | pg + pe + ch |
| chi + gl | chi + dx | ch + gl | ch + dx | gl + dx | | chi | |
| ZK265 | ZK266 | ZK267 | ZK268 | ZK269 | ZK270 | ZK271 | ZK272 |
| pg + pe + gl | pg + pe + dx | pg + cl + chi | pg + cl + ch | pg + cl + gl | pg + cl + dx | pg + chi + ch | pg + chi + gl |
| ZK273 | ZK274 | ZK275 | ZK276 | ZK277 | ZK278 | ZK279 | ZK280 |
| pg + chi + dx | pg + ch + gl | pg + ch + dx | pg + gl + dx | pe + cl + chi | pe + cl + ch | pe + cl + gl | pe + cl + dx |
| ZK281 | ZK282 | ZK283 | ZK284 | ZK285 | ZK286 | ZK287 | ZK288 |
| pe + chi + ch | pe + chi + gl | pe + chi + | pe + ch + gl | pe + ch + dx | pe + gl + dx | cl + chi + ch | cl + chi + gl |
| | | dx | | | | | |
| ZK289 | ZK290 | ZK291 | ZK292 | ZK293 | ZK294 | ZK295 | ZK296 |
| cl + chi + dx | cl + ch + gl | cl + ch + dx | cl + gl + dx | chi + ch + gl | chi + ch + | ch + gl + dx | dh |
| | | | | | dx | | |
| ZK297 | ZK298 | ZK299 | ZK300 | ZK301 | ZK302 | ZK303 | ZK304 |
| be + sa + dh | be + zn + dh | be + pb¹⁾ + | be + pg + dh | be + cl + dh | be + pe + dh | be + pr + dh | be + pe + |
| | | dh | | | | | dh |
| ZK305 | ZK306 | ZK307 | ZK308 | ZK309 | ZK310 | ZK311 | ZK312 |
| be + cl + dh | be + chi + dh | pr + sa + dh | pr + za + dh | pr + pb¹⁾ + | pr + pg + dh | pr + pe + dh | pr + cl + dh |
| | | | | dh | | | |
| ZK313 | ZK314 | ZK315 | ZK316 | ZK317 | ZK318 | ZK319 | ZK320 |
| pr + chi + dh | pr + ch + dh | pr + gl + dh | be + dh | pr + dh | sa + dh | zn + dh | pg + dh |
| ZK321 | ZK322 | ZK323 | ZK324 | ZK325 | ZK326 | ZK327 | ZK328 |
| pb¹⁾ + dh | pe + dh | cl + dh | chi + dh | ch + dh | gl + dh | sa + pb¹⁾ + | sa + zn + dh |
| | | | | | | dh | |
| ZK329 | ZK330 | ZK331 | ZK332 | ZK333 | ZK334 | ZK335 | ZK336 |
| sa + pg + dh | sa + pe + dh | sa + cl + dh | sa + chi + dh | sa + ch + dh | sa + gl + dh | zn + pb¹⁾ + | zn + pg + dh |
| | | | | | | dh | |
| ZK337 | ZK338 | ZK339 | ZK340 | ZK341 | ZK342 | ZK343 | ZK344 |
| zn + pe + dh | zn + cl + dh | zn + chi + | zn + ch + dh | zn + gl + dh | pb¹⁾ + | pb¹⁾ + | pb¹⁾ + |
| | | dh | | | pe + dh | cl + dh | chi + dh |
| ZK345 | ZK346 | ZK347 | ZK348 | ZK349 | ZK350 | ZK351 | ZK352 |
| pb¹⁾ + | pb¹⁾ + | pg + pe + dh | pg + cl + dh | pg + chi + dh | pg + ch + dh | pg + gl + dh | pe + cl + dh |
| ch + dh | gl + dh | | | | | | |

| ZK353 | ZK354 | ZK355 | ZK356 | ZK357 |
|---|---|---|---|---|
| pe + chi + | pe + ch + dh | pe + gl + dh | cl + chi + dh | cl + ch + dh |
| dh | | | | |

| ZK358 | ZK359 | ZK360 | ZK361 |
|---|---|---|---|
| cl + gl + dh | chi + ch + dh | ch + gl + dh | gl + dx + dh |

¹⁾Paraben is selected from the group consisting of methylparaben, ethylparaben, propylparaben, butylparaben and the mixtures thereof

In this context, it is advantageous if the second preservative (B) is used in a certain total amount. It is thus preferred according to the invention if the cosmetic agents, based on the total weight thereof, comprise 0.001 to 12 wt. %, especially 0.005 to 7.0 wt. %, preferably 0.05 to 6.0 wt. %, and in particular 0.1 to 5.0 wt. % of at least one second preservative (B). If more than one preservative (B) is used, the above-mentioned total amounts refer to the mixture of these preservatives. The use of these amounts of the second preservative (B), in combination with the first preservative (A), results in a synergistic increase in the preserving performance.

The following Tables 2 to 4 each list particularly preferred embodiments AF1 to AF504 of the cosmetic agents according to the invention, and Table 5 lists particularly preferred embodiments AF1 to AF131 (all information is in wt. %, based on the propellant-free cosmetic agent). In Tables 2 to 5, the following meanings apply: Al salt=antiperspirant aluminum salt, Al—Zr salt=antiperspirant aluminum-zirconium salt, and Al salt+Al-Zr salt=a mixture of antiperspirant aluminum salt and an antiperspirant aluminum-zirconium salt. KM in each case denotes the preservative(s) (A) listed in Table 6, or mixtures of these preservatives, and ZK1 to ZK13, ZK66, ZK113 to ZK361 denote the second preservative (B) listed above in Table 1, or mixtures of these preservatives.

TABLE 2
Particularly preferred agents (information in
wt. %, based on the propellant-free agent)

| | AF1 | | AF2 | | AF3 |
|---|---|---|---|---|---|
| Deodorant | 0.1-2.0 | Deodorant | 0.1-2.0 | Deodorant | 0.1-2.0 |
| active | | active | | active | |
| ingredient | | ingredient | | ingredient | |
| KM | 0.05-2.0 | KM | 0.05-2.0 | KM | 0.05-2.0 |
| ZK1 | 0.1-5.0 | ZK2 | 0.1-5.0 | ZK3 | 0.1-5.0 |
| Carrier 1) | to make | Carrier 1) | to make | Carrier 1) | to make |
| | 100 | | 100 | | 100 |
| | AF4 | | AF5 | | AF6 |
| Deodorant | 0.1-2.0 | Deodorant | 0.1-2.0 | Deodorant | 0.1-2.0 |
| active | | active | | active | |
| ingredient | | ingredient | | ingredient | |
| KM | 0.05-2.0 | KM | 0.05-2.0 | KM | 0.05-2.0 |
| ZK4 | 0.1-5.0 | ZK5 | 0.1-5.0 | ZK6 | 0.1-5.0 |
| Carrier 1) | to make | Carrier 1) | to make | Carrier 1) | to make |
| | 100 | | 100 | | 100 |
| | AF7 | | AF8 | | AF9 |
| Deodorant | 0.1-2.0 | Deodorant | 0.1-2.0 | Deodorant | 0.1-2.0 |
| active | | active | | active | |
| ingredient | | ingredient | | ingredient | |
| KM | 0.05-2.0 | KM | 0.05-2.0 | KM | 0.05-2.0 |
| ZK8 | 0.1-5.0 | ZK9 | 0.1-5.0 | ZK10 | 0.1-5.0 |
| Carrier 1) | to make | Carrier 1) | to make | Carrier 1) | to make |
| | 100 | | 100 | | 100 |
| | AF10 | | AF11 | | AF12 |
| Deodorant | 0.1-2.0 | Deodorant | 0.1-2.0 | Deodorant | 0.1-2.0 |
| active | | active | | active | |
| ingredient | | ingredient | | ingredient | |
| KM | 0.05-2.0 | KM | 0.05-2.0 | KM | 0.05-2.0 |
| ZK11 | 0.1-5.0 | ZK12 | 0.1-5.0 | ZK13 | 0.1-5.0 |
| Carrier 1) | to make | Carrier 1) | to make | Carrier 1) | to make |
| | 100 | | 100 | | 100 |
| | AF13 | | AF14 | | AF15 |
| Deodorant | 0.1-2.0 | Deodorant | 0.1-2.0 | Deodorant | 0.1-2.0 |
| active | | active | | active | |
| ingredient | | ingredient | | ingredient | |
| KM | 0.05-2.0 | KM | 0.05-2.0 | KM | 0.05-2.0 |
| ZK66 | 0.1-5.0 | ZK112 | 0.1-5.0 | ZK113 | 0.1-5.0 |
| Carrier 1) | to make | Carrier 1) | to make | Carrier 1) | to make |
| | 100 | | 100 | | 100 |
| | AF16 | | AF17 | | AF18 |
| Deodorant | 0.1-2.0 | Deodorant | 0.1-2.0 | Deodorant | 0.1-2.0 |
| active | | active | | active | |
| ingredient | | ingredient | | ingredient | |
| KM | 0.05-2.0 | KM | 0.05-2.0 | KM | 0.05-2.0 |
| ZK114 | 0.1-5.0 | ZK115 | 0.1-5.0 | ZK116 | 0.1-5.0 |
| Carrier 1) | to make | Carrier 1) | to make | Carrier 1) | to make |
| | 100 | | 100 | | 100 |
| | AF19 | | AF20 | | AF21 |
| Deodorant | 0.1-2.0 | Deodorant | 0.1-2.0 | Deodorant | 0.1-2.0 |
| active | | active | | active | |
| ingredient | | ingredient | | ingredient | |
| KM | 0.05-2.0 | KM | 0.05-2.0 | KM | 0.05-2.0 |
| ZK117 | 0.1-5.0 | ZK118 | 0.1-5.0 | ZK119 | 0.1-5.0 |
| Carrier 1) | to make | Carrier 1) | to make | Carrier 1) | to make |
| | 100 | | 100 | | 100 |
| | AF22 | | AF23 | | AF24 |
| Deodorant | 0.1-2.0 | Deodorant | 0.1-2.0 | Deodorant | 0.1-2.0 |
| active | | active | | active | |
| ingredient | | ingredient | | ingredient | |
| KM | 0.05-2.0 | KM | 0.05-2.0 | KM | 0.05-2.0 |
| ZK120 | 0.1-5.0 | ZK121 | 0.1-5.0 | ZK122 | 0.1-5.0 |
| Carrier 1) | to make | Carrier 1) | to make | Carrier 1) | to make |
| | 100 | | 100 | | 100 |
| | AF25 | | AF26 | | AF27 |
| Deodorant | 0.1-2.0 | Deodorant | 0.1-2.0 | Deodorant | 0.1-2.0 |
| active | | active | | active | |
| ingredient | | ingredient | | ingredient | |
| KM | 0.05-2.0 | KM | 0.05-2.0 | KM | 0.05-2.0 |
| ZK123 | 0.1-5.0 | ZK124 | 0.1-5.0 | ZK125 | 0.1-5.0 |
| Carrier 1) | to make | Carrier 1) | to make | Carrier 1) | to make |
| | 100 | | 100 | | 100 |
| | AF28 | | AF29 | | AF30 |
| Deodorant | 0.1-2.0 | Deodorant | 0.1-2.0 | Deodorant | 0.1-2.0 |
| active | | active | | active | |
| ingredient | | ingredient | | ingredient | |
| KM | 0.05-2.0 | KM | 0.05-2.0 | KM | 0.05-2.0 |
| ZK126 | 0.1-5.0 | ZK127 | 0.1-5.0 | ZK128 | 0.1-5.0 |
| Carrier 1) | to make | Carrier 1) | to make | Carrier 1) | to make |
| | 100 | | 100 | | 100 |
| | AF31 | | AF32 | | AF33 |
| Deodorant | 0.1-2.0 | Deodorant | 0.1-2.0 | Deodorant | 0.1-2.0 |
| active | | active | | active | |
| ingredient | | ingredient | | ingredient | |
| KM | 0.05-2.0 | KM | 0.05-2.0 | KM | 0.05-2.0 |
| ZK129 | 0.1-5.0 | ZK130 | 0.1-5.0 | ZK131 | 0.1-5.0 |
| Carrier 1) | to make | Carrier 1) | to make | Carrier 1) | to make |
| | 100 | | 100 | | 100 |
| | AF34 | | AF35 | | AF36 |
| Deodorant | 0.1-2.0 | Deodorant | 0.1-2.0 | Deodorant | 0.1-2.0 |
| active | | active | | active | |
| ingredient | | ingredient | | ingredient | |
| KM | 0.05-2.0 | KM | 0.05-2.0 | KM | 0.05-2.0 |
| ZK132 | 0.1-5.0 | ZK133 | 0.1-5.0 | ZK134 | 0.1-5.0 |
| Carrier 1) | to make | Carrier 1) | to make | Carrier 1) | to make |
| | 100 | | 100 | | 100 |
| | AF37 | | AF38 | | AF39 |
| Deodorant | 0.1-2.0 | Deodorant | 0.1-2.0 | Deodorant | 0.1-2.0 |
| active | | active | | active | |
| ingredient | | ingredient | | ingredient | |
| KM | 0.05-2.0 | KM | 0.05-2.0 | KM | 0.05-2.0 |
| ZK135 | 0.1-5.0 | ZK136 | 0.1-5.0 | ZK137 | 0.1-5.0 |
| Carrier 1) | to make | Carrier 1) | to make | Carrier 1) | to make |
| | 100 | | 100 | | 100 |
| | AF40 | | AF41 | | AF42 |
| Deodorant | 0.1-2.0 | Deodorant | 0.1-2.0 | Deodorant | 0.1-2.0 |
| active | | active | | active | |
| ingredient | | ingredient | | ingredient | |
| KM | 0.05-2.0 | KM | 0.05-2.0 | KM | 0.05-2.0 |
| ZK138 | 0.1-5.0 | ZK139 | 0.1-5.0 | ZK140 | 0.1-5.0 |
| Carrier 1) | to make | Carrier 1) | to make | Carrier 1) | to make |
| | 100 | | 100 | | 100 |
| | AF43 | | AF44 | | AF45 |
| Deodorant | 0.1-2.0 | Deodorant | 0.1-2.0 | Deodorant | 0.1-2.0 |
| active | | active | | active | |
| ingredient | | ingredient | | ingredient | |
| KM | 0.05-2.0 | KM | 0.05-2.0 | KM | 0.05-2.0 |
| ZK141 | 0.1-5.0 | ZK142 | 0.1-5.0 | ZK143 | 0.1-5.0 |
| Carrier 1) | to make | Carrier 1) | to make | Carrier 1) | to make |
| | 100 | | 100 | | 100 |
| | AF46 | | AF47 | | AF48 |
| Deodorant | 0.1-2.0 | Deodorant | 0.1-2.0 | Deodorant | 0.1-2.0 |
| active | | active | | active | |
| ingredient | | ingredient | | ingredient | |
| KM | 0.05-2.0 | KM | 0.05-2.0 | KM | 0.05-2.0 |
| ZK144 | 0.1-5.0 | ZK145 | 0.1-5.0 | ZK146 | 0.1-5.0 |
| Carrier 1) | to make | Carrier 1) | to make | Carrier 1) | to make |
| | 100 | | 100 | | 100 |
| | AF49 | | AF50 | | AF51 |
| Deodorant | 0.1-2.0 | Deodorant | 0.1-2.0 | Deodorant | 0.1-2.0 |
| active | | active | | active | |
| ingredient | | ingredient | | ingredient | |
| KM | 0.05-2.0 | KM | 0.05-2.0 | KM | 0.05-2.0 |
| ZK147 | 0.1-5.0 | ZK148 | 0.1-5.0 | ZK149 | 0.1-5.0 |
| Carrier 1) | to make | Carrier 1) | to make | Carrier 1) | to make |
| | 100 | | 100 | | 100 |
| | AF52 | | AF53 | | AF54 |
| Deodorant | 0.1-2.0 | Deodorant | 0.1-2.0 | Deodorant | 0.1-2.0 |
| active | | active | | active | |
| ingredient | | ingredient | | ingredient | |
| KM | 0.05-2.0 | KM | 0.05-2.0 | KM | 0.05-2.0 |
| ZK150 | 0.1-5.0 | ZK151 | 0.1-5.0 | ZK152 | 0.1-5.0 |
| Carrier 1) | to make | Carrier 1) | to make | Carrier 1) | to make |
| | 100 | | 100 | | 100 |
| | AF55 | | AF59 | | AF57 |
| Deodorant | 0.1-2.0 | Deodorant | 0.1-2.0 | Deodorant | 0.1-2.0 |
| active | | active | | active | |
| ingredient | | ingredient | | ingredient | |
| KM | 0.05-2.0 | KM | 0.05-2.0 | KM | 0.05-2.0 |
| ZK153 | 0.1-5.0 | ZK154 | 0.1-5.0 | ZK155 | 0.1-5.0 |
| Carrier 1) | to make | Carrier 1) | to make | Carrier 1) | to make |
| | 100 | | 100 | | 100 |
| | AF58 | | AF59 | | AF60 |
| Deodorant | 0.1-2.0 | Deodorant | 0.1-2.0 | Deodorant | 0.1-2.0 |
| active | | active | | active | |
| ingredient | | ingredient | | ingredient | |
| KM | 0.05-2.0 | KM | 0.05-2.0 | KM | 0.05-2.0 |
| ZK156 | 0.1-5.0 | ZK157 | 0.1-5.0 | ZK158 | 0.1-5.0 |
| Carrier 1) | to make | Carrier 1) | to make | Carrier 1) | to make |
| | 100 | | 100 | | 100 |
| | AF61 | | AF62 | | AF63 |
| Deodorant | 0.1-2.0 | Deodorant | 0.1-2.0 | Deodorant | 0.1-2.0 |
| active | | active | | active | |
| ingredient | | ingredient | | ingredient | |
| KM | 0.05-2.0 | KM | 0.05-2.0 | KM | 0.05-2.0 |
| ZK159 | 0.1-5.0 | ZK160 | 0.1-5.0 | ZK161 | 0.1-5.0 |
| Carrier 1) | to make | Carrier 1) | to make | Carrier 1) | to make |
| | 100 | | 100 | | 100 |
| | AF64 | | AF65 | | AF66 |
| Deodorant | 0.1-2.0 | Deodorant | 0.1-2.0 | Deodorant | 0.1-2.0 |
| active | | active | | active | |
| ingredient | | ingredient | | ingredient | |
| KM | 0.05-2.0 | KM | 0.05-2.0 | KM | 0.05-2.0 |
| ZK162 | 0.1-5.0 | ZK163 | 0.1-5.0 | ZK164 | 0.1-5.0 |
| Carrier 1) | to make | Carrier 1) | to make | Carrier 1) | to make |
| | 100 | | 100 | | 100 |
| | AF67 | | AF68 | | AF69 |
| Deodorant | 0.1-2.0 | Deodorant | 0.1-2.0 | Deodorant | 0.1-2.0 |
| active | | active | | active | |
| ingredient | | ingredient | | ingredient | |
| KM | 0.05-2.0 | KM | 0.05-2.0 | KM | 0.05-2.0 |
| ZK165 | 0.1-5.0 | ZK166 | 0.1-5.0 | ZK167 | 0.1-5.0 |
| Carrier 1) | to make | Carrier 1) | to make | Carrier 1) | to make |
| | 100 | | 100 | | 100 |
| | AF70 | | AF71 | | AF72 |
| Deodorant | 0.1-2.0 | Deodorant | 0.1-2.0 | Deodorant | 0.1-2.0 |
| active | | active | | active | |
| ingredient | | ingredient | | ingredient | |
| KM | 0.05-2.0 | KM | 0.05-2.0 | KM | 0.05-2.0 |
| ZK168 | 0.1-5.0 | ZK169 | 0.1-5.0 | ZK170 | 0.1-5.0 |
| Carrier 1) | to make | Carrier 1) | to make | Carrier 1) | to make |
| | 100 | | 100 | | 100 |
| | AF73 | | AF74 | | AF75 |
| Deodorant | 0.1-2.0 | Deodorant | 0.1-2.0 | Deodorant | 0.1-2.0 |
| active | | active | | active | |
| ingredient | | ingredient | | ingredient | |
| KM | 0.05-2.0 | KM | 0.05-2.0 | KM | 0.05-2.0 |
| ZK171 | 0.1-5.0 | ZK172 | 0.1-5.0 | ZK173 | 0.1-5.0 |
| Carrier 1) | to make | Carrier 1) | to make | Carrier 1) | to make |
| | 100 | | 100 | | 100 |
| | AF76 | | AF77 | | AF78 |
| Deodorant | 0.1-2.0 | Deodorant | 0.1-2.0 | Deodorant | 0.1-2.0 |
| active | | active | | active | |
| ingredient | | ingredient | | ingredient | |
| KM | 0.05-2.0 | KM | 0.05-2.0 | KM | 0.05-2.0 |
| ZK174 | 0.1-5.0 | ZK175 | 0.1-5.0 | ZK176 | 0.1-5.0 |
| Carrier 1) | to make | Carrier 1) | to make | Carrier 1) | to make |
| | 100 | | 100 | | 100 |
| | AF79 | | AF80 | | AF81 |
| Deodorant | 0.1-2.0 | Deodorant | 0.1-2.0 | Deodorant | 0.1-2.0 |
| active | | active | | active | |
| ingredient | | ingredient | | ingredient | |
| KM | 0.05-2.0 | KM | 0.05-2.0 | KM | 0.05-2.0 |
| ZK177 | 0.1-5.0 | ZK178 | 0.1-5.0 | ZK179 | 0.1-5.0 |
| Carrier 1) | to make | Carrier 1) | to make | Carrier 1) | to make |
| | 100 | | 100 | | 100 |
| | AF82 | | AF83 | | AF84 |
| Deodorant | 0.1-2.0 | Deodorant | 0.1-2.0 | Deodorant | 0.1-2.0 |
| active | | active | | active | |
| ingredient | | ingredient | | ingredient | |
| KM | 0.05-2.0 | KM | 0.05-2.0 | KM | 0.05-2.0 |
| ZK180 | 0.1-5.0 | ZK181 | 0.1-5.0 | ZK182 | 0.1-5.0 |
| Carrier 1) | to make | Carrier 1) | to make | Carrier 1) | to make |
| | 100 | | 100 | | 100 |
| KM | 0.1-7.0 | KM | 0.1-7.0 | KM | 0.1-7.0 |
| | AF85 | | AF86 | | AF87 |
| Al salt | 2.0-8.0 | Al salt | 2.0-8.0 | Al salt | 2.0-8.0 |
| KM | 0.05-2.0 | KM | 0.05-2.0 | KM | 0.05-2.0 |
| ZK1 | 0.1-5.0 | ZK2 | 0.1-5.0 | ZK3 | 0.1-5.0 |
| Carrier 1) | to make | Carrier 1) | to make | Carrier 1) | to make |
| | 100 | | 100 | | 100 |
| | AF88 | | AF89 | | AF90 |
| Al salt | 2.0-8.0 | Al salt | 2.0-8.0 | Al salt | 2.0-8.0 |
| KM | 0.05-2.0 | KM | 0.05-2.0 | KM | 0.05-2.0 |
| ZK4 | 0.1-5.0 | ZK5 | 0.1-5.0 | ZK6 | 0.1-5.0 |
| Carrier 1) | to make | Carrier 1) | to make | Carrier 1) | to make |
| | 100 | | 100 | | 100 |
| | AF91 | | AF92 | | AF93 |
| Al salt | 2.0-8.0 | Al salt | 2.0-8.0 | Al salt | 2.0-8.0 |
| KM | 0.05-2.0 | KM | 0.05-2.0 | KM | 0.05-2.0 |
| ZK8 | 0.1-5.0 | ZK9 | 0.1-5.0 | ZK10 | 0.1-5.0 |
| Carrier 1) | to make | Carrier 1) | to make | Carrier 1) | to make |
| | 100 | | 100 | | 100 |
| | AF94 | | AF95 | | AF96 |
| Al salt | 2.0-8.0 | Al salt | 2.0-8.0 | Al salt | 2.0-8.0 |
| KM | 0.05-2.0 | KM | 0.05-2.0 | KM | 0.05-2.0 |
| ZK11 | 0.1-5.0 | ZK12 | 0.1-5.0 | ZK13 | 0.1-5.0 |
| Carrier 1) | to make | Carrier 1) | to make | Carrier 1) | to make |
| | 100 | | 100 | | 100 |
| | AF97 | | AF98 | | AF99 |
| Al salt | 2.0-8.0 | Al salt | 2.0-8.0 | Al salt | 2.0-8.0 |
| KM | 0.05-2.0 | KM | 0.05-2.0 | KM | 0.05-2.0 |
| ZK66 | 0.1-5.0 | ZK112 | 0.1-5.0 | ZK113 | 0.1-5.0 |
| Carrier 1) | to make | Carrier 1) | to make | Carrier 1) | to make |
| | 100 | | 100 | | 100 |
| | AF100 | | AF101 | | AF102 |
| Al salt | 2.0-8.0 | Al salt | 2.0-8.0 | Al salt | 2.0-8.0 |
| KM | 0.05-2.0 | KM | 0.05-2.0 | KM | 0.05-2.0 |
| ZK114 | 0.1-5.0 | ZK115 | 0.1-5.0 | ZK116 | 0.1-5.0 |
| Carrier 1) | to make | Carrier 1) | to make | Carrier 1) | to make |
| | 100 | | 100 | | 100 |
| | AF103 | | AF104 | | AF105 |
| Al salt | 2.0-8.0 | Al salt | 2.0-8.0 | Al salt | 2.0-8.0 |
| KM | 0.05-2.0 | KM | 0.05-2.0 | KM | 0.05-2.0 |
| ZK117 | 0.1-5.0 | ZK118 | 0.1-5.0 | ZK119 | 0.1-5.0 |
| Carrier 1) | to make | Carrier 1) | to make | Carrier 1) | to make |
| | 100 | | 100 | | 100 |
| | AF106 | | AF107 | | AF108 |
| Al salt | 2.0-8.0 | Al salt | 2.0-8.0 | Al salt | 2.0-8.0 |
| KM | 0.05-2.0 | KM | 0.05-2.0 | KM | 0.05-2.0 |
| ZK120 | 0.1-5.0 | ZK121 | 0.1-5.0 | ZK122 | 0.1-5.0 |
| Carrier 1) | to make | Carrier 1) | to make | Carrier 1) | to make |
| | 100 | | 100 | | 100 |
| | AF109 | | AF110 | | AF111 |
| Al salt | 2.0-8.0 | Al salt | 2.0-8.0 | Al salt | 2.0-8.0 |
| KM | 0.05-2.0 | KM | 0.05-2.0 | KM | 0.05-2.0 |
| ZK123 | 0.1-5.0 | ZK124 | 0.1-5.0 | ZK125 | 0.1-5.0 |
| Carrier 1) | to make | Carrier 1) | to make | Carrier 1) | to make |
| | 100 | | 100 | | 100 |
| | AF112 | | AF113 | | AF114 |
| Al salt | 2.0-8.0 | Al salt | 2.0-8.0 | Al salt | 2.0-8.0 |
| KM | 0.05-2.0 | KM | 0.05-2.0 | KM | 0.05-2.0 |
| ZK126 | 0.1-5.0 | ZK127 | 0.1-5.0 | ZK128 | 0.1-5.0 |
| Carrier 1) | to make | Carrier 1) | to make | Carrier 1) | to make |
| | 100 | | 100 | | 100 |
| | AF115 | | AF116 | | AF117 |
| Al salt | 2.0-8.0 | Al salt | 2.0-8.0 | Al salt | 2.0-8.0 |
| KM | 0.05-2.0 | KM | 0.05-2.0 | KM | 0.05-2.0 |
| ZK129 | 0.1-5.0 | ZK130 | 0.1-5.0 | ZK131 | 0.1-5.0 |
| Carrier 1) | to make | Carrier 1) | to make | Carrier 1) | to make |
| | 100 | | 100 | | 100 |
| | AF118 | | AF119 | | AF120 |
| Al salt | 2.0-8.0 | Al salt | 2.0-8.0 | Al salt | 2.0-8.0 |
| KM | 0.05-2.0 | KM | 0.05-2.0 | KM | 0.05-2.0 |
| ZK132 | 0.1-5.0 | ZK133 | 0.1-5.0 | ZK134 | 0.1-5.0 |
| Carrier 1) | to make | Carrier 1) | to make | Carrier 1) | to make |
| | 100 | | 100 | | 100 |
| | AF121 | | AF122 | | AF123 |
| Al salt | 2.0-8.0 | Al salt | 2.0-8.0 | Al salt | 2.0-8.0 |
| KM | 0.05-2.0 | KM | 0.05-2.0 | KM | 0.05-2.0 |
| ZK135 | 0.1-5.0 | ZK136 | 0.1-5.0 | ZK137 | 0.1-5.0 |
| Carrier 1) | to make | Carrier 1) | to make | Carrier 1) | to make |
| | 100 | | 100 | | 100 |
| | AF124 | | AF125 | | AF126 |
| Al salt | 2.0-8.0 | Al salt | 2.0-8.0 | Al salt | 2.0-8.0 |
| KM | 0.05-2.0 | KM | 0.05-2.0 | KM | 0.05-2.0 |
| ZK138 | 0.1-5.0 | ZK139 | 0.1-5.0 | ZK140 | 0.1-5.0 |
| Carrier 1) | to make | Carrier 1) | to make | Carrier 1) | to make |
| | 100 | | 100 | | 100 |
| | AF127 | | AF128 | | AF129 |
| Al salt | 2.0-8.0 | Al salt | 2.0-8.0 | Al salt | 2.0-8.0 |
| KM | 0.05-2.0 | KM | 0.05-2.0 | KM | 0.05-2.0 |
| ZK141 | 0.1-5.0 | ZK142 | 0.1-5.0 | ZK143 | 0.1-5.0 |
| Carrier 1) | to make | Carrier 1) | to make | Carrier 1) | to make |
| | 100 | | 100 | | 100 |
| | AF130 | | AF131 | | AF132 |
| Al salt | 2.0-8.0 | Al salt | 2.0-8.0 | Al salt | 2.0-8.0 |
| KM | 0.05-2.0 | KM | 0.05-2.0 | KM | 0.05-2.0 |
| ZK144 | 0.1-5.0 | ZK145 | 0.1-5.0 | ZK146 | 0.1-5.0 |
| Carrier 1) | to make | Carrier 1) | to make | Carrier 1) | to make |
| | 100 | | 100 | | 100 |
| | AF133 | | AF134 | | AF135 |
| Al salt | 2.0-8.0 | Al salt | 2.0-8.0 | Al salt | 2.0-8.0 |
| KM | 0.05-2.0 | KM | 0.05-2.0 | KM | 0.05-2.0 |
| ZK147 | 0.1-5.0 | ZK148 | 0.1-5.0 | ZK149 | 0.1-5.0 |
| Carrier 1) | to make | Carrier 1) | to make | Carrier 1) | to make |
| | 100 | | 100 | | 100 |
| | AF136 | | AF137 | | AF138 |
| Al salt | 2.0-8.0 | Al salt | 2.0-8.0 | Al salt | 2.0-8.0 |
| KM | 0.05-2.0 | KM | 0.05-2.0 | KM | 0.05-2.0 |
| ZK150 | 0.1-5.0 | ZK151 | 0.1-5.0 | ZK152 | 0.1-5.0 |
| Carrier 1) | to make | Carrier 1) | to make | Carrier 1) | to make |
| | 100 | | 100 | | 100 |
| | AF139 | | AF140 | | AF141 |
| Al salt | 2.0-8.0 | Al salt | 2.0-8.0 | Al salt | 2.0-8.0 |
| KM | 0.05-2.0 | KM | 0.05-2.0 | KM | 0.05-2.0 |
| ZK153 | 0.1-5.0 | ZK154 | 0.1-5.0 | ZK155 | 0.1-5.0 |
| Carrier 1) | to make | Carrier 1) | to make | Carrier 1) | to make |
| | 100 | | 100 | | 100 |
| | AF142 | | AF143 | | AF144 |
| Al salt | 2.0-8.0 | Al salt | 2.0-8.0 | Al salt | 2.0-8.0 |
| KM | 0.05-2.0 | KM | 0.05-2.0 | KM | 0.05-2.0 |
| ZK156 | 0.1-5.0 | ZK157 | 0.1-5.0 | ZK158 | 0.1-5.0 |
| Carrier 1) | to make | Carrier 1) | to make | Carrier 1) | to make |
| | 100 | | 100 | | 100 |
| | AF145 | | AF146 | | AF147 |
| Al salt | 2.0-8.0 | Al salt | 2.0-8.0 | Al salt | 2.0-8.0 |
| KM | 0.05-2.0 | KM | 0.05-2.0 | KM | 0.05-2.0 |
| ZK159 | 0.1-5.0 | ZK160 | 0.1-5.0 | ZK161 | 0.1-5.0 |
| Carrier 1) | to make | Carrier 1) | to make | Carrier 1) | to make |
| | 100 | | 100 | | 100 |
| | AF148 | | AF149 | | AF150 |
| Al salt | 2.0-8.0 | Al salt | 2.0-8.0 | Al salt | 2.0-8.0 |
| KM | 0.05-2.0 | KM | 0.05-2.0 | KM | 0.05-2.0 |
| ZK162 | 0.1-5.0 | ZK163 | 0.1-5.0 | ZK164 | 0.1-5.0 |
| Carrier 1) | to make | Carrier 1) | to make | Carrier 1) | to make |
| | 100 | | 100 | | 100 |
| | AF151 | | AF152 | | AF153 |
| Al salt | 2.0-8.0 | Al salt | 2.0-8.0 | Al salt | 2.0-8.0 |
| KM | 0.05-2.0 | KM | 0.05-2.0 | KM | 0.05-2.0 |
| ZK165 | 0.1-5.0 | ZK166 | 0.1-5.0 | ZK167 | 0.1-5.0 |
| Carrier 1) | to make | Carrier 1) | to make | Carrier 1) | to make |
| | 100 | | 100 | | 100 |
| | AF154 | | AF155 | | AF156 |
| Al salt | 2.0-8.0 | Al salt | 2.0-8.0 | Al salt | 2.0-8.0 |
| KM | 0.05-2.0 | KM | 0.05-2.0 | KM | 0.05-2.0 |
| ZK168 | 0.1-5.0 | ZK169 | 0.1-5.0 | ZK170 | 0.1-5.0 |
| Carrier 1) | to make | Carrier 1) | to make | Carrier 1) | to make |
| | 100 | | 100 | | 100 |
| | AF157 | | AF158 | | AF159 |
| Al salt | 2.0-8.0 | Al salt | 2.0-8.0 | Al salt | 2.0-8.0 |
| KM | 0.05-2.0 | KM | 0.05-2.0 | KM | 0.05-2.0 |
| ZK171 | 0.1-5.0 | ZK172 | 0.1-5.0 | ZK173 | 0.1-5.0 |
| Carrier 1) | to make | Carrier 1) | to make | Carrier 1) | to make |
| | 100 | | 100 | | 100 |
| | AF160 | | AF161 | | AF162 |
| Al salt | 2.0-8.0 | Al salt | 2.0-8.0 | Al salt | 2.0-8.0 |
| KM | 0.05-2.0 | KM | 0.05-2.0 | KM | 0.05-2.0 |
| ZK174 | 0.1-5.0 | ZK175 | 0.1-5.0 | ZK176 | 0.1-5.0 |
| Carrier 1) | to make | Carrier 1) | to make | Carrier 1) | to make |
| | 100 | | 100 | | 100 |
| | AF163 | | AF164 | | AF165 |
| Al salt | 2.0-8.0 | Al salt | 2.0-8.0 | Al salt | 2.0-8.0 |
| KM | 0.05-2.0 | KM | 0.05-2.0 | KM | 0.05-2.0 |
| ZK177 | 0.1-5.0 | ZK178 | 0.1-5.0 | ZK179 | 0.1-5.0 |
| Carrier 1) | to make | Carrier 1) | to make | Carrier 1) | to make |
| | 100 | | 100 | | 100 |
| | AF166 | | AF167 | | AF168 |
| Al salt | 2.0-8.0 | Al salt | 2.0-8.0 | Al salt | 2.0-8.0 |
| KM | 0.05-2.0 | KM | 0.05-2.0 | KM | 0.05-2.0 |
| ZK180 | 0.1-5.0 | ZK181 | 0.1-5.0 | ZK182 | 0.1-5.0 |
| Carrier 1) | to make | Carrier 1) | to make | Carrier 1) | to make |
| | 100 | | 100 | | 100 |
| | AF169 | | AF170 | | AF171 |
| Deodorant | 0.1-2.0 | Deodorant | 0.1-2.0 | Deodorant | 0.1-2.0 |
| active | | active | | active | |
| ingredient | | ingredient | | ingredient | |
| Al salt | 2.0-8.0 | Al salt | 2.0-8.0 | Al salt | 2.0-8.0 |
| KM | 0.05-2.0 | KM | 0.05-2.0 | KM | 0.05-2.0 |
| ZK1 | 0.1-5.0 | ZK2 | 0.1-5.0 | ZK3 | 0.1-5.0 |
| Carrier 1) | to make | Carrier 1) | to make | Carrier 1) | to make |
| | 100 | | 100 | | 100 |
| | AF172 | | AF173 | | AF174 |
| Deodorant | 0.1-2.0 | Deodorant | 0.1-2.0 | Deodorant | 0.1-2.0 |
| active | | active | | active | |
| ingredient | | ingredient | | ingredient | |
| Al salt | 2.0-8.0 | Al salt | 2.0-8.0 | Al salt | 2.0-8.0 |
| KM | 0.05-2.0 | KM | 0.05-2.0 | KM | 0.05-2.0 |
| ZK4 | 0.1-5.0 | ZK5 | 0.1-5.0 | ZK6 | 0.1-5.0 |
| Carrier 1) | to make | Carrier 1) | to make | Carrier 1) | to make |
| | 100 | | 100 | | 100 |
| | AF175 | | AF176 | | AF177 |
| Deodorant | 0.1-2.0 | Deodorant | 0.1-2.0 | Deodorant | 0.1-2.0 |
| active | | active | | active | |
| ingredient | | ingredient | | ingredient | |
| Al salt | 2.0-8.0 | Al salt | 2.0-8.0 | Al salt | 2.0-8.0 |
| KM | 0.05-2.0 | KM | 0.05-2.0 | KM | 0.05-2.0 |
| ZK8 | 0.1-5.0 | ZK9 | 0.1-5.0 | ZK10 | 0.1-5.0 |
| Carrier 1) | to make | Carrier 1) | to make | Carrier 1) | to make |
| | 100 | | 100 | | 100 |
| | AF178 | | AF179 | | AF180 |
| Deodorant | 0.1-2.0 | Deodorant | 0.1-2.0 | Deodorant | 0.1-2.0 |
| active | | active | | active | |
| ingredient | | ingredient | | ingredient | |
| Al salt | 2.0-8.0 | Al salt | 2.0-8.0 | Al salt | 2.0-8.0 |
| KM | 0.05-2.0 | KM | 0.05-2.0 | KM | 0.05-2.0 |
| ZK11 | 0.1-5.0 | ZK12 | 0.1-5.0 | ZK13 | 0.1-5.0 |
| Carrier 1) | to make | Carrier 1) | to make | Carrier 1) | to make |
| | 100 | | 100 | | 100 |
| | AF181 | | AF182 | | AF183 |
| Deodorant | 0.1-2.0 | Deodorant | 0.1-2.0 | Deodorant | 0.1-2.0 |
| active | | active | | active | |
| ingredient | | ingredient | | ingredient | |
| Al salt | 2.0-8.0 | Al salt | 2.0-8.0 | Al salt | 2.0-8.0 |
| KM | 0.05-2.0 | KM | 0.05-2.0 | KM | 0.05-2.0 |
| ZK66 | 0.1-5.0 | ZK112 | 0.1-5.0 | ZK113 | 0.1-5.0 |
| Carrier 1) | to make | Carrier 1) | to make | Carrier 1) | to make |
| | 100 | | 100 | | 100 |
| | AF184 | | AF185 | | AF186 |
| Deodorant | 0.1-2.0 | Deodorant | 0.1-2.0 | Deodorant | 0.1-2.0 |
| active | | active | | active | |
| ingredient | | ingredient | | ingredient | |
| Al salt | 2.0-8.0 | Al salt | 2.0-8.0 | Al salt | 2.0-8.0 |
| KM | 0.05-2.0 | KM | 0.05-2.0 | KM | 0.05-2.0 |
| ZK114 | 0.1-5.0 | ZK115 | 0.1-5.0 | ZK116 | 0.1-5.0 |
| Carrier 1) | to make | Carrier 1) | to make | Carrier 1) | to make |
| | 100 | | 100 | | 100 |
| | AF187 | | AF188 | | AF189 |
| Deodorant | 0.1-2.0 | Deodorant | 0.1-2.0 | Deodorant | 0.1-2.0 |
| active | | active | | active | |
| ingredient | | ingredient | | ingredient | |
| Al salt | 2.0-8.0 | Al salt | 2.0-8.0 | Al salt | 2.0-8.0 |
| KM | 0.05-2.0 | KM | 0.05-2.0 | KM | 0.05-2.0 |
| ZK117 | 0.1-5.0 | ZK118 | 0.1-5.0 | ZK119 | 0.1-5.0 |
| Carrier 1) | to make | Carrier 1) | to make | Carrier 1) | to make |
| | 100 | | 100 | | 100 |
| | AF190 | | AF191 | | AF192 |
| Deodorant | 0.1-2.0 | Deodorant | 0.1-2.0 | Deodorant | 0.1-2.0 |
| active | | active | | active | |
| ingredient | | ingredient | | ingredient | |
| Al salt | 2.0-8.0 | Al salt | 2.0-8.0 | Al salt | 2.0-8.0 |
| KM | 0.05-2.0 | KM | 0.05-2.0 | KM | 0.05-2.0 |
| ZK120 | 0.1-5.0 | ZK121 | 0.1-5.0 | ZK122 | 0.1-5.0 |
| Carrier 1) | to make | Carrier 1) | to make | Carrier 1) | to make |
| | 100 | | 100 | | 100 |
| | AF193 | | AF194 | | AF195 |
| Deodorant | 0.1-2.0 | Deodorant | 0.1-2.0 | Deodorant | 0.1-2.0 |
| active | | active | | active | |
| ingredient | | ingredient | | ingredient | |
| Al salt | 2.0-8.0 | Al salt | 2.0-8.0 | Al salt | 2.0-8.0 |
| KM | 0.05-2.0 | KM | 0.05-2.0 | KM | 0.05-2.0 |
| ZK123 | 0.1-5.0 | ZK124 | 0.1-5.0 | ZK125 | 0.1-5.0 |
| Carrier 1) | to make | Carrier 1) | to make | Carrier 1) | to make |
| | 100 | | 100 | | 100 |
| | AF196 | | AF197 | | AF198 |
| Deodorant | 0.1-2.0 | Deodorant | 0.1-2.0 | Deodorant | 0.1-2.0 |
| active | | active | | active | |
| ingredient | | ingredient | | ingredient | |
| Al salt | 2.0-8.0 | Al salt | 2.0-8.0 | Al salt | 2.0-8.0 |
| KM | 0.05-2.0 | KM | 0.05-2.0 | KM | 0.05-2.0 |
| ZK126 | 0.1-5.0 | ZK127 | 0.1-5.0 | ZK128 | 0.1-5.0 |
| Carrier 1) | to make | Carrier 1) | to make | Carrier 1) | to make |
| | 100 | | 100 | | 100 |
| | AF199 | | AF200 | | AF201 |
| Deodorant | 0.1-2.0 | Deodorant | 0.1-2.0 | Deodorant | 0.1-2.0 |
| active | | active | | active | |
| ingredient | | ingredient | | ingredient | |
| Al salt | 2.0-8.0 | Al salt | 2.0-8.0 | Al salt | 2.0-8.0 |
| KM | 0.05-2.0 | KM | 0.05-2.0 | KM | 0.05-2.0 |
| ZK129 | 0.1-5.0 | ZK130 | 0.1-5.0 | ZK131 | 0.1-5.0 |
| Carrier 1) | to make | Carrier 1) | to make | Carrier 1) | to make |
| | 100 | | 100 | | 100 |
| | AF202 | | AF203 | | AF204 |
| Deodorant | 0.1-2.0 | Deodorant | 0.1-2.0 | Deodorant | 0.1-2.0 |
| active | | active | | active | |
| ingredient | | ingredient | | ingredient | |
| Al salt | 2.0-8.0 | Al salt | 2.0-8.0 | Al salt | 2.0-8.0 |
| KM | 0.05-2.0 | KM | 0.05-2.0 | KM | 0.05-2.0 |
| ZK132 | 0.1-5.0 | ZK133 | 0.1-5.0 | ZK134 | 0.1-5.0 |
| Carrier 1) | to make | Carrier 1) | to make | Carrier 1) | to make |
| | 100 | | 100 | | 100 |
| | AF205 | | AF206 | | AF207 |
| Deodorant | 0.1-2.0 | Deodorant | 0.1-2.0 | Deodorant | 0.1-2.0 |
| active | | active | | active | |
| ingredient | | ingredient | | ingredient | |
| Al salt | 2.0-8.0 | Al salt | 2.0-8.0 | Al salt | 2.0-8.0 |
| KM | 0.05-2.0 | KM | 0.05-2.0 | KM | 0.05-2.0 |
| ZK135 | 0.1-5.0 | ZK136 | 0.1-5.0 | ZK137 | 0.1-5.0 |
| Carrier 1) | to make | Carrier 1) | to make | Carrier 1) | to make |
| | 100 | | 100 | | 100 |
| | AF208 | | AF209 | | AF210 |
| Deodorant | 0.1-2.0 | Deodorant | 0.1-2.0 | Deodorant | 0.1-2.0 |
| active | | active | | active | |
| ingredient | | ingredient | | ingredient | |
| Al salt | 2.0-8.0 | Al salt | 2.0-8.0 | Al salt | 2.0-8.0 |
| KM | 0.05-2.0 | KM | 0.05-2.0 | KM | 0.05-2.0 |
| ZK138 | 0.1-5.0 | ZK139 | 0.1-5.0 | ZK140 | 0.1-5.0 |
| Carrier 1) | to make | Carrier 1) | to make | Carrier 1) | to make |
| | 100 | | 100 | | 100 |
| | AF211 | | AF212 | | AF213 |
| Deodorant | 0.1-2.0 | Deodorant | 0.1-2.0 | Deodorant | 0.1-2.0 |
| active | | active | | active | |
| ingredient | | ingredient | | ingredient | |
| Al salt | 2.0-8.0 | Al salt | 2.0-8.0 | Al salt | 2.0-8.0 |
| KM | 0.05-2.0 | KM | 0.05-2.0 | KM | 0.05-2.0 |
| ZK141 | 0.1-5.0 | ZK142 | 0.1-5.0 | ZK143 | 0.1-5.0 |
| Carrier 1) | to make | Carrier 1) | to make | Carrier 1) | to make |
| | 100 | | 100 | | 100 |
| | AF214 | | AF215 | | AF216 |
| Deodorant | 0.1-2.0 | Deodorant | 0.1-2.0 | Deodorant | 0.1-2.0 |
| active | | active | | active | |
| ingredient | | ingredient | | ingredient | |
| Al salt | 2.0-8.0 | Al salt | 2.0-8.0 | Al salt | 2.0-8.0 |
| KM | 0.05-2.0 | KM | 0.05-2.0 | KM | 0.05-2.0 |
| ZK144 | 0.1-5.0 | ZK145 | 0.1-5.0 | ZK146 | 0.1-5.0 |
| Carrier 1) | to make | Carrier 1) | to make | Carrier 1) | to make |
| | 100 | | 100 | | 100 |
| | AF217 | | AF218 | | AF219 |
| Deodorant | 0.1-2.0 | Deodorant | 0.1-2.0 | Deodorant | 0.1-2.0 |
| active | | active | | active | |
| ingredient | | ingredient | | ingredient | |
| Al salt | 2.0-8.0 | Al salt | 2.0-8.0 | Al salt | 2.0-8.0 |
| KM | 0.05-2.0 | KM | 0.05-2.0 | KM | 0.05-2.0 |
| ZK147 | 0.1-5.0 | ZK148 | 0.1-5.0 | ZK149 | 0.1-5.0 |
| Carrier 1) | to make | Carrier 1) | to make | Carrier 1) | to make |
| | 100 | | 100 | | 100 |
| | AF220 | | AF221 | | AF222 |
| Deodorant | 0.1-2.0 | Deodorant | 0.1-2.0 | Deodorant | 0.1-2.0 |
| active | | active | | active | |
| ingredient | | ingredient | | ingredient | |
| Al salt | 2.0-8.0 | Al salt | 2.0-8.0 | Al salt | 2.0-8.0 |
| KM | 0.05-2.0 | KM | 0.05-2.0 | KM | 0.05-2.0 |
| ZK150 | 0.1-5.0 | ZK151 | 0.1-5.0 | ZK152 | 0.1-5.0 |
| Carrier 1) | to make | Carrier 1) | to make | Carrier 1) | to make |
| | 100 | | 100 | | 100 |
| | AF223 | | AF224 | | AF225 |
| Deodorant | 0.1-2.0 | Deodorant | 0.1-2.0 | Deodorant | 0.1-2.0 |
| active | | active | | active | |
| ingredient | | ingredient | | ingredient | |
| Al salt | 2.0-8.0 | Al salt | 2.0-8.0 | Al salt | 2.0-8.0 |
| KM | 0.05-2.0 | KM | 0.05-2.0 | KM | 0.05-2.0 |
| ZK153 | 0.1-5.0 | ZK154 | 0.1-5.0 | ZK155 | 0.1-5.0 |
| Carrier 1) | to make | Carrier 1) | to make | Carrier 1) | to make |
| | 100 | | 100 | | 100 |
| | AF226 | | AF227 | | AF228 |
| Deodorant | 0.1-2.0 | Deodorant | 0.1-2.0 | Deodorant | 0.1-2.0 |
| active | | active | | active | |
| ingredient | | ingredient | | ingredient | |
| Al salt | 2.0-8.0 | Al salt | 2.0-8.0 | Al salt | 2.0-8.0 |
| KM | 0.05-2.0 | KM | 0.05-2.0 | KM | 0.05-2.0 |
| ZK156 | 0.1-5.0 | ZK157 | 0.1-5.0 | ZK158 | 0.1-5.0 |
| Carrier 1) | to make | Carrier 1) | to make | Carrier 1) | to make |
| | 100 | | 100 | | 100 |
| | AF229 | | AF230 | | AF231 |
| Deodorant | 0.1-2.0 | Deodorant | 0.1-2.0 | Deodorant | 0.1-2.0 |
| active | | active | | active | |
| ingredient | | ingredient | | ingredient | |
| Al salt | 2.0-8.0 | Al salt | 2.0-8.0 | Al salt | 2.0-8.0 |
| KM | 0.05-2.0 | KM | 0.05-2.0 | KM | 0.05-2.0 |
| ZK159 | 0.1-5.0 | ZK160 | 0.1-5.0 | ZK161 | 0.1-5.0 |
| Carrier 1) | to make | Carrier 1) | to make | Carrier 1) | to make |
| | 100 | | 100 | | 100 |
| | AF232 | | AF233 | | AF234 |
| Deodorant | 0.1-2.0 | Deodorant | 0.1-2.0 | Deodorant | 0.1-2.0 |
| active | | active | | active | |
| ingredient | | ingredient | | ingredient | |
| Al salt | 2.0-8.0 | Al salt | 2.0-8.0 | Al salt | 2.0-8.0 |
| KM | 0.05-2.0 | KM | 0.05-2.0 | KM | 0.05-2.0 |
| ZK162 | 0.1-5.0 | ZK163 | 0.1-5.0 | ZK164 | 0.1-5.0 |
| Carrier 1) | to make | Carrier 1) | to make | Carrier 1) | to make |
| | 100 | | 100 | | 100 |
| | AF235 | | AF236 | | AF237 |
| Deodorant | 0.1-2.0 | Deodorant | 0.1-2.0 | Deodorant | 0.1-2.0 |
| active | | active | | active | |
| ingredient | | ingredient | | ingredient | |
| Al salt | 2.0-8.0 | Al salt | 2.0-8.0 | Al salt | 2.0-8.0 |
| KM | 0.05-2.0 | KM | 0.05-2.0 | KM | 0.05-2.0 |
| ZK165 | 0.1-5.0 | ZK166 | 0.1-5.0 | ZK167 | 0.1-5.0 |
| Carrier 1) | to make | Carrier 1) | to make | Carrier 1) | to make |
| | 100 | | 100 | | 100 |
| | AF238 | | AF239 | | AF240 |
| Deodorant | 0.1-2.0 | Deodorant | 0.1-2.0 | Deodorant | 0.1-2.0 |
| active | | active | | active | |
| ingredient | | ingredient | | ingredient | |
| Al salt | 2.0-8.0 | Al salt | 2.0-8.0 | Al salt | 2.0-8.0 |
| KM | 0.05-2.0 | KM | 0.05-2.0 | KM | 0.05-2.0 |
| ZK168 | 0.1-5.0 | ZK169 | 0.1-5.0 | ZK170 | 0.1-5.0 |
| Carrier 1) | to make | Carrier 1) | to make | Carrier 1) | to make |
| | 100 | | 100 | | 100 |
| | AF241 | | AF242 | | AF243 |
| Deodorant | 0.1-2.0 | Deodorant | 0.1-2.0 | Deodorant | 0.1-2.0 |
| active | | active | | active | |
| ingredient | | ingredient | | ingredient | |
| Al salt | 2.0-8.0 | Al salt | 2.0-8.0 | Al salt | 2.0-8.0 |
| KM | 0.05-2.0 | KM | 0.05-2.0 | KM | 0.05-2.0 |
| ZK171 | 0.1-5.0 | ZK172 | 0.1-5.0 | ZK173 | 0.1-5.0 |
| Carrier 1) | to make | Carrier 1) | to make | Carrier 1) | to make |
| | 100 | | 100 | | 100 |
| | AF244 | | AF245 | | AF246 |
| Deodorant | 0.1-2.0 | Deodorant | 0.1-2.0 | Deodorant | 0.1-2.0 |
| active | | active | | active | |
| ingredient | | ingredient | | ingredient | |
| Al salt | 2.0-8.0 | Al salt | 2.0-8.0 | Al salt | 2.0-8.0 |
| KM | 0.05-2.0 | KM | 0.05-2.0 | KM | 0.05-2.0 |
| ZK174 | 0.1-5.0 | ZK175 | 0.1-5.0 | ZK176 | 0.1-5.0 |
| Carrier 1) | to make | Carrier 1) | to make | Carrier 1) | to make |
| | 100 | | 100 | | 100 |
| | AF247 | | AF248 | | AF249 |
| Deodorant | 0.1-2.0 | Deodorant | 0.1-2.0 | Deodorant | 0.1-2.0 |
| active | | active | | active | |
| ingredient | | ingredient | | ingredient | |
| Al salt | 2.0-8.0 | Al salt | 2.0-8.0 | Al salt | 2.0-8.0 |
| KM | 0.05-2.0 | KM | 0.05-2.0 | KM | 0.05-2.0 |
| ZK177 | 0.1-5.0 | ZK178 | 0.1-5.0 | ZK179 | 0.1-5.0 |
| Carrier 1) | to make | Carrier 1) | to make | Carrier 1) | to make |
| | 100 | | 100 | | 100 |
| | AF250 | | AF251 | | AF252 |
| Deodorant | 0.1-2.0 | Deodorant | 0.1-2.0 | Deodorant | 0.1-2.0 |
| active | | active | | active | |
| ingredient | | ingredient | | ingredient | |
| Al salt | 2.0-8.0 | Al salt | 2.0-8.0 | Al salt | 2.0-8.0 |
| KM | 0.05-2.0 | KM | 0.05-2.0 | KM | 0.05-2.0 |
| ZK180 | 0.1-5.0 | ZK181 | 0.1-5.0 | ZK182 | 0.1-5.0 |
| Carrier 1) | to make | Carrier 1) | to make | Carrier 1) | to make |
| | 100 | | 100 | | 100 |
| | AF253 | | AF254 | | AF255 |
| Deodorant | 0.1-2.0 | Deodorant | 0.1-2.0 | Deodorant | 0.1-2.0 |
| active | | active | | active | |
| ingredient 2) | | ingredient 2) | | ingredient 2) | |
| KM | 0.05-2.0 | KM | 0.05-2.0 | KM | 0.05-2.0 |
| ZK1 | 0.1-5.0 | ZK2 | 0.1-5.0 | ZK3 | 0.1-5.0 |
| Carrier 1) | to make | Carrier 1) | to make | Carrier 1) | to make |
| | 100 | | 100 | | 100 |
| | AF256 | | AF257 | | AF258 |
| Deodorant | 0.1-2.0 | Deodorant | 0.1-2.0 | Deodorant | 0.1-2.0 |
| active | | active | | active | |
| ingredient 2) | | ingredient 2) | | ingredient 2) | |
| KM | 0.05-2.0 | KM | 0.05-2.0 | KM | 0.05-2.0 |
| ZK4 | 0.1-5.0 | ZK5 | 0.1-5.0 | ZK6 | 0.1-5.0 |
| Carrier 1) | to make | Carrier 1) | to make | Carrier 1) | to make |
| | 100 | | 100 | | 100 |
| | AF259 | | AF260 | | AF261 |
| Deodorant | 0.1-2.0 | Deodorant | 0.1-2.0 | Deodorant | 0.1-2.0 |
| active | | active | | active | |
| ingredient 2) | | ingredient 2) | | ingredient 2) | |
| KM | 0.05-2.0 | KM | 0.05-2.0 | KM | 0.05-2.0 |
| ZK8 | 0.1-5.0 | ZK9 | 0.1-5.0 | ZK10 | 0.1-5.0 |
| Carrier 1) | to make | Carrier 1) | to make | Carrier 1) | to make |
| | 100 | | 100 | | 100 |
| | AF262 | | AF263 | | AF264 |
| Deodorant | 0.1-2.0 | Deodorant | 0.1-2.0 | Deodorant | 0.1-2.0 |
| active | | active | | active | |
| ingredient 2) | | ingredient 2) | | ingredient 2) | |
| KM | 0.05-2.0 | KM | 0.05-2.0 | KM | 0.05-2.0 |
| ZK11 | 0.1-5.0 | ZK12 | 0.1-5.0 | ZK13 | 0.1-5.0 |
| Carrier 1) | to make | Carrier 1) | to make | Carrier 1) | to make |
| | 100 | | 100 | | 100 |
| | AF265 | | AF266 | | AF267 |
| Deodorant | 0.1-2.0 | Deodorant | 0.1-2.0 | Deodorant | 0.1-2.0 |
| active | | active | | active | |
| ingredient 2) | | ingredient 2) | | ingredient 2) | |
| KM | 0.05-2.0 | KM | 0.05-2.0 | KM | 0.05-2.0 |
| ZK66 | 0.1-5.0 | ZK112 | 0.1-5.0 | ZK113 | 0.1-5.0 |
| Carrier 1) | to make | Carrier 1) | to make | Carrier 1) | to make |
| | 100 | | 100 | | 100 |
| | AF268 | | AF269 | | AF270 |
| Deodorant | 0.1-2.0 | Deodorant | 0.1-2.0 | Deodorant | 0.1-2.0 |
| active | | active | | active | |
| ingredient 2) | | ingredient 2) | | ingredient 2) | |
| KM | 0.05-2.0 | KM | 0.05-2.0 | KM | 0.05-2.0 |
| ZK114 | 0.1-5.0 | ZK115 | 0.1-5.0 | ZK116 | 0.1-5.0 |
| Carrier 1) | to make | Carrier 1) | to make | Carrier 1) | to make |
| | 100 | | 100 | | 100 |
| | AF271 | | AF272 | | AF273 |
| Deodorant | 0.1-2.0 | Deodorant | 0.1-2.0 | Deodorant | 0.1-2.0 |
| active | | active | | active | |
| ingredient 2) | | ingredient 2) | | ingredient 2) | |
| KM | 0.05-2.0 | KM | 0.05-2.0 | KM | 0.05-2.0 |
| ZK117 | 0.1-5.0 | ZK118 | 0.1-5.0 | ZK119 | 0.1-5.0 |
| Carrier 1) | to make | Carrier 1) | to make | Carrier 1) | to make |
| | 100 | | 100 | | 100 |
| | AF274 | | AF275 | | AF276 |
| Deodorant | 0.1-2.0 | Deodorant | 0.1-2.0 | Deodorant | 0.1-2.0 |
| active | | active | | active | |
| ingredient 2) | | ingredient 2) | | ingredient 2) | |
| KM | 0.05-2.0 | KM | 0.05-2.0 | KM | 0.05-2.0 |
| ZK120 | 0.1-5.0 | ZK121 | 0.1-5.0 | ZK122 | 0.1-5.0 |
| Carrier 1) | to make | Carrier 1) | to make | Carrier 1) | to make |
| | 100 | | 100 | | 100 |
| | AF277 | | AF278 | | AF279 |
| Deodorant | 0.1-2.0 | Deodorant | 0.1-2.0 | Deodorant | 0.1-2.0 |
| active | | active | | active | |
| ingredient 2) | | ingredient 2) | | ingredient 2) | |
| KM | 0.05-2.0 | KM | 0.05-2.0 | KM | 0.05-2.0 |
| ZK123 | 0.1-5.0 | ZK124 | 0.1-5.0 | ZK125 | 0.1-5.0 |
| Carrier 1) | to make | Carrier 1) | to make | Carrier 1) | to make |
| | 100 | | 100 | | 100 |
| | AF280 | | AF281 | | AF282 |
| Deodorant | 0.1-2.0 | Deodorant | 0.1-2.0 | Deodorant | 0.1-2.0 |
| active | | active | | active | |
| ingredient 2) | | ingredient 2) | | ingredient 2) | |
| KM | 0.05-2.0 | KM | 0.05-2.0 | KM | 0.05-2.0 |
| ZK126 | 0.1-5.0 | ZK127 | 0.1-5.0 | ZK128 | 0.1-5.0 |
| Carrier 1) | to make | Carrier 1) | to make | Carrier 1) | to make |
| | 100 | | 100 | | 100 |
| | AF283 | | AF284 | | AF285 |
| Deodorant | 0.1-2.0 | Deodorant | 0.1-2.0 | Deodorant | 0.1-2.0 |
| active | | active | | active | |
| ingredient 2) | | ingredient 2) | | ingredient 2) | |
| KM | 0.05-2.0 | KM | 0.05-2.0 | KM | 0.05-2.0 |
| ZK129 | 0.1-5.0 | ZK130 | 0.1-5.0 | ZK131 | 0.1-5.0 |
| Carrier 1) | to make | Carrier 1) | to make | Carrier 1) | to make |
| | 100 | | 100 | | 100 |
| | AF286 | | AF287 | | AF288 |
| Deodorant | 0.1-2.0 | Deodorant | 0.1-2.0 | Deodorant | 0.1-2.0 |
| active | | active | | active | |
| ingredient 2) | | ingredient 2) | | ingredient 2) | |
| KM | 0.05-2.0 | KM | 0.05-2.0 | KM | 0.05-2.0 |
| ZK132 | 0.1-5.0 | ZK133 | 0.1-5.0 | ZK134 | 0.1-5.0 |
| Carrier 1) | to make | Carrier 1) | to make | Carrier 1) | to make |
| | 100 | | 100 | | 100 |
| | AF289 | | AF290 | | AF291 |
| Deodorant | 0.1-2.0 | Deodorant | 0.1-2.0 | Deodorant | 0.1-2.0 |
| active | | active | | active | |
| ingredient 2) | | ingredient 2) | | ingredient 2) | |
| KM | 0.05-2.0 | KM | 0.05-2.0 | KM | 0.05-2.0 |
| ZK135 | 0.1-5.0 | ZK136 | 0.1-5.0 | ZK137 | 0.1-5.0 |
| Carrier 1) | to make | Carrier 1) | to make | Carrier 1) | to make |
| | 100 | | 100 | | 100 |
| | AF292 | | AF293 | | AF294 |
| Deodorant | 0.1-2.0 | Deodorant | 0.1-2.0 | Deodorant | 0.1-2.0 |
| active | | active | | active | |
| ingredient 2) | | ingredient 2) | | ingredient 2) | |
| KM | 0.05-2.0 | KM | 0.05-2.0 | KM | 0.05-2.0 |
| ZK138 | 0.1-5.0 | ZK139 | 0.1-5.0 | ZK140 | 0.1-5.0 |
| Carrier 1) | to make | Carrier 1) | to make | Carrier 1) | to make |
| | 100 | | 100 | | 100 |
| | AF295 | | AF296 | | AF297 |
| Deodorant | 0.1-2.0 | Deodorant | 0.1-2.0 | Deodorant | 0.1-2.0 |
| active | | active | | active | |
| ingredient 2) | | ingredient 2) | | ingredient 2) | |
| KM | 0.05-2.0 | KM | 0.05-2.0 | KM | 0.05-2.0 |
| ZK141 | 0.1-5.0 | ZK142 | 0.1-5.0 | ZK143 | 0.1-5.0 |
| Carrier 1) | to make | Carrier 1) | to make | Carrier 1) | to make |
| | 100 | | 100 | | 100 |
| | AF298 | | AF299 | | AF300 |
| Deodorant | 0.1-2.0 | Deodorant | 0.1-2.0 | Deodorant | 0.1-2.0 |
| active | | active | | active | |
| ingredient 2) | | ingredient 2) | | ingredient 2) | |
| KM | 0.05-2.0 | KM | 0.05-2.0 | KM | 0.05-2.0 |
| ZK144 | 0.1-5.0 | ZK145 | 0.1-5.0 | ZK146 | 0.1-5.0 |
| Carrier 1) | to make | Carrier 1) | to make | Carrier 1) | to make |
| | 100 | | 100 | | 100 |
| | AF301 | | AF302 | | AF303 |
| Deodorant | 0.1-2.0 | Deodorant | 0.1-2.0 | Deodorant | 0.1-2.0 |
| active | | active | | active | |
| ingredient 2) | | ingredient 2) | | ingredient 2) | |
| KM | 0.05-2.0 | KM | 0.05-2.0 | KM | 0.05-2.0 |
| ZK147 | 0.1-5.0 | ZK148 | 0.1-5.0 | ZK149 | 0.1-5.0 |
| Carrier 1) | to make | Carrier 1) | to make | Carrier 1) | to make |
| | 100 | | 100 | | 100 |
| | AF304 | | AF305 | | AF306 |
| Deodorant | 0.1-2.0 | Deodorant | 0.1-2.0 | Deodorant | 0.1-2.0 |
| active | | active | | active | |
| ingredient 2) | | ingredient 2) | | ingredient 2) | |
| KM | 0.05-2.0 | KM | 0.05-2.0 | KM | 0.05-2.0 |
| ZK150 | 0.1-5.0 | ZK151 | 0.1-5.0 | ZK152 | 0.1-5.0 |
| Carrier 1) | to make | Carrier 1) | to make | Carrier 1) | to make |
| | 100 | | 100 | | 100 |
| | AF307 | | AF308 | | AF309 |
| Deodorant | 0.1-2.0 | Deodorant | 0.1-2.0 | Deodorant | 0.1-2.0 |
| active | | active | | active | |
| ingredient 2) | | ingredient 2) | | ingredient 2) | |
| KM | 0.05-2.0 | KM | 0.05-2.0 | KM | 0.05-2.0 |
| ZK153 | 0.1-5.0 | ZK154 | 0.1-5.0 | ZK155 | 0.1-5.0 |
| Carrier 1) | to make | Carrier 1) | to make | Carrier 1) | to make |
| | 100 | | 100 | | 100 |
| | AF310 | | AF311 | | AF312 |
| Deodorant | 0.1-2.0 | Deodorant | 0.1-2.0 | Deodorant | 0.1-2.0 |
| active | | active | | active | |
| ingredient 2) | | ingredient 2) | | ingredient 2) | |
| KM | 0.05-2.0 | KM | 0.05-2.0 | KM | 0.05-2.0 |
| ZK156 | 0.1-5.0 | ZK157 | 0.1-5.0 | ZK158 | 0.1-5.0 |
| Carrier 1) | to make | Carrier 1) | to make | Carrier 1) | to make |
| | 100 | | 100 | | 100 |
| | AF313 | | AF314 | | AF315 |
| Deodorant | 0.1-2.0 | Deodorant | 0.1-2.0 | Deodorant | 0.1-2.0 |
| active | | active | | active | |
| ingredient 2) | | ingredient 2) | | ingredient 2) | |
| KM | 0.05-2.0 | KM | 0.05-2.0 | KM | 0.05-2.0 |
| ZK159 | 0.1-5.0 | ZK160 | 0.1-5.0 | ZK161 | 0.1-5.0 |
| Carrier 1) | to make | Carrier 1) | to make | Carrier 1) | to make |
| | 100 | | 100 | | 100 |
| | AF316 | | AF317 | | AF318 |
| Deodorant | 0.1-2.0 | Deodorant | 0.1-2.0 | Deodorant | 0.1-2.0 |
| active | | active | | active | |
| ingredient 2) | | ingredient 2) | | ingredient 2) | |
| KM | 0.05-2.0 | KM | 0.05-2.0 | KM | 0.05-2.0 |
| ZK162 | 0.1-5.0 | ZK163 | 0.1-5.0 | ZK164 | 0.1-5.0 |
| Carrier 1) | to make | Carrier 1) | to make | Carrier 1) | to make |
| | 100 | | 100 | | 100 |
| | AF319 | | AF320 | | AF321 |
| Deodorant | 0.1-2.0 | Deodorant | 0.1-2.0 | Deodorant | 0.1-2.0 |
| active | | active | | active | |
| ingredient 2) | | ingredient 2) | | ingredient 2) | |
| KM | 0.05-2.0 | KM | 0.05-2.0 | KM | 0.05-2.0 |
| ZK165 | 0.1-5.0 | ZK166 | 0.1-5.0 | ZK167 | 0.1-5.0 |
| Carrier 1) | to make | Carrier 1) | to make | Carrier 1) | to make |
| | 100 | | 100 | | 100 |
| | AF322 | | AF323 | | AF324 |
| Deodorant | 0.1-2.0 | Deodorant | 0.1-2.0 | Deodorant | 0.1-2.0 |
| active | | active | | active | |
| ingredient 2) | | ingredient 2) | | ingredient 2) | |
| KM | 0.05-2.0 | KM | 0.05-2.0 | KM | 0.05-2.0 |
| ZK168 | 0.1-5.0 | ZK169 | 0.1-5.0 | ZK170 | 0.1-5.0 |
| Carrier 1) | to make | Carrier 1) | to make | Carrier 1) | to make |
| | 100 | | 100 | | 100 |
| | AF325 | | AF326 | | AF327 |
| Deodorant | 0.1-2.0 | Deodorant | 0.1-2.0 | Deodorant | 0.1-2.0 |
| active | | active | | active | |
| ingredient 2) | | ingredient 2) | | ingredient 2) | |
| KM | 0.05-2.0 | KM | 0.05-2.0 | KM | 0.05-2.0 |
| ZK171 | 0.1-5.0 | ZK172 | 0.1-5.0 | ZK173 | 0.1-5.0 |
| Carrier 1) | to make | Carrier 1) | to make | Carrier 1) | to make |
| | 100 | | 100 | | 100 |
| | AF328 | | AF329 | | AF330 |
| Deodorant | 0.1-2.0 | Deodorant | 0.1-2.0 | Deodorant | 0.1-2.0 |
| active | | active | | active | |
| ingredient 2) | | ingredient 2) | | ingredient 2) | |
| KM | 0.05-2.0 | KM | 0.05-2.0 | KM | 0.05-2.0 |
| ZK174 | 0.1-5.0 | ZK175 | 0.1-5.0 | ZK176 | 0.1-5.0 |
| Carrier 1) | to make | Carrier 1) | to make | Carrier 1) | to make |
| | 100 | | 100 | | 100 |
| | AF331 | | AF332 | | AF333 |
| Deodorant | 0.1-2.0 | Deodorant | 0.1-2.0 | Deodorant | 0.1-2.0 |
| active | | active | | active | |
| ingredient 2) | | ingredient 2) | | ingredient 2) | |
| KM | 0.05-2.0 | KM | 0.05-2.0 | KM | 0.05-2.0 |
| ZK177 | 0.1-5.0 | ZK178 | 0.1-5.0 | ZK179 | 0.1-5.0 |
| Carrier 1) | to make | Carrier 1) | to make | Carrier 1) | to make |
| | 100 | | 100 | | 100 |
| | AF334 | | AF335 | | AF336 |
| Deodorant | 0.1-2.0 | Deodorant | 0.1-2.0 | Deodorant | 0.1-2.0 |
| active | | active | | active | |
| ingredient 2) | | ingredient 2) | | ingredient 2) | |
| KM | 0.05-2.0 | KM | 0.05-2.0 | KM | 0.05-2.0 |
| ZK180 | 0.1-5.0 | ZK181 | 0.1-5.0 | ZK182 | 0.1-5.0 |
| Carrier 1) | to make | Carrier 1) | to make | Carrier 1) | to make |
| | 100 | | 100 | | 100 |
| KM | 0.1-7.0 | KM | 0.1-7.0 | KM | 0.1-7.0 |
| | AF337 | | AF338 | | AF339 |
| Al salt 3) | 2.0-8.0 | Al salt 3) | 2.0-8.0 | Al salt 3) | 2.0-8.0 |
| KM | 0.05-2.0 | KM | 0.05-2.0 | KM | 0.05-2.0 |
| ZK1 | 0.1-5.0 | ZK2 | 0.1-5.0 | ZK3 | 0.1-5.0 |
| Carrier 1) | to make | Carrier 1) | to make | Carrier 1) | to make |
| | 100 | | 100 | | 100 |
| | AF340 | | AF341 | | AF342 |
| Al salt 3) | 2.0-8.0 | Al salt 3) | 2.0-8.0 | Al salt 3) | 2.0-8.0 |
| KM | 0.05-2.0 | KM | 0.05-2.0 | KM | 0.05-2.0 |
| ZK4 | 0.1-5.0 | ZK5 | 0.1-5.0 | ZK6 | 0.1-5.0 |
| Carrier 1) | to make | Carrier 1) | to make | Carrier 1) | to make |
| | 100 | | 100 | | 100 |
| | AF343 | | AF344 | | AF345 |
| Al salt 3) | 2.0-8.0 | Al salt 3) | 2.0-8.0 | Al salt 3) | 2.0-8.0 |
| KM | 0.05-2.0 | KM | 0.05-2.0 | KM | 0.05-2.0 |
| ZK8 | 0.1-5.0 | ZK9 | 0.1-5.0 | ZK10 | 0.1-5.0 |
| Carrier 1) | to make | Carrier 1) | to make | Carrier 1) | to make |
| | 100 | | 100 | | 100 |
| | AF346 | | AF347 | | AF348 |
| Al salt 3) | 2.0-8.0 | Al salt 3) | 2.0-8.0 | Al salt 3) | 2.0-8.0 |
| KM | 0.05-2.0 | KM | 0.05-2.0 | KM | 0.05-2.0 |
| ZK11 | 0.1-5.0 | ZK12 | 0.1-5.0 | ZK13 | 0.1-5.0 |
| Carrier 1) | to make | Carrier 1) | to make | Carrier 1) | to make |
| | 100 | | 100 | | 100 |
| | AF349 | | AF350 | | AF351 |
| Al salt 3) | 2.0-8.0 | Al salt 3) | 2.0-8.0 | Al salt 3) | 2.0-8.0 |
| KM | 0.05-2.0 | KM | 0.05-2.0 | KM | 0.05-2.0 |
| ZK66 | 0.1-5.0 | ZK112 | 0.1-5.0 | ZK113 | 0.1-5.0 |
| Carrier 1) | to make | Carrier 1) | to make | Carrier 1) | to make |
| | 100 | | 100 | | 100 |
| | AF352 | | AF353 | | AF354 |
| Al salt 3) | 2.0-8.0 | Al salt 3) | 2.0-8.0 | Al salt 3) | 2.0-8.0 |
| KM | 0.05-2.0 | KM | 0.05-2.0 | KM | 0.05-2.0 |
| ZK114 | 0.1-5.0 | ZK115 | 0.1-5.0 | ZK116 | 0.1-5.0 |
| Carrier 1) | to make | Carrier 1) | to make | Carrier 1) | to make |
| | 100 | | 100 | | 100 |
| | AF355 | | AF356 | | AF357 |
| Al salt 3) | 2.0-8.0 | Al salt 3) | 2.0-8.0 | Al salt 3) | 2.0-8.0 |
| KM | 0.05-2.0 | KM | 0.05-2.0 | KM | 0.05-2.0 |
| ZK117 | 0.1-5.0 | ZK118 | 0.1-5.0 | ZK119 | 0.1-5.0 |
| Carrier 1) | to make | Carrier 1) | to make | Carrier 1) | to make |
| | 100 | | 100 | | 100 |
| | AF358 | | AF359 | | AF360 |
| Al salt 3) | 2.0-8.0 | Al salt 3) | 2.0-8.0 | Al salt 3) | 2.0-8.0 |
| KM | 0.05-2.0 | KM | 0.05-2.0 | KM | 0.05-2.0 |
| ZK120 | 0.1-5.0 | ZK121 | 0.1-5.0 | ZK122 | 0.1-5.0 |
| Carrier 1) | to make | Carrier 1) | to make | Carrier 1) | to make |
| | 100 | | 100 | | 100 |
| | AF361 | | AF362 | | AF363 |
| Al salt 3) | 2.0-8.0 | Al salt 3) | 2.0-8.0 | Al salt 3) | 2.0-8.0 |
| KM | 0.05-2.0 | KM | 0.05-2.0 | KM | 0.05-2.0 |
| ZK123 | 0.1-5.0 | ZK124 | 0.1-5.0 | ZK125 | 0.1-5.0 |
| Carrier 1) | to make | Carrier 1) | to make | Carrier 1) | to make |
| | 100 | | 100 | | 100 |
| | AF364 | | AF365 | | AF366 |
| Al salt 3) | 2.0-8.0 | Al salt 3) | 2.0-8.0 | Al salt 3) | 2.0-8.0 |
| KM | 0.05-2.0 | KM | 0.05-2.0 | KM | 0.05-2.0 |
| ZK126 | 0.1-5.0 | ZK127 | 0.1-5.0 | ZK128 | 0.1-5.0 |
| Carrier 1) | to make | Carrier 1) | to make | Carrier 1) | to make |
| | 100 | | 100 | | 100 |
| | AF367 | | AF368 | | AF369 |
| Al salt 3) | 2.0-8.0 | Al salt 3) | 2.0-8.0 | Al salt 3) | 2.0-8.0 |
| KM | 0.05-2.0 | KM | 0.05-2.0 | KM | 0.05-2.0 |
| ZK129 | 0.1-5.0 | ZK130 | 0.1-5.0 | ZK131 | 0.1-5.0 |
| Carrier 1) | to make | Carrier 1) | to make | Carrier 1) | to make |
| | 100 | | 100 | | 100 |
| | AF370 | | AF371 | | AF372 |
| Al salt 3) | 2.0-8.0 | Al salt 3) | 2.0-8.0 | Al salt 3) | 2.0-8.0 |
| KM | 0.05-2.0 | KM | 0.05-2.0 | KM | 0.05-2.0 |
| ZK132 | 0.1-5.0 | ZK133 | 0.1-5.0 | ZK134 | 0.1-5.0 |
| Carrier 1) | to make | Carrier 1) | to make | Carrier 1) | to make |
| | 100 | | 100 | | 100 |
| | AF373 | | AF374 | | AF375 |
| Al salt 3) | 2.0-8.0 | Al salt 3) | 2.0-8.0 | Al salt 3) | 2.0-8.0 |
| KM | 0.05-2.0 | KM | 0.05-2.0 | KM | 0.05-2.0 |
| ZK135 | 0.1-5.0 | ZK136 | 0.1-5.0 | ZK137 | 0.1-5.0 |
| Carrier 1) | to make | Carrier 1) | to make | Carrier 1) | to make |
| | 100 | | 100 | | 100 |
| | AF376 | | AF377 | | AF378 |
| Al salt 3) | 2.0-8.0 | Al salt 3) | 2.0-8.0 | Al salt 3) | 2.0-8.0 |
| KM | 0.05-2.0 | KM | 0.05-2.0 | KM | 0.05-2.0 |
| ZK138 | 0.1-5.0 | ZK139 | 0.1-5.0 | ZK140 | 0.1-5.0 |
| Carrier 1) | to make | Carrier 1) | to make | Carrier 1) | to make |
| | 100 | | 100 | | 100 |
| | AF379 | | AF380 | | AF381 |
| Al salt 3) | 2.0-8.0 | Al salt 3) | 2.0-8.0 | Al salt 3) | 2.0-8.0 |
| KM | 0.05-2.0 | KM | 0.05-2.0 | KM | 0.05-2.0 |
| ZK141 | 0.1-5.0 | ZK142 | 0.1-5.0 | ZK143 | 0.1-5.0 |
| Carrier 1) | to make | Carrier 1) | to make | Carrier 1) | to make |
| | 100 | | 100 | | 100 |
| | AF382 | | AF383 | | AF384 |
| Al salt 3) | 2.0-8.0 | Al salt 3) | 2.0-8.0 | Al salt 3) | 2.0-8.0 |
| KM | 0.05-2.0 | KM | 0.05-2.0 | KM | 0.05-2.0 |
| ZK144 | 0.1-5.0 | ZK145 | 0.1-5.0 | ZK146 | 0.1-5.0 |
| Carrier 1) | to make | Carrier 1) | to make | Carrier 1) | to make |
| | 100 | | 100 | | 100 |
| | AF385 | | AF386 | | AF387 |
| Al salt 3) | 2.0-8.0 | Al salt 3) | 2.0-8.0 | Al salt 3) | 2.0-8.0 |
| KM | 0.05-2.0 | KM | 0.05-2.0 | KM | 0.05-2.0 |
| ZK147 | 0.1-5.0 | ZK148 | 0.1-5.0 | ZK149 | 0.1-5.0 |
| Carrier 1) | to make | Carrier 1) | to make | Carrier 1) | to make |
| | 100 | | 100 | | 100 |
| | AF388 | | AF389 | | AF390 |
| Al salt 3) | 2.0-8.0 | Al salt 3) | 2.0-8.0 | Al salt 3) | 2.0-8.0 |
| KM | 0.05-2.0 | KM | 0.05-2.0 | KM | 0.05-2.0 |
| ZK150 | 0.1-5.0 | ZK151 | 0.1-5.0 | ZK152 | 0.1-5.0 |
| Carrier 1) | to make | Carrier 1) | to make | Carrier 1) | to make |
| | 100 | | 100 | | 100 |
| | AF391 | | AF392 | | AF393 |
| Al salt 3) | 2.0-8.0 | Al salt 3) | 2.0-8.0 | Al salt 3) | 2.0-8.0 |
| KM | 0.05-2.0 | KM | 0.05-2.0 | KM | 0.05-2.0 |
| ZK153 | 0.1-5.0 | ZK154 | 0.1-5.0 | ZK155 | 0.1-5.0 |
| Carrier 1) | to make | Carrier 1) | to make | Carrier 1) | to make |
| | 100 | | 100 | | 100 |
| | AF394 | | AF395 | | AF396 |
| Al salt 3) | 2.0-8.0 | Al salt 3) | 2.0-8.0 | Al salt 3) | 2.0-8.0 |
| KM | 0.05-2.0 | KM | 0.05-2.0 | KM | 0.05-2.0 |
| ZK156 | 0.1-5.0 | ZK157 | 0.1-5.0 | ZK158 | 0.1-5.0 |
| Carrier 1) | to make | Carrier 1) | to make | Carrier 1) | to make |
| | 100 | | 100 | | 100 |
| | AF397 | | AF398 | | AF399 |
| Al salt 3) | 2.0-8.0 | Al salt 3) | 2.0-8.0 | Al salt 3) | 2.0-8.0 |
| KM | 0.05-2.0 | KM | 0.05-2.0 | KM | 0.05-2.0 |
| ZK159 | 0.1-5.0 | ZK160 | 0.1-5.0 | ZK161 | 0.1-5.0 |
| Carrier 1) | to make | Carrier 1) | to make | Carrier 1) | to make |
| | 100 | | 100 | | 100 |
| | AF400 | | AF401 | | AF402 |
| Al salt 3) | 2.0-8.0 | Al salt 3) | 2.0-8.0 | Al salt 3) | 2.0-8.0 |
| KM | 0.05-2.0 | KM | 0.05-2.0 | KM | 0.05-2.0 |
| ZK162 | 0.1-5.0 | ZK163 | 0.1-5.0 | ZK164 | 0.1-5.0 |
| Carrier 1) | to make | Carrier 1) | to make | Carrier 1) | to make |
| | 100 | | 100 | | 100 |
| | AF403 | | AF404 | | AF405 |
| Al salt 3) | 2.0-8.0 | Al salt 3) | 2.0-8.0 | Al salt 3) | 2.0-8.0 |
| KM | 0.05-2.0 | KM | 0.05-2.0 | KM | 0.05-2.0 |
| ZK165 | 0.1-5.0 | ZK166 | 0.1-5.0 | ZK167 | 0.1-5.0 |
| Carrier 1) | to make | Carrier 1) | to make | Carrier 1) | to make |
| | 100 | | 100 | | 100 |
| | AF406 | | AF407 | | AF408 |
| Al salt 3) | 2.0-8.0 | Al salt 3) | 2.0-8.0 | Al salt 3) | 2.0-8.0 |
| KM | 0.05-2.0 | KM | 0.05-2.0 | KM | 0.05-2.0 |
| ZK168 | 0.1-5.0 | ZK169 | 0.1-5.0 | ZK170 | 0.1-5.0 |
| Carrier 1) | to make | Carrier 1) | to make | Carrier 1) | to make |
| | 100 | | 100 | | 100 |
| | AF409 | | AF410 | | AF411 |
| Al salt 3) | 2.0-8.0 | Al salt 3) | 2.0-8.0 | Al salt 3) | 2.0-8.0 |
| KM | 0.05-2.0 | KM | 0.05-2.0 | KM | 0.05-2.0 |
| ZK171 | 0.1-5.0 | ZK172 | 0.1-5.0 | ZK173 | 0.1-5.0 |
| Carrier 1) | to make | Carrier 1) | to make | Carrier 1) | to make |
| | 100 | | 100 | | 100 |
| | AF412 | | AF413 | | AF414 |
| Al salt 3) | 2.0-8.0 | Al salt 3) | 2.0-8.0 | Al salt 3) | 2.0-8.0 |
| KM | 0.05-2.0 | KM | 0.05-2.0 | KM | 0.05-2.0 |
| ZK174 | 0.1-5.0 | ZK175 | 0.1-5.0 | ZK176 | 0.1-5.0 |
| Carrier 1) | to make | Carrier 1) | to make | Carrier 1) | to make |
| | 100 | | 100 | | 100 |
| | AF415 | | AF416 | | AF417 |
| Al salt 3) | 2.0-8.0 | Al salt 3) | 2.0-8.0 | Al salt 3) | 2.0-8.0 |
| KM | 0.05-2.0 | KM | 0.05-2.0 | KM | 0.05-2.0 |
| ZK177 | 0.1-5.0 | ZK178 | 0.1-5.0 | ZK179 | 0.1-5.0 |
| Carrier 1) | to make | Carrier 1) | to make | Carrier 1) | to make |
| | 100 | | 100 | | 100 |
| | AF418 | | AF419 | | AF420 |
| Al salt 3) | 2.0-8.0 | Al salt 3) | 2.0-8.0 | Al salt 3) | 2.0-8.0 |
| KM | 0.05-2.0 | KM | 0.05-2.0 | KM | 0.05-2.0 |
| ZK180 | 0.1-5.0 | ZK181 | 0.1-5.0 | ZK182 | 0.1-5.0 |
| Carrier 1) | to make | Carrier 1) | to make | Carrier 1) | to make |
| | 100 | | 100 | | 100 |
| | AF421 | | AF422 | | AF423 |
| Deodorant | 0.1-2.0 | Deodorant | 0.1-2.0 | Deodorant | 0.1-2.0 |
| active | | active | | active | |
| ingredient 2) | | ingredient 2) | | ingredient 2) | |
| Al salt 3) | 2.0-8.0 | Al salt 3) | 2.0-8.0 | Al salt 3) | 2.0-8.0 |
| KM | 0.05-2.0 | KM | 0.05-2.0 | KM | 0.05-2.0 |
| ZK1 | 0.1-5.0 | ZK2 | 0.1-5.0 | ZK3 | 0.1-5.0 |
| Carrier 1) | to make | Carrier 1) | to make | Carrier 1) | to make |
| | 100 | | 100 | | 100 |
| | AF424 | | AF425 | | AF426 |
| Deodorant | 0.1-2.0 | Deodorant | 0.1-2.0 | Deodorant | 0.1-2.0 |
| active | | active | | active | |
| ingredient 2) | | ingredient 2) | | ingredient 2) | |
| Al salt 3) | 2.0-8.0 | Al salt 3) | 2.0-8.0 | Al salt 3) | 2.0-8.0 |
| KM | 0.05-2.0 | KM | 0.05-2.0 | KM | 0.05-2.0 |
| ZK4 | 0.1-5.0 | ZK5 | 0.1-5.0 | ZK6 | 0.1-5.0 |
| Carrier 1) | to make | Carrier 1) | to make | Carrier 1) | to make |
| | 100 | | 100 | | 100 |
| | AF427 | | AF428 | | AF429 |
| Deodorant | 0.1-2.0 | Deodorant | 0.1-2.0 | Deodorant | 0.1-2.0 |
| active | | active | | active | |
| ingredient 2) | | ingredient 2) | | ingredient 2) | |
| Al salt 3) | 2.0-8.0 | Al salt 3) | 2.0-8.0 | Al salt 3) | 2.0-8.0 |
| KM | 0.05-2.0 | KM | 0.05-2.0 | KM | 0.05-2.0 |
| ZK8 | 0.1-5.0 | ZK9 | 0.1-5.0 | ZK10 | 0.1-5.0 |
| Carrier 1) | to make | Carrier 1) | to make | Carrier 1) | to make |
| | 100 | | 100 | | 100 |
| | AF430 | | AF431 | | AF432 |
| Deodorant | 0.1-2.0 | Deodorant | 0.1-2.0 | Deodorant | 0.1-2.0 |
| active | | active | | active | |
| ingredient 2) | | ingredient 2) | | ingredient 2) | |
| Al salt 3) | 2.0-8.0 | Al salt 3) | 2.0-8.0 | Al salt 3) | 2.0-8.0 |
| KM | 0.05-2.0 | KM | 0.05-2.0 | KM | 0.05-2.0 |
| ZK11 | 0.1-5.0 | ZK12 | 0.1-5.0 | ZK13 | 0.1-5.0 |
| Carrier 1) | to make | Carrier 1) | to make | Carrier 1) | to make |
| | 100 | | 100 | | 100 |
| | AF433 | | AF434 | | AF435 |
| Deodorant | 0.1-2.0 | Deodorant | 0.1-2.0 | Deodorant | 0.1-2.0 |
| active | | active | | active | |
| ingredient 2) | | ingredient 2) | | ingredient 2) | |
| Al salt 3) | 2.0-8.0 | Al salt 3) | 2.0-8.0 | Al salt 3) | 2.0-8.0 |
| KM | 0.05-2.0 | KM | 0.05-2.0 | KM | 0.05-2.0 |
| ZK66 | 0.1-5.0 | ZK112 | 0.1-5.0 | ZK113 | 0.1-5.0 |
| Carrier 1) | to make | Carrier 1) | to make | Carrier 1) | to make |
| | 100 | | 100 | | 100 |
| | AF436 | | AF437 | | AF438 |
| Deodorant | 0.1-2.0 | Deodorant | 0.1-2.0 | Deodorant | 0.1-2.0 |
| active | | active | | active | |
| ingredient 2) | | ingredient 2) | | ingredient 2) | |
| Al salt 3) | 2.0-8.0 | Al salt 3) | 2.0-8.0 | Al salt 3) | 2.0-8.0 |
| KM | 0.05-2.0 | KM | 0.05-2.0 | KM | 0.05-2.0 |
| ZK114 | 0.1-5.0 | ZK115 | 0.1-5.0 | ZK116 | 0.1-5.0 |
| Carrier 1) | to make | Carrier 1) | to make | Carrier 1) | to make |
| | 100 | | 100 | | 100 |
| | AF439 | | AF440 | | AF441 |
| Deodorant | 0.1-2.0 | Deodorant | 0.1-2.0 | Deodorant | 0.1-2.0 |
| active | | active | | active | |
| ingredient 2) | | ingredient 2) | | ingredient 2) | |
| Al salt 3) | 2.0-8.0 | Al salt 3) | 2.0-8.0 | Al salt 3) | 2.0-8.0 |
| KM | 0.05-2.0 | KM | 0.05-2.0 | KM | 0.05-2.0 |
| ZK117 | 0.1-5.0 | ZK118 | 0.1-5.0 | ZK119 | 0.1-5.0 |
| Carrier 1) | to make | Carrier 1) | to make | Carrier 1) | to make |
| | 100 | | 100 | | 100 |
| | AF442 | | AF443 | | AF444 |
| Deodorant | 0.1-2.0 | Deodorant | 0.1-2.0 | Deodorant | 0.1-2.0 |
| active | | active | | active | |
| ingredient 2) | | ingredient 2) | | ingredient 2) | |
| Al salt 3) | 2.0-8.0 | Al salt 3) | 2.0-8.0 | Al salt 3) | 2.0-8.0 |
| KM | 0.05-2.0 | KM | 0.05-2.0 | KM | 0.05-2.0 |
| ZK120 | 0.1-5.0 | ZK121 | 0.1-5.0 | ZK122 | 0.1-5.0 |
| Carrier 1) | to make | Carrier 1) | to make | Carrier 1) | to make |
| | 100 | | 100 | | 100 |
| | AF445 | | AF446 | | AF447 |
| Deodorant | 0.1-2.0 | Deodorant | 0.1-2.0 | Deodorant | 0.1-2.0 |
| active | | active | | active | |
| ingredient 2) | | ingredient 2) | | ingredient 2) | |
| Al salt 3) | 2.0-8.0 | Al salt 3) | 2.0-8.0 | Al salt 3) | 2.0-8.0 |
| KM | 0.05-2.0 | KM | 0.05-2.0 | KM | 0.05-2.0 |
| ZK123 | 0.1-5.0 | ZK124 | 0.1-5.0 | ZK125 | 0.1-5.0 |
| Carrier 1) | to make | Carrier 1) | to make | Carrier 1) | to make |
| | 100 | | 100 | | 100 |
| | AF784 | | AF785 | | AF786 |
| Deodorant | 0.1-2.0 | Deodorant | 0.1-2.0 | Deodorant | 0.1-2.0 |
| active | | active | | active | |
| ingredient 2) | | ingredient 2) | | ingredient 2) | |
| Al salt 3) | 2.0-8.0 | Al salt 3) | 2.0-8.0 | Al salt 3) | 2.0-8.0 |
| KM | 0.05-2.0 | KM | 0.05-2.0 | KM | 0.05-2.0 |
| ZK126 | 0.1-5.0 | ZK127 | 0.1-5.0 | ZK128 | 0.1-5.0 |
| Carrier 1) | to make | Carrier 1) | to make | Carrier 1) | to make |
| | 100 | | 100 | | 100 |
| | AF448 | | AF449 | | AF450 |
| Deodorant | 0.1-2.0 | Deodorant | 0.1-2.0 | Deodorant | 0.1-2.0 |
| active | | active | | active | |
| ingredient 2) | | ingredient 2) | | ingredient 2) | |
| Al salt 3) | 2.0-8.0 | Al salt 3) | 2.0-8.0 | Al salt 3) | 2.0-8.0 |
| KM | 0.05-2.0 | KM | 0.05-2.0 | KM | 0.05-2.0 |
| ZK129 | 0.1-5.0 | ZK130 | 0.1-5.0 | ZK131 | 0.1-5.0 |
| Carrier 1) | to make | Carrier 1) | to make | Carrier 1) | to make |
| | 100 | | 100 | | 100 |
| | AF451 | | AF452 | | AF453 |
| Deodorant | 0.1-2.0 | Deodorant | 0.1-2.0 | Deodorant | 0.1-2.0 |
| active | | active | | active | |
| ingredient 2) | | ingredient 2) | | ingredient 2) | |
| Al salt 3) | 2.0-8.0 | Al salt 3) | 2.0-8.0 | Al salt 3) | 2.0-8.0 |
| KM | 0.05-2.0 | KM | 0.05-2.0 | KM | 0.05-2.0 |
| ZK132 | 0.1-5.0 | ZK133 | 0.1-5.0 | ZK134 | 0.1-5.0 |
| Carrier 1) | to make | Carrier 1) | to make | Carrier 1) | to make |
| | 100 | | 100 | | 100 |
| | AF454 | | AF455 | | AF456 |
| Deodorant | 0.1-2.0 | Deodorant | 0.1-2.0 | Deodorant | 0.1-2.0 |
| active | | active | | active | |
| ingredient 2) | | ingredient 2) | | ingredient 2) | |
| Al salt 3) | 2.0-8.0 | Al salt 3) | 2.0-8.0 | Al salt 3) | 2.0-8.0 |
| KM | 0.05-2.0 | KM | 0.05-2.0 | KM | 0.05-2.0 |
| ZK135 | 0.1-5.0 | ZK136 | 0.1-5.0 | ZK137 | 0.1-5.0 |
| Carrier 1) | to make | Carrier 1) | to make | Carrier 1) | to make |
| | 100 | | 100 | | 100 |
| | AF457 | | AF458 | | AF459 |
| Deodorant | 0.1-2.0 | Deodorant | 0.1-2.0 | Deodorant | 0.1-2.0 |
| active | | active | | active | |
| ingredient 2) | | ingredient 2) | | ingredient 2) | |
| Al salt 3) | 2.0-8.0 | Al salt 3) | 2.0-8.0 | Al salt 3) | 2.0-8.0 |
| KM | 0.05-2.0 | KM | 0.05-2.0 | KM | 0.05-2.0 |
| ZK138 | 0.1-5.0 | ZK139 | 0.1-5.0 | ZK140 | 0.1-5.0 |
| Carrier 1) | to make | Carrier 1) | to make | Carrier 1) | to make |
| | 100 | | 100 | | 100 |
| | AF460 | | AF461 | | AF462 |
| Deodorant | 0.1-2.0 | Deodorant | 0.1-2.0 | Deodorant | 0.1-2.0 |
| active | | active | | active | |
| ingredient 2) | | ingredient 2) | | ingredient 2) | |
| Al salt 3) | 2.0-8.0 | Al salt 3) | 2.0-8.0 | Al salt 3) | 2.0-8.0 |
| KM | 0.05-2.0 | KM | 0.05-2.0 | KM | 0.05-2.0 |
| ZK141 | 0.1-5.0 | ZK142 | 0.1-5.0 | ZK143 | 0.1-5.0 |
| Carrier 1) | to make | Carrier 1) | to make | Carrier 1) | to make |
| | 100 | | 100 | | 100 |
| | AF463 | | AF464 | | AF465 |
| Deodorant | 0.1-2.0 | Deodorant | 0.1-2.0 | Deodorant | 0.1-2.0 |
| active | | active | | active | |
| ingredient 2) | | ingredient 2) | | ingredient 2) | |
| Al salt 3) | 2.0-8.0 | Al salt 3) | 2.0-8.0 | Al salt 3) | 2.0-8.0 |
| KM | 0.05-2.0 | KM | 0.05-2.0 | KM | 0.05-2.0 |
| ZK144 | 0.1-5.0 | ZK145 | 0.1-5.0 | ZK146 | 0.1-5.0 |
| Carrier 1) | to make | Carrier 1) | to make | Carrier 1) | to make |
| | 100 | | 100 | | 100 |
| | AF466 | | AF467 | | AF468 |
| Deodorant | 0.1-2.0 | Deodorant | 0.1-2.0 | Deodorant | 0.1-2.0 |
| active | | active | | active | |
| ingredient 2) | | ingredient 2) | | ingredient 2) | |
| Al salt 3) | 2.0-8.0 | Al salt 3) | 2.0-8.0 | Al salt 3) | 2.0-8.0 |
| KM | 0.05-2.0 | KM | 0.05-2.0 | KM | 0.05-2.0 |
| ZK147 | 0.1-5.0 | ZK148 | 0.1-5.0 | ZK149 | 0.1-5.0 |
| Carrier 1) | to make | Carrier 1) | to make | Carrier 1) | to make |
| | 100 | | 100 | | 100 |
| | AF469 | | AF470 | | AF471 |
| Deodorant | 0.1-2.0 | Deodorant | 0.1-2.0 | Deodorant | 0.1-2.0 |
| active | | active | | active | |
| ingredient 2) | | ingredient 2) | | ingredient 2) | |
| Al salt 3) | 2.0-8.0 | Al salt 3) | 2.0-8.0 | Al salt 3) | 2.0-8.0 |
| KM | 0.05-2.0 | KM | 0.05-2.0 | KM | 0.05-2.0 |
| ZK150 | 0.1-5.0 | ZK151 | 0.1-5.0 | ZK152 | 0.1-5.0 |
| Carrier 1) | to make | Carrier 1) | to make | Carrier 1) | to make |
| | 100 | | 100 | | 100 |
| | AF472 | | AF473 | | AF474 |
| Deodorant | 0.1-2.0 | Deodorant | 0.1-2.0 | Deodorant | 0.1-2.0 |
| active | | active | | active | |
| ingredient 2) | | ingredient 2) | | ingredient 2) | |
| Al salt 3) | 2.0-8.0 | Al salt 3) | 2.0-8.0 | Al salt 3) | 2.0-8.0 |
| KM | 0.05-2.0 | KM | 0.05-2.0 | KM | 0.05-2.0 |
| ZK153 | 0.1-5.0 | ZK154 | 0.1-5.0 | ZK155 | 0.1-5.0 |
| Carrier 1) | to make | Carrier 1) | to make | Carrier 1) | to make |
| | 100 | | 100 | | 100 |
| | AF475 | | AF476 | | AF477 |
| Deodorant | 0.1-2.0 | Deodorant | 0.1-2.0 | Deodorant | 0.1-2.0 |
| active | | active | | active | |
| ingredient 2) | | ingredient 2) | | ingredient 2) | |
| Al salt 3) | 2.0-8.0 | Al salt 3) | 2.0-8.0 | Al salt 3) | 2.0-8.0 |
| KM | 0.05-2.0 | KM | 0.05-2.0 | KM | 0.05-2.0 |
| ZK156 | 0.1-5.0 | ZK157 | 0.1-5.0 | ZK158 | 0.1-5.0 |
| Carrier 1) | to make | Carrier 1) | to make | Carrier 1) | to make |
| | 100 | | 100 | | 100 |
| | AF478 | | AF479 | | AF480 |
| Deodorant | 0.1-2.0 | Deodorant | 0.1-2.0 | Deodorant | 0.1-2.0 |
| active | | active | | active | |
| ingredient 2) | | ingredient 2) | | ingredient 2) | |
| Al salt 3) | 2.0-8.0 | Al salt 3) | 2.0-8.0 | Al salt 3) | 2.0-8.0 |
| KM | 0.05-2.0 | KM | 0.05-2.0 | KM | 0.05-2.0 |
| ZK159 | 0.1-5.0 | ZK160 | 0.1-5.0 | ZK161 | 0.1-5.0 |
| Carrier 1) | to make | Carrier 1) | to make | Carrier 1) | to make |
| | 100 | | 100 | | 100 |
| | AF481 | | AF482 | | AF483 |
| Deodorant | 0.1-2.0 | Deodorant | 0.1-2.0 | Deodorant | 0.1-2.0 |
| active | | active | | active | |
| ingredient 2) | | ingredient 2) | | ingredient 2) | |
| Al salt 3) | 2.0-8.0 | Al salt 3) | 2.0-8.0 | Al salt 3) | 2.0-8.0 |
| KM | 0.05-2.0 | KM | 0.05-2.0 | KM | 0.05-2.0 |
| ZK162 | 0.1-5.0 | ZK163 | 0.1-5.0 | ZK164 | 0.1-5.0 |
| Carrier 1) | to make | Carrier 1) | to make | Carrier 1) | to make |
| | 100 | | 100 | | 100 |
| | AF484 | | AF485 | | AF486 |
| Deodorant | 0.1-2.0 | Deodorant | 0.1-2.0 | Deodorant | 0.1-2.0 |
| active | | active | | active | |
| ingredient 2) | | ingredient 2) | | ingredient 2) | |
| Al salt 3) | 2.0-8.0 | Al salt 3) | 2.0-8.0 | Al salt 3) | 2.0-8.0 |
| KM | 0.05-2.0 | KM | 0.05-2.0 | KM | 0.05-2.0 |
| ZK165 | 0.1-5.0 | ZK166 | 0.1-5.0 | ZK167 | 0.1-5.0 |
| Carrier 1) | to make | Carrier 1) | to make | Carrier 1) | to make |
| | 100 | | 100 | | 100 |
| | AF487 | | AF488 | | AF489 |
| Deodorant | 0.1-2.0 | Deodorant | 0.1-2.0 | Deodorant | 0.1-2.0 |
| active | | active | | active | |
| ingredient 2) | | ingredient 2) | | ingredient 2) | |
| Al salt 3) | 2.0-8.0 | Al salt 3) | 2.0-8.0 | Al salt 3) | 2.0-8.0 |
| KM | 0.05-2.0 | KM | 0.05-2.0 | KM | 0.05-2.0 |
| ZK168 | 0.1-5.0 | ZK169 | 0.1-5.0 | ZK170 | 0.1-5.0 |
| Carrier 1) | to make | Carrier 1) | to make | Carrier 1) | to make |
| | 100 | | 100 | | 100 |
| | AF490 | | AF491 | | AF492 |
| Deodorant | 0.1-2.0 | Deodorant | 0.1-2.0 | Deodorant | 0.1-2.0 |
| active | | active | | active | |
| ingredient 2) | | ingredient 2) | | ingredient 2) | |
| Al salt 3) | 2.0-8.0 | Al salt 3) | 2.0-8.0 | Al salt 3) | 2.0-8.0 |
| KM | 0.05-2.0 | KM | 0.05-2.0 | KM | 0.05-2.0 |
| ZK171 | 0.1-5.0 | ZK172 | 0.1-5.0 | ZK173 | 0.1-5.0 |
| Carrier 1) | to make | Carrier 1) | to make | Carrier 1) | to make |
| | 100 | | 100 | | 100 |
| | AF493 | | AF494 | | AF495 |
| Deodorant | 0.1-2.0 | Deodorant | 0.1-2.0 | Deodorant | 0.1-2.0 |
| active | | active | | active | |
| ingredient 2) | | ingredient 2) | | ingredient 2) | |
| Al salt 3) | 2.0-8.0 | Al salt 3) | 2.0-8.0 | Al salt 3) | 2.0-8.0 |
| KM | 0.05-2.0 | KM | 0.05-2.0 | KM | 0.05-2.0 |
| ZK174 | 0.1-5.0 | ZK175 | 0.1-5.0 | ZK176 | 0.1-5.0 |
| Carrier 1) | to make | Carrier 1) | to make | Carrier 1) | to make |
| | 100 | | 100 | | 100 |
| | AF496 | | AF497 | | AF498 |
| Deodorant | 0.1-2.0 | Deodorant | 0.1-2.0 | Deodorant | 0.1-2.0 |
| active | | active | | active | |
| ingredient 2) | | ingredient 2) | | ingredient 2) | |
| Al salt 3) | 2.0-8.0 | Al salt 3) | 2.0-8.0 | Al salt 3) | 2.0-8.0 |
| KM | 0.05-2.0 | KM | 0.05-2.0 | KM | 0.05-2.0 |
| ZK177 | 0.1-5.0 | ZK178 | 0.1-5.0 | ZK179 | 0.1-5.0 |
| Carrier 1) | to make | Carrier 1) | to make | Carrier 1) | to make |
| | 100 | | 100 | | 100 |
| | AF499 | | AF500 | | AF501 |
| Deodorant | 0.1-2.0 | Deodorant | 0.1-2.0 | Deodorant | 0.1-2.0 |
| active | | active | | active | |
| ingredient 2) | | ingredient 2) | | ingredient 2) | |
| Al salt 3) | 2.0-8.0 | Al salt 3) | 2.0-8.0 | Al salt 3) | 2.0-8.0 |
| KM | 0.05-2.0 | KM | 0.05-2.0 | KM | 0.05-2.0 |
| ZK180 | 0.1-5.0 | ZK181 | 0.1-5.0 | ZK182 | 0.1-5.0 |
| Carrier 1) | to make | Carrier 1) | to make | Carrier 1) | to make |
| | 100 | | 100 | | 100 |

1) aqueous, aqueous-alcoholic or anhydrous carrier,
2) selected from mixture comprising 1,2-hexanediol and 1,2-octanediol,
3) aluminum chlorohydrate,
4) aluminum-zirconium tetrachlorohydrate, stabilized with glycine

TABLE 3
Particularly preferred cosmetic agents (information in wt.
%, based on the total weight of the propellant-free agent)

| | AF1 | | AF2 | | AF3 |
|---|---|---|---|---|---|
| Deodorant | 0.1-2.0 | Deodorant | 0.1-2.0 | Deodorant | 0.1-2.0 |
| active | | active | | active | |
| ingredient | | ingredient | | ingredient | |
| KM | 0.05-2.0 | KM | 0.05-2.0 | KM | 0.05-2.0 |
| ZK183 | 0.1-5.0 | ZK184 | 0.1-5.0 | ZK185 | 0.1-5.0 |
| Carrier 1) | to make | Carrier 1) | to make | Carrier 1) | to make |
| | 100 | | 100 | | 100 |
| | AF4 | | AF5 | | AF6 |
| Deodorant | 0.1-2.0 | Deodorant | 0.1-2.0 | Deodorant | 0.1-2.0 |
| active | | active | | active | |
| ingredient | | ingredient | | ingredient | |
| KM | 0.05-2.0 | KM | 0.05-2.0 | KM | 0.05-2.0 |
| ZK186 | 0.1-5.0 | ZK187 | 0.1-5.0 | ZK188 | 0.1-5.0 |
| Carrier 1) | to make | Carrier 1) | to make | Carrier 1) | to make |
| | 100 | | 100 | | 100 |
| | AF7 | | AF8 | | AF9 |
| Deodorant | 0.1-2.0 | Deodorant | 0.1-2.0 | Deodorant | 0.1-2.0 |
| active | | active | | active | |
| ingredient | | ingredient | | ingredient | |
| KM | 0.05-2.0 | KM | 0.05-2.0 | KM | 0.05-2.0 |
| ZK189 | 0.1-5.0 | ZK190 | 0.1-5.0 | ZK191 | 0.1-5.0 |
| Carrier 1) | to make | Carrier 1) | to make | Carrier 1) | to make |
| | 100 | | 100 | | 100 |
| | AF10 | | AF11 | | AF12 |
| Deodorant | 0.1-2.0 | Deodorant | 0.1-2.0 | Deodorant | 0.1-2.0 |
| active | | active | | active | |
| ingredient | | ingredient | | ingredient | |
| KM | 0.05-2.0 | KM | 0.05-2.0 | KM | 0.05-2.0 |
| ZK192 | 0.1-5.0 | ZK193 | 0.1-5.0 | ZK194 | 0.1-5.0 |
| Carrier 1) | to make | Carrier 1) | to make | Carrier 1) | to make |
| | 100 | | 100 | | 100 |
| | AF13 | | AF14 | | AF15 |
| Deodorant | 0.1-2.0 | Deodorant | 0.1-2.0 | Deodorant | 0.1-2.0 |
| active | | active | | active | |
| ingredient | | ingredient | | ingredient | |
| KM | 0.05-2.0 | KM | 0.05-2.0 | KM | 0.05-2.0 |
| ZK195 | 0.1-5.0 | ZK196 | 0.1-5.0 | ZK197 | 0.1-5.0 |
| Carrier 1) | to make | Carrier 1) | to make | Carrier 1) | to make |
| | 100 | | 100 | | 100 |
| | AF16 | | AF17 | | AF18 |
| Deodorant | 0.1-2.0 | Deodorant | 0.1-2.0 | Deodorant | 0.1-2.0 |
| active | | active | | active | |
| ingredient | | ingredient | | ingredient | |
| KM | 0.05-2.0 | KM | 0.05-2.0 | KM | 0.05-2.0 |
| ZK198 | 0.1-5.0 | ZK199 | 0.1-5.0 | ZK200 | 0.1-5.0 |
| Carrier 1) | to make | Carrier 1) | to make | Carrier 1) | to make |
| | 100 | | 100 | | 100 |
| | AF19 | | AF20 | | AF21 |
| Deodorant | 0.1-2.0 | Deodorant | 0.1-2.0 | Deodorant | 0.1-2.0 |
| active | | active | | active | |
| ingredient | | ingredient | | ingredient | |
| KM | 0.05-2.0 | KM | 0.05-2.0 | KM | 0.05-2.0 |
| ZK201 | 0.1-5.0 | ZK202 | 0.1-5.0 | ZK203 | 0.1-5.0 |
| Carrier 1) | to make | Carrier 1) | to make | Carrier 1) | to make |
| | 100 | | 100 | | 100 |
| | AF22 | | AF23 | | AF24 |
| Deodorant | 0.1-2.0 | Deodorant | 0.1-2.0 | Deodorant | 0.1-2.0 |
| active | | active | | active | |
| ingredient | | ingredient | | ingredient | |
| KM | 0.05-2.0 | KM | 0.05-2.0 | KM | 0.05-2.0 |
| ZK204 | 0.1-5.0 | ZK205 | 0.1-5.0 | ZK206 | 0.1-5.0 |
| Carrier 1) | to make | Carrier 1) | to make | Carrier 1) | to make |
| | 100 | | 100 | | 100 |
| | AF25 | | AF26 | | AF27 |
| Deodorant | 0.1-2.0 | Deodorant | 0.1-2.0 | Deodorant | 0.1-2.0 |
| active | | active | | active | |
| ingredient | | ingredient | | ingredient | |
| KM | 0.05-2.0 | KM | 0.05-2.0 | KM | 0.05-2.0 |
| ZK207 | 0.1-5.0 | ZK208 | 0.1-5.0 | ZK209 | 0.1-5.0 |
| Carrier 1) | to make | Carrier 1) | to make | Carrier 1) | to make |
| | 100 | | 100 | | 100 |
| | AF28 | | AF29 | | AF30 |
| Deodorant | 0.1-2.0 | Deodorant | 0.1-2.0 | Deodorant | 0.1-2.0 |
| active | | active | | active | |
| ingredient | | ingredient | | ingredient | |
| KM | 0.05-2.0 | KM | 0.05-2.0 | KM | 0.05-2.0 |
| ZK210 | 0.1-5.0 | ZK211 | 0.1-5.0 | ZK212 | 0.1-5.0 |
| Carrier 1) | to make | Carrier 1) | to make | Carrier 1) | to make |
| | 100 | | 100 | | 100 |
| | AF31 | | AF32 | | AF33 |
| Deodorant | 0.1-2.0 | Deodorant | 0.1-2.0 | Deodorant | 0.1-2.0 |
| active | | active | | active | |
| ingredient | | ingredient | | ingredient | |
| KM | 0.05-2.0 | KM | 0.05-2.0 | KM | 0.05-2.0 |
| ZK213 | 0.1-5.0 | ZK214 | 0.1-5.0 | ZK215 | 0.1-5.0 |
| Carrier 1) | to make | Carrier 1) | to make | Carrier 1) | to make |
| | 100 | | 100 | | 100 |
| | AF34 | | AF35 | | AF36 |
| Deodorant | 0.1-2.0 | Deodorant | 0.1-2.0 | Deodorant | 0.1-2.0 |
| active | | active | | active | |
| ingredient | | ingredient | | ingredient | |
| KM | 0.05-2.0 | KM | 0.05-2.0 | KM | 0.05-2.0 |
| ZK216 | 0.1-5.0 | ZK217 | 0.1-5.0 | ZK218 | 0.1-5.0 |
| Carrier 1) | to make | Carrier 1) | to make | Carrier 1) | to make |
| | 100 | | 100 | | 100 |
| | AF37 | | AF38 | | AF39 |
| Deodorant | 0.1-2.0 | Deodorant | 0.1-2.0 | Deodorant | 0.1-2.0 |
| active | | active | | active | |
| ingredient | | ingredient | | ingredient | |
| KM | 0.05-2.0 | KM | 0.05-2.0 | KM | 0.05-2.0 |
| ZK219 | 0.1-5.0 | ZK220 | 0.1-5.0 | ZK221 | 0.1-5.0 |
| Carrier 1) | to make | Carrier 1) | to make | Carrier 1) | to make |
| | 100 | | 100 | | 100 |
| | AF40 | | AF41 | | AF42 |
| Deodorant | 0.1-2.0 | Deodorant | 0.1-2.0 | Deodorant | 0.1-2.0 |
| active | | active | | active | |
| ingredient | | ingredient | | ingredient | |
| KM | 0.05-2.0 | KM | 0.05-2.0 | KM | 0.05-2.0 |
| ZK222 | 0.1-5.0 | ZK223 | 0.1-5.0 | ZK224 | 0.1-5.0 |
| Carrier 1) | to make | Carrier 1) | to make | Carrier 1) | to make |
| | 100 | | 100 | | 100 |
| | AF43 | | AF44 | | AF45 |
| Deodorant | 0.1-2.0 | Deodorant | 0.1-2.0 | Deodorant | 0.1-2.0 |
| active | | active | | active | |
| ingredient | | ingredient | | ingredient | |
| KM | 0.05-2.0 | KM | 0.05-2.0 | KM | 0.05-2.0 |
| ZK225 | 0.1-5.0 | ZK226 | 0.1-5.0 | ZK227 | 0.1-5.0 |
| Carrier 1) | to make | Carrier 1) | to make | Carrier 1) | to make |
| | 100 | | 100 | | 100 |
| | AF46 | | AF47 | | AF48 |
| Deodorant | 0.1-2.0 | Deodorant | 0.1-2.0 | Deodorant | 0.1-2.0 |
| active | | active | | active | |
| ingredient | | ingredient | | ingredient | |
| KM | 0.05-2.0 | KM | 0.05-2.0 | KM | 0.05-2.0 |
| ZK228 | 0.1-5.0 | ZK229 | 0.1-5.0 | ZK230 | 0.1-5.0 |
| Carrier 1) | to make | Carrier 1) | to make | Carrier 1) | to make |
| | 100 | | 100 | | 100 |
| | AF49 | | AF50 | | AF51 |
| Deodorant | 0.1-2.0 | Deodorant | 0.1-2.0 | Deodorant | 0.1-2.0 |
| active | | active | | active | |
| ingredient | | ingredient | | ingredient | |
| KM | 0.05-2.0 | KM | 0.05-2.0 | KM | 0.05-2.0 |
| ZK231 | 0.1-5.0 | ZK232 | 0.1-5.0 | ZK233 | 0.1-5.0 |
| Carrier 1) | to make | Carrier 1) | to make | Carrier 1) | to make |
| | 100 | | 100 | | 100 |
| | AF52 | | AF53 | | AF54 |
| Deodorant | 0.1-2.0 | Deodorant | 0.1-2.0 | Deodorant | 0.1-2.0 |
| active | | active | | active | |
| ingredient | | ingredient | | ingredient | |
| KM | 0.05-2.0 | KM | 0.05-2.0 | KM | 0.05-2.0 |
| ZK234 | 0.1-5.0 | ZK235 | 0.1-5.0 | ZK236 | 0.1-5.0 |
| Carrier 1) | to make | Carrier 1) | to make | Carrier 1) | to make |
| | 100 | | 100 | | 100 |
| | AF55 | | AF59 | | AF57 |
| Deodorant | 0.1-2.0 | Deodorant | 0.1-2.0 | Deodorant | 0.1-2.0 |
| active | | active | | active | |
| ingredient | | ingredient | | ingredient | |
| KM | 0.05-2.0 | KM | 0.05-2.0 | KM | 0.05-2.0 |
| ZK237 | 0.1-5.0 | ZK238 | 0.1-5.0 | ZK239 | 0.1-5.0 |
| Carrier 1) | to make | Carrier 1) | to make | Carrier 1) | to make |
| | 100 | | 100 | | 100 |
| | AF58 | | AF59 | | AF60 |
| Deodorant | 0.1-2.0 | Deodorant | 0.1-2.0 | Deodorant | 0.1-2.0 |
| active | | active | | active | |
| ingredient | | ingredient | | ingredient | |
| KM | 0.05-2.0 | KM | 0.05-2.0 | KM | 0.05-2.0 |
| ZK240 | 0.1-5.0 | ZK241 | 0.1-5.0 | ZK242 | 0.1-5.0 |
| Carrier 1) | to make | Carrier 1) | to make | Carrier 1) | to make |
| | 100 | | 100 | | 100 |
| | AF61 | | AF62 | | AF63 |
| Deodorant | 0.1-2.0 | Deodorant | 0.1-2.0 | Deodorant | 0.1-2.0 |
| active | | active | | active | |
| ingredient | | ingredient | | ingredient | |
| KM | 0.05-2.0 | KM | 0.05-2.0 | KM | 0.05-2.0 |
| ZK243 | 0.1-5.0 | ZK244 | 0.1-5.0 | ZK245 | 0.1-5.0 |
| Carrier 1) | to make | Carrier 1) | to make | Carrier 1) | to make |
| | 100 | | 100 | | 100 |
| | AF64 | | AF65 | | AF66 |
| Deodorant | 0.1-2.0 | Deodorant | 0.1-2.0 | Deodorant | 0.1-2.0 |
| active | | active | | active | |
| ingredient | | ingredient | | ingredient | |
| KM | 0.05-2.0 | KM | 0.05-2.0 | KM | 0.05-2.0 |
| ZK246 | 0.1-5.0 | ZK247 | 0.1-5.0 | ZK248 | 0.1-5.0 |
| Carrier 1) | to make | Carrier 1) | to make | Carrier 1) | to make |
| | 100 | | 100 | | 100 |
| | AF67 | | AF68 | | AF69 |
| Deodorant | 0.1-2.0 | Deodorant | 0.1-2.0 | Deodorant | 0.1-2.0 |
| active | | active | | active | |
| ingredient | | ingredient | | ingredient | |
| KM | 0.05-2.0 | KM | 0.05-2.0 | KM | 0.05-2.0 |
| ZK249 | 0.1-5.0 | ZK250 | 0.1-5.0 | ZK251 | 0.1-5.0 |
| Carrier 1) | to make | Carrier 1) | to make | Carrier 1) | to make |
| | 100 | | 100 | | 100 |
| | AF70 | | AF71 | | AF72 |
| Deodorant | 0.1-2.0 | Deodorant | 0.1-2.0 | Deodorant | 0.1-2.0 |
| active | | active | | active | |
| ingredient | | ingredient | | ingredient | |
| KM | 0.05-2.0 | KM | 0.05-2.0 | KM | 0.05-2.0 |
| ZK252 | 0.1-5.0 | ZK253 | 0.1-5.0 | ZK254 | 0.1-5.0 |
| Carrier 1) | to make | Carrier 1) | to make | Carrier 1) | to make |
| | 100 | | 100 | | 100 |
| | AF73 | | AF74 | | AF75 |
| Deodorant | 0.1-2.0 | Deodorant | 0.1-2.0 | Deodorant | 0.1-2.0 |
| active | | active | | active | |
| ingredient | | ingredient | | ingredient | |
| KM | 0.05-2.0 | KM | 0.05-2.0 | KM | 0.05-2.0 |
| ZK255 | 0.1-5.0 | ZK256 | 0.1-5.0 | ZK257 | 0.1-5.0 |
| Carrier 1) | to make | Carrier 1) | to make | Carrier 1) | to make |
| | 100 | | 100 | | 100 |
| | AF76 | | AF77 | | AF78 |
| Deodorant | 0.1-2.0 | Deodorant | 0.1-2.0 | Deodorant | 0.1-2.0 |
| active | | active | | active | |
| ingredient | | ingredient | | ingredient | |
| KM | 0.05-2.0 | KM | 0.05-2.0 | KM | 0.05-2.0 |
| ZK258 | 0.1-5.0 | ZK259 | 0.1-5.0 | ZK260 | 0.1-5.0 |
| Carrier 1) | to make | Carrier 1) | to make | Carrier 1) | to make |
| | 100 | | 100 | | 100 |
| | AF79 | | AF80 | | AF81 |
| Deodorant | 0.1-2.0 | Deodorant | 0.1-2.0 | Deodorant | 0.1-2.0 |
| active | | active | | active | |
| ingredient | | ingredient | | ingredient | |
| KM | 0.05-2.0 | KM | 0.05-2.0 | KM | 0.05-2.0 |
| ZK261 | 0.1-5.0 | ZK262 | 0.1-5.0 | ZK263 | 0.1-5.0 |
| Carrier 1) | to make | Carrier 1) | to make | Carrier 1) | to make |
| | 100 | | 100 | | 100 |
| | AF82 | | AF83 | | AF84 |
| Deodorant | 0.1-2.0 | Deodorant | 0.1-2.0 | Deodorant | 0.1-2.0 |
| active | | active | | active | |
| ingredient | | ingredient | | ingredient | |
| KM | 0.05-2.0 | KM | 0.05-2.0 | KM | 0.05-2.0 |
| ZK264 | 0.1-5.0 | ZK265 | 0.1-5.0 | ZK266 | 0.1-5.0 |
| Carrier 1) | to make | Carrier 1) | to make | Carrier 1) | to make |
| | 100 | | 100 | | 100 |
| KM | 0.1-7.0 | KM | 0.1-7.0 | KM | 0.1-7.0 |
| | AF85 | | AF86 | | AF87 |
| Al salt | 2.0-8.0 | Al salt | 2.0-8.0 | Al salt | 2.0-8.0 |
| KM | 0.05-2.0 | KM | 0.05-2.0 | KM | 0.05-2.0 |
| ZK183 | 0.1-5.0 | ZK184 | 0.1-5.0 | ZK185 | 0.1-5.0 |
| Carrier 1) | to make | Carrier 1) | to make | Carrier 1) | to make |
| | 100 | | 100 | | 100 |
| | AF88 | | AF89 | | AF90 |
| Al salt | 2.0-8.0 | Al salt | 2.0-8.0 | Al salt | 2.0-8.0 |
| KM | 0.05-2.0 | KM | 0.05-2.0 | KM | 0.05-2.0 |
| ZK186 | 0.1-5.0 | ZK187 | 0.1-5.0 | ZK188 | 0.1-5.0 |
| Carrier 1) | to make | Carrier 1) | to make | Carrier 1) | to make |
| | 100 | | 100 | | 100 |
| | AF91 | | AF92 | | AF93 |
| Al salt | 2.0-8.0 | Al salt | 2.0-8.0 | Al salt | 2.0-8.0 |
| KM | 0.05-2.0 | KM | 0.05-2.0 | KM | 0.05-2.0 |
| ZK189 | 0.1-5.0 | ZK190 | 0.1-5.0 | ZK191 | 0.1-5.0 |
| Carrier 1) | to make | Carrier 1) | to make | Carrier 1) | to make |
| | 100 | | 100 | | 100 |
| | AF94 | | AF95 | | AF96 |
| Al salt | 2.0-8.0 | Al salt | 2.0-8.0 | Al salt | 2.0-8.0 |
| KM | 0.05-2.0 | KM | 0.05-2.0 | KM | 0.05-2.0 |
| ZK192 | 0.1-5.0 | ZK193 | 0.1-5.0 | ZK194 | 0.1-5.0 |
| Carrier 1) | to make | Carrier 1) | to make | Carrier 1) | to make |
| | 100 | | 100 | | 100 |
| | AF97 | | AF98 | | AF99 |
| Al salt | 2.0-8.0 | Al salt | 2.0-8.0 | Al salt | 2.0-8.0 |
| KM | 0.05-2.0 | KM | 0.05-2.0 | KM | 0.05-2.0 |
| ZK195 | 0.1-5.0 | ZK196 | 0.1-5.0 | ZK197 | 0.1-5.0 |
| Carrier 1) | to make | Carrier 1) | to make | Carrier 1) | to make |
| | 100 | | 100 | | 100 |
| | AF100 | | AF101 | | AF102 |
| Al salt | 2.0-8.0 | Al salt | 2.0-8.0 | Al salt | 2.0-8.0 |
| KM | 0.05-2.0 | KM | 0.05-2.0 | KM | 0.05-2.0 |
| ZK198 | 0.1-5.0 | ZK199 | 0.1-5.0 | ZK200 | 0.1-5.0 |
| Carrier 1) | to make | Carrier 1) | to make | Carrier 1) | to make |
| | 100 | | 100 | | 100 |
| | AF103 | | AF104 | | AF105 |
| Al salt | 2.0-8.0 | Al salt | 2.0-8.0 | Al salt | 2.0-8.0 |
| KM | 0.05-2.0 | KM | 0.05-2.0 | KM | 0.05-2.0 |
| ZK201 | 0.1-5.0 | ZK202 | 0.1-5.0 | ZK203 | 0.1-5.0 |
| Carrier 1) | to make | Carrier 1) | to make | Carrier 1) | to make |
| | 100 | | 100 | | 100 |
| | AF106 | | AF107 | | AF108 |
| Al salt | 2.0-8.0 | Al salt | 2.0-8.0 | Al salt | 2.0-8.0 |
| KM | 0.05-2.0 | KM | 0.05-2.0 | KM | 0.05-2.0 |
| ZK204 | 0.1-5.0 | ZK205 | 0.1-5.0 | ZK206 | 0.1-5.0 |
| Carrier 1) | to make | Carrier 1) | to make | Carrier 1) | to make |
| | 100 | | 100 | | 100 |
| | AF109 | | AF110 | | AF111 |
| Al salt | 2.0-8.0 | Al salt | 2.0-8.0 | Al salt | 2.0-8.0 |
| KM | 0.05-2.0 | KM | 0.05-2.0 | KM | 0.05-2.0 |
| ZK207 | 0.1-5.0 | ZK208 | 0.1-5.0 | ZK209 | 0.1-5.0 |
| Carrier 1) | to make | Carrier 1) | to make | Carrier 1) | to make |
| | 100 | | 100 | | 100 |
| | AF112 | | AF113 | | AF114 |
| Al salt | 2.0-8.0 | Al salt | 2.0-8.0 | Al salt | 2.0-8.0 |
| KM | 0.05-2.0 | KM | 0.05-2.0 | KM | 0.05-2.0 |
| ZK210 | 0.1-5.0 | ZK211 | 0.1-5.0 | ZK212 | 0.1-5.0 |
| Carrier 1) | to make | Carrier 1) | to make | Carrier 1) | to make |
| | 100 | | 100 | | 100 |
| | AF115 | | AF116 | | AF117 |
| Al salt | 2.0-8.0 | Al salt | 2.0-8.0 | Al salt | 2.0-8.0 |
| KM | 0.05-2.0 | KM | 0.05-2.0 | KM | 0.05-2.0 |
| ZK213 | 0.1-5.0 | ZK214 | 0.1-5.0 | ZK215 | 0.1-5.0 |
| Carrier 1) | to make | Carrier 1) | to make | Carrier 1) | to make |
| | 100 | | 100 | | 100 |
| | AF118 | | AF119 | | AF120 |
| Al salt | 2.0-8.0 | Al salt | 2.0-8.0 | Al salt | 2.0-8.0 |
| KM | 0.05-2.0 | KM | 0.05-2.0 | KM | 0.05-2.0 |
| ZK216 | 0.1-5.0 | ZK217 | 0.1-5.0 | ZK218 | 0.1-5.0 |
| Carrier 1) | to make | Carrier 1) | to make | Carrier 1) | to make |
| | 100 | | 100 | | 100 |
| | AF121 | | AF122 | | AF123 |
| Al salt | 2.0-8.0 | Al salt | 2.0-8.0 | Al salt | 2.0-8.0 |
| KM | 0.05-2.0 | KM | 0.05-2.0 | KM | 0.05-2.0 |
| ZK219 | 0.1-5.0 | ZK220 | 0.1-5.0 | ZK221 | 0.1-5.0 |
| Carrier 1) | to make | Carrier 1) | to make | Carrier 1) | to make |
| | 100 | | 100 | | 100 |
| | AF124 | | AF125 | | AF126 |
| Al salt | 2.0-8.0 | Al salt | 2.0-8.0 | Al salt | 2.0-8.0 |
| KM | 0.05-2.0 | KM | 0.05-2.0 | KM | 0.05-2.0 |
| ZK222 | 0.1-5.0 | ZK223 | 0.1-5.0 | ZK224 | 0.1-5.0 |
| Carrier 1) | to make | Carrier 1) | to make | Carrier 1) | to make |
| | 100 | | 100 | | 100 |
| | AF127 | | AF128 | | AF129 |
| Al salt | 2.0-8.0 | Al salt | 2.0-8.0 | Al salt | 2.0-8.0 |
| KM | 0.05-2.0 | KM | 0.05-2.0 | KM | 0.05-2.0 |
| ZK225 | 0.1-5.0 | ZK226 | 0.1-5.0 | ZK227 | 0.1-5.0 |
| Carrier 1) | to make | Carrier 1) | to make | Carrier 1) | to make |
| | 100 | | 100 | | 100 |
| | AF130 | | AF131 | | AF132 |
| Al salt | 2.0-8.0 | Al salt | 2.0-8.0 | Al salt | 2.0-8.0 |
| KM | 0.05-2.0 | KM | 0.05-2.0 | KM | 0.05-2.0 |
| ZK228 | 0.1-5.0 | ZK229 | 0.1-5.0 | ZK230 | 0.1-5.0 |
| Carrier 1) | to make | Carrier 1) | to make | Carrier 1) | to make |
| | 100 | | 100 | | 100 |
| | AF133 | | AF134 | | AF135 |
| Al salt | 2.0-8.0 | Al salt | 2.0-8.0 | Al salt | 2.0-8.0 |
| KM | 0.05-2.0 | KM | 0.05-2.0 | KM | 0.05-2.0 |
| ZK231 | 0.1-5.0 | ZK232 | 0.1-5.0 | ZK233 | 0.1-5.0 |
| Carrier 1) | to make | Carrier 1) | to make | Carrier 1) | to make |
| | 100 | | 100 | | 100 |
| | AF136 | | AF137 | | AF138 |
| Al salt | 2.0-8.0 | Al salt | 2.0-8.0 | Al salt | 2.0-8.0 |
| KM | 0.05-2.0 | KM | 0.05-2.0 | KM | 0.05-2.0 |
| ZK234 | 0.1-5.0 | ZK235 | 0.1-5.0 | ZK236 | 0.1-5.0 |
| Carrier 1) | to make | Carrier 1) | to make | Carrier 1) | to make |
| | 100 | | 100 | | 100 |
| | AF139 | | AF140 | | AF141 |
| Al salt | 2.0-8.0 | Al salt | 2.0-8.0 | Al salt | 2.0-8.0 |
| KM | 0.05-2.0 | KM | 0.05-2.0 | KM | 0.05-2.0 |
| ZK237 | 0.1-5.0 | ZK238 | 0.1-5.0 | ZK239 | 0.1-5.0 |
| Carrier 1) | to make | Carrier 1) | to make | Carrier 1) | to make |
| | 100 | | 100 | | 100 |
| | AF142 | | AF143 | | AF144 |
| Al salt | 2.0-8.0 | Al salt | 2.0-8.0 | Al salt | 2.0-8.0 |
| KM | 0.05-2.0 | KM | 0.05-2.0 | KM | 0.05-2.0 |
| ZK240 | 0.1-5.0 | ZK241 | 0.1-5.0 | ZK242 | 0.1-5.0 |
| Carrier 1) | to make | Carrier 1) | to make | Carrier 1) | to make |
| | 100 | | 100 | | 100 |
| | AF145 | | AF146 | | AF147 |
| Al salt | 2.0-8.0 | Al salt | 2.0-8.0 | Al salt | 2.0-8.0 |
| KM | 0.05-2.0 | KM | 0.05-2.0 | KM | 0.05-2.0 |
| ZK243 | 0.1-5.0 | ZK244 | 0.1-5.0 | ZK245 | 0.1-5.0 |
| Carrier 1) | to make | Carrier 1) | to make | Carrier 1) | to make |
| | 100 | | 100 | | 100 |
| | AF148 | | AF149 | | AF150 |
| Al salt | 2.0-8.0 | Al salt | 2.0-8.0 | Al salt | 2.0-8.0 |
| KM | 0.05-2.0 | KM | 0.05-2.0 | KM | 0.05-2.0 |
| ZK246 | 0.1-5.0 | ZK247 | 0.1-5.0 | ZK248 | 0.1-5.0 |
| Carrier 1) | to make | Carrier 1) | to make | Carrier 1) | to make |
| | 100 | | 100 | | 100 |
| | AF151 | | AF152 | | AF153 |
| Al salt | 2.0-8.0 | Al salt | 2.0-8.0 | Al salt | 2.0-8.0 |
| KM | 0.05-2.0 | KM | 0.05-2.0 | KM | 0.05-2.0 |
| ZK249 | 0.1-5.0 | ZK250 | 0.1-5.0 | ZK251 | 0.1-5.0 |
| Carrier 1) | to make | Carrier 1) | to make | Carrier 1) | to make |
| | 100 | | 100 | | 100 |
| | AF154 | | AF155 | | AF156 |
| Al salt | 2.0-8.0 | Al salt | 2.0-8.0 | Al salt | 2.0-8.0 |
| KM | 0.05-2.0 | KM | 0.05-2.0 | KM | 0.05-2.0 |
| ZK252 | 0.1-5.0 | ZK253 | 0.1-5.0 | ZK254 | 0.1-5.0 |
| Carrier 1) | to make | Carrier 1) | to make | Carrier 1) | to make |
| | 100 | | 100 | | 100 |
| | AF157 | | AF158 | | AF159 |
| Al salt | 2.0-8.0 | Al salt | 2.0-8.0 | Al salt | 2.0-8.0 |
| KM | 0.05-2.0 | KM | 0.05-2.0 | KM | 0.05-2.0 |
| ZK255 | 0.1-5.0 | ZK256 | 0.1-5.0 | ZK257 | 0.1-5.0 |
| Carrier 1) | to make | Carrier 1) | to make | Carrier 1) | to make |
| | 100 | | 100 | | 100 |
| | AF160 | | AF161 | | AF162 |
| Al salt | 2.0-8.0 | Al salt | 2.0-8.0 | Al salt | 2.0-8.0 |
| KM | 0.05-2.0 | KM | 0.05-2.0 | KM | 0.05-2.0 |
| ZK258 | 0.1-5.0 | ZK259 | 0.1-5.0 | ZK260 | 0.1-5.0 |
| Carrier 1) | to make | Carrier 1) | to make | Carrier 1) | to make |
| | 100 | | 100 | | 100 |
| | AF163 | | AF164 | | AF165 |
| Al salt | 2.0-8.0 | Al salt | 2.0-8.0 | Al salt | 2.0-8.0 |
| KM | 0.05-2.0 | KM | 0.05-2.0 | KM | 0.05-2.0 |
| ZK261 | 0.1-5.0 | ZK262 | 0.1-5.0 | ZK263 | 0.1-5.0 |
| Carrier 1) | to make | Carrier 1) | to make | Carrier 1) | to make |
| | 100 | | 100 | | 100 |
| | AF166 | | AF167 | | AF168 |
| Al salt | 2.0-8.0 | Al salt | 2.0-8.0 | Al salt | 2.0-8.0 |
| KM | 0.05-2.0 | KM | 0.05-2.0 | KM | 0.05-2.0 |
| ZK264 | 0.1-5.0 | ZK265 | 0.1-5.0 | ZK266 | 0.1-5.0 |
| Carrier 1) | to make | Carrier 1) | to make | Carrier 1) | to make |
| | 100 | | 100 | | 100 |
| | AF169 | | AF170 | | AF171 |
| Deodorant | 0.1-2.0 | Deodorant | 0.1-2.0 | Deodorant | 0.1-2.0 |
| active | | active | | active | |
| ingredient | | ingredient | | ingredient | |
| Al salt | 2.0-8.0 | Al salt | 2.0-8.0 | Al salt | 2.0-8.0 |
| KM | 0.05-2.0 | KM | 0.05-2.0 | KM | 0.05-2.0 |
| ZK183 | 0.1-5.0 | ZK184 | 0.1-5.0 | ZK185 | 0.1-5.0 |
| Carrier 1) | to make | Carrier 1) | to make | Carrier 1) | to make |
| | 100 | | 100 | | 100 |
| | AF172 | | AF173 | | AF174 |
| Deodorant | 0.1-2.0 | Deodorant | 0.1-2.0 | Deodorant | 0.1-2.0 |
| active | | active | | active | |
| ingredient | | ingredient | | ingredient | |
| Al salt | 2.0-8.0 | Al salt | 2.0-8.0 | Al salt | 2.0-8.0 |
| KM | 0.05-2.0 | KM | 0.05-2.0 | KM | 0.05-2.0 |
| ZK186 | 0.1-5.0 | ZK187 | 0.1-5.0 | ZK188 | 0.1-5.0 |
| Carrier 1) | to make | Carrier 1) | to make | Carrier 1) | to make |
| | 100 | | 100 | | 100 |
| | AF175 | | AF176 | | AF177 |
| Deodorant | 0.1-2.0 | Deodorant | 0.1-2.0 | Deodorant | 0.1-2.0 |
| active | | active | | active | |
| ingredient | | ingredient | | ingredient | |
| Al salt | 2.0-8.0 | Al salt | 2.0-8.0 | Al salt | 2.0-8.0 |
| KM | 0.05-2.0 | KM | 0.05-2.0 | KM | 0.05-2.0 |
| ZK189 | 0.1-5.0 | ZK190 | 0.1-5.0 | ZK191 | 0.1-5.0 |
| Carrier 1) | to make | Carrier 1) | to make | Carrier 1) | to make |
| | 100 | | 100 | | 100 |
| | AF178 | | AF179 | | AF180 |
| Deodorant | 0.1-2.0 | Deodorant | 0.1-2.0 | Deodorant | 0.1-2.0 |
| active | | active | | active | |
| ingredient | | ingredient | | ingredient | |
| Al salt | 2.0-8.0 | Al salt | 2.0-8.0 | Al salt | 2.0-8.0 |
| KM | 0.05-2.0 | KM | 0.05-2.0 | KM | 0.05-2.0 |
| ZK192 | 0.1-5.0 | ZK193 | 0.1-5.0 | ZK194 | 0.1-5.0 |
| Carrier 1) | to make | Carrier 1) | to make | Carrier 1) | to make |
| | 100 | | 100 | | 100 |
| | AF181 | | AF182 | | AF183 |
| Deodorant | 0.1-2.0 | Deodorant | 0.1-2.0 | Deodorant | 0.1-2.0 |
| active | | active | | active | |
| ingredient | | ingredient | | ingredient | |
| Al salt | 2.0-8.0 | Al salt | 2.0-8.0 | Al salt | 2.0-8.0 |
| KM | 0.05-2.0 | KM | 0.05-2.0 | KM | 0.05-2.0 |
| ZK195 | 0.1-5.0 | ZK196 | 0.1-5.0 | ZK197 | 0.1-5.0 |
| Carrier 1) | to make | Carrier 1) | to make | Carrier 1) | to make |
| | 100 | | 100 | | 100 |
| | AF184 | | AF185 | | AF186 |
| Deodorant | 0.1-2.0 | Deodorant | 0.1-2.0 | Deodorant | 0.1-2.0 |
| active | | active | | active | |
| ingredient | | ingredient | | ingredient | |
| Al salt | 2.0-8.0 | Al salt | 2.0-8.0 | Al salt | 2.0-8.0 |
| KM | 0.05-2.0 | KM | 0.05-2.0 | KM | 0.05-2.0 |
| ZK198 | 0.1-5.0 | ZK199 | 0.1-5.0 | ZK200 | 0.1-5.0 |
| Carrier 1) | to make | Carrier 1) | to make | Carrier 1) | to make |
| | 100 | | 100 | | 100 |
| | AF187 | | AF188 | | AF189 |
| Deodorant | 0.1-2.0 | Deodorant | 0.1-2.0 | Deodorant | 0.1-2.0 |
| active | | active | | active | |
| ingredient | | ingredient | | ingredient | |
| Al salt | 2.0-8.0 | Al salt | 2.0-8.0 | Al salt | 2.0-8.0 |
| KM | 0.05-2.0 | KM | 0.05-2.0 | KM | 0.05-2.0 |
| ZK201 | 0.1-5.0 | ZK202 | 0.1-5.0 | ZK203 | 0.1-5.0 |
| Carrier 1) | to make | Carrier 1) | to make | Carrier 1) | to make |
| | 100 | | 100 | | 100 |
| | AF190 | | AF191 | | AF192 |
| Deodorant | 0.1-2.0 | Deodorant | 0.1-2.0 | Deodorant | 0.1-2.0 |
| active | | active | | active | |
| ingredient | | ingredient | | ingredient | |
| Al salt | 2.0-8.0 | Al salt | 2.0-8.0 | Al salt | 2.0-8.0 |
| KM | 0.05-2.0 | KM | 0.05-2.0 | KM | 0.05-2.0 |
| ZK204 | 0.1-5.0 | ZK205 | 0.1-5.0 | ZK206 | 0.1-5.0 |
| Carrier 1) | to make | Carrier 1) | to make | Carrier 1) | to make |
| | 100 | | 100 | | 100 |
| | AF193 | | AF194 | | AF195 |
| Deodorant | 0.1-2.0 | Deodorant | 0.1-2.0 | Deodorant | 0.1-2.0 |
| active | | active | | active | |
| ingredient | | ingredient | | ingredient | |
| Al salt | 2.0-8.0 | Al salt | 2.0-8.0 | Al salt | 2.0-8.0 |
| KM | 0.05-2.0 | KM | 0.05-2.0 | KM | 0.05-2.0 |
| ZK207 | 0.1-5.0 | ZK208 | 0.1-5.0 | ZK209 | 0.1-5.0 |
| Carrier 1) | to make | Carrier 1) | to make | Carrier 1) | to make |
| | 100 | | 100 | | 100 |
| | AF196 | | AF197 | | AF198 |
| Deodorant | 0.1-2.0 | Deodorant | 0.1-2.0 | Deodorant | 0.1-2.0 |
| active | | active | | active | |
| ingredient | | ingredient | | ingredient | |
| Al salt | 2.0-8.0 | Al salt | 2.0-8.0 | Al salt | 2.0-8.0 |
| KM | 0.05-2.0 | KM | 0.05-2.0 | KM | 0.05-2.0 |
| ZK210 | 0.1-5.0 | ZK211 | 0.1-5.0 | ZK212 | 0.1-5.0 |
| Carrier 1) | to make | Carrier 1) | to make | Carrier 1) | to make |
| | 100 | | 100 | | 100 |
| | AF199 | | AF200 | | AF201 |
| Deodorant | 0.1-2.0 | Deodorant | 0.1-2.0 | Deodorant | 0.1-2.0 |
| active | | active | | active | |
| ingredient | | ingredient | | ingredient | |
| Al salt | 2.0-8.0 | Al salt | 2.0-8.0 | Al salt | 2.0-8.0 |
| KM | 0.05-2.0 | KM | 0.05-2.0 | KM | 0.05-2.0 |
| ZK213 | 0.1-5.0 | ZK214 | 0.1-5.0 | ZK215 | 0.1-5.0 |
| Carrier 1) | to make | Carrier 1) | to make | Carrier 1) | to make |
| | 100 | | 100 | | 100 |
| | AF202 | | AF203 | | AF204 |
| Deodorant | 0.1-2.0 | Deodorant | 0.1-2.0 | Deodorant | 0.1-2.0 |
| active | | active | | active | |
| ingredient | | ingredient | | ingredient | |
| Al salt | 2.0-8.0 | Al salt | 2.0-8.0 | Al salt | 2.0-8.0 |
| KM | 0.05-2.0 | KM | 0.05-2.0 | KM | 0.05-2.0 |
| ZK216 | 0.1-5.0 | ZK217 | 0.1-5.0 | ZK218 | 0.1-5.0 |
| Carrier 1) | to make | Carrier 1) | to make | Carrier 1) | to make |
| | 100 | | 100 | | 100 |
| | AF205 | | AF206 | | AF207 |
| Deodorant | 0.1-2.0 | Deodorant | 0.1-2.0 | Deodorant | 0.1-2.0 |
| active | | active | | active | |
| ingredient | | ingredient | | ingredient | |
| Al salt | 2.0-8.0 | Al salt | 2.0-8.0 | Al salt | 2.0-8.0 |
| KM | 0.05-2.0 | KM | 0.05-2.0 | KM | 0.05-2.0 |
| ZK219 | 0.1-5.0 | ZK220 | 0.1-5.0 | ZK221 | 0.1-5.0 |
| Carrier 1) | to make | Carrier 1) | to make | Carrier 1) | to make |
| | 100 | | 100 | | 100 |
| | AF208 | | AF209 | | AF210 |
| Deodorant | 0.1-2.0 | Deodorant | 0.1-2.0 | Deodorant | 0.1-2.0 |
| active | | active | | active | |
| ingredient | | ingredient | | ingredient | |
| Al salt | 2.0-8.0 | Al salt | 2.0-8.0 | Al salt | 2.0-8.0 |
| KM | 0.05-2.0 | KM | 0.05-2.0 | KM | 0.05-2.0 |
| ZK222 | 0.1-5.0 | ZK223 | 0.1-5.0 | ZK224 | 0.1-5.0 |
| Carrier 1) | to make | Carrier 1) | to make | Carrier 1) | to make |
| | 100 | | 100 | | 100 |
| | AF211 | | AF212 | | AF213 |
| Deodorant | 0.1-2.0 | Deodorant | 0.1-2.0 | Deodorant | 0.1-2.0 |
| active | | active | | active | |
| ingredient | | ingredient | | ingredient | |
| Al salt | 2.0-8.0 | Al salt | 2.0-8.0 | Al salt | 2.0-8.0 |
| KM | 0.05-2.0 | KM | 0.05-2.0 | KM | 0.05-2.0 |
| ZK225 | 0.1-5.0 | ZK226 | 0.1-5.0 | ZK227 | 0.1-5.0 |
| Carrier 1) | to make | Carrier 1) | to make | Carrier 1) | to make |
| | 100 | | 100 | | 100 |
| | AF214 | | AF215 | | AF216 |
| Deodorant | 0.1-2.0 | Deodorant | 0.1-2.0 | Deodorant | 0.1-2.0 |
| active | | active | | active | |
| ingredient | | ingredient | | ingredient | |
| Al salt | 2.0-8.0 | Al salt | 2.0-8.0 | Al salt | 2.0-8.0 |
| KM | 0.05-2.0 | KM | 0.05-2.0 | KM | 0.05-2.0 |
| ZK228 | 0.1-5.0 | ZK229 | 0.1-5.0 | ZK230 | 0.1-5.0 |
| Carrier 1) | to make | Carrier 1) | to make | Carrier 1) | to make |
| | 100 | | 100 | | 100 |
| | AF217 | | AF218 | | AF219 |
| Deodorant | 0.1-2.0 | Deodorant | 0.1-2.0 | Deodorant | 0.1-2.0 |
| active | | active | | active | |
| ingredient | | ingredient | | ingredient | |
| Al salt | 2.0-8.0 | Al salt | 2.0-8.0 | Al salt | 2.0-8.0 |
| KM | 0.05-2.0 | KM | 0.05-2.0 | KM | 0.05-2.0 |
| ZK231 | 0.1-5.0 | ZK232 | 0.1-5.0 | ZK233 | 0.1-5.0 |
| Carrier 1) | to make | Carrier 1) | to make | Carrier 1) | to make |
| | 100 | | 100 | | 100 |
| | AF220 | | AF221 | | AF222 |
| Deodorant | 0.1-2.0 | Deodorant | 0.1-2.0 | Deodorant | 0.1-2.0 |
| active | | active | | active | |
| ingredient | | ingredient | | ingredient | |
| Al salt | 2.0-8.0 | Al salt | 2.0-8.0 | Al salt | 2.0-8.0 |
| KM | 0.05-2.0 | KM | 0.05-2.0 | KM | 0.05-2.0 |
| ZK234 | 0.1-5.0 | ZK235 | 0.1-5.0 | ZK236 | 0.1-5.0 |
| Carrier 1) | to make | Carrier 1) | to make | Carrier 1) | to make |
| | 100 | | 100 | | 100 |
| | AF223 | | AF224 | | AF225 |
| Deodorant | 0.1-2.0 | Deodorant | 0.1-2.0 | Deodorant | 0.1-2.0 |
| active | | active | | active | |
| ingredient | | ingredient | | ingredient | |
| Al salt | 2.0-8.0 | Al salt | 2.0-8.0 | Al salt | 2.0-8.0 |
| KM | 0.05-2.0 | KM | 0.05-2.0 | KM | 0.05-2.0 |
| ZK237 | 0.1-5.0 | ZK238 | 0.1-5.0 | ZK239 | 0.1-5.0 |
| Carrier 1) | to make | Carrier 1) | to make | Carrier 1) | to make |
| | 100 | | 100 | | 100 |
| | AF226 | | AF227 | | AF228 |
| Deodorant | 0.1-2.0 | Deodorant | 0.1-2.0 | Deodorant | 0.1-2.0 |
| active | | active | | active | |
| ingredient | | ingredient | | ingredient | |
| Al salt | 2.0-8.0 | Al salt | 2.0-8.0 | Al salt | 2.0-8.0 |
| KM | 0.05-2.0 | KM | 0.05-2.0 | KM | 0.05-2.0 |
| ZK240 | 0.1-5.0 | ZK241 | 0.1-5.0 | ZK242 | 0.1-5.0 |
| Carrier 1) | to make | Carrier 1) | to make | Carrier 1) | to make |
| | 100 | | 100 | | 100 |
| | AF229 | | AF230 | | AF231 |
| Deodorant | 0.1-2.0 | Deodorant | 0.1-2.0 | Deodorant | 0.1-2.0 |
| active | | active | | active | |
| ingredient | | ingredient | | ingredient | |
| Al salt | 2.0-8.0 | Al salt | 2.0-8.0 | Al salt | 2.0-8.0 |
| KM | 0.05-2.0 | KM | 0.05-2.0 | KM | 0.05-2.0 |
| ZK243 | 0.1-5.0 | ZK244 | 0.1-5.0 | ZK245 | 0.1-5.0 |
| Carrier 1) | to make | Carrier 1) | to make | Carrier 1) | to make |
| | 100 | | 100 | | 100 |
| | AF232 | | AF233 | | AF234 |
| Deodorant | 0.1-2.0 | Deodorant | 0.1-2.0 | Deodorant | 0.1-2.0 |
| active | | active | | active | |
| ingredient | | ingredient | | ingredient | |
| Al salt | 2.0-8.0 | Al salt | 2.0-8.0 | Al salt | 2.0-8.0 |
| KM | 0.05-2.0 | KM | 0.05-2.0 | KM | 0.05-2.0 |
| ZK246 | 0.1-5.0 | ZK247 | 0.1-5.0 | ZK248 | 0.1-5.0 |
| Carrier 1) | to make | Carrier 1) | to make | Carrier 1) | to make |
| | 100 | | 100 | | 100 |
| | AF235 | | AF236 | | AF237 |
| Deodorant | 0.1-2.0 | Deodorant | 0.1-2.0 | Deodorant | 0.1-2.0 |
| active | | active | | active | |
| ingredient | | ingredient | | ingredient | |
| Al salt | 2.0-8.0 | Al salt | 2.0-8.0 | Al salt | 2.0-8.0 |
| KM | 0.05-2.0 | KM | 0.05-2.0 | KM | 0.05-2.0 |
| ZK249 | 0.1-5.0 | ZK250 | 0.1-5.0 | ZK251 | 0.1-5.0 |
| Carrier 1) | to make | Carrier 1) | to make | Carrier 1) | to make |
| | 100 | | 100 | | 100 |
| | AF238 | | AF239 | | AF240 |
| Deodorant | 0.1-2.0 | Deodorant | 0.1-2.0 | Deodorant | 0.1-2.0 |
| active | | active | | active | |
| ingredient | | ingredient | | ingredient | |
| Al salt | 2.0-8.0 | Al salt | 2.0-8.0 | Al salt | 2.0-8.0 |
| KM | 0.05-2.0 | KM | 0.05-2.0 | KM | 0.05-2.0 |
| ZK252 | 0.1-5.0 | ZK253 | 0.1-5.0 | ZK254 | 0.1-5.0 |
| Carrier 1) | to make | Carrier 1) | to make | Carrier 1) | to make |
| | 100 | | 100 | | 100 |
| | AF241 | | AF242 | | AF243 |
| Deodorant | 0.1-2.0 | Deodorant | 0.1-2.0 | Deodorant | 0.1-2.0 |
| active | | active | | active | |
| ingredient | | ingredient | | ingredient | |
| Al salt | 2.0-8.0 | Al salt | 2.0-8.0 | Al salt | 2.0-8.0 |
| KM | 0.05-2.0 | KM | 0.05-2.0 | KM | 0.05-2.0 |
| ZK255 | 0.1-5.0 | ZK256 | 0.1-5.0 | ZK257 | 0.1-5.0 |
| Carrier 1) | to make | Carrier 1) | to make | Carrier 1) | to make |
| | 100 | | 100 | | 100 |
| | AF244 | | AF245 | | AF246 |
| Deodorant | 0.1-2.0 | Deodorant | 0.1-2.0 | Deodorant | 0.1-2.0 |
| active | | active | | active | |
| ingredient | | ingredient | | ingredient | |
| Al salt | 2.0-8.0 | Al salt | 2.0-8.0 | Al salt | 2.0-8.0 |
| KM | 0.05-2.0 | KM | 0.05-2.0 | KM | 0.05-2.0 |
| ZK258 | 0.1-5.0 | ZK259 | 0.1-5.0 | ZK260 | 0.1-5.0 |
| Carrier 1) | to make | Carrier 1) | to make | Carrier 1) | to make |
| | 100 | | 100 | | 100 |
| | AF247 | | AF248 | | AF249 |
| Deodorant | 0.1-2.0 | Deodorant | 0.1-2.0 | Deodorant | 0.1-2.0 |
| active | | active | | active | |
| ingredient | | ingredient | | ingredient | |
| Al salt | 2.0-8.0 | Al salt | 2.0-8.0 | Al salt | 2.0-8.0 |
| KM | 0.05-2.0 | KM | 0.05-2.0 | KM | 0.05-2.0 |
| ZK261 | 0.1-5.0 | ZK262 | 0.1-5.0 | ZK263 | 0.1-5.0 |
| Carrier 1) | to make | Carrier 1) | to make | Carrier 1) | to make |
| | 100 | | 100 | | 100 |
| | AF250 | | AF251 | | AF252 |
| Deodorant | 0.1-2.0 | Deodorant | 0.1-2.0 | Deodorant | 0.1-2.0 |
| active | | active | | active | |
| ingredient | | ingredient | | ingredient | |
| Al salt | 2.0-8.0 | Al salt | 2.0-8.0 | Al salt | 2.0-8.0 |
| KM | 0.05-2.0 | KM | 0.05-2.0 | KM | 0.05-2.0 |
| ZK264 | 0.1-5.0 | ZK265 | 0.1-5.0 | ZK266 | 0.1-5.0 |
| Carrier 1) | to make | Carrier 1) | to make | Carrier 1) | to make |
| | 100 | | 100 | | 100 |
| KM | 0.1-7.0 | KM | 0.1-7.0 | KM | 0.1-7.0 |
| | AF253 | | AF254 | | AF255 |
| Deodorant | 0.1-2.0 | Deodorant | 0.1-2.0 | Deodorant | 0.1-2.0 |
| active | | active | | active | |
| ingredient 2) | | ingredient 2) | | ingredient 2) | |
| KM | 0.05-2.0 | KM | 0.05-2.0 | KM | 0.05-2.0 |
| ZK183 | 0.1-5.0 | ZK184 | 0.1-5.0 | ZK185 | 0.1-5.0 |
| Carrier 1) | to make | Carrier 1) | to make | Carrier 1) | to make |
| | 100 | | 100 | | 100 |
| | AF256 | | AF257 | | AF258 |
| Deodorant | 0.1-2.0 | Deodorant | 0.1-2.0 | Deodorant | 0.1-2.0 |
| active | | active | | active | |
| ingredient 2) | | ingredient 2) | | ingredient 2) | |
| KM | 0.05-2.0 | KM | 0.05-2.0 | KM | 0.05-2.0 |
| ZK186 | 0.1-5.0 | ZK187 | 0.1-5.0 | ZK188 | 0.1-5.0 |
| Carrier 1) | to make | Carrier 1) | to make | Carrier 1) | to make |
| | 100 | | 100 | | 100 |
| | AF259 | | AF260 | | AF261 |
| Deodorant | 0.1-2.0 | Deodorant | 0.1-2.0 | Deodorant | 0.1-2.0 |
| active | | active | | active | |
| ingredient 2) | | ingredient 2) | | ingredient 2) | |
| KM | 0.05-2.0 | KM | 0.05-2.0 | KM | 0.05-2.0 |
| ZK189 | 0.1-5.0 | ZK190 | 0.1-5.0 | ZK191 | 0.1-5.0 |
| Carrier 1) | to make | Carrier 1) | to make | Carrier 1) | to make |
| | 100 | | 100 | | 100 |
| | AF262 | | AF263 | | AF264 |
| Deodorant | 0.1-2.0 | Deodorant | 0.1-2.0 | Deodorant | 0.1-2.0 |
| active | | active | | active | |
| ingredient 2) | | ingredient 2) | | ingredient 2) | |
| KM | 0.05-2.0 | KM | 0.05-2.0 | KM | 0.05-2.0 |
| ZK192 | 0.1-5.0 | ZK193 | 0.1-5.0 | ZK194 | 0.1-5.0 |
| Carrier 1) | to make | Carrier 1) | to make | Carrier 1) | to make |
| | 100 | | 100 | | 100 |
| | AF265 | | AF266 | | AF267 |
| Deodorant | 0.1-2.0 | Deodorant | 0.1-2.0 | Deodorant | 0.1-2.0 |
| active | | active | | active | |
| ingredient 2) | | ingredient 2) | | ingredient 2) | |
| KM | 0.05-2.0 | KM | 0.05-2.0 | KM | 0.05-2.0 |
| ZK195 | 0.1-5.0 | ZK196 | 0.1-5.0 | ZK197 | 0.1-5.0 |
| Carrier 1) | to make | Carrier 1) | to make | Carrier 1) | to make |
| | 100 | | 100 | | 100 |
| | AF268 | | AF269 | | AF270 |
| Deodorant | 0.1-2.0 | Deodorant | 0.1-2.0 | Deodorant | 0.1-2.0 |
| active | | active | | active | |
| ingredient 2) | | ingredient 2) | | ingredient 2) | |
| KM | 0.05-2.0 | KM | 0.05-2.0 | KM | 0.05-2.0 |
| ZK198 | 0.1-5.0 | ZK199 | 0.1-5.0 | ZK200 | 0.1-5.0 |
| Carrier 1) | to make | Carrier 1) | to make | Carrier 1) | to make |
| | 100 | | 100 | | 100 |
| | AF271 | | AF272 | | AF273 |
| Deodorant | 0.1-2.0 | Deodorant | 0.1-2.0 | Deodorant | 0.1-2.0 |
| active | | active | | active | |
| ingredient 2) | | ingredient 2) | | ingredient 2) | |
| KM | 0.05-2.0 | KM | 0.05-2.0 | KM | 0.05-2.0 |
| ZK201 | 0.1-5.0 | ZK202 | 0.1-5.0 | ZK203 | 0.1-5.0 |
| Carrier 1) | to make | Carrier 1) | to make | Carrier 1) | to make |
| | 100 | | 100 | | 100 |
| | AF274 | | AF275 | | AF276 |
| Deodorant | 0.1-2.0 | Deodorant | 0.1-2.0 | Deodorant | 0.1-2.0 |
| active | | active | | active | |
| ingredient 2) | | ingredient 2) | | ingredient 2) | |
| KM | 0.05-2.0 | KM | 0.05-2.0 | KM | 0.05-2.0 |
| ZK204 | 0.1-5.0 | ZK205 | 0.1-5.0 | ZK206 | 0.1-5.0 |
| Carrier 1) | to make | Carrier 1) | to make | Carrier 1) | to make |
| | 100 | | 100 | | 100 |
| | AF277 | | AF278 | | AF279 |
| Deodorant | 0.1-2.0 | Deodorant | 0.1-2.0 | Deodorant | 0.1-2.0 |
| active | | active | | active | |
| ingredient 2) | | ingredient 2) | | ingredient 2) | |
| KM | 0.05-2.0 | KM | 0.05-2.0 | KM | 0.05-2.0 |
| ZK207 | 0.1-5.0 | ZK208 | 0.1-5.0 | ZK209 | 0.1-5.0 |
| Carrier 1) | to make | Carrier 1) | to make | Carrier 1) | to make |
| | 100 | | 100 | | 100 |
| | AF280 | | AF281 | | AF282 |
| Deodorant | 0.1-2.0 | Deodorant | 0.1-2.0 | Deodorant | 0.1-2.0 |
| active | | active | | active | |
| ingredient 2) | | ingredient 2) | | ingredient 2) | |
| KM | 0.05-2.0 | KM | 0.05-2.0 | KM | 0.05-2.0 |
| ZK210 | 0.1-5.0 | ZK211 | 0.1-5.0 | ZK212 | 0.1-5.0 |
| Carrier 1) | to make | Carrier 1) | to make | Carrier 1) | to make |
| | 100 | | 100 | | 100 |
| | AF283 | | AF284 | | AF285 |
| Deodorant | 0.1-2.0 | Deodorant | 0.1-2.0 | Deodorant | 0.1-2.0 |
| active | | active | | active | |
| ingredient 2) | | ingredient 2) | | ingredient 2) | |
| KM | 0.05-2.0 | KM | 0.05-2.0 | KM | 0.05-2.0 |
| ZK213 | 0.1-5.0 | ZK214 | 0.1-5.0 | ZK215 | 0.1-5.0 |
| Carrier 1) | to make | Carrier 1) | to make | Carrier 1) | to make |
| | 100 | | 100 | | 100 |
| | AF286 | | AF287 | | AF288 |
| Deodorant | 0.1-2.0 | Deodorant | 0.1-2.0 | Deodorant | 0.1-2.0 |
| active | | active | | active | |
| ingredient 2) | | ingredient 2) | | ingredient 2) | |
| KM | 0.05-2.0 | KM | 0.05-2.0 | KM | 0.05-2.0 |
| ZK216 | 0.1-5.0 | ZK217 | 0.1-5.0 | ZK218 | 0.1-5.0 |
| Carrier 1) | to make | Carrier 1) | to make | Carrier 1) | to make |
| | 100 | | 100 | | 100 |
| | AF289 | | AF290 | | AF291 |
| Deodorant | 0.1-2.0 | Deodorant | 0.1-2.0 | Deodorant | 0.1-2.0 |
| active | | active | | active | |
| ingredient 2) | | ingredient 2) | | ingredient 2) | |
| KM | 0.05-2.0 | KM | 0.05-2.0 | KM | 0.05-2.0 |
| ZK219 | 0.1-5.0 | ZK220 | 0.1-5.0 | ZK221 | 0.1-5.0 |
| Carrier 1) | to make | Carrier 1) | to make | Carrier 1) | to make |
| | 100 | | 100 | | 100 |
| | AF292 | | AF293 | | AF294 |
| Deodorant | 0.1-2.0 | Deodorant | 0.1-2.0 | Deodorant | 0.1-2.0 |
| active | | active | | active | |
| ingredient 2) | | ingredient 2) | | ingredient 2) | |
| KM | 0.05-2.0 | KM | 0.05-2.0 | KM | 0.05-2.0 |
| ZK222 | 0.1-5.0 | ZK223 | 0.1-5.0 | ZK224 | 0.1-5.0 |
| Carrier 1) | to make | Carrier 1) | to make | Carrier 1) | to make |
| | 100 | | 100 | | 100 |
| | AF295 | | AF296 | | AF297 |
| Deodorant | 0.1-2.0 | Deodorant | 0.1-2.0 | Deodorant | 0.1-2.0 |
| active | | active | | active | |
| ingredient 2) | | ingredient 2) | | ingredient 2) | |
| KM | 0.05-2.0 | KM | 0.05-2.0 | KM | 0.05-2.0 |
| ZK225 | 0.1-5.0 | ZK226 | 0.1-5.0 | ZK227 | 0.1-5.0 |
| Carrier 1) | to make | Carrier 1) | to make | Carrier 1) | to make |
| | 100 | | 100 | | 100 |
| | AF298 | | AF299 | | AF300 |
| Deodorant | 0.1-2.0 | Deodorant | 0.1-2.0 | Deodorant | 0.1-2.0 |
| active | | active | | active | |
| ingredient 2) | | ingredient 2) | | ingredient 2) | |
| KM | 0.05-2.0 | KM | 0.05-2.0 | KM | 0.05-2.0 |
| ZK228 | 0.1-5.0 | ZK229 | 0.1-5.0 | ZK230 | 0.1-5.0 |
| Carrier 1) | to make | Carrier 1) | to make | Carrier 1) | to make |
| | 100 | | 100 | | 100 |
| | AF301 | | AF302 | | AF303 |
| Deodorant | 0.1-2.0 | Deodorant | 0.1-2.0 | Deodorant | 0.1-2.0 |
| active | | active | | active | |
| ingredient 2) | | ingredient 2) | | ingredient 2) | |
| KM | 0.05-2.0 | KM | 0.05-2.0 | KM | 0.05-2.0 |
| ZK231 | 0.1-5.0 | ZK232 | 0.1-5.0 | ZK233 | 0.1-5.0 |
| Carrier 1) | to make | Carrier 1) | to make | Carrier 1) | to make |
| | 100 | | 100 | | 100 |
| | AF304 | | AF305 | | AF306 |
| Deodorant | 0.1-2.0 | Deodorant | 0.1-2.0 | Deodorant | 0.1-2.0 |
| active | | active | | active | |
| ingredient 2) | | ingredient 2) | | ingredient 2) | |
| KM | 0.05-2.0 | KM | 0.05-2.0 | KM | 0.05-2.0 |
| ZK234 | 0.1-5.0 | ZK235 | 0.1-5.0 | ZK236 | 0.1-5.0 |
| Carrier 1) | to make | Carrier 1) | to make | Carrier 1) | to make |
| | 100 | | 100 | | 100 |
| | AF307 | | AF308 | | AF309 |
| Deodorant | 0.1-2.0 | Deodorant | 0.1-2.0 | Deodorant | 0.1-2.0 |
| active | | active | | active | |
| ingredient 2) | | ingredient 2) | | ingredient 2) | |
| KM | 0.05-2.0 | KM | 0.05-2.0 | KM | 0.05-2.0 |
| ZK237 | 0.1-5.0 | ZK238 | 0.1-5.0 | ZK239 | 0.1-5.0 |
| Carrier 1) | to make | Carrier 1) | to make | Carrier 1) | to make |
| | 100 | | 100 | | 100 |
| | AF310 | | AF311 | | AF312 |
| Deodorant | 0.1-2.0 | Deodorant | 0.1-2.0 | Deodorant | 0.1-2.0 |
| active | | active | | active | |
| ingredient 2) | | ingredient 2) | | ingredient 2) | |
| KM | 0.05-2.0 | KM | 0.05-2.0 | KM | 0.05-2.0 |
| ZK240 | 0.1-5.0 | ZK241 | 0.1-5.0 | ZK242 | 0.1-5.0 |
| Carrier 1) | to make | Carrier 1) | to make | Carrier 1) | to make |
| | 100 | | 100 | | 100 |
| | AF313 | | AF314 | | AF315 |
| Deodorant | 0.1-2.0 | Deodorant | 0.1-2.0 | Deodorant | 0.1-2.0 |
| active | | active | | active | |
| ingredient 2) | | ingredient 2) | | ingredient 2) | |
| KM | 0.05-2.0 | KM | 0.05-2.0 | KM | 0.05-2.0 |
| ZK243 | 0.1-5.0 | ZK244 | 0.1-5.0 | ZK245 | 0.1-5.0 |
| Carrier 1) | to make | Carrier 1) | to make | Carrier 1) | to make |
| | 100 | | 100 | | 100 |
| | AF316 | | AF317 | | AF318 |
| Deodorant | 0.1-2.0 | Deodorant | 0.1-2.0 | Deodorant | 0.1-2.0 |
| active | | active | | active | |
| ingredient 2) | | ingredient 2) | | ingredient 2) | |
| KM | 0.05-2.0 | KM | 0.05-2.0 | KM | 0.05-2.0 |
| ZK246 | 0.1-5.0 | ZK247 | 0.1-5.0 | ZK248 | 0.1-5.0 |
| Carrier 1) | to make | Carrier 1) | to make | Carrier 1) | to make |
| | 100 | | 100 | | 100 |
| | AF319 | | AF320 | | AF321 |
| Deodorant | 0.1-2.0 | Deodorant | 0.1-2.0 | Deodorant | 0.1-2.0 |
| active | | active | | active | |
| ingredient 2) | | ingredient 2) | | ingredient 2) | |
| KM | 0.05-2.0 | KM | 0.05-2.0 | KM | 0.05-2.0 |
| ZK249 | 0.1-5.0 | ZK250 | 0.1-5.0 | ZK251 | 0.1-5.0 |
| Carrier 1) | to make | Carrier 1) | to make | Carrier 1) | to make |
| | 100 | | 100 | | 100 |
| | AF322 | | AF323 | | AF324 |
| Deodorant | 0.1-2.0 | Deodorant | 0.1-2.0 | Deodorant | 0.1-2.0 |
| active | | active | | active | |
| ingredient 2) | | ingredient 2) | | ingredient 2) | |
| KM | 0.05-2.0 | KM | 0.05-2.0 | KM | 0.05-2.0 |
| ZK252 | 0.1-5.0 | ZK253 | 0.1-5.0 | ZK254 | 0.1-5.0 |
| Carrier 1) | to make | Carrier 1) | to make | Carrier 1) | to make |
| | 100 | | 100 | | 100 |
| | AF325 | | AF326 | | AF327 |
| Deodorant | 0.1-2.0 | Deodorant | 0.1-2.0 | Deodorant | 0.1-2.0 |
| active | | active | | active | |
| ingredient 2) | | ingredient 2) | | ingredient 2) | |
| KM | 0.05-2.0 | KM | 0.05-2.0 | KM | 0.05-2.0 |
| ZK255 | 0.1-5.0 | ZK256 | 0.1-5.0 | ZK257 | 0.1-5.0 |
| Carrier 1) | to make | Carrier 1) | to make | Carrier 1) | to make |
| | 100 | | 100 | | 100 |
| | AF328 | | AF329 | | AF330 |
| Deodorant | 0.1-2.0 | Deodorant | 0.1-2.0 | Deodorant | 0.1-2.0 |
| active | | active | | active | |
| ingredient 2) | | ingredient 2) | | ingredient 2) | |
| KM | 0.05-2.0 | KM | 0.05-2.0 | KM | 0.05-2.0 |
| ZK258 | 0.1-5.0 | ZK259 | 0.1-5.0 | ZK260 | 0.1-5.0 |
| Carrier 1) | to make | Carrier 1) | to make | Carrier 1) | to make |
| | 100 | | 100 | | 100 |
| | AF331 | | AF332 | | AF333 |
| Deodorant | 0.1-2.0 | Deodorant | 0.1-2.0 | Deodorant | 0.1-2.0 |
| active | | active | | active | |
| ingredient 2) | | ingredient 2) | | ingredient 2) | |
| KM | 0.05-2.0 | KM | 0.05-2.0 | KM | 0.05-2.0 |
| ZK261 | 0.1-5.0 | ZK262 | 0.1-5.0 | ZK263 | 0.1-5.0 |
| Carrier 1) | to make | Carrier 1) | to make | Carrier 1) | to make |
| | 100 | | 100 | | 100 |
| | AF334 | | AF335 | | AF336 |
| Deodorant | 0.1-2.0 | Deodorant | 0.1-2.0 | Deodorant | 0.1-2.0 |
| active | | active | | active | |
| ingredient 2) | | ingredient 2) | | ingredient 2) | |
| KM | 0.05-2.0 | KM | 0.05-2.0 | KM | 0.05-2.0 |
| ZK264 | 0.1-5.0 | ZK265 | 0.1-5.0 | ZK266 | 0.1-5.0 |
| Carrier 1) | to make | Carrier 1) | to make | Carrier 1) | to make |
| | 100 | | 100 | | 100 |
| | AF337 | | AF338 | | AF339 |
| Al salt 3) | 2.0-8.0 | Al salt 3) | 2.0-8.0 | Al salt 3) | 2.0-8.0 |
| KM | 0.05-2.0 | KM | 0.05-2.0 | KM | 0.05-2.0 |
| ZK183 | 0.1-5.0 | ZK184 | 0.1-5.0 | ZK185 | 0.1-5.0 |
| Carrier 1) | to make | Carrier 1) | to make | Carrier 1) | to make |
| | 100 | | 100 | | 100 |
| | AF340 | | AF341 | | AF342 |
| Al salt 3) | 2.0-8.0 | Al salt 3) | 2.0-8.0 | Al salt 3) | 2.0-8.0 |
| KM | 0.05-2.0 | KM | 0.05-2.0 | KM | 0.05-2.0 |
| ZK186 | 0.1-5.0 | ZK187 | 0.1-5.0 | ZK188 | 0.1-5.0 |
| Carrier 1) | to make | Carrier 1) | to make | Carrier 1) | to make |
| | 100 | | 100 | | 100 |
| | AF343 | | AF344 | | AF345 |
| Al salt 3) | 2.0-8.0 | Al salt 3) | 2.0-8.0 | Al salt 3) | 2.0-8.0 |
| KM | 0.05-2.0 | KM | 0.05-2.0 | KM | 0.05-2.0 |
| ZK189 | 0.1-5.0 | ZK190 | 0.1-5.0 | ZK191 | 0.1-5.0 |
| Carrier 1) | to make | Carrier 1) | to make | Carrier 1) | to make |
| | 100 | | 100 | | 100 |
| | AF346 | | AF347 | | AF348 |
| Al salt 3) | 2.0-8.0 | Al salt 3) | 2.0-8.0 | Al salt 3) | 2.0-8.0 |
| KM | 0.05-2.0 | KM | 0.05-2.0 | KM | 0.05-2.0 |
| ZK192 | 0.1-5.0 | ZK193 | 0.1-5.0 | ZK194 | 0.1-5.0 |
| Carrier 1) | to make | Carrier 1) | to make | Carrier 1) | to make |
| | 100 | | 100 | | 100 |
| | AF349 | | AF350 | | AF351 |
| Al salt 3) | 2.0-8.0 | Al salt 3) | 2.0-8.0 | Al salt 3) | 2.0-8.0 |
| KM | 0.05-2.0 | KM | 0.05-2.0 | KM | 0.05-2.0 |
| ZK195 | 0.1-5.0 | ZK196 | 0.1-5.0 | ZK197 | 0.1-5.0 |
| Carrier 1) | to make | Carrier 1) | to make | Carrier 1) | to make |
| | 100 | | 100 | | 100 |
| | AF352 | | AF353 | | AF354 |
| Al salt 3) | 2.0-8.0 | Al salt 3) | 2.0-8.0 | Al salt 3) | 2.0-8.0 |
| KM | 0.05-2.0 | KM | 0.05-2.0 | KM | 0.05-2.0 |
| ZK198 | 0.1-5.0 | ZK199 | 0.1-5.0 | ZK200 | 0.1-5.0 |
| Carrier 1) | to make | Carrier 1) | to make | Carrier 1) | to make |
| | 100 | | 100 | | 100 |
| | AF355 | | AF356 | | AF357 |
| Al salt 3) | 2.0-8.0 | Al salt 3) | 2.0-8.0 | Al salt 3) | 2.0-8.0 |
| KM | 0.05-2.0 | KM | 0.05-2.0 | KM | 0.05-2.0 |
| ZK201 | 0.1-5.0 | ZK202 | 0.1-5.0 | ZK203 | 0.1-5.0 |
| Carrier 1) | to make | Carrier 1) | to make | Carrier 1) | to make |
| | 100 | | 100 | | 100 |
| | AF358 | | AF359 | | AF360 |
| Al salt 3) | 2.0-8.0 | Al salt 3) | 2.0-8.0 | Al salt 3) | 2.0-8.0 |
| KM | 0.05-2.0 | KM | 0.05-2.0 | KM | 0.05-2.0 |
| ZK204 | 0.1-5.0 | ZK205 | 0.1-5.0 | ZK206 | 0.1-5.0 |
| Carrier 1) | to make | Carrier 1) | to make | Carrier 1) | to make |
| | 100 | | 100 | | 100 |
| | AF361 | | AF362 | | AF363 |
| Al salt 3) | 2.0-8.0 | Al salt 3) | 2.0-8.0 | Al salt 3) | 2.0-8.0 |
| KM | 0.05-2.0 | KM | 0.05-2.0 | KM | 0.05-2.0 |
| ZK207 | 0.1-5.0 | ZK208 | 0.1-5.0 | ZK209 | 0.1-5.0 |
| Carrier 1) | to make | Carrier 1) | to make | Carrier 1) | to make |
| | 100 | | 100 | | 100 |
| | AF364 | | AF365 | | AF366 |
| Al salt 3) | 2.0-8.0 | Al salt 3) | 2.0-8.0 | Al salt 3) | 2.0-8.0 |
| KM | 0.05-2.0 | KM | 0.05-2.0 | KM | 0.05-2.0 |
| ZK210 | 0.1-5.0 | ZK211 | 0.1-5.0 | ZK212 | 0.1-5.0 |
| Carrier 1) | to make | Carrier 1) | to make | Carrier 1) | to make |
| | 100 | | 100 | | 100 |
| | AF367 | | AF368 | | AF369 |
| Al salt 3) | 2.0-8.0 | Al salt 3) | 2.0-8.0 | Al salt 3) | 2.0-8.0 |
| KM | 0.05-2.0 | KM | 0.05-2.0 | KM | 0.05-2.0 |
| ZK213 | 0.1-5.0 | ZK214 | 0.1-5.0 | ZK215 | 0.1-5.0 |
| Carrier 1) | to make | Carrier 1) | to make | Carrier 1) | to make |
| | 100 | | 100 | | 100 |
| | AF370 | | AF371 | | AF372 |
| Al salt 3) | 2.0-8.0 | Al salt 3) | 2.0-8.0 | Al salt 3) | 2.0-8.0 |
| KM | 0.05-2.0 | KM | 0.05-2.0 | KM | 0.05-2.0 |
| ZK216 | 0.1-5.0 | ZK217 | 0.1-5.0 | ZK218 | 0.1-5.0 |
| Carrier 1) | to make | Carrier 1) | to make | Carrier 1) | to make |
| | 100 | | 100 | | 100 |
| | AF373 | | AF374 | | AF375 |
| Al salt 3) | 2.0-8.0 | Al salt 3) | 2.0-8.0 | Al salt 3) | 2.0-8.0 |
| KM | 0.05-2.0 | KM | 0.05-2.0 | KM | 0.05-2.0 |
| ZK219 | 0.1-5.0 | ZK220 | 0.1-5.0 | ZK221 | 0.1-5.0 |
| Carrier 1) | to make | Carrier 1) | to make | Carrier 1) | to make |
| | 100 | | 100 | | 100 |
| | AF376 | | AF377 | | AF378 |
| Al salt 3) | 2.0-8.0 | Al salt 3) | 2.0-8.0 | Al salt 3) | 2.0-8.0 |
| KM | 0.05-2.0 | KM | 0.05-2.0 | KM | 0.05-2.0 |
| ZK222 | 0.1-5.0 | ZK223 | 0.1-5.0 | ZK224 | 0.1-5.0 |
| Carrier 1) | to make | Carrier 1) | to make | Carrier 1) | to make |
| | 100 | | 100 | | 100 |
| | AF379 | | AF380 | | AF381 |
| Al salt 3) | 2.0-8.0 | Al salt 3) | 2.0-8.0 | Al salt 3) | 2.0-8.0 |
| KM | 0.05-2.0 | KM | 0.05-2.0 | KM | 0.05-2.0 |
| ZK225 | 0.1-5.0 | ZK226 | 0.1-5.0 | ZK227 | 0.1-5.0 |
| Carrier 1) | to make | Carrier 1) | to make | Carrier 1) | to make |
| | 100 | | 100 | | 100 |
| | AF382 | | AF383 | | AF384 |
| Al salt 3) | 2.0-8.0 | Al salt 3) | 2.0-8.0 | Al salt 3) | 2.0-8.0 |
| KM | 0.05-2.0 | KM | 0.05-2.0 | KM | 0.05-2.0 |
| ZK228 | 0.1-5.0 | ZK229 | 0.1-5.0 | ZK230 | 0.1-5.0 |
| Carrier 1) | to make | Carrier 1) | to make | Carrier 1) | to make |
| | 100 | | 100 | | 100 |
| | AF385 | | AF386 | | AF387 |
| Al salt 3) | 2.0-8.0 | Al salt 3) | 2.0-8.0 | Al salt 3) | 2.0-8.0 |
| KM | 0.05-2.0 | KM | 0.05-2.0 | KM | 0.05-2.0 |
| ZK231 | 0.1-5.0 | ZK232 | 0.1-5.0 | ZK233 | 0.1-5.0 |
| Carrier 1) | to make | Carrier 1) | to make | Carrier 1) | to make |
| | 100 | | 100 | | 100 |
| | AF388 | | AF389 | | AF390 |
| Al salt 3) | 2.0-8.0 | Al salt 3) | 2.0-8.0 | Al salt 3) | 2.0-8.0 |
| KM | 0.05-2.0 | KM | 0.05-2.0 | KM | 0.05-2.0 |
| ZK234 | 0.1-5.0 | ZK235 | 0.1-5.0 | ZK236 | 0.1-5.0 |
| Carrier 1) | to make | Carrier 1) | to make | Carrier 1) | to make |
| | 100 | | 100 | | 100 |
| | AF391 | | AF392 | | AF393 |
| Al salt 3) | 2.0-8.0 | Al salt 3) | 2.0-8.0 | Al salt 3) | 2.0-8.0 |
| KM | 0.05-2.0 | KM | 0.05-2.0 | KM | 0.05-2.0 |
| ZK237 | 0.1-5.0 | ZK238 | 0.1-5.0 | ZK239 | 0.1-5.0 |
| Carrier 1) | to make | Carrier 1) | to make | Carrier 1) | to make |
| | 100 | | 100 | | 100 |
| | AF394 | | AF395 | | AF396 |
| Al salt 3) | 2.0-8.0 | Al salt 3) | 2.0-8.0 | Al salt 3) | 2.0-8.0 |
| KM | 0.05-2.0 | KM | 0.05-2.0 | KM | 0.05-2.0 |
| ZK243 | 0.1-5.0 | ZK244 | 0.1-5.0 | ZK245 | 0.1-5.0 |
| Carrier 1) | to make | Carrier 1) | to make | Carrier 1) | to make |
| | 100 | | 100 | | 100 |
| | AF397 | | AF398 | | AF399 |
| Al salt 3) | 2.0-8.0 | Al salt 3) | 2.0-8.0 | Al salt 3) | 2.0-8.0 |
| KM | 0.05-2.0 | KM | 0.05-2.0 | KM | 0.05-2.0 |
| ZK246 | 0.1-5.0 | ZK247 | 0.1-5.0 | ZK248 | 0.1-5.0 |
| Carrier 1) | to make | Carrier 1) | to make | Carrier 1) | to make |
| | 100 | | 100 | | 100 |
| | AF400 | | AF401 | | AF402 |
| Al salt 3) | 2.0-8.0 | Al salt 3) | 2.0-8.0 | Al salt 3) | 2.0-8.0 |
| KM | 0.05-2.0 | KM | 0.05-2.0 | KM | 0.05-2.0 |
| ZK249 | 0.1-5.0 | ZK250 | 0.1-5.0 | ZK251 | 0.1-5.0 |
| Carrier 1) | to make | Carrier 1) | to make | Carrier 1) | to make |
| | 100 | | 100 | | 100 |
| | AF403 | | AF404 | | AF405 |
| Al salt 3) | 2.0-8.0 | Al salt 3) | 2.0-8.0 | Al salt 3) | 2.0-8.0 |
| KM | 0.05-2.0 | KM | 0.05-2.0 | KM | 0.05-2.0 |
| ZK252 | 0.1-5.0 | ZK253 | 0.1-5.0 | ZK254 | 0.1-5.0 |
| Carrier 1) | to make | Carrier 1) | to make | Carrier 1) | to make |
| | 100 | | 100 | | 100 |
| | AF406 | | AF407 | | AF408 |
| Al salt 3) | 2.0-8.0 | Al salt 3) | 2.0-8.0 | Al salt 3) | 2.0-8.0 |
| KM | 0.05-2.0 | KM | 0.05-2.0 | KM | 0.05-2.0 |
| ZK255 | 0.1-5.0 | ZK256 | 0.1-5.0 | ZK257 | 0.1-5.0 |
| Carrier 1) | to make | Carrier 1) | to make | Carrier 1) | to make |
| | 100 | | 100 | | 100 |
| | AF409 | | AF410 | | AF411 |
| Al salt 3) | 2.0-8.0 | Al salt 3) | 2.0-8.0 | Al salt 3) | 2.0-8.0 |
| KM | 0.05-2.0 | KM | 0.05-2.0 | KM | 0.05-2.0 |
| ZK258 | 0.1-5.0 | ZK259 | 0.1-5.0 | ZK260 | 0.1-5.0 |
| Carrier 1) | to make | Carrier 1) | to make | Carrier 1) | to make |
| | 100 | | 100 | | 100 |
| | AF412 | | AF413 | | AF414 |
| Al salt 3) | 2.0-8.0 | Al salt 3) | 2.0-8.0 | Al salt 3) | 2.0-8.0 |
| KM | 0.05-2.0 | KM | 0.05-2.0 | KM | 0.05-2.0 |
| ZK261 | 0.1-5.0 | ZK262 | 0.1-5.0 | ZK263 | 0.1-5.0 |
| Carrier 1) | to make | Carrier 1) | to make | Carrier 1) | to make |
| | 100 | | 100 | | 100 |
| | AF415 | | AF416 | | AF417 |
| Al salt 3) | 2.0-8.0 | Al salt 3) | 2.0-8.0 | Al salt 3) | 2.0-8.0 |
| KM | 0.05-2.0 | KM | 0.05-2.0 | KM | 0.05-2.0 |
| ZK264 | 0.1-5.0 | ZK265 | 0.1-5.0 | ZK266 | 0.1-5.0 |
| Carrier 1) | to make | Carrier 1) | to make | Carrier 1) | to make |
| | 100 | | 100 | | 100 |
| | AF418 | | AF419 | | AF420 |
| Al salt 3) | 2.0-8.0 | Al salt 3) | 2.0-8.0 | Al salt 3) | 2.0-8.0 |
| KM | 0.05-2.0 | KM | 0.05-2.0 | KM | 0.05-2.0 |
| ZK240 | 0.1-5.0 | ZK241 | 0.1-5.0 | ZK242 | 0.1-5.0 |
| Carrier 1) | to make | Carrier 1) | to make | Carrier 1) | to make |
| | 100 | | 100 | | 100 |
| | AF421 | | AF422 | | AF423 |
| Deodorant | 0.1-2.0 | Deodorant | 0.1-2.0 | Deodorant | 0.1-2.0 |
| active | | active | | active | |
| ingredient 2) | | ingredient 2) | | ingredient 2) | |
| Al salt 3) | 2.0-8.0 | Al salt 3) | 2.0-8.0 | Al salt 3) | 2.0-8.0 |
| KM | 0.05-2.0 | KM | 0.05-2.0 | KM | 0.05-2.0 |
| ZK183 | 0.1-5.0 | ZK184 | 0.1-5.0 | ZK185 | 0.1-5.0 |
| Carrier 1) | to make | Carrier 1) | to make | Carrier 1) | to make |
| | 100 | | 100 | | 100 |
| | AF424 | | AF425 | | AF426 |
| Deodorant | 0.1-2.0 | Deodorant | 0.1-2.0 | Deodorant | 0.1-2.0 |
| active | | active | | active | |
| ingredient 2) | | ingredient 2) | | ingredient 2) | |
| Al salt 3) | 2.0-8.0 | Al salt 3) | 2.0-8.0 | Al salt 3) | 2.0-8.0 |
| KM | 0.05-2.0 | KM | 0.05-2.0 | KM | 0.05-2.0 |
| ZK186 | 0.1-5.0 | ZK187 | 0.1-5.0 | ZK188 | 0.1-5.0 |
| Carrier 1) | to make | Carrier 1) | to make | Carrier 1) | to make |
| | 100 | | 100 | | 100 |
| | AF427 | | AF428 | | AF429 |
| Deodorant | 0.1-2.0 | Deodorant | 0.1-2.0 | Deodorant | 0.1-2.0 |
| active | | active | | active | |
| ingredient 2) | | ingredient 2) | | ingredient 2) | |
| Al salt 3) | 2.0-8.0 | Al salt 3) | 2.0-8.0 | Al salt 3) | 2.0-8.0 |
| KM | 0.05-2.0 | KM | 0.05-2.0 | KM | 0.05-2.0 |
| ZK186 | 0.1-5.0 | ZK187 | 0.1-5.0 | ZK188 | 0.1-5.0 |
| Carrier 1) | to make | Carrier 1) | to make | Carrier 1) | to make |
| | 100 | | 100 | | 100 |
| | AF430 | | AF431 | | AF432 |
| Deodorant | 0.1-2.0 | Deodorant | 0.1-2.0 | Deodorant | 0.1-2.0 |
| active | | active | | active | |
| ingredient 2) | | ingredient 2) | | ingredient 2) | |
| Al salt 3) | 2.0-8.0 | Al salt 3) | 2.0-8.0 | Al salt 3) | 2.0-8.0 |
| KM | 0.05-2.0 | KM | 0.05-2.0 | KM | 0.05-2.0 |
| ZK192 | 0.1-5.0 | ZK193 | 0.1-5.0 | ZK194 | 0.1-5.0 |
| Carrier 1) | to make | Carrier 1) | to make | Carrier 1) | to make |
| | 100 | | 100 | | 100 |
| | AF433 | | AF434 | | AF435 |
| Deodorant | 0.1-2.0 | Deodorant | 0.1-2.0 | Deodorant | 0.1-2.0 |
| active | | active | | active | |
| ingredient 2) | | ingredient 2) | | ingredient 2) | |
| Al salt 3) | 2.0-8.0 | Al salt 3) | 2.0-8.0 | Al salt 3) | 2.0-8.0 |
| KM | 0.05-2.0 | KM | 0.05-2.0 | KM | 0.05-2.0 |
| ZK195 | 0.1-5.0 | ZK196 | 0.1-5.0 | ZK197 | 0.1-5.0 |
| Carrier 1) | to make | Carrier 1) | to make | Carrier 1) | to make |
| | 100 | | 100 | | 100 |
| | AF436 | | AF437 | | AF438 |
| Deodorant | 0.1-2.0 | Deodorant | 0.1-2.0 | Deodorant | 0.1-2.0 |
| active | | active | | active | |
| ingredient 2) | | ingredient 2) | | ingredient 2) | |
| Al salt 3) | 2.0-8.0 | Al salt 3) | 2.0-8.0 | Al salt 3) | 2.0-8.0 |
| KM | 0.05-2.0 | KM | 0.05-2.0 | KM | 0.05-2.0 |
| ZK198 | 0.1-5.0 | ZK199 | 0.1-5.0 | ZK200 | 0.1-5.0 |
| Carrier 1) | to make | Carrier 1) | to make | Carrier 1) | to make |
| | 100 | | 100 | | 100 |
| | AF439 | | AF440 | | AF441 |
| Deodorant | 0.1-2.0 | Deodorant | 0.1-2.0 | Deodorant | 0.1-2.0 |
| active | | active | | active | |
| ingredient 2) | | ingredient 2) | | ingredient 2) | |
| Al salt 3) | 2.0-8.0 | Al salt 3) | 2.0-8.0 | Al salt 3) | 2.0-8.0 |
| KM | 0.05-2.0 | KM | 0.05-2.0 | KM | 0.05-2.0 |
| ZK201 | 0.1-5.0 | ZK202 | 0.1-5.0 | ZK203 | 0.1-5.0 |
| Carrier 1) | to make | Carrier 1) | to make | Carrier 1) | to make |
| | 100 | | 100 | | 100 |
| | AF442 | | AF443 | | AF444 |
| Deodorant | 0.1-2.0 | Deodorant | 0.1-2.0 | Deodorant | 0.1-2.0 |
| active | | active | | active | |
| ingredient 2) | | ingredient 2) | | ingredient 2) | |
| Al salt 3) | 2.0-8.0 | Al salt 3) | 2.0-8.0 | Al salt 3) | 2.0-8.0 |
| KM | 0.05-2.0 | KM | 0.05-2.0 | KM | 0.05-2.0 |
| ZK204 | 0.1-5.0 | ZK205 | 0.1-5.0 | ZK206 | 0.1-5.0 |
| Carrier 1) | to make | Carrier 1) | to make | Carrier 1) | to make |
| | 100 | | 100 | | 100 |
| | AF445 | | AF446 | | AF447 |
| Deodorant | 0.1-2.0 | Deodorant | 0.1-2.0 | Deodorant | 0.1-2.0 |
| active | | active | | active | |
| ingredient 2) | | ingredient 2) | | ingredient 2) | |
| Al salt 3) | 2.0-8.0 | Al salt 3) | 2.0-8.0 | Al salt 3) | 2.0-8.0 |
| KM | 0.05-2.0 | KM | 0.05-2.0 | KM | 0.05-2.0 |
| ZK207 | 0.1-5.0 | ZK208 | 0.1-5.0 | ZK209 | 0.1-5.0 |
| Carrier 1) | to make | Carrier 1) | to make | Carrier 1) | to make |
| | 100 | | 100 | | 100 |
| | AF448 | | AF449 | | AF450 |
| Deodorant | 0.1-2.0 | Deodorant | 0.1-2.0 | Deodorant | 0.1-2.0 |
| active | | active | | active | |
| ingredient 2) | | ingredient 2) | | ingredient 2) | |
| Al salt 3) | 2.0-8.0 | Al salt 3) | 2.0-8.0 | Al salt 3) | 2.0-8.0 |
| KM | 0.05-2.0 | KM | 0.05-2.0 | KM | 0.05-2.0 |
| ZK210 | 0.1-5.0 | ZK211 | 0.1-5.0 | ZK212 | 0.1-5.0 |
| Carrier 1) | to make | Carrier 1) | to make | Carrier 1) | to make |
| | 100 | | 100 | | 100 |
| | AF451 | | AF452 | | AF453 |
| Deodorant | 0.1-2.0 | Deodorant | 0.1-2.0 | Deodorant | 0.1-2.0 |
| active | | active | | active | |
| ingredient 2) | | ingredient 2) | | ingredient 2) | |
| Al salt 3) | 2.0-8.0 | Al salt 3) | 2.0-8.0 | Al salt 3) | 2.0-8.0 |
| KM | 0.05-2.0 | KM | 0.05-2.0 | KM | 0.05-2.0 |
| ZK213 | 0.1-5.0 | ZK214 | 0.1-5.0 | ZK215 | 0.1-5.0 |
| Carrier 1) | to make | Carrier 1) | to make | Carrier 1) | to make |
| | 100 | | 100 | | 100 |
| | AF454 | | AF455 | | AF456 |
| Deodorant | 0.1-2.0 | Deodorant | 0.1-2.0 | Deodorant | 0.1-2.0 |
| active | | active | | active | |
| ingredient 2) | | ingredient 2) | | ingredient 2) | |
| Al salt 3) | 2.0-8.0 | Al salt 3) | 2.0-8.0 | Al salt 3) | 2.0-8.0 |
| KM | 0.05-2.0 | KM | 0.05-2.0 | KM | 0.05-2.0 |
| ZK216 | 0.1-5.0 | ZK217 | 0.1-5.0 | ZK218 | 0.1-5.0 |
| Carrier 1) | to make | Carrier 1) | to make | Carrier 1) | to make |
| | 100 | | 100 | | 100 |
| | AF457 | | AF458 | | AF459 |
| Deodorant | 0.1-2.0 | Deodorant | 0.1-2.0 | Deodorant | 0.1-2.0 |
| active | | active | | active | |
| ingredient 2) | | ingredient 2) | | ingredient 2) | |
| Al salt 3) | 2.0-8.0 | Al salt 3) | 2.0-8.0 | Al salt 3) | 2.0-8.0 |
| KM | 0.05-2.0 | KM | 0.05-2.0 | KM | 0.05-2.0 |
| ZK219 | 0.1-5.0 | ZK220 | 0.1-5.0 | ZK221 | 0.1-5.0 |
| Carrier 1) | to make | Carrier 1) | to make | Carrier 1) | to make |
| | 100 | | 100 | | 100 |
| | AF460 | | AF461 | | AF462 |
| Deodorant | 0.1-2.0 | Deodorant | 0.1-2.0 | Deodorant | 0.1-2.0 |
| active | | active | | active | |
| ingredient 2) | | ingredient 2) | | ingredient 2) | |
| Al salt 3) | 2.0-8.0 | Al salt 3) | 2.0-8.0 | Al salt 3) | 2.0-8.0 |
| KM | 0.05-2.0 | KM | 0.05-2.0 | KM | 0.05-2.0 |
| ZK222 | 0.1-5.0 | ZK223 | 0.1-5.0 | ZK224 | 0.1-5.0 |
| Carrier 1) | to make | Carrier 1) | to make | Carrier 1) | to make |
| | 100 | | 100 | | 100 |
| | AF463 | | AF464 | | AF465 |
| Deodorant | 0.1-2.0 | Deodorant | 0.1-2.0 | Deodorant | 0.1-2.0 |
| active | | active | | active | |
| ingredient 2) | | ingredient 2) | | ingredient 2) | |
| Al salt 3) | 2.0-8.0 | Al salt 3) | 2.0-8.0 | Al salt 3) | 2.0-8.0 |
| KM | 0.05-2.0 | KM | 0.05-2.0 | KM | 0.05-2.0 |
| ZK225 | 0.1-5.0 | ZK226 | 0.1-5.0 | ZK227 | 0.1-5.0 |
| Carrier 1) | to make | Carrier 1) | to make | Carrier 1) | to make |
| | 100 | | 100 | | 100 |
| | AF466 | | AF467 | | AF468 |
| Deodorant | 0.1-2.0 | Deodorant | 0.1-2.0 | Deodorant | 0.1-2.0 |
| active | | active | | active | |
| ingredient 2) | | ingredient 2) | | ingredient 2) | |
| Al salt 3) | 2.0-8.0 | Al salt 3) | 2.0-8.0 | Al salt 3) | 2.0-8.0 |
| KM | 0.05-2.0 | KM | 0.05-2.0 | KM | 0.05-2.0 |
| ZK228 | 0.1-5.0 | ZK229 | 0.1-5.0 | ZK230 | 0.1-5.0 |
| Carrier 1) | to make | Carrier 1) | to make | Carrier 1) | to make |
| | 100 | | 100 | | 100 |
| | AF469 | | AF470 | | AF471 |
| Deodorant | 0.1-2.0 | Deodorant | 0.1-2.0 | Deodorant | 0.1-2.0 |
| active | | active | | active | |
| ingredient 2) | | ingredient 2) | | ingredient 2) | |
| Al salt 3) | 2.0-8.0 | Al salt 3) | 2.0-8.0 | Al salt 3) | 2.0-8.0 |
| KM | 0.05-2.0 | KM | 0.05-2.0 | KM | 0.05-2.0 |
| ZK231 | 0.1-5.0 | ZK232 | 0.1-5.0 | ZK233 | 0.1-5.0 |
| Carrier 1) | to make | Carrier 1) | to make | Carrier 1) | to make |
| | 100 | | 100 | | 100 |
| | AF472 | | AF473 | | AF474 |
| Deodorant | 0.1-2.0 | Deodorant | 0.1-2.0 | Deodorant | 0.1-2.0 |
| active | | active | | active | |
| ingredient 2) | | ingredient 2) | | ingredient 2) | |
| Al salt 3) | 2.0-8.0 | Al salt 3) | 2.0-8.0 | Al salt 3) | 2.0-8.0 |
| KM | 0.05-2.0 | KM | 0.05-2.0 | KM | 0.05-2.0 |
| ZK234 | 0.1-5.0 | ZK235 | 0.1-5.0 | ZK236 | 0.1-5.0 |
| Carrier 1) | to make | Carrier 1) | to make | Carrier 1) | to make |
| | 100 | | 100 | | 100 |
| | AF475 | | AF476 | | AF477 |
| Deodorant | 0.1-2.0 | Deodorant | 0.1-2.0 | Deodorant | 0.1-2.0 |
| active | | active | | active | |
| ingredient 2) | | ingredient 2) | | ingredient 2) | |
| Al salt 3) | 2.0-8.0 | Al salt 3) | 2.0-8.0 | Al salt 3) | 2.0-8.0 |
| KM | 0.05-2.0 | KM | 0.05-2.0 | KM | 0.05-2.0 |
| ZK237 | 0.1-5.0 | ZK238 | 0.1-5.0 | ZK239 | 0.1-5.0 |
| Carrier 1) | to make | Carrier 1) | to make | Carrier 1) | to make |
| | 100 | | 100 | | 100 |
| | AF478 | | AF479 | | AF480 |
| Deodorant | 0.1-2.0 | Deodorant | 0.1-2.0 | Deodorant | 0.1-2.0 |
| active | | active | | active | |
| ingredient 2) | | ingredient 2) | | ingredient 2) | |
| Al salt 3) | 2.0-8.0 | Al salt 3) | 2.0-8.0 | Al salt 3) | 2.0-8.0 |
| KM | 0.05-2.0 | KM | 0.05-2.0 | KM | 0.05-2.0 |
| ZK240 | 0.1-5.0 | ZK241 | 0.1-5.0 | ZK242 | 0.1-5.0 |
| Carrier 1) | to make | Carrier 1) | to make | Carrier 1) | to make |
| | 100 | | 100 | | 100 |
| | AF481 | | AF482 | | AF483 |
| Deodorant | 0.1-2.0 | Deodorant | 0.1-2.0 | Deodorant | 0.1-2.0 |
| active | | active | | active | |
| ingredient 2) | | ingredient 2) | | ingredient 2) | |
| Al salt 3) | 2.0-8.0 | Al salt 3) | 2.0-8.0 | Al salt 3) | 2.0-8.0 |
| KM | 0.05-2.0 | KM | 0.05-2.0 | KM | 0.05-2.0 |
| ZK243 | 0.1-5.0 | ZK244 | 0.1-5.0 | ZK245 | 0.1-5.0 |
| Carrier 1) | to make | Carrier 1) | to make | Carrier 1) | to make |
| | 100 | | 100 | | 100 |
| | AF484 | | AF485 | | AF486 |
| Deodorant | 0.1-2.0 | Deodorant | 0.1-2.0 | Deodorant | 0.1-2.0 |
| active | | active | | active | |
| ingredient 2) | | ingredient 2) | | ingredient 2) | |
| Al salt 3) | 2.0-8.0 | Al salt 3) | 2.0-8.0 | Al salt 3) | 2.0-8.0 |
| KM | 0.05-2.0 | KM | 0.05-2.0 | KM | 0.05-2.0 |
| ZK246 | 0.1-5.0 | ZK247 | 0.1-5.0 | ZK248 | 0.1-5.0 |
| Carrier 1) | to make | Carrier 1) | to make | Carrier 1) | to make |
| | 100 | | 100 | | 100 |
| | AF487 | | AF488 | | AF489 |
| Deodorant | 0.1-2.0 | Deodorant | 0.1-2.0 | Deodorant | 0.1-2.0 |
| active | | active | | active | |
| ingredient 2) | | ingredient 2) | | ingredient 2) | |
| Al salt 3) | 2.0-8.0 | Al salt 3) | 2.0-8.0 | Al salt 3) | 2.0-8.0 |
| KM | 0.05-2.0 | KM | 0.05-2.0 | KM | 0.05-2.0 |
| ZK249 | 0.1-5.0 | ZK250 | 0.1-5.0 | ZK251 | 0.1-5.0 |
| Carrier 1) | to make | Carrier 1) | to make | Carrier 1) | to make |
| | 100 | | 100 | | 100 |
| | AF490 | | AF491 | | AF492 |
| Deodorant | 0.1-2.0 | Deodorant | 0.1-2.0 | Deodorant | 0.1-2.0 |
| active | | active | | active | |
| ingredient 2) | | ingredient 2) | | ingredient 2) | |
| Al salt 3) | 2.0-8.0 | Al salt 3) | 2.0-8.0 | Al salt 3) | 2.0-8.0 |
| KM | 0.05-2.0 | KM | 0.05-2.0 | KM | 0.05-2.0 |
| ZK252 | 0.1-5.0 | ZK253 | 0.1-5.0 | ZK254 | 0.1-5.0 |
| Carrier 1) | to make | Carrier 1) | to make | Carrier 1) | to make |
| | 100 | | 100 | | 100 |
| | AF493 | | AF494 | | AF495 |
| Deodorant | 0.1-2.0 | Deodorant | 0.1-2.0 | Deodorant | 0.1-2.0 |
| active | | active | | active | |
| ingredient 2) | | ingredient 2) | | ingredient 2) | |
| Al salt 3) | 2.0-8.0 | Al salt 3) | 2.0-8.0 | Al salt 3) | 2.0-8.0 |
| KM | 0.05-2.0 | KM | 0.05-2.0 | KM | 0.05-2.0 |
| ZK255 | 0.1-5.0 | ZK256 | 0.1-5.0 | ZK257 | 0.1-5.0 |
| Carrier 1) | to make | Carrier 1) | to make | Carrier 1) | to make |
| | 100 | | 100 | | 100 |
| | AF496 | | AF497 | | AF498 |
| Deodorant | 0.1-2.0 | Deodorant | 0.1-2.0 | Deodorant | 0.1-2.0 |
| active | | active | | active | |
| ingredient 2) | | ingredient 2) | | ingredient 2) | |
| Al salt 3) | 2.0-8.0 | Al salt 3) | 2.0-8.0 | Al salt 3) | 2.0-8.0 |
| KM | 0.05-2.0 | KM | 0.05-2.0 | KM | 0.05-2.0 |
| ZK258 | 0.1-5.0 | ZK259 | 0.1-5.0 | ZK260 | 0.1-5.0 |
| Carrier 1) | to make | Carrier 1) | to make | Carrier 1) | to make |
| | 100 | | 100 | | 100 |
| | AF499 | | AF500 | | AF501 |
| Deodorant | 0.1-2.0 | Deodorant | 0.1-2.0 | Deodorant | 0.1-2.0 |
| active | | active | | active | |
| ingredient 2) | | ingredient 2) | | ingredient 2) | |
| Al salt 3) | 2.0-8.0 | Al salt 3) | 2.0-8.0 | Al salt 3) | 2.0-8.0 |
| KM | 0.05-2.0 | KM | 0.05-2.0 | KM | 0.05-2.0 |
| ZK261 | 0.1-5.0 | ZK262 | 0.1-5.0 | ZK263 | 0.1-5.0 |
| Carrier 1) | to make | Carrier 1) | to make | Carrier 1) | to make |
| | 100 | | 100 | | 100 |
| | AF502 | | AF503 | | AF504 |
| Deodorant | 0.1-2.0 | Deodorant | 0.1-2.0 | Deodorant | 0.1-2.0 |
| active | | active | | active | |
| ingredient 2) | | ingredient 2) | | ingredient 2) | |
| Al salt 3) | 2.0-8.0 | Al salt 3) | 2.0-8.0 | Al salt 3) | 2.0-8.0 |
| KM | 0.05-2.0 | KM | 0.05-2.0 | KM | 0.05-2.0 |
| ZK264 | 0.1-5.0 | ZK265 | 0.1-5.0 | ZK266 | 0.1-5.0 |
| Carrier 1) | to make | Carrier 1) | to make | Carrier 1) | to make |
| | 100 | | 100 | | 100 |

1) aqueous, aqueous-alcoholic or anhydrous carrier,
2) selected from mixture comprising 1,2-hexanediol and 1,2-octanediol,
3) aluminum chlorohydrate,
4) aluminum-zirconium tetrachlorohydrate, stabilized with glycine

TABLE 4
Particularly preferred cosmetic agents (information in wt.
%, based on total weight of the propellant-free agent)

| | AF1 | | AF2 | | AF3 |
|---|---|---|---|---|---|
| Deodorant | 0.1-2.0 | Deodorant | 0.1-2.0 | Deodorant | 0.1-2.0 |
| active | | active | | active | |
| ingredient | | ingredient | | ingredient | |
| KM | 0.05-2.0 | KM | 0.05-2.0 | KM | 0.05-2.0 |
| ZK267 | 0.1-5.0 | ZK268 | 0.1-5.0 | ZK269 | 0.1-5.0 |
| Carrier 1) | to make | Carrier 1) | to make | Carrier 1) | to make |
| | 100 | | 100 | | 100 |
| | AF4 | | AF5 | | AF6 |
| Deodorant | 0.1-2.0 | Deodorant | 0.1-2.0 | Deodorant | 0.1-2.0 |
| active | | active | | active | |
| ingredient | | ingredient | | ingredient | |
| KM | 0.05-2.0 | KM | 0.05-2.0 | KM | 0.05-2.0 |
| ZK270 | 0.1-5.0 | ZK271 | 0.1-5.0 | ZK272 | 0.1-5.0 |
| Carrier 1) | to make | Carrier 1) | to make | Carrier 1) | to make |
| | 100 | | 100 | | 100 |
| | AF7 | | AF8 | | AF9 |
| Deodorant | 0.1-2.0 | Deodorant | 0.1-2.0 | Deodorant | 0.1-2.0 |
| active | | active | | active | |
| ingredient | | ingredient | | ingredient | |
| KM | 0.05-2.0 | KM | 0.05-2.0 | KM | 0.05-2.0 |
| ZK273 | 0.1-5.0 | ZK274 | 0.1-5.0 | ZK275 | 0.1-5.0 |
| Carrier 1) | to make | Carrier 1) | to make | Carrier 1) | to make |
| | 100 | | 100 | | 100 |
| | AF10 | | AF11 | | AF12 |
| Deodorant | 0.1-2.0 | Deodorant | 0.1-2.0 | Deodorant | 0.1-2.0 |
| active | | active | | active | |
| ingredient | | ingredient | | ingredient | |
| KM | 0.05-2.0 | KM | 0.05-2.0 | KM | 0.05-2.0 |
| ZK276 | 0.1-5.0 | ZK277 | 0.1-5.0 | ZK278 | 0.1-5.0 |
| Carrier 1) | to make | Carrier 1) | to make | Carrier 1) | to make |
| | 100 | | 100 | | 100 |
| | AF13 | | AF14 | | AF15 |
| Deodorant | 0.1-2.0 | Deodorant | 0.1-2.0 | Deodorant | 0.1-2.0 |
| active | | active | | active | |
| ingredient | | ingredient | | ingredient | |
| KM | 0.05-2.0 | KM | 0.05-2.0 | KM | 0.05-2.0 |
| ZK279 | 0.1-5.0 | ZK280 | 0.1-5.0 | ZK281 | 0.1-5.0 |
| Carrier 1) | to make | Carrier 1) | to make | Carrier 1) | to make |
| | 100 | | 100 | | 100 |
| | AF16 | | AF17 | | AF18 |
| Deodorant | 0.1-2.0 | Deodorant | 0.1-2.0 | Deodorant | 0.1-2.0 |
| active | | active | | active | |
| ingredient | | ingredient | | ingredient | |
| KM | 0.05-2.0 | KM | 0.05-2.0 | KM | 0.05-2.0 |
| ZK282 | 0.1-5.0 | ZK283 | 0.1-5.0 | ZK284 | 0.1-5.0 |
| Carrier 1) | to make | Carrier 1) | to make | Carrier 1) | to make |
| | 100 | | 100 | | 100 |
| | AF19 | | AF20 | | AF21 |
| Deodorant | 0.1-2.0 | Deodorant | 0.1-2.0 | Deodorant | 0.1-2.0 |
| active | | active | | active | |
| ingredient | | ingredient | | ingredient | |
| KM | 0.05-2.0 | KM | 0.05-2.0 | KM | 0.05-2.0 |
| ZK285 | 0.1-5.0 | ZK286 | 0.1-5.0 | ZK287 | 0.1-5.0 |
| Carrier 1) | to make | Carrier 1) | to make | Carrier 1) | to make |
| | 100 | | 100 | | 100 |
| | AF22 | | AF23 | | AF24 |
| Deodorant | 0.1-2.0 | Deodorant | 0.1-2.0 | Deodorant | 0.1-2.0 |
| active | | active | | active | |
| ingredient | | ingredient | | ingredient | |
| KM | 0.05-2.0 | KM | 0.05-2.0 | KM | 0.05-2.0 |
| ZK288 | 0.1-5.0 | ZK289 | 0.1-5.0 | ZK290 | 0.1-5.0 |
| Carrier 1) | to make | Carrier 1) | to make | Carrier 1) | to make |
| | 100 | | 100 | | 100 |
| | AF25 | | AF26 | | AF27 |
| Deodorant | 0.1-2.0 | Deodorant | 0.1-2.0 | Deodorant | 0.1-2.0 |
| active | | active | | active | |
| ingredient | | ingredient | | ingredient | |
| KM | 0.05-2.0 | KM | 0.05-2.0 | KM | 0.05-2.0 |
| ZK291 | 0.1-5.0 | ZK292 | 0.1-5.0 | ZK293 | 0.1-5.0 |
| Carrier 1) | to make | Carrier 1) | to make | Carrier 1) | to make |
| | 100 | | 100 | | 100 |
| | AF28 | | AF29 | | AF30 |
| Deodorant | 0.1-2.0 | Deodorant | 0.1-2.0 | Deodorant | 0.1-2.0 |
| active | | active | | active | |
| ingredient | | ingredient | | ingredient | |
| KM | 0.05-2.0 | KM | 0.05-2.0 | KM | 0.05-2.0 |
| ZK294 | 0.1-5.0 | ZK295 | 0.1-5.0 | ZK296 | 0.1-5.0 |
| Carrier 1) | to make | Carrier 1) | to make | Carrier 1) | to make |
| | 100 | | 100 | | 100 |
| | AF31 | | AF32 | | AF33 |
| Deodorant | 0.1-2.0 | Deodorant | 0.1-2.0 | Deodorant | 0.1-2.0 |
| active | | active | | active | |
| ingredient | | ingredient | | ingredient | |
| KM | 0.05-2.0 | KM | 0.05-2.0 | KM | 0.05-2.0 |
| ZK297 | 0.1-5.0 | ZK298 | 0.1-5.0 | ZK299 | 0.1-5.0 |
| Carrier 1) | to make | Carrier 1) | to make | Carrier 1) | to make |
| | 100 | | 100 | | 100 |
| | AF34 | | AF35 | | AF36 |
| Deodorant | 0.1-2.0 | Deodorant | 0.1-2.0 | Deodorant | 0.1-2.0 |
| active | | active | | active | |
| ingredient | | ingredient | | ingredient | |
| KM | 0.05-2.0 | KM | 0.05-2.0 | KM | 0.05-2.0 |
| ZK299 | 0.1-5.0 | ZK300 | 0.1-5.0 | ZK301 | 0.1-5.0 |
| Carrier 1) | to make | Carrier 1) | to make | Carrier 1) | to make |
| | 100 | | 100 | | 100 |
| | AF37 | | AF38 | | AF39 |
| Deodorant | 0.1-2.0 | Deodorant | 0.1-2.0 | Deodorant | 0.1-2.0 |
| active | | active | | active | |
| ingredient | | ingredient | | ingredient | |
| KM | 0.05-2.0 | KM | 0.05-2.0 | KM | 0.05-2.0 |
| ZK300 | 0.1-5.0 | ZK301 | 0.1-5.0 | ZK302 | 0.1-5.0 |
| Carrier 1) | to make | Carrier 1) | to make | Carrier 1) | to make |
| | 100 | | 100 | | 100 |
| | AF40 | | AF41 | | AF42 |
| Deodorant | 0.1-2.0 | Deodorant | 0.1-2.0 | Deodorant | 0.1-2.0 |
| active | | active | | active | |
| ingredient | | ingredient | | ingredient | |
| KM | 0.05-2.0 | KM | 0.05-2.0 | KM | 0.05-2.0 |
| ZK303 | 0.1-5.0 | ZK304 | 0.1-5.0 | ZK305 | 0.1-5.0 |
| Carrier 1) | to make | Carrier 1) | to make | Carrier 1) | to make |
| | 100 | | 100 | | 100 |
| | AF43 | | AF44 | | AF45 |
| Deodorant | 0.1-2.0 | Deodorant | 0.1-2.0 | Deodorant | 0.1-2.0 |
| active | | active | | active | |
| ingredient | | ingredient | | ingredient | |
| KM | 0.05-2.0 | KM | 0.05-2.0 | KM | 0.05-2.0 |
| ZK306 | 0.1-5.0 | ZK307 | 0.1-5.0 | ZK308 | 0.1-5.0 |
| Carrier 1) | to make | Carrier 1) | to make | Carrier 1) | to make |
| | 100 | | 100 | | 100 |
| | AF46 | | AF47 | | AF48 |
| Deodorant | 0.1-2.0 | Deodorant | 0.1-2.0 | Deodorant | 0.1-2.0 |
| active | | active | | active | |
| ingredient | | ingredient | | ingredient | |
| KM | 0.05-2.0 | KM | 0.05-2.0 | KM | 0.05-2.0 |
| ZK309 | 0.1-5.0 | ZK310 | 0.1-5.0 | ZK311 | 0.1-5.0 |
| Carrier 1) | to make | Carrier 1) | to make | Carrier 1) | to make |
| | 100 | | 100 | | 100 |
| | AF49 | | AF50 | | AF51 |
| Deodorant | 0.1-2.0 | Deodorant | 0.1-2.0 | Deodorant | 0.1-2.0 |
| active | | active | | active | |
| ingredient | | ingredient | | ingredient | |
| KM | 0.05-2.0 | KM | 0.05-2.0 | KM | 0.05-2.0 |
| ZK312 | 0.1-5.0 | ZK313 | 0.1-5.0 | ZK314 | 0.1-5.0 |
| Carrier 1) | to make | Carrier 1) | to make | Carrier 1) | to make |
| | 100 | | 100 | | 100 |
| | AF52 | | AF53 | | AF54 |
| Deodorant | 0.1-2.0 | Deodorant | 0.1-2.0 | Deodorant | 0.1-2.0 |
| active | | active | | active | |
| ingredient | | ingredient | | ingredient | |
| KM | 0.05-2.0 | KM | 0.05-2.0 | KM | 0.05-2.0 |
| ZK315 | 0.1-5.0 | ZK316 | 0.1-5.0 | ZK317 | 0.1-5.0 |
| Carrier 1) | to make | Carrier 1) | to make | Carrier 1) | to make |
| | 100 | | 100 | | 100 |
| | AF55 | | AF59 | | AF57 |
| Deodorant | 0.1-2.0 | Deodorant | 0.1-2.0 | Deodorant | 0.1-2.0 |
| active | | active | | active | |
| ingredient | | ingredient | | ingredient | |
| KM | 0.05-2.0 | KM | 0.05-2.0 | KM | 0.05-2.0 |
| ZK318 | 0.1-5.0 | ZK319 | 0.1-5.0 | ZK320 | 0.1-5.0 |
| Carrier 1) | to make | Carrier 1) | to make | Carrier 1) | to make |
| | 100 | | 100 | | 100 |
| | AF58 | | AF59 | | AF60 |
| Deodorant | 0.1-2.0 | Deodorant | 0.1-2.0 | Deodorant | 0.1-2.0 |
| active | | active | | active | |
| ingredient | | ingredient | | ingredient | |
| KM | 0.05-2.0 | KM | 0.05-2.0 | KM | 0.05-2.0 |
| ZK321 | 0.1-5.0 | ZK322 | 0.1-5.0 | ZK323 | 0.1-5.0 |
| Carrier 1) | to make | Carrier 1) | to make | Carrier 1) | to make |
| | 100 | | 100 | | 100 |
| | AF61 | | AF62 | | AF63 |
| Deodorant | 0.1-2.0 | Deodorant | 0.1-2.0 | Deodorant | 0.1-2.0 |
| active | | active | | active | |
| ingredient | | ingredient | | ingredient | |
| KM | 0.05-2.0 | KM | 0.05-2.0 | KM | 0.05-2.0 |
| ZK324 | 0.1-5.0 | ZK325 | 0.1-5.0 | ZK326 | 0.1-5.0 |
| Carrier 1) | to make | Carrier 1) | to make | Carrier 1) | to make |
| | 100 | | 100 | | 100 |
| | AF64 | | AF65 | | AF66 |
| Deodorant | 0.1-2.0 | Deodorant | 0.1-2.0 | Deodorant | 0.1-2.0 |
| active | | active | | active | |
| ingredient | | ingredient | | ingredient | |
| KM | 0.05-2.0 | KM | 0.05-2.0 | KM | 0.05-2.0 |
| ZK327 | 0.1-5.0 | ZK328 | 0.1-5.0 | ZK329 | 0.1-5.0 |
| Carrier 1) | to make | Carrier 1) | to make | Carrier 1) | to make |
| | 100 | | 100 | | 100 |
| | AF67 | | AF68 | | AF69 |
| Deodorant | 0.1-2.0 | Deodorant | 0.1-2.0 | Deodorant | 0.1-2.0 |
| active | | active | | active | |
| ingredient | | ingredient | | ingredient | |
| KM | 0.05-2.0 | KM | 0.05-2.0 | KM | 0.05-2.0 |
| ZK330 | 0.1-5.0 | ZK331 | 0.1-5.0 | ZK332 | 0.1-5.0 |
| Carrier 1) | to make | Carrier 1) | to make | Carrier 1) | to make |
| | 100 | | 100 | | 100 |
| | AF70 | | AF71 | | AF72 |
| Deodorant | 0.1-2.0 | Deodorant | 0.1-2.0 | Deodorant | 0.1-2.0 |
| active | | active | | active | |
| ingredient | | ingredient | | ingredient | |
| KM | 0.05-2.0 | KM | 0.05-2.0 | KM | 0.05-2.0 |
| ZK333 | 0.1-5.0 | ZK334 | 0.1-5.0 | ZK335 | 0.1-5.0 |
| Carrier 1) | to make | Carrier 1) | to make | Carrier 1) | to make |
| | 100 | | 100 | | 100 |
| | AF73 | | AF74 | | AF75 |
| Deodorant | 0.1-2.0 | Deodorant | 0.1-2.0 | Deodorant | 0.1-2.0 |
| active | | active | | active | |
| ingredient | | ingredient | | ingredient | |
| KM | 0.05-2.0 | KM | 0.05-2.0 | KM | 0.05-2.0 |
| ZK336 | 0.1-5.0 | ZK337 | 0.1-5.0 | ZK338 | 0.1-5.0 |
| Carrier 1) | to make | Carrier 1) | to make | Carrier 1) | to make |
| | 100 | | 100 | | 100 |
| | AF76 | | AF77 | | AF78 |
| Deodorant | 0.1-2.0 | Deodorant | 0.1-2.0 | Deodorant | 0.1-2.0 |
| active | | active | | active | |
| ingredient | | ingredient | | ingredient | |
| KM | 0.05-2.0 | KM | 0.05-2.0 | KM | 0.05-2.0 |
| ZK339 | 0.1-5.0 | ZK340 | 0.1-5.0 | ZK341 | 0.1-5.0 |
| Carrier 1) | to make | Carrier 1) | to make | Carrier 1) | to make |
| | 100 | | 100 | | 100 |
| | AF79 | | AF80 | | AF81 |
| Deodorant | 0.1-2.0 | Deodorant | 0.1-2.0 | Deodorant | 0.1-2.0 |
| active | | active | | active | |
| ingredient | | ingredient | | ingredient | |
| KM | 0.05-2.0 | KM | 0.05-2.0 | KM | 0.05-2.0 |
| ZK342 | 0.1-5.0 | ZK343 | 0.1-5.0 | ZK344 | 0.1-5.0 |
| Carrier 1) | to make | Carrier 1) | to make | Carrier 1) | to make |
| | 100 | | 100 | | 100 |
| | AF82 | | AF83 | | AF84 |
| Deodorant | 0.1-2.0 | Deodorant | 0.1-2.0 | Deodorant | 0.1-2.0 |
| active | | active | | active | |
| ingredient | | ingredient | | ingredient | |
| KM | 0.05-2.0 | KM | 0.05-2.0 | KM | 0.05-2.0 |
| ZK345 | 0.1-5.0 | ZK346 | 0.1-5.0 | ZK347 | 0.1-5.0 |
| Carrier 1) | to make | Carrier 1) | to make | Carrier 1) | to make |
| | 100 | | 100 | | 100 |
| KM | 0.1-7.0 | KM | 0.1-7.0 | KM | 0.1-7.0 |
| | AF85 | | AF86 | | AF87 |
| Al salt | 2.0-8.0 | Al salt | 2.0-8.0 | Al salt | 2.0-8.0 |
| KM | 0.05-2.0 | KM | 0.05-2.0 | KM | 0.05-2.0 |
| ZK267 | 0.1-5.0 | ZK268 | 0.1-5.0 | ZK269 | 0.1-5.0 |
| Carrier 1) | to make | Carrier 1) | to make | Carrier 1) | to make |
| | 100 | | 100 | | 100 |
| | AF88 | | AF89 | | AF90 |
| Al salt | 2.0-8.0 | Al salt | 2.0-8.0 | Al salt | 2.0-8.0 |
| KM | 0.05-2.0 | KM | 0.05-2.0 | KM | 0.05-2.0 |
| ZK270 | 0.1-5.0 | ZK271 | 0.1-5.0 | ZK272 | 0.1-5.0 |
| Carrier 1) | to make | Carrier 1) | to make | Carrier 1) | to make |
| | 100 | | 100 | | 100 |
| | AF91 | | AF92 | | AF93 |
| Al salt | 2.0-8.0 | Al salt | 2.0-8.0 | Al salt | 2.0-8.0 |
| KM | 0.05-2.0 | KM | 0.05-2.0 | KM | 0.05-2.0 |
| ZK273 | 0.1-5.0 | ZK274 | 0.1-5.0 | ZK275 | 0.1-5.0 |
| Carrier 1) | to make | Carrier 1) | to make | Carrier 1) | to make |
| | 100 | | 100 | | 100 |
| | AF94 | | AF95 | | AF96 |
| Al salt | 2.0-8.0 | Al salt | 2.0-8.0 | Al salt | 2.0-8.0 |
| KM | 0.05-2.0 | KM | 0.05-2.0 | KM | 0.05-2.0 |
| ZK276 | 0.1-5.0 | ZK277 | 0.1-5.0 | ZK278 | 0.1-5.0 |
| Carrier 1) | to make | Carrier 1) | to make | Carrier 1) | to make |
| | 100 | | 100 | | 100 |
| | AF97 | | AF98 | | AF99 |
| Al salt | 2.0-8.0 | Al salt | 2.0-8.0 | Al salt | 2.0-8.0 |
| KM | 0.05-2.0 | KM | 0.05-2.0 | KM | 0.05-2.0 |
| ZK279 | 0.1-5.0 | ZK280 | 0.1-5.0 | ZK280 | 0.1-5.0 |
| Carrier 1) | to make | Carrier 1) | to make | Carrier 1) | to make |
| | 100 | | 100 | | 100 |
| | AF100 | | AF101 | | AF102 |
| Al salt | 2.0-8.0 | Al salt | 2.0-8.0 | Al salt | 2.0-8.0 |
| KM | 0.05-2.0 | KM | 0.05-2.0 | KM | 0.05-2.0 |
| ZK281 | 0.1-5.0 | ZK282 | 0.1-5.0 | ZK283 | 0.1-5.0 |
| Carrier 1) | to make | Carrier 1) | to make | Carrier 1) | to make |
| | 100 | | 100 | | 100 |
| | AF103 | | AF104 | | AF105 |
| Al salt | 2.0-8.0 | Al salt | 2.0-8.0 | Al salt | 2.0-8.0 |
| KM | 0.05-2.0 | KM | 0.05-2.0 | KM | 0.05-2.0 |
| ZK284 | 0.1-5.0 | ZK285 | 0.1-5.0 | ZK286 | 0.1-5.0 |
| Carrier 1) | to make | Carrier 1) | to make | Carrier 1) | to make |
| | 100 | | 100 | | 100 |
| | AF106 | | AF107 | | AF108 |
| Al salt | 2.0-8.0 | Al salt | 2.0-8.0 | Al salt | 2.0-8.0 |
| KM | 0.05-2.0 | KM | 0.05-2.0 | KM | 0.05-2.0 |
| ZK287 | 0.1-5.0 | ZK288 | 0.1-5.0 | ZK289 | 0.1-5.0 |
| Carrier 1) | to make | Carrier 1) | to make | Carrier 1) | to make |
| | 100 | | 100 | | 100 |
| | AF109 | | AF110 | | AF111 |
| Al salt | 2.0-8.0 | Al salt | 2.0-8.0 | Al salt | 2.0-8.0 |
| KM | 0.05-2.0 | KM | 0.05-2.0 | KM | 0.05-2.0 |
| ZK290 | 0.1-5.0 | ZK291 | 0.1-5.0 | ZK292 | 0.1-5.0 |
| Carrier 1) | to make | Carrier 1) | to make | Carrier 1) | to make |
| | 100 | | 100 | | 100 |
| | AF112 | | AF113 | | AF114 |
| Al salt | 2.0-8.0 | Al salt | 2.0-8.0 | Al salt | 2.0-8.0 |
| KM | 0.05-2.0 | KM | 0.05-2.0 | KM | 0.05-2.0 |
| ZK293 | 0.1-5.0 | ZK294 | 0.1-5.0 | ZK295 | 0.1-5.0 |
| Carrier 1) | to make | Carrier 1) | to make | Carrier 1) | to make |
| | 100 | | 100 | | 100 |
| | AF115 | | AF116 | | AF117 |
| Al salt | 2.0-8.0 | Al salt | 2.0-8.0 | Al salt | 2.0-8.0 |
| KM | 0.05-2.0 | KM | 0.05-2.0 | KM | 0.05-2.0 |
| ZK296 | 0.1-5.0 | ZK297 | 0.1-5.0 | ZK298 | 0.1-5.0 |
| Carrier 1) | to make | Carrier 1) | to make | Carrier 1) | to make |
| | 100 | | 100 | | 100 |
| | AF118 | | AF119 | | AF120 |
| Al salt | 2.0-8.0 | Al salt | 2.0-8.0 | Al salt | 2.0-8.0 |
| KM | 0.05-2.0 | KM | 0.05-2.0 | KM | 0.05-2.0 |
| ZK299 | 0.1-5.0 | ZK300 | 0.1-5.0 | ZK301 | 0.1-5.0 |
| Carrier 1) | to make | Carrier 1) | to make | Carrier 1) | to make |
| | 100 | | 100 | | 100 |
| | AF121 | | AF122 | | AF123 |
| Al salt | 2.0-8.0 | Al salt | 2.0-8.0 | Al salt | 2.0-8.0 |
| KM | 0.05-2.0 | KM | 0.05-2.0 | KM | 0.05-2.0 |
| ZK302 | 0.1-5.0 | ZK303 | 0.1-5.0 | ZK304 | 0.1-5.0 |
| Carrier 1) | to make | Carrier 1) | to make | Carrier 1) | to make |
| | 100 | | 100 | | 100 |
| | AF124 | | AF125 | | AF126 |
| Al salt | 2.0-8.0 | Al salt | 2.0-8.0 | Al salt | 2.0-8.0 |
| KM | 0.05-2.0 | KM | 0.05-2.0 | KM | 0.05-2.0 |
| ZK305 | 0.1-5.0 | ZK306 | 0.1-5.0 | ZK307 | 0.1-5.0 |
| Carrier 1) | to make | Carrier 1) | to make | Carrier 1) | to make |
| | 100 | | 100 | | 100 |
| | AF127 | | AF128 | | AF129 |
| Al salt | 2.0-8.0 | Al salt | 2.0-8.0 | Al salt | 2.0-8.0 |
| KM | 0.05-2.0 | KM | 0.05-2.0 | KM | 0.05-2.0 |
| ZK308 | 0.1-5.0 | ZK309 | 0.1-5.0 | ZK310 | 0.1-5.0 |
| Carrier 1) | to make | Carrier 1) | to make | Carrier 1) | to make |
| | 100 | | 100 | | 100 |
| | AF130 | | AF131 | | AF132 |
| Al salt | 2.0-8.0 | Al salt | 2.0-8.0 | Al salt | 2.0-8.0 |
| KM | 0.05-2.0 | KM | 0.05-2.0 | KM | 0.05-2.0 |
| ZK311 | 0.1-5.0 | ZK312 | 0.1-5.0 | ZK313 | 0.1-5.0 |
| Carrier 1) | to make | Carrier 1) | to make | Carrier 1) | to make |
| | 100 | | 100 | | 100 |
| | AF133 | | AF134 | | AF135 |
| Al salt | 2.0-8.0 | Al salt | 2.0-8.0 | Al salt | 2.0-8.0 |
| KM | 0.05-2.0 | KM | 0.05-2.0 | KM | 0.05-2.0 |
| ZK314 | 0.1-5.0 | ZK315 | 0.1-5.0 | ZK316 | 0.1-5.0 |
| Carrier 1) | to make | Carrier 1) | to make | Carrier 1) | to make |
| | 100 | | 100 | | 100 |
| | AF136 | | AF137 | | AF138 |
| Al salt | 2.0-8.0 | Al salt | 2.0-8.0 | Al salt | 2.0-8.0 |
| KM | 0.05-2.0 | KM | 0.05-2.0 | KM | 0.05-2.0 |
| ZK317 | 0.1-5.0 | ZK318 | 0.1-5.0 | ZK319 | 0.1-5.0 |
| Carrier 1) | to make | Carrier 1) | to make | Carrier 1) | to make |
| | 100 | | 100 | | 100 |
| | AF139 | | AF140 | | AF141 |
| Al salt | 2.0-8.0 | Al salt | 2.0-8.0 | Al salt | 2.0-8.0 |
| KM | 0.05-2.0 | KM | 0.05-2.0 | KM | 0.05-2.0 |
| ZK320 | 0.1-5.0 | ZK321 | 0.1-5.0 | ZK322 | 0.1-5.0 |
| Carrier 1) | to make | Carrier 1) | to make | Carrier 1) | to make |
| | 100 | | 100 | | 100 |
| | AF142 | | AF143 | | AF144 |
| Al salt | 2.0-8.0 | Al salt | 2.0-8.0 | Al salt | 2.0-8.0 |
| KM | 0.05-2.0 | KM | 0.05-2.0 | KM | 0.05-2.0 |
| ZK323 | 0.1-5.0 | ZK324 | 0.1-5.0 | ZK325 | 0.1-5.0 |
| Carrier 1) | to make | Carrier 1) | to make | Carrier 1) | to make |
| | 100 | | 100 | | 100 |
| | AF145 | | AF146 | | AF147 |
| Al salt | 2.0-8.0 | Al salt | 2.0-8.0 | Al salt | 2.0-8.0 |
| KM | 0.05-2.0 | KM | 0.05-2.0 | KM | 0.05-2.0 |
| ZK326 | 0.1-5.0 | ZK327 | 0.1-5.0 | ZK328 | 0.1-5.0 |
| Carrier 1) | to make | Carrier 1) | to make | Carrier 1) | to make |
| | 100 | | 100 | | 100 |
| | AF148 | | AF149 | | AF150 |
| Al salt | 2.0-8.0 | Al salt | 2.0-8.0 | Al salt | 2.0-8.0 |
| KM | 0.05-2.0 | KM | 0.05-2.0 | KM | 0.05-2.0 |
| ZK329 | 0.1-5.0 | ZK330 | 0.1-5.0 | ZK331 | 0.1-5.0 |
| Carrier 1) | to make | Carrier 1) | to make | Carrier 1) | to make |
| | 100 | | 100 | | 100 |
| | AF151 | | AF152 | | AF153 |
| Al salt | 2.0-8.0 | Al salt | 2.0-8.0 | Al salt | 2.0-8.0 |
| KM | 0.05-2.0 | KM | 0.05-2.0 | KM | 0.05-2.0 |
| ZK332 | 0.1-5.0 | ZK333 | 0.1-5.0 | ZK334 | 0.1-5.0 |
| Carrier 1) | to make | Carrier 1) | to make | Carrier 1) | to make |
| | 100 | | 100 | | 100 |
| | AF154 | | AF155 | | AF156 |
| Al salt | 2.0-8.0 | Al salt | 2.0-8.0 | Al salt | 2.0-8.0 |
| KM | 0.05-2.0 | KM | 0.05-2.0 | KM | 0.05-2.0 |
| ZK335 | 0.1-5.0 | ZK336 | 0.1-5.0 | ZK337 | 0.1-5.0 |
| Carrier 1) | to make | Carrier 1) | to make | Carrier 1) | to make |
| | 100 | | 100 | | 100 |
| | AF157 | | AF158 | | AF159 |
| Al salt | 2.0-8.0 | Al salt | 2.0-8.0 | Al salt | 2.0-8.0 |
| KM | 0.05-2.0 | KM | 0.05-2.0 | KM | 0.05-2.0 |
| ZK338 | 0.1-5.0 | ZK339 | 0.1-5.0 | ZK340 | 0.1-5.0 |
| Carrier 1) | to make | Carrier 1) | to make | Carrier 1) | to make |
| | 100 | | 100 | | 100 |
| | AF160 | | AF161 | | AF162 |
| Al salt | 2.0-8.0 | Al salt | 2.0-8.0 | Al salt | 2.0-8.0 |
| KM | 0.05-2.0 | KM | 0.05-2.0 | KM | 0.05-2.0 |
| ZK341 | 0.1-5.0 | ZK342 | 0.1-5.0 | ZK343 | 0.1-5.0 |
| Carrier 1) | to make | Carrier 1) | to make | Carrier 1) | to make |
| | 100 | | 100 | | 100 |
| | AF163 | | AF164 | | AF165 |
| Al salt | 2.0-8.0 | Al salt | 2.0-8.0 | Al salt | 2.0-8.0 |
| KM | 0.05-2.0 | KM | 0.05-2.0 | KM | 0.05-2.0 |
| ZK344 | 0.1-5.0 | ZK345 | 0.1-5.0 | ZK346 | 0.1-5.0 |
| Carrier 1) | to make | Carrier 1) | to make | Carrier 1) | to make |
| | 100 | | 100 | | 100 |
| | AF166 | | AF167 | | AF168 |
| Al salt | 2.0-8.0 | Al salt | 2.0-8.0 | Al salt | 2.0-8.0 |
| KM | 0.05-2.0 | KM | 0.05-2.0 | KM | 0.05-2.0 |
| ZK347 | 0.1-5.0 | ZK265 | 0.1-5.0 | ZK266 | 0.1-5.0 |
| Carrier 1) | to make | Carrier 1) | to make | Carrier 1) | to make |
| | 100 | | 100 | | 100 |
| | AF169 | | AF170 | | AF171 |
| Deodorant | 0.1-2.0 | Deodorant | 0.1-2.0 | Deodorant | 0.1-2.0 |
| active | | active | | active | |
| ingredient | | ingredient | | ingredient | |
| Al salt | 2.0-8.0 | Al salt | 2.0-8.0 | Al salt | 2.0-8.0 |
| KM | 0.05-2.0 | KM | 0.05-2.0 | KM | 0.05-2.0 |
| ZK267 | 0.1-5.0 | ZK268 | 0.1-5.0 | ZK269 | 0.1-5.0 |
| Carrier 1) | to make | Carrier 1) | to make | Carrier 1) | to make |
| | 100 | | 100 | | 100 |
| | AF172 | | AF173 | | AF174 |
| Deodorant | 0.1-2.0 | Deodorant | 0.1-2.0 | Deodorant | 0.1-2.0 |
| active | | active | | active | |
| ingredient | | ingredient | | ingredient | |
| Al salt | 2.0-8.0 | Al salt | 2.0-8.0 | Al salt | 2.0-8.0 |
| KM | 0.05-2.0 | KM | 0.05-2.0 | KM | 0.05-2.0 |
| ZK270 | 0.1-5.0 | ZK271 | 0.1-5.0 | ZK272 | 0.1-5.0 |
| Carrier 1) | to make | Carrier 1) | to make | Carrier 1) | to make |
| | 100 | | 100 | | 100 |
| | AF175 | | AF176 | | AF177 |
| Deodorant | 0.1-2.0 | Deodorant | 0.1-2.0 | Deodorant | 0.1-2.0 |
| active | | active | | active | |
| ingredient | | ingredient | | ingredient | |
| Al salt | 2.0-8.0 | Al salt | 2.0-8.0 | Al salt | 2.0-8.0 |
| KM | 0.05-2.0 | KM | 0.05-2.0 | KM | 0.05-2.0 |
| ZK273 | 0.1-5.0 | ZK274 | 0.1-5.0 | ZK275 | 0.1-5.0 |
| Carrier 1) | to make | Carrier 1) | to make | Carrier 1) | to make |
| | 100 | | 100 | | 100 |
| | AF178 | | AF179 | | AF180 |
| Deodorant | 0.1-2.0 | Deodorant | 0.1-2.0 | Deodorant | 0.1-2.0 |
| active | | active | | active | |
| ingredient | | ingredient | | ingredient | |
| Al salt | 2.0-8.0 | Al salt | 2.0-8.0 | Al salt | 2.0-8.0 |
| KM | 0.05-2.0 | KM | 0.05-2.0 | KM | 0.05-2.0 |
| ZK276 | 0.1-5.0 | ZK277 | 0.1-5.0 | ZK278 | 0.1-5.0 |
| Carrier 1) | to make | Carrier 1) | to make | Carrier 1) | to make |
| | 100 | | 100 | | 100 |
| | AF181 | | AF182 | | AF183 |
| Deodorant | 0.1-2.0 | Deodorant | 0.1-2.0 | Deodorant | 0.1-2.0 |
| active | | active | | active | |
| ingredient | | ingredient | | ingredient | |
| Al salt | 2.0-8.0 | Al salt | 2.0-8.0 | Al salt | 2.0-8.0 |
| KM | 0.05-2.0 | KM | 0.05-2.0 | KM | 0.05-2.0 |
| ZK279 | 0.1-5.0 | ZK280 | 0.1-5.0 | ZK280 | 0.1-5.0 |
| Carrier 1) | to make | Carrier 1) | to make | Carrier 1) | to make |
| | 100 | | 100 | | 100 |
| | AF184 | | AF185 | | AF186 |
| Deodorant | 0.1-2.0 | Deodorant | 0.1-2.0 | Deodorant | 0.1-2.0 |
| active | | active | | active | |
| ingredient | | ingredient | | ingredient | |
| Al salt | 2.0-8.0 | Al salt | 2.0-8.0 | Al salt | 2.0-8.0 |
| KM | 0.05-2.0 | KM | 0.05-2.0 | KM | 0.05-2.0 |
| ZK281 | 0.1-5.0 | ZK282 | 0.1-5.0 | ZK283 | 0.1-5.0 |
| Carrier 1) | to make | Carrier 1) | to make | Carrier 1) | to make |
| | 100 | | 100 | | 100 |
| | AF187 | | AF188 | | AF189 |
| Deodorant | 0.1-2.0 | Deodorant | 0.1-2.0 | Deodorant | 0.1-2.0 |
| active | | active | | active | |
| ingredient | | ingredient | | ingredient | |
| Al salt | 2.0-8.0 | Al salt | 2.0-8.0 | Al salt | 2.0-8.0 |
| KM | 0.05-2.0 | KM | 0.05-2.0 | KM | 0.05-2.0 |
| ZK284 | 0.1-5.0 | ZK285 | 0.1-5.0 | ZK286 | 0.1-5.0 |
| Carrier 1) | to make | Carrier 1) | to make | Carrier 1) | to make |
| | 100 | | 100 | | 100 |
| | AF190 | | AF191 | | AF192 |
| Deodorant | 0.1-2.0 | Deodorant | 0.1-2.0 | Deodorant | 0.1-2.0 |
| active | | active | | active | |
| ingredient | | ingredient | | ingredient | |
| Al salt | 2.0-8.0 | Al salt | 2.0-8.0 | Al salt | 2.0-8.0 |
| KM | 0.05-2.0 | KM | 0.05-2.0 | KM | 0.05-2.0 |
| ZK287 | 0.1-5.0 | ZK288 | 0.1-5.0 | ZK289 | 0.1-5.0 |
| Carrier 1) | to make | Carrier 1) | to make | Carrier 1) | to make |
| | 100 | | 100 | | 100 |
| | AF193 | | AF194 | | AF195 |
| Deodorant | 0.1-2.0 | Deodorant | 0.1-2.0 | Deodorant | 0.1-2.0 |
| active | | active | | active | |
| ingredient | | ingredient | | ingredient | |
| Al salt | 2.0-8.0 | Al salt | 2.0-8.0 | Al salt | 2.0-8.0 |
| KM | 0.05-2.0 | KM | 0.05-2.0 | KM | 0.05-2.0 |
| ZK290 | 0.1-5.0 | ZK291 | 0.1-5.0 | ZK292 | 0.1-5.0 |
| Carrier 1) | to make | Carrier 1) | to make | Carrier 1) | to make |
| | 100 | | 100 | | 100 |
| | AF196 | | AF197 | | AF198 |
| Deodorant | 0.1-2.0 | Deodorant | 0.1-2.0 | Deodorant | 0.1-2.0 |
| active | | active | | active | |
| ingredient | | ingredient | | ingredient | |
| Al salt | 2.0-8.0 | Al salt | 2.0-8.0 | Al salt | 2.0-8.0 |
| KM | 0.05-2.0 | KM | 0.05-2.0 | KM | 0.05-2.0 |
| ZK293 | 0.1-5.0 | ZK294 | 0.1-5.0 | ZK295 | 0.1-5.0 |
| Carrier 1) | to make | Carrier 1) | to make | Carrier 1) | to make |
| | 100 | | 100 | | 100 |
| | AF199 | | AF200 | | AF201 |
| Deodorant | 0.1-2.0 | Deodorant | 0.1-2.0 | Deodorant | 0.1-2.0 |
| active | | active | | active | |
| ingredient | | ingredient | | ingredient | |
| Al salt | 2.0-8.0 | Al salt | 2.0-8.0 | Al salt | 2.0-8.0 |
| KM | 0.05-2.0 | KM | 0.05-2.0 | KM | 0.05-2.0 |
| ZK296 | 0.1-5.0 | ZK297 | 0.1-5.0 | ZK298 | 0.1-5.0 |
| Carrier 1) | to make | Carrier 1) | to make | Carrier 1) | to make |
| | 100 | | 100 | | 100 |
| | AF202 | | AF203 | | AF204 |
| Deodorant | 0.1-2.0 | Deodorant | 0.1-2.0 | Deodorant | 0.1-2.0 |
| active | | active | | active | |
| ingredient | | ingredient | | ingredient | |
| Al salt | 2.0-8.0 | Al salt | 2.0-8.0 | Al salt | 2.0-8.0 |
| KM | 0.05-2.0 | KM | 0.05-2.0 | KM | 0.05-2.0 |
| ZK299 | 0.1-5.0 | ZK300 | 0.1-5.0 | ZK301 | 0.1-5.0 |
| Carrier 1) | to make | Carrier 1) | to make | Carrier 1) | to make |
| | 100 | | 100 | | 100 |
| | AF205 | | AF206 | | AF207 |
| Deodorant | 0.1-2.0 | Deodorant | 0.1-2.0 | Deodorant | 0.1-2.0 |
| active | | active | | active | |
| ingredient | | ingredient | | ingredient | |
| Al salt | 2.0-8.0 | Al salt | 2.0-8.0 | Al salt | 2.0-8.0 |
| KM | 0.05-2.0 | KM | 0.05-2.0 | KM | 0.05-2.0 |
| ZK302 | 0.1-5.0 | ZK303 | 0.1-5.0 | ZK304 | 0.1-5.0 |
| Carrier 1) | to make | Carrier 1) | to make | Carrier 1) | to make |
| | 100 | | 100 | | 100 |
| | AF208 | | AF209 | | AF210 |
| Deodorant | 0.1-2.0 | Deodorant | 0.1-2.0 | Deodorant | 0.1-2.0 |
| active | | active | | active | |
| ingredient | | ingredient | | ingredient | |
| Al salt | 2.0-8.0 | Al salt | 2.0-8.0 | Al salt | 2.0-8.0 |
| KM | 0.05-2.0 | KM | 0.05-2.0 | KM | 0.05-2.0 |
| ZK305 | 0.1-5.0 | ZK306 | 0.1-5.0 | ZK307 | 0.1-5.0 |
| Carrier 1) | to make | Carrier 1) | to make | Carrier 1) | to make |
| | 100 | | 100 | | 100 |
| | AF211 | | AF212 | | AF213 |
| Deodorant | 0.1-2.0 | Deodorant | 0.1-2.0 | Deodorant | 0.1-2.0 |
| active | | active | | active | |
| ingredient | | ingredient | | ingredient | |
| Al salt | 2.0-8.0 | Al salt | 2.0-8.0 | Al salt | 2.0-8.0 |
| KM | 0.05-2.0 | KM | 0.05-2.0 | KM | 0.05-2.0 |
| ZK308 | 0.1-5.0 | ZK309 | 0.1-5.0 | ZK310 | 0.1-5.0 |
| Carrier 1) | to make | Carrier 1) | to make | Carrier 1) | to make |
| | 100 | | 100 | | 100 |
| | AF214 | | AF215 | | AF216 |
| Deodorant | 0.1-2.0 | Deodorant | 0.1-2.0 | Deodorant | 0.1-2.0 |
| active | | active | | active | |
| ingredient | | ingredient | | ingredient | |
| Al salt | 2.0-8.0 | Al salt | 2.0-8.0 | Al salt | 2.0-8.0 |
| KM | 0.05-2.0 | KM | 0.05-2.0 | KM | 0.05-2.0 |
| ZK311 | 0.1-5.0 | ZK312 | 0.1-5.0 | ZK313 | 0.1-5.0 |
| Carrier 1) | to make | Carrier 1) | to make | Carrier 1) | to make |
| | 100 | | 100 | | 100 |
| | AF217 | | AF218 | | AF219 |
| Deodorant | 0.1-2.0 | Deodorant | 0.1-2.0 | Deodorant | 0.1-2.0 |
| active | | active | | active | |
| ingredient | | ingredient | | ingredient | |
| Al salt | 2.0-8.0 | Al salt | 2.0-8.0 | Al salt | 2.0-8.0 |
| KM | 0.05-2.0 | KM | 0.05-2.0 | KM | 0.05-2.0 |
| ZK314 | 0.1-5.0 | ZK315 | 0.1-5.0 | ZK316 | 0.1-5.0 |
| Carrier 1) | to make | Carrier 1) | to make | Carrier 1) | to make |
| | 100 | | 100 | | 100 |
| | AF220 | | AF221 | | AF222 |
| Deodorant | 0.1-2.0 | Deodorant | 0.1-2.0 | Deodorant | 0.1-2.0 |
| active | | active | | active | |
| ingredient | | ingredient | | ingredient | |
| Al salt | 2.0-8.0 | Al salt | 2.0-8.0 | Al salt | 2.0-8.0 |
| KM | 0.05-2.0 | KM | 0.05-2.0 | KM | 0.05-2.0 |
| ZK317 | 0.1-5.0 | ZK318 | 0.1-5.0 | ZK319 | 0.1-5.0 |
| Carrier 1) | to make | Carrier 1) | to make | Carrier 1) | to make |
| | 100 | | 100 | | 100 |
| | AF223 | | AF224 | | AF225 |
| Deodorant | 0.1-2.0 | Deodorant | 0.1-2.0 | Deodorant | 0.1-2.0 |
| active | | active | | active | |
| ingredient | | ingredient | | ingredient | |
| Al salt | 2.0-8.0 | Al salt | 2.0-8.0 | Al salt | 2.0-8.0 |
| KM | 0.05-2.0 | KM | 0.05-2.0 | KM | 0.05-2.0 |
| ZK320 | 0.1-5.0 | ZK321 | 0.1-5.0 | ZK322 | 0.1-5.0 |
| Carrier 1) | to make | Carrier 1) | to make | Carrier 1) | to make |
| | 100 | | 100 | | 100 |
| | AF226 | | AF227 | | AF228 |
| Deodorant | 0.1-2.0 | Deodorant | 0.1-2.0 | Deodorant | 0.1-2.0 |
| active | | active | | active | |
| ingredient | | ingredient | | ingredient | |
| Al salt | 2.0-8.0 | Al salt | 2.0-8.0 | Al salt | 2.0-8.0 |
| KM | 0.05-2.0 | KM | 0.05-2.0 | KM | 0.05-2.0 |
| ZK323 | 0.1-5.0 | ZK324 | 0.1-5.0 | ZK325 | 0.1-5.0 |
| Carrier 1) | to make | Carrier 1) | to make | Carrier 1) | to make |
| | 100 | | 100 | | 100 |
| | AF229 | | AF230 | | AF231 |
| Deodorant | 0.1-2.0 | Deodorant | 0.1-2.0 | Deodorant | 0.1-2.0 |
| active | | active | | active | |
| ingredient | | ingredient | | ingredient | |
| Al salt | 2.0-8.0 | Al salt | 2.0-8.0 | Al salt | 2.0-8.0 |
| KM | 0.05-2.0 | KM | 0.05-2.0 | KM | 0.05-2.0 |
| ZK326 | 0.1-5.0 | ZK327 | 0.1-5.0 | ZK328 | 0.1-5.0 |
| Carrier 1) | to make | Carrier 1) | to make | Carrier 1) | to make |
| | 100 | | 100 | | 100 |
| | AF232 | | AF233 | | AF234 |
| Deodorant | 0.1-2.0 | Deodorant | 0.1-2.0 | Deodorant | 0.1-2.0 |
| active | | active | | active | |
| ingredient | | ingredient | | ingredient | |
| Al salt | 2.0-8.0 | Al salt | 2.0-8.0 | Al salt | 2.0-8.0 |
| KM | 0.05-2.0 | KM | 0.05-2.0 | KM | 0.05-2.0 |
| ZK329 | 0.1-5.0 | ZK330 | 0.1-5.0 | ZK331 | 0.1-5.0 |
| Carrier 1) | to make | Carrier 1) | to make | Carrier 1) | to make |
| | 100 | | 100 | | 100 |
| | AF235 | | AF236 | | AF237 |
| Deodorant | 0.1-2.0 | Deodorant | 0.1-2.0 | Deodorant | 0.1-2.0 |
| active | | active | | active | |
| ingredient | | ingredient | | ingredient | |
| Al salt | 2.0-8.0 | Al salt | 2.0-8.0 | Al salt | 2.0-8.0 |
| KM | 0.05-2.0 | KM | 0.05-2.0 | KM | 0.05-2.0 |
| ZK332 | 0.1-5.0 | ZK333 | 0.1-5.0 | ZK334 | 0.1-5.0 |
| Carrier 1) | to make | Carrier 1) | to make | Carrier 1) | to make |
| | 100 | | 100 | | 100 |
| | AF238 | | AF239 | | AF240 |
| Deodorant | 0.1-2.0 | Deodorant | 0.1-2.0 | Deodorant | 0.1-2.0 |
| active | | active | | active | |
| ingredient | | ingredient | | ingredient | |
| Al salt | 2.0-8.0 | Al salt | 2.0-8.0 | Al salt | 2.0-8.0 |
| KM | 0.05-2.0 | KM | 0.05-2.0 | KM | 0.05-2.0 |
| ZK335 | 0.1-5.0 | ZK336 | 0.1-5.0 | ZK337 | 0.1-5.0 |
| Carrier 1) | to make | Carrier 1) | to make | Carrier 1) | to make |
| | 100 | | 100 | | 100 |
| | AF241 | | AF242 | | AF243 |
| Deodorant | 0.1-2.0 | Deodorant | 0.1-2.0 | Deodorant | 0.1-2.0 |
| active | | active | | active | |
| ingredient | | ingredient | | ingredient | |
| Al salt | 2.0-8.0 | Al salt | 2.0-8.0 | Al salt | 2.0-8.0 |
| KM | 0.05-2.0 | KM | 0.05-2.0 | KM | 0.05-2.0 |
| ZK338 | 0.1-5.0 | ZK339 | 0.1-5.0 | ZK340 | 0.1-5.0 |
| Carrier 1) | to make | Carrier 1) | to make | Carrier 1) | to make |
| | 100 | | 100 | | 100 |
| | AF244 | | AF245 | | AF246 |
| Deodorant | 0.1-2.0 | Deodorant | 0.1-2.0 | Deodorant | 0.1-2.0 |
| active | | active | | active | |
| ingredient | | ingredient | | ingredient | |
| Al salt | 2.0-8.0 | Al salt | 2.0-8.0 | Al salt | 2.0-8.0 |
| KM | 0.05-2.0 | KM | 0.05-2.0 | KM | 0.05-2.0 |
| ZK341 | 0.1-5.0 | ZK342 | 0.1-5.0 | ZK343 | 0.1-5.0 |
| Carrier 1) | to make | Carrier 1) | to make | Carrier 1) | to make |
| | 100 | | 100 | | 100 |
| | AF247 | | AF248 | | AF249 |
| Deodorant | 0.1-2.0 | Deodorant | 0.1-2.0 | Deodorant | 0.1-2.0 |
| active | | active | | active | |
| ingredient | | ingredient | | ingredient | |
| Al salt | 2.0-8.0 | Al salt | 2.0-8.0 | Al salt | 2.0-8.0 |
| KM | 0.05-2.0 | KM | 0.05-2.0 | KM | 0.05-2.0 |
| ZK344 | 0.1-5.0 | ZK345 | 0.1-5.0 | ZK346 | 0.1-5.0 |
| Carrier 1) | to make | Carrier 1) | to make | Carrier 1) | to make |
| | 100 | | 100 | | 100 |
| | AF250 | | AF251 | | AF252 |
| Deodorant | 0.1-2.0 | Deodorant | 0.1-2.0 | Deodorant | 0.1-2.0 |
| active | | active | | active | |
| ingredient | | ingredient | | ingredient | |
| Al salt | 2.0-8.0 | Al salt | 2.0-8.0 | Al salt | 2.0-8.0 |
| KM | 0.05-2.0 | KM | 0.05-2.0 | KM | 0.05-2.0 |
| ZK347 | 0.1-5.0 | ZK265 | 0.1-5.0 | ZK266 | 0.1-5.0 |
| Carrier 1) | to make | Carrier 1) | to make | Carrier 1) | to make |
| | 100 | | 100 | | 100 |
| | AF253 | | AF254 | | AF255 |
| Deodorant | 0.1-2.0 | Deodorant | 0.1-2.0 | Deodorant | 0.1-2.0 |
| active | | active | | active | |
| ingredient 2) | | ingredient 2) | | ingredient 2) | |
| KM | 0.05-2.0 | KM | 0.05-2.0 | KM | 0.05-2.0 |
| ZK267 | 0.1-5.0 | ZK268 | 0.1-5.0 | ZK269 | 0.1-5.0 |
| Carrier 1) | to make | Carrier 1) | to make | Carrier 1) | to make |
| | 100 | | 100 | | 100 |
| | AF256 | | AF257 | | AF258 |
| Deodorant | 0.1-2.0 | Deodorant | 0.1-2.0 | Deodorant | 0.1-2.0 |
| active | | active | | active | |
| ingredient 2) | | ingredient 2) | | ingredient 2) | |
| KM | 0.05-2.0 | KM | 0.05-2.0 | KM | 0.05-2.0 |
| ZK270 | 0.1-5.0 | ZK271 | 0.1-5.0 | ZK272 | 0.1-5.0 |
| Carrier 1) | to make | Carrier 1) | to make | Carrier 1) | to make |
| | 100 | | 100 | | 100 |
| | AF259 | | AF260 | | AF261 |
| Deodorant | 0.1-2.0 | Deodorant | 0.1-2.0 | Deodorant | 0.1-2.0 |
| active | | active | | active | |
| ingredient 2) | | ingredient 2) | | ingredient 2) | |
| KM | 0.05-2.0 | KM | 0.05-2.0 | KM | 0.05-2.0 |
| ZK273 | 0.1-5.0 | ZK274 | 0.1-5.0 | ZK275 | 0.1-5.0 |
| Carrier 1) | to make | Carrier 1) | to make | Carrier 1) | to make |
| | 100 | | 100 | | 100 |
| | AF262 | | AF263 | | AF264 |
| Deodorant | 0.1-2.0 | Deodorant | 0.1-2.0 | Deodorant | 0.1-2.0 |
| active | | active | | active | |
| ingredient 2) | | ingredient 2) | | ingredient 2) | |
| KM | 0.05-2.0 | KM | 0.05-2.0 | KM | 0.05-2.0 |
| ZK276 | 0.1-5.0 | ZK277 | 0.1-5.0 | ZK278 | 0.1-5.0 |
| Carrier 1) | to make | Carrier 1) | to make | Carrier 1) | to make |
| | 100 | | 100 | | 100 |
| | AF265 | | AF266 | | AF267 |
| Deodorant | 0.1-2.0 | Deodorant | 0.1-2.0 | Deodorant | 0.1-2.0 |
| active | | active | | active | |
| ingredient 2) | | ingredient 2) | | ingredient 2) | |
| KM | 0.05-2.0 | KM | 0.05-2.0 | KM | 0.05-2.0 |
| ZK279 | 0.1-5.0 | ZK280 | 0.1-5.0 | ZK280 | 0.1-5.0 |
| Carrier 1) | to make | Carrier 1) | to make | Carrier 1) | to make |
| | 100 | | 100 | | 100 |
| | AF268 | | AF269 | | AF270 |
| Deodorant | 0.1-2.0 | Deodorant | 0.1-2.0 | Deodorant | 0.1-2.0 |
| active | | active | | active | |
| ingredient 2) | | ingredient 2) | | ingredient 2) | |
| KM | 0.05-2.0 | KM | 0.05-2.0 | KM | 0.05-2.0 |
| ZK281 | 0.1-5.0 | ZK282 | 0.1-5.0 | ZK283 | 0.1-5.0 |
| Carrier 1) | to make | Carrier 1) | to make | Carrier 1) | to make |
| | 100 | | 100 | | 100 |
| | AF271 | | AF272 | | AF273 |
| Deodorant | 0.1-2.0 | Deodorant | 0.1-2.0 | Deodorant | 0.1-2.0 |
| active | | active | | active | |
| ingredient 2) | | ingredient 2) | | ingredient 2) | |
| KM | 0.05-2.0 | KM | 0.05-2.0 | KM | 0.05-2.0 |
| ZK284 | 0.1-5.0 | ZK285 | 0.1-5.0 | ZK286 | 0.1-5.0 |
| Carrier 1) | to make | Carrier 1) | to make | Carrier 1) | to make |
| | 100 | | 100 | | 100 |
| | AF274 | | AF275 | | AF276 |
| Deodorant | 0.1-2.0 | Deodorant | 0.1-2.0 | Deodorant | 0.1-2.0 |
| active | | active | | active | |
| ingredient 2) | | ingredient 2) | | ingredient 2) | |
| KM | 0.05-2.0 | KM | 0.05-2.0 | KM | 0.05-2.0 |
| ZK287 | 0.1-5.0 | ZK288 | 0.1-5.0 | ZK289 | 0.1-5.0 |
| Carrier 1) | to make | Carrier 1) | to make | Carrier 1) | to make |
| | 100 | | 100 | | 100 |
| | AF277 | | AF278 | | AF279 |
| Deodorant | 0.1-2.0 | Deodorant | 0.1-2.0 | Deodorant | 0.1-2.0 |
| active | | active | | active | |
| ingredient 2) | | ingredient 2) | | ingredient 2) | |
| KM | 0.05-2.0 | KM | 0.05-2.0 | KM | 0.05-2.0 |
| ZK290 | 0.1-5.0 | ZK291 | 0.1-5.0 | ZK292 | 0.1-5.0 |
| Carrier 1) | to make | Carrier 1) | to make | Carrier 1) | to make |
| | 100 | | 100 | | 100 |
| | AF280 | | AF281 | | AF282 |
| Deodorant | 0.1-2.0 | Deodorant | 0.1-2.0 | Deodorant | 0.1-2.0 |
| active | | active | | active | |
| ingredient 2) | | ingredient 2) | | ingredient 2) | |
| KM | 0.05-2.0 | KM | 0.05-2.0 | KM | 0.05-2.0 |
| ZK293 | 0.1-5.0 | ZK294 | 0.1-5.0 | ZK295 | 0.1-5.0 |
| Carrier 1) | to make | Carrier 1) | to make | Carrier 1) | to make |
| | 100 | | 100 | | 100 |
| | AF283 | | AF284 | | AF285 |
| Deodorant | 0.1-2.0 | Deodorant | 0.1-2.0 | Deodorant | 0.1-2.0 |
| active | | active | | active | |
| ingredient 2) | | ingredient 2) | | ingredient 2) | |
| KM | 0.05-2.0 | KM | 0.05-2.0 | KM | 0.05-2.0 |
| ZK296 | 0.1-5.0 | ZK297 | 0.1-5.0 | ZK298 | 0.1-5.0 |
| Carrier 1) | to make | Carrier 1) | to make | Carrier 1) | to make |
| | 100 | | 100 | | 100 |
| | AF286 | | AF287 | | AF288 |
| Deodorant | 0.1-2.0 | Deodorant | 0.1-2.0 | Deodorant | 0.1-2.0 |
| active | | active | | active | |
| ingredient 2) | | ingredient 2) | | ingredient 2) | |
| KM | 0.05-2.0 | KM | 0.05-2.0 | KM | 0.05-2.0 |
| ZK299 | 0.1-5.0 | ZK300 | 0.1-5.0 | ZK301 | 0.1-5.0 |
| Carrier 1) | to make | Carrier 1) | to make | Carrier 1) | to make |
| | 100 | | 100 | | 100 |
| | AF289 | | AF290 | | AF291 |
| Deodorant | 0.1-2.0 | Deodorant | 0.1-2.0 | Deodorant | 0.1-2.0 |
| active | | active | | active | |
| ingredient 2) | | ingredient 2) | | ingredient 2) | |
| KM | 0.05-2.0 | KM | 0.05-2.0 | KM | 0.05-2.0 |
| ZK302 | 0.1-5.0 | ZK303 | 0.1-5.0 | ZK304 | 0.1-5.0 |
| Carrier 1) | to make | Carrier 1) | to make | Carrier 1) | to make |
| | 100 | | 100 | | 100 |
| | AF292 | | AF293 | | AF294 |
| Deodorant | 0.1-2.0 | Deodorant | 0.1-2.0 | Deodorant | 0.1-2.0 |
| active | | active | | active | |
| ingredient 2) | | ingredient 2) | | ingredient 2) | |
| KM | 0.05-2.0 | KM | 0.05-2.0 | KM | 0.05-2.0 |
| ZK305 | 0.1-5.0 | ZK306 | 0.1-5.0 | ZK307 | 0.1-5.0 |
| Carrier 1) | to make | Carrier 1) | to make | Carrier 1) | to make |
| | 100 | | 100 | | 100 |
| | AF295 | | AF296 | | AF297 |
| Deodorant | 0.1-2.0 | Deodorant | 0.1-2.0 | Deodorant | 0.1-2.0 |
| active | | active | | active | |
| ingredient 2) | | ingredient 2) | | ingredient 2) | |
| KM | 0.05-2.0 | KM | 0.05-2.0 | KM | 0.05-2.0 |
| ZK308 | 0.1-5.0 | ZK309 | 0.1-5.0 | ZK310 | 0.1-5.0 |
| Carrier 1) | to make | Carrier 1) | to make | Carrier 1) | to make |
| | 100 | | 100 | | 100 |
| | AF298 | | AF299 | | AF300 |
| Deodorant | 0.1-2.0 | Deodorant | 0.1-2.0 | Deodorant | 0.1-2.0 |
| active | | active | | active | |
| ingredient 2) | | ingredient 2) | | ingredient 2) | |
| KM | 0.05-2.0 | KM | 0.05-2.0 | KM | 0.05-2.0 |
| ZK311 | 0.1-5.0 | ZK312 | 0.1-5.0 | ZK313 | 0.1-5.0 |
| Carrier 1) | to make | Carrier 1) | to make | Carrier 1) | to make |
| | 100 | | 100 | | 100 |
| | AF301 | | AF302 | | AF303 |
| Deodorant | 0.1-2.0 | Deodorant | 0.1-2.0 | Deodorant | 0.1-2.0 |
| active | | active | | active | |
| ingredient 2) | | ingredient 2) | | ingredient 2) | |
| KM | 0.05-2.0 | KM | 0.05-2.0 | KM | 0.05-2.0 |
| ZK314 | 0.1-5.0 | ZK315 | 0.1-5.0 | ZK316 | 0.1-5.0 |
| Carrier 1) | to make | Carrier 1) | to make | Carrier 1) | to make |
| | 100 | | 100 | | 100 |
| | AF304 | | AF305 | | AF306 |
| Deodorant | 0.1-2.0 | Deodorant | 0.1-2.0 | Deodorant | 0.1-2.0 |
| active | | active | | active | |
| ingredient 2) | | ingredient 2) | | ingredient 2) | |
| KM | 0.05-2.0 | KM | 0.05-2.0 | KM | 0.05-2.0 |
| ZK317 | 0.1-5.0 | ZK318 | 0.1-5.0 | ZK319 | 0.1-5.0 |
| Carrier 1) | to make | Carrier 1) | to make | Carrier 1) | to make |
| | 100 | | 100 | | 100 |
| | AF307 | | AF308 | | AF309 |
| Deodorant | 0.1-2.0 | Deodorant | 0.1-2.0 | Deodorant | 0.1-2.0 |
| active | | active | | active | |
| ingredient 2) | | ingredient 2) | | ingredient 2) | |
| KM | 0.05-2.0 | KM | 0.05-2.0 | KM | 0.05-2.0 |
| ZK320 | 0.1-5.0 | ZK321 | 0.1-5.0 | ZK322 | 0.1-5.0 |
| Carrier 1) | to make | Carrier 1) | to make | Carrier 1) | to make |
| | 100 | | 100 | | 100 |
| | AF310 | | AF311 | | AF312 |
| Deodorant | 0.1-2.0 | Deodorant | 0.1-2.0 | Deodorant | 0.1-2.0 |
| active | | active | | active | |
| ingredient 2) | | ingredient 2) | | ingredient 2) | |
| KM | 0.05-2.0 | KM | 0.05-2.0 | KM | 0.05-2.0 |
| ZK323 | 0.1-5.0 | ZK324 | 0.1-5.0 | ZK325 | 0.1-5.0 |
| Carrier 1) | to make | Carrier 1) | to make | Carrier 1) | to make |
| | 100 | | 100 | | 100 |
| | AF313 | | AF314 | | AF315 |
| Deodorant | 0.1-2.0 | Deodorant | 0.1-2.0 | Deodorant | 0.1-2.0 |
| active | | active | | active | |
| ingredient 2) | | ingredient 2) | | ingredient 2) | |
| KM | 0.05-2.0 | KM | 0.05-2.0 | KM | 0.05-2.0 |
| ZK326 | 0.1-5.0 | ZK327 | 0.1-5.0 | ZK328 | 0.1-5.0 |
| Carrier 1) | to make | Carrier 1) | to make | Carrier 1) | to make |
| | 100 | | 100 | | 100 |
| | AF316 | | AF317 | | AF318 |
| Deodorant | 0.1-2.0 | Deodorant | 0.1-2.0 | Deodorant | 0.1-2.0 |
| active | | active | | active | |
| ingredient 2) | | ingredient 2) | | ingredient 2) | |
| KM | 0.05-2.0 | KM | 0.05-2.0 | KM | 0.05-2.0 |
| ZK329 | 0.1-5.0 | ZK330 | 0.1-5.0 | ZK331 | 0.1-5.0 |
| Carrier 1) | to make | Carrier 1) | to make | Carrier 1) | to make |
| | 100 | | 100 | | 100 |
| | AF319 | | AF320 | | AF321 |
| Deodorant | 0.1-2.0 | Deodorant | 0.1-2.0 | Deodorant | 0.1-2.0 |
| active | | active | | active | |
| ingredient 2) | | ingredient 2) | | ingredient 2) | |
| KM | 0.05-2.0 | KM | 0.05-2.0 | KM | 0.05-2.0 |
| ZK332 | 0.1-5.0 | ZK333 | 0.1-5.0 | ZK334 | 0.1-5.0 |
| Carrier 1) | to make | Carrier 1) | to make | Carrier 1) | to make |
| | 100 | | 100 | | 100 |
| | AF322 | | AF323 | | AF324 |
| Deodorant | 0.1-2.0 | Deodorant | 0.1-2.0 | Deodorant | 0.1-2.0 |
| active | | active | | active | |
| ingredient 2) | | ingredient 2) | | ingredient 2) | |
| KM | 0.05-2.0 | KM | 0.05-2.0 | KM | 0.05-2.0 |
| ZK335 | 0.1-5.0 | ZK336 | 0.1-5.0 | ZK337 | 0.1-5.0 |
| Carrier 1) | to make | Carrier 1) | to make | Carrier 1) | to make |
| | 100 | | 100 | | 100 |
| | AF325 | | AF326 | | AF327 |
| Deodorant | 0.1-2.0 | Deodorant | 0.1-2.0 | Deodorant | 0.1-2.0 |
| active | | active | | active | |
| ingredient 2) | | ingredient 2) | | ingredient 2) | |
| KM | 0.05-2.0 | KM | 0.05-2.0 | KM | 0.05-2.0 |
| ZK338 | 0.1-5.0 | ZK339 | 0.1-5.0 | ZK340 | 0.1-5.0 |
| Carrier 1) | to make | Carrier 1) | to make | Carrier 1) | to make |
| | 100 | | 100 | | 100 |
| | AF328 | | AF329 | | AF330 |
| Deodorant | 0.1-2.0 | Deodorant | 0.1-2.0 | Deodorant | 0.1-2.0 |
| active | | active | | active | |
| ingredient 2) | | ingredient 2) | | ingredient 2) | |
| KM | 0.05-2.0 | KM | 0.05-2.0 | KM | 0.05-2.0 |
| ZK341 | 0.1-5.0 | ZK342 | 0.1-5.0 | ZK343 | 0.1-5.0 |
| Carrier 1) | to make | Carrier 1) | to make | Carrier 1) | to make |
| | 100 | | 100 | | 100 |
| | AF331 | | AF332 | | AF333 |
| Deodorant | 0.1-2.0 | Deodorant | 0.1-2.0 | Deodorant | 0.1-2.0 |
| active | | active | | active | |
| ingredient 2) | | ingredient 2) | | ingredient 2) | |
| KM | 0.05-2.0 | KM | 0.05-2.0 | KM | 0.05-2.0 |
| ZK344 | 0.1-5.0 | ZK345 | 0.1-5.0 | ZK346 | 0.1-5.0 |
| Carrier 1) | to make | Carrier 1) | to make | Carrier 1) | to make |
| | 100 | | 100 | | 100 |
| | AF334 | | AF335 | | AF336 |
| Deodorant | 0.1-2.0 | Deodorant | 0.1-2.0 | Deodorant | 0.1-2.0 |
| active | | active | | active | |
| ingredient 2) | | ingredient 2) | | ingredient 2) | |
| KM | 0.05-2.0 | KM | 0.05-2.0 | KM | 0.05-2.0 |
| ZK347 | 0.1-5.0 | ZK265 | 0.1-5.0 | ZK266 | 0.1-5.0 |
| Carrier 1) | to make | Carrier 1) | to make | Carrier 1) | to make |
| | 100 | | 100 | | 100 |
| | AF337 | | AF338 | | AF339 |
| Al salt 3) | 2.0-8.0 | Al salt 3) | 2.0-8.0 | Al salt 3) | 2.0-8.0 |
| KM | 0.05-2.0 | KM | 0.05-2.0 | KM | 0.05-2.0 |
| ZK267 | 0.1-5.0 | ZK268 | 0.1-5.0 | ZK269 | 0.1-5.0 |
| Carrier 1) | to make | Carrier 1) | to make | Carrier 1) | to make |
| | 100 | | 100 | | 100 |
| | AF340 | | AF341 | | AF342 |
| Al salt 3) | 2.0-8.0 | Al salt 3) | 2.0-8.0 | Al salt 3) | 2.0-8.0 |
| KM | 0.05-2.0 | KM | 0.05-2.0 | KM | 0.05-2.0 |
| ZK270 | 0.1-5.0 | ZK271 | 0.1-5.0 | ZK272 | 0.1-5.0 |
| Carrier 1) | to make | Carrier 1) | to make | Carrier 1) | to make |
| | 100 | | 100 | | 100 |
| | AF343 | | AF344 | | AF345 |
| Al salt 3) | 2.0-8.0 | Al salt 3) | 2.0-8.0 | Al salt 3) | 2.0-8.0 |
| KM | 0.05-2.0 | KM | 0.05-2.0 | KM | 0.05-2.0 |
| ZK273 | 0.1-5.0 | ZK274 | 0.1-5.0 | ZK275 | 0.1-5.0 |
| Carrier 1) | to make | Carrier 1) | to make | Carrier 1) | to make |
| | 100 | | 100 | | 100 |
| | AF346 | | AF347 | | AF348 |
| Al salt 3) | 2.0-8.0 | Al salt 3) | 2.0-8.0 | Al salt 3) | 2.0-8.0 |
| KM | 0.05-2.0 | KM | 0.05-2.0 | KM | 0.05-2.0 |
| ZK276 | 0.1-5.0 | ZK277 | 0.1-5.0 | ZK278 | 0.1-5.0 |
| Carrier 1) | to make | Carrier 1) | to make | Carrier 1) | to make |
| | 100 | | 100 | | 100 |
| | AF349 | | AF350 | | AF351 |
| Al salt 3) | 2.0-8.0 | Al salt 3) | 2.0-8.0 | Al salt 3) | 2.0-8.0 |
| KM | 0.05-2.0 | KM | 0.05-2.0 | KM | 0.05-2.0 |
| ZK279 | 0.1-5.0 | ZK280 | 0.1-5.0 | ZK280 | 0.1-5.0 |
| Carrier 1) | to make | Carrier 1) | to make | Carrier 1) | to make |
| | 100 | | 100 | | 100 |
| | AF352 | | AF353 | | AF354 |
| Al salt 3) | 2.0-8.0 | Al salt 3) | 2.0-8.0 | Al salt 3) | 2.0-8.0 |
| KM | 0.05-2.0 | KM | 0.05-2.0 | KM | 0.05-2.0 |
| ZK281 | 0.1-5.0 | ZK282 | 0.1-5.0 | ZK283 | 0.1-5.0 |
| Carrier 1) | to make | Carrier 1) | to make | Carrier 1) | to make |
| | 100 | | 100 | | 100 |
| | AF355 | | AF356 | | AF357 |
| Al salt 3) | 2.0-8.0 | Al salt 3) | 2.0-8.0 | Al salt 3) | 2.0-8.0 |
| KM | 0.05-2.0 | KM | 0.05-2.0 | KM | 0.05-2.0 |
| ZK284 | 0.1-5.0 | ZK285 | 0.1-5.0 | ZK286 | 0.1-5.0 |
| Carrier 1) | to make | Carrier 1) | to make | Carrier 1) | to make |
| | 100 | | 100 | | 100 |
| | AF358 | | AF359 | | AF360 |
| Al salt 3) | 2.0-8.0 | Al salt 3) | 2.0-8.0 | Al salt 3) | 2.0-8.0 |
| KM | 0.05-2.0 | KM | 0.05-2.0 | KM | 0.05-2.0 |
| ZK287 | 0.1-5.0 | ZK288 | 0.1-5.0 | ZK289 | 0.1-5.0 |
| Carrier 1) | to make | Carrier 1) | to make | Carrier 1) | to make |
| | 100 | | 100 | | 100 |
| | AF361 | | AF362 | | AF363 |
| Al salt 3) | 2.0-8.0 | Al salt 3) | 2.0-8.0 | Al salt 3) | 2.0-8.0 |
| KM | 0.05-2.0 | KM | 0.05-2.0 | KM | 0.05-2.0 |
| ZK290 | 0.1-5.0 | ZK291 | 0.1-5.0 | ZK292 | 0.1-5.0 |
| Carrier 1) | to make | Carrier 1) | to make | Carrier 1) | to make |
| | 100 | | 100 | | 100 |
| | AF364 | | AF365 | | AF366 |
| Al salt 3) | 2.0-8.0 | Al salt 3) | 2.0-8.0 | Al salt 3) | 2.0-8.0 |
| KM | 0.05-2.0 | KM | 0.05-2.0 | KM | 0.05-2.0 |
| ZK293 | 0.1-5.0 | ZK294 | 0.1-5.0 | ZK295 | 0.1-5.0 |
| Carrier 1) | to make | Carrier 1) | to make | Carrier 1) | to make |
| | 100 | | 100 | | 100 |
| | AF367 | | AF368 | | AF369 |
| Al salt 3) | 2.0-8.0 | Al salt 3) | 2.0-8.0 | Al salt 3) | 2.0-8.0 |
| KM | 0.05-2.0 | KM | 0.05-2.0 | KM | 0.05-2.0 |
| ZK296 | 0.1-5.0 | ZK297 | 0.1-5.0 | ZK298 | 0.1-5.0 |
| Carrier 1) | to make | Carrier 1) | to make | Carrier 1) | to make |
| | 100 | | 100 | | 100 |
| | AF370 | | AF371 | | AF372 |
| Al salt 3) | 2.0-8.0 | Al salt 3) | 2.0-8.0 | Al salt 3) | 2.0-8.0 |
| KM | 0.05-2.0 | KM | 0.05-2.0 | KM | 0.05-2.0 |
| ZK299 | 0.1-5.0 | ZK300 | 0.1-5.0 | ZK301 | 0.1-5.0 |
| Carrier 1) | to make | Carrier 1) | to make | Carrier 1) | to make |
| | 100 | | 100 | | 100 |
| | AF373 | | AF374 | | AF375 |
| Al salt 3) | 2.0-8.0 | Al salt 3) | 2.0-8.0 | Al salt 3) | 2.0-8.0 |
| KM | 0.05-2.0 | KM | 0.05-2.0 | KM | 0.05-2.0 |
| ZK302 | 0.1-5.0 | ZK303 | 0.1-5.0 | ZK304 | 0.1-5.0 |
| Carrier 1) | to make | Carrier 1) | to make | Carrier 1) | to make |
| | 100 | | 100 | | 100 |
| | AF376 | | AF377 | | AF378 |
| Al salt 3) | 2.0-8.0 | Al salt 3) | 2.0-8.0 | Al salt 3) | 2.0-8.0 |
| KM | 0.05-2.0 | KM | 0.05-2.0 | KM | 0.05-2.0 |
| ZK305 | 0.1-5.0 | ZK306 | 0.1-5.0 | ZK307 | 0.1-5.0 |
| Carrier 1) | to make | Carrier 1) | to make | Carrier 1) | to make |
| | 100 | | 100 | | 100 |
| | AF379 | | AF380 | | AF381 |
| Al salt 3) | 2.0-8.0 | Al salt 3) | 2.0-8.0 | Al salt 3) | 2.0-8.0 |
| KM | 0.05-2.0 | KM | 0.05-2.0 | KM | 0.05-2.0 |
| ZK308 | 0.1-5.0 | ZK309 | 0.1-5.0 | ZK310 | 0.1-5.0 |
| Carrier 1) | to make | Carrier 1) | to make | Carrier 1) | to make |
| | 100 | | 100 | | 100 |
| | AF382 | | AF383 | | AF384 |
| Al salt 3) | 2.0-8.0 | Al salt 3) | 2.0-8.0 | Al salt 3) | 2.0-8.0 |
| KM | 0.05-2.0 | KM | 0.05-2.0 | KM | 0.05-2.0 |
| ZK311 | 0.1-5.0 | ZK312 | 0.1-5.0 | ZK313 | 0.1-5.0 |
| Carrier 1) | to make | Carrier 1) | to make | Carrier 1) | to make |
| | 100 | | 100 | | 100 |
| | AF385 | | AF386 | | AF387 |
| Al salt 3) | 2.0-8.0 | Al salt 3) | 2.0-8.0 | Al salt 3) | 2.0-8.0 |
| KM | 0.05-2.0 | KM | 0.05-2.0 | KM | 0.05-2.0 |
| ZK314 | 0.1-5.0 | ZK315 | 0.1-5.0 | ZK316 | 0.1-5.0 |
| Carrier 1) | to make | Carrier 1) | to make | Carrier 1) | to make |
| | 100 | | 100 | | 100 |
| | AF388 | | AF389 | | AF390 |
| Al salt 3) | 2.0-8.0 | Al salt 3) | 2.0-8.0 | Al salt 3) | 2.0-8.0 |
| KM | 0.05-2.0 | KM | 0.05-2.0 | KM | 0.05-2.0 |
| ZK317 | 0.1-5.0 | ZK318 | 0.1-5.0 | ZK319 | 0.1-5.0 |
| Carrier 1) | to make | Carrier 1) | to make | Carrier 1) | to make |
| | 100 | | 100 | | 100 |
| | AF391 | | AF392 | | AF393 |
| Al salt 3) | 2.0-8.0 | Al salt 3) | 2.0-8.0 | Al salt 3) | 2.0-8.0 |
| KM | 0.05-2.0 | KM | 0.05-2.0 | KM | 0.05-2.0 |
| ZK320 | 0.1-5.0 | ZK321 | 0.1-5.0 | ZK322 | 0.1-5.0 |
| Carrier 1) | to make | Carrier 1) | to make | Carrier 1) | to make |
| | 100 | | 100 | | 100 |
| | AF394 | | AF395 | | AF396 |
| Al salt 3) | 2.0-8.0 | Al salt 3) | 2.0-8.0 | Al salt 3) | 2.0-8.0 |
| KM | 0.05-2.0 | KM | 0.05-2.0 | KM | 0.05-2.0 |
| ZK326 | 0.1-5.0 | ZK327 | 0.1-5.0 | ZK328 | 0.1-5.0 |
| Carrier 1) | to make | Carrier 1) | to make | Carrier 1) | to make |
| | 100 | | 100 | | 100 |
| | AF397 | | AF398 | | AF399 |
| Al salt 3) | 2.0-8.0 | Al salt 3) | 2.0-8.0 | Al salt 3) | 2.0-8.0 |
| KM | 0.05-2.0 | KM | 0.05-2.0 | KM | 0.05-2.0 |
| ZK329 | 0.1-5.0 | ZK330 | 0.1-5.0 | ZK331 | 0.1-5.0 |
| Carrier 1) | to make | Carrier 1) | to make | Carrier 1) | to make |
| | 100 | | 100 | | 100 |
| | AF400 | | AF401 | | AF402 |
| Al salt 3) | 2.0-8.0 | Al salt 3) | 2.0-8.0 | Al salt 3) | 2.0-8.0 |
| KM | 0.05-2.0 | KM | 0.05-2.0 | KM | 0.05-2.0 |
| ZK332 | 0.1-5.0 | ZK333 | 0.1-5.0 | ZK334 | 0.1-5.0 |
| Carrier 1) | to make | Carrier 1) | to make | Carrier 1) | to make |
| | 100 | | 100 | | 100 |
| | AF403 | | AF404 | | AF405 |
| Al salt 3) | 2.0-8.0 | Al salt 3) | 2.0-8.0 | Al salt 3) | 2.0-8.0 |
| KM | 0.05-2.0 | KM | 0.05-2.0 | KM | 0.05-2.0 |
| ZK335 | 0.1-5.0 | ZK336 | 0.1-5.0 | ZK337 | 0.1-5.0 |
| Carrier 1) | to make | Carrier 1) | to make | Carrier 1) | to make |
| | 100 | | 100 | | 100 |
| | AF406 | | AF407 | | AF408 |
| Al salt 3) | 2.0-8.0 | Al salt 3) | 2.0-8.0 | Al salt 3) | 2.0-8.0 |
| KM | 0.05-2.0 | KM | 0.05-2.0 | KM | 0.05-2.0 |
| ZK338 | 0.1-5.0 | ZK339 | 0.1-5.0 | ZK340 | 0.1-5.0 |
| Carrier 1) | to make | Carrier 1) | to make | Carrier 1) | to make |
| | 100 | | 100 | | 100 |
| | AF409 | | AF410 | | AF411 |
| Al salt 3) | 2.0-8.0 | Al salt 3) | 2.0-8.0 | Al salt 3) | 2.0-8.0 |
| KM | 0.05-2.0 | KM | 0.05-2.0 | KM | 0.05-2.0 |
| ZK341 | 0.1-5.0 | ZK342 | 0.1-5.0 | ZK343 | 0.1-5.0 |
| Carrier 1) | to make | Carrier 1) | to make | Carrier 1) | to make |
| | 100 | | 100 | | 100 |
| | AF412 | | AF413 | | AF414 |
| Al salt 3) | 2.0-8.0 | Al salt 3) | 2.0-8.0 | Al salt 3) | 2.0-8.0 |
| KM | 0.05-2.0 | KM | 0.05-2.0 | KM | 0.05-2.0 |
| ZK344 | 0.1-5.0 | ZK345 | 0.1-5.0 | ZK346 | 0.1-5.0 |
| Carrier 1) | to make | Carrier 1) | to make | Carrier 1) | to make |
| | 100 | | 100 | | 100 |
| | AF415 | | AF416 | | AF417 |
| Al salt 3) | 2.0-8.0 | Al salt 3) | 2.0-8.0 | Al salt 3) | 2.0-8.0 |
| KM | 0.05-2.0 | KM | 0.05-2.0 | KM | 0.05-2.0 |
| ZK347 | 0.1-5.0 | ZK265 | 0.1-5.0 | ZK266 | 0.1-5.0 |
| Carrier 1) | to make | Carrier 1) | to make | Carrier 1) | to make |
| | 100 | | 100 | | 100 |
| | AF418 | | AF419 | | AF420 |
| Al salt 3) | 2.0-8.0 | Al salt 3) | 2.0-8.0 | Al salt 3) | 2.0-8.0 |
| KM | 0.05-2.0 | KM | 0.05-2.0 | KM | 0.05-2.0 |
| ZK323 | 0.1-5.0 | ZK324 | 0.1-5.0 | ZK325 | 0.1-5.0 |
| Carrier 1) | to make | Carrier 1) | to make | Carrier 1) | to make |
| | 100 | | 100 | | 100 |
| | AF421 | | AF422 | | AF423 |
| Deodorant | 0.1-2.0 | Deodorant | 0.1-2.0 | Deodorant | 0.1-2.0 |
| active | | active | | active | |
| ingredient 2) | | ingredient 2) | | ingredient 2) | |
| Al salt 3) | 2.0-8.0 | Al salt 3) | 2.0-8.0 | Al salt 3) | 2.0-8.0 |
| KM | 0.05-2.0 | KM | 0.05-2.0 | KM | 0.05-2.0 |
| ZK267 | 0.1-5.0 | ZK268 | 0.1-5.0 | ZK269 | 0.1-5.0 |
| Carrier 1) | to make | Carrier 1) | to make | Carrier 1) | to make |
| | 100 | | 100 | | 100 |
| | AF424 | | AF425 | | AF426 |
| Deodorant | 0.1-2.0 | Deodorant | 0.1-2.0 | Deodorant | 0.1-2.0 |
| active | | active | | active | |
| ingredient 2) | | ingredient 2) | | ingredient 2) | |
| Al salt 3) | 2.0-8.0 | Al salt 3) | 2.0-8.0 | Al salt 3) | 2.0-8.0 |
| KM | 0.05-2.0 | KM | 0.05-2.0 | KM | 0.05-2.0 |
| ZK270 | 0.1-5.0 | ZK271 | 0.1-5.0 | ZK272 | 0.1-5.0 |
| Carrier 1) | to make | Carrier 1) | to make | Carrier 1) | to make |
| | 100 | | 100 | | 100 |
| | AF427 | | AF428 | | AF429 |
| Deodorant | 0.1-2.0 | Deodorant | 0.1-2.0 | Deodorant | 0.1-2.0 |
| active | | active | | active | |
| ingredient 2) | | ingredient 2) | | ingredient 2) | |
| Al salt 3) | 2.0-8.0 | Al salt 3) | 2.0-8.0 | Al salt 3) | 2.0-8.0 |
| KM | 0.05-2.0 | KM | 0.05-2.0 | KM | 0.05-2.0 |
| ZK270 | 0.1-5.0 | ZK271 | 0.1-5.0 | ZK272 | 0.1-5.0 |
| Carrier 1) | to make | Carrier 1) | to make | Carrier 1) | to make |
| | 100 | | 100 | | 100 |
| | AF430 | | AF431 | | AF432 |
| Deodorant | 0.1-2.0 | Deodorant | 0.1-2.0 | Deodorant | 0.1-2.0 |
| active | | active | | active | |
| ingredient 2) | | ingredient 2) | | ingredient 2) | |
| Al salt 3) | 2.0-8.0 | Al salt 3) | 2.0-8.0 | Al salt 3) | 2.0-8.0 |
| KM | 0.05-2.0 | KM | 0.05-2.0 | KM | 0.05-2.0 |
| ZK276 | 0.1-5.0 | ZK277 | 0.1-5.0 | ZK278 | 0.1-5.0 |
| Carrier 1) | to make | Carrier 1) | to make | Carrier 1) | to make |
| | 100 | | 100 | | 100 |
| | AF433 | | AF434 | | AF435 |
| Deodorant | 0.1-2.0 | Deodorant | 0.1-2.0 | Deodorant | 0.1-2.0 |
| active | | active | | active | |
| ingredient 2) | | ingredient 2) | | ingredient 2) | |
| Al salt 3) | 2.0-8.0 | Al salt 3) | 2.0-8.0 | Al salt 3) | 2.0-8.0 |
| KM | 0.05-2.0 | KM | 0.05-2.0 | KM | 0.05-2.0 |
| ZK279 | 0.1-5.0 | ZK280 | 0.1-5.0 | ZK280 | 0.1-5.0 |
| Carrier 1) | to make | Carrier 1) | to make | Carrier 1) | to make |
| | 100 | | 100 | | 100 |
| | AF436 | | AF437 | | AF438 |
| Deodorant | 0.1-2.0 | Deodorant | 0.1-2.0 | Deodorant | 0.1-2.0 |
| active | | active | | active | |
| ingredient 2) | | ingredient 2) | | ingredient 2) | |
| Al salt 3) | 2.0-8.0 | Al salt 3) | 2.0-8.0 | Al salt 3) | 2.0-8.0 |
| KM | 0.05-2.0 | KM | 0.05-2.0 | KM | 0.05-2.0 |
| ZK281 | 0.1-5.0 | ZK282 | 0.1-5.0 | ZK283 | 0.1-5.0 |
| Carrier 1) | to make | Carrier 1) | to make | Carrier 1) | to make |
| | 100 | | 100 | | 100 |
| | AF439 | | AF440 | | AF441 |
| Deodorant | 0.1-2.0 | Deodorant | 0.1-2.0 | Deodorant | 0.1-2.0 |
| active | | active | | active | |
| ingredient 2) | | ingredient 2) | | ingredient 2) | |
| Al salt 3) | 2.0-8.0 | Al salt 3) | 2.0-8.0 | Al salt 3) | 2.0-8.0 |
| KM | 0.05-2.0 | KM | 0.05-2.0 | KM | 0.05-2.0 |
| ZK284 | 0.1-5.0 | ZK285 | 0.1-5.0 | ZK286 | 0.1-5.0 |
| Carrier 1) | to make | Carrier 1) | to make | Carrier 1) | to make |
| | 100 | | 100 | | 100 |
| | AF442 | | AF443 | | AF444 |
| Deodorant | 0.1-2.0 | Deodorant | 0.1-2.0 | Deodorant | 0.1-2.0 |
| active | | active | | active | |
| ingredient 2) | | ingredient 2) | | ingredient 2) | |
| Al salt 3) | 2.0-8.0 | Al salt 3) | 2.0-8.0 | Al salt 3) | 2.0-8.0 |
| KM | 0.05-2.0 | KM | 0.05-2.0 | KM | 0.05-2.0 |
| ZK287 | 0.1-5.0 | ZK288 | 0.1-5.0 | ZK289 | 0.1-5.0 |
| Carrier 1) | to make | Carrier 1) | to make | Carrier 1) | to make |
| | 100 | | 100 | | 100 |
| | AF445 | | AF446 | | AF447 |
| Deodorant | 0.1-2.0 | Deodorant | 0.1-2.0 | Deodorant | 0.1-2.0 |
| active | | active | | active | |
| ingredient 2) | | ingredient 2) | | ingredient 2) | |
| Al salt 3) | 2.0-8.0 | Al salt 3) | 2.0-8.0 | Al salt 3) | 2.0-8.0 |
| KM | 0.05-2.0 | KM | 0.05-2.0 | KM | 0.05-2.0 |
| ZK290 | 0.1-5.0 | ZK291 | 0.1-5.0 | ZK292 | 0.1-5.0 |
| Carrier 1) | to make | Carrier 1) | to make | Carrier 1) | to make |
| | 100 | | 100 | | 100 |
| | AF448 | | AF449 | | AF450 |
| Deodorant | 0.1-2.0 | Deodorant | 0.1-2.0 | Deodorant | 0.1-2.0 |
| active | | active | | active | |
| ingredient 2) | | ingredient 2) | | ingredient 2) | |
| Al salt 3) | 2.0-8.0 | Al salt 3) | 2.0-8.0 | Al salt 3) | 2.0-8.0 |
| KM | 0.05-2.0 | KM | 0.05-2.0 | KM | 0.05-2.0 |
| ZK293 | 0.1-5.0 | ZK294 | 0.1-5.0 | ZK295 | 0.1-5.0 |
| Carrier 1) | to make | Carrier 1) | to make | Carrier 1) | to make |
| | 100 | | 100 | | 100 |
| | AF451 | | AF452 | | AF453 |
| Deodorant | 0.1-2.0 | Deodorant | 0.1-2.0 | Deodorant | 0.1-2.0 |
| active | | active | | active | |
| ingredient 2) | | ingredient 2) | | ingredient 2) | |
| Al salt 3) | 2.0-8.0 | Al salt 3) | 2.0-8.0 | Al salt 3) | 2.0-8.0 |
| KM | 0.05-2.0 | KM | 0.05-2.0 | KM | 0.05-2.0 |
| ZK296 | 0.1-5.0 | ZK297 | 0.1-5.0 | ZK298 | 0.1-5.0 |
| Carrier 1) | to make | Carrier 1) | to make | Carrier 1) | to make |
| | 100 | | 100 | | 100 |
| | AF454 | | AF455 | | AF456 |
| Deodorant | 0.1-2.0 | Deodorant | 0.1-2.0 | Deodorant | 0.1-2.0 |
| active | | active | | active | |
| ingredient 2) | | ingredient 2) | | ingredient 2) | |
| Al salt 3) | 2.0-8.0 | Al salt 3) | 2.0-8.0 | Al salt 3) | 2.0-8.0 |
| KM | 0.05-2.0 | KM | 0.05-2.0 | KM | 0.05-2.0 |
| ZK299 | 0.1-5.0 | ZK300 | 0.1-5.0 | ZK301 | 0.1-5.0 |
| Carrier 1) | to make | Carrier 1) | to make | Carrier 1) | to make |
| | 100 | | 100 | | 100 |
| | AF457 | | AF458 | | AF459 |
| Deodorant | 0.1-2.0 | Deodorant | 0.1-2.0 | Deodorant | 0.1-2.0 |
| active | | active | | active | |
| ingredient 2) | | ingredient 2) | | ingredient 2) | |
| Al salt 3) | 2.0-8.0 | Al salt 3) | 2.0-8.0 | Al salt 3) | 2.0-8.0 |
| KM | 0.05-2.0 | KM | 0.05-2.0 | KM | 0.05-2.0 |
| ZK302 | 0.1-5.0 | ZK303 | 0.1-5.0 | ZK304 | 0.1-5.0 |
| Carrier 1) | to make | Carrier 1) | to make | Carrier 1) | to make |
| | 100 | | 100 | | 100 |
| | AF460 | | AF461 | | AF462 |
| Deodorant | 0.1-2.0 | Deodorant | 0.1-2.0 | Deodorant | 0.1-2.0 |
| active | | active | | active | |
| ingredient 2) | | ingredient 2) | | ingredient 2) | |
| Al salt 3) | 2.0-8.0 | Al salt 3) | 2.0-8.0 | Al salt 3) | 2.0-8.0 |
| KM | 0.05-2.0 | KM | 0.05-2.0 | KM | 0.05-2.0 |
| ZK305 | 0.1-5.0 | ZK306 | 0.1-5.0 | ZK307 | 0.1-5.0 |
| Carrier 1) | to make | Carrier 1) | to make | Carrier 1) | to make |
| | 100 | | 100 | | 100 |
| | AF463 | | AF464 | | AF465 |
| Deodorant | 0.1-2.0 | Deodorant | 0.1-2.0 | Deodorant | 0.1-2.0 |
| active | | active | | active | |
| ingredient 2) | | ingredient 2) | | ingredient 2) | |
| Al salt 3) | 2.0-8.0 | Al salt 3) | 2.0-8.0 | Al salt 3) | 2.0-8.0 |
| KM | 0.05-2.0 | KM | 0.05-2.0 | KM | 0.05-2.0 |
| ZK308 | 0.1-5.0 | ZK309 | 0.1-5.0 | ZK310 | 0.1-5.0 |
| Carrier 1) | to make | Carrier 1) | to make | Carrier 1) | to make |
| | 100 | | 100 | | 100 |
| | AF466 | | AF467 | | AF468 |
| Deodorant | 0.1-2.0 | Deodorant | 0.1-2.0 | Deodorant | 0.1-2.0 |
| active | | active | | active | |
| ingredient 2) | | ingredient 2) | | ingredient 2) | |
| Al salt 3) | 2.0-8.0 | Al salt 3) | 2.0-8.0 | Al salt 3) | 2.0-8.0 |
| KM | 0.05-2.0 | KM | 0.05-2.0 | KM | 0.05-2.0 |
| ZK311 | 0.1-5.0 | ZK312 | 0.1-5.0 | ZK313 | 0.1-5.0 |
| Carrier 1) | to make | Carrier 1) | to make | Carrier 1) | to make |
| | 100 | | 100 | | 100 |
| | AF469 | | AF470 | | AF471 |
| Deodorant | 0.1-2.0 | Deodorant | 0.1-2.0 | Deodorant | 0.1-2.0 |
| active | | active | | active | |
| ingredient 2) | | ingredient 2) | | ingredient 2) | |
| Al salt 3) | 2.0-8.0 | Al salt 3) | 2.0-8.0 | Al salt 3) | 2.0-8.0 |
| KM | 0.05-2.0 | KM | 0.05-2.0 | KM | 0.05-2.0 |
| ZK314 | 0.1-5.0 | ZK315 | 0.1-5.0 | ZK316 | 0.1-5.0 |
| Carrier 1) | to make | Carrier 1) | to make | Carrier 1) | to make |
| | 100 | | 100 | | 100 |
| | AF472 | | AF473 | | AF474 |
| Deodorant | 0.1-2.0 | Deodorant | 0.1-2.0 | Deodorant | 0.1-2.0 |
| active | | active | | active | |
| ingredient 2) | | ingredient 2) | | ingredient 2) | |
| Al salt 3) | 2.0-8.0 | Al salt 3) | 2.0-8.0 | Al salt 3) | 2.0-8.0 |
| KM | 0.05-2.0 | KM | 0.05-2.0 | KM | 0.05-2.0 |
| ZK317 | 0.1-5.0 | ZK318 | 0.1-5.0 | ZK319 | 0.1-5.0 |
| Carrier 1) | to make | Carrier 1) | to make | Carrier 1) | to make |
| | 100 | | 100 | | 100 |
| | AF475 | | AF476 | | AF477 |
| Deodorant | 0.1-2.0 | Deodorant | 0.1-2.0 | Deodorant | 0.1-2.0 |
| active | | active | | active | |
| ingredient 2) | | ingredient 2) | | ingredient 2) | |
| Al salt 3) | 2.0-8.0 | Al salt 3) | 2.0-8.0 | Al salt 3) | 2.0-8.0 |
| KM | 0.05-2.0 | KM | 0.05-2.0 | KM | 0.05-2.0 |
| ZK320 | 0.1-5.0 | ZK321 | 0.1-5.0 | ZK322 | 0.1-5.0 |
| Carrier 1) | to make | Carrier 1) | to make | Carrier 1) | to make |
| | 100 | | 100 | | 100 |
| | AF478 | | AF479 | | AF480 |
| Deodorant | 0.1-2.0 | Deodorant | 0.1-2.0 | Deodorant | 0.1-2.0 |
| active | | active | | active | |
| ingredient 2) | | ingredient 2) | | ingredient 2) | |
| Al salt 3) | 2.0-8.0 | Al salt 3) | 2.0-8.0 | Al salt 3) | 2.0-8.0 |
| KM | 0.05-2.0 | KM | 0.05-2.0 | KM | 0.05-2.0 |
| ZK323 | 0.1-5.0 | ZK324 | 0.1-5.0 | ZK325 | 0.1-5.0 |
| Carrier 1) | to make | Carrier 1) | to make | Carrier 1) | to make |
| | 100 | | 100 | | 100 |
| | AF481 | | AF482 | | AF483 |
| Deodorant | 0.1-2.0 | Deodorant | 0.1-2.0 | Deodorant | 0.1-2.0 |
| active | | active | | active | |
| ingredient 2) | | ingredient 2) | | ingredient 2) | |
| Al salt 3) | 2.0-8.0 | Al salt 3) | 2.0-8.0 | Al salt 3) | 2.0-8.0 |
| KM | 0.05-2.0 | KM | 0.05-2.0 | KM | 0.05-2.0 |
| ZK326 | 0.1-5.0 | ZK327 | 0.1-5.0 | ZK328 | 0.1-5.0 |
| Carrier 1) | to make | Carrier 1) | to make | Carrier 1) | to make |
| | 100 | | 100 | | 100 |
| | AF484 | | AF485 | | AF486 |
| Deodorant | 0.1-2.0 | Deodorant | 0.1-2.0 | Deodorant | 0.1-2.0 |
| active | | active | | active | |
| ingredient 2) | | ingredient 2) | | ingredient 2) | |
| Al salt 3) | 2.0-8.0 | Al salt 3) | 2.0-8.0 | Al salt 3) | 2.0-8.0 |
| KM | 0.05-2.0 | KM | 0.05-2.0 | KM | 0.05-2.0 |
| ZK329 | 0.1-5.0 | ZK330 | 0.1-5.0 | ZK331 | 0.1-5.0 |
| Carrier 1) | to make | Carrier 1) | to make | Carrier 1) | to make |
| | 100 | | 100 | | 100 |
| | AF487 | | AF488 | | AF489 |
| Deodorant | 0.1-2.0 | Deodorant | 0.1-2.0 | Deodorant | 0.1-2.0 |
| active | | active | | active | |
| ingredient 2) | | ingredient 2) | | ingredient 2) | |
| Al salt 3) | 2.0-8.0 | Al salt 3) | 2.0-8.0 | Al salt 3) | 2.0-8.0 |
| KM | 0.05-2.0 | KM | 0.05-2.0 | KM | 0.05-2.0 |
| ZK332 | 0.1-5.0 | ZK333 | 0.1-5.0 | ZK334 | 0.1-5.0 |
| Carrier 1) | to make | Carrier 1) | to make | Carrier 1) | to make |
| | 100 | | 100 | | 100 |
| | AF490 | | AF491 | | AF492 |
| Deodorant | 0.1-2.0 | Deodorant | 0.1-2.0 | Deodorant | 0.1-2.0 |
| active | | active | | active | |
| ingredient 2) | | ingredient 2) | | ingredient 2) | |
| Al salt 3) | 2.0-8.0 | Al salt 3) | 2.0-8.0 | Al salt 3) | 2.0-8.0 |
| KM | 0.05-2.0 | KM | 0.05-2.0 | KM | 0.05-2.0 |
| ZK335 | 0.1-5.0 | ZK336 | 0.1-5.0 | ZK337 | 0.1-5.0 |
| Carrier 1) | to make | Carrier 1) | to make | Carrier 1) | to make |
| | 100 | | 100 | | 100 |
| | AF493 | | AF494 | | AF495 |
| Deodorant | 0.1-2.0 | Deodorant | 0.1-2.0 | Deodorant | 0.1-2.0 |
| active | | active | | active | |
| ingredient 2) | | ingredient 2) | | ingredient 2) | |
| Al salt 3) | 2.0-8.0 | Al salt 3) | 2.0-8.0 | Al salt 3) | 2.0-8.0 |
| KM | 0.05-2.0 | KM | 0.05-2.0 | KM | 0.05-2.0 |
| ZK338 | 0.1-5.0 | ZK339 | 0.1-5.0 | ZK340 | 0.1-5.0 |
| Carrier 1) | to make | Carrier 1) | to make | Carrier 1) | to make |
| | 100 | | 100 | | 100 |
| | AF496 | | AF497 | | AF498 |
| Deodorant | 0.1-2.0 | Deodorant | 0.1-2.0 | Deodorant | 0.1-2.0 |
| active | | active | | active | |
| ingredient 2) | | ingredient 2) | | ingredient 2) | |
| Al salt 3) | 2.0-8.0 | Al salt 3) | 2.0-8.0 | Al salt 3) | 2.0-8.0 |
| KM | 0.05-2.0 | KM | 0.05-2.0 | KM | 0.05-2.0 |
| ZK341 | 0.1-5.0 | ZK342 | 0.1-5.0 | ZK343 | 0.1-5.0 |
| Carrier 1) | to make | Carrier 1) | to make | Carrier 1) | to make |
| | 100 | | 100 | | 100 |
| | AF499 | | AF500 | | AF501 |
| Deodorant | 0.1-2.0 | Deodorant | 0.1-2.0 | Deodorant | 0.1-2.0 |
| active | | active | | active | |
| ingredient 2) | | ingredient 2) | | ingredient 2) | |
| Al salt 3) | 2.0-8.0 | Al salt 3) | 2.0-8.0 | Al salt 3) | 2.0-8.0 |
| KM | 0.05-2.0 | KM | 0.05-2.0 | KM | 0.05-2.0 |
| ZK344 | 0.1-5.0 | ZK345 | 0.1-5.0 | ZK346 | 0.1-5.0 |
| Carrier 1) | to make | Carrier 1) | to make | Carrier 1) | to make |
| | 100 | | 100 | | 100 |
| | AF502 | | AF503 | | AF504 |
| Deodorant | 0.1-2.0 | Deodorant | 0.1-2.0 | Deodorant | 0.1-2.0 |
| active | | active | | active | |
| ingredient 2) | | ingredient 2) | | ingredient 2) | |
| Al salt 3) | 2.0-8.0 | Al salt 3) | 2.0-8.0 | Al salt 3) | 2.0-8.0 |
| KM | 0.05-2.0 | KM | 0.05-2.0 | KM | 0.05-2.0 |
| ZK347 | 0.1-5.0 | ZK265 | 0.1-5.0 | ZK266 | 0.1-5.0 |
| Carrier 1) | to make | Carrier 1) | to make | Carrier 1) | to make |
| | 100 | | 100 | | 100 |

1) aqueous, aqueous-alcoholic or anhydrous carrier,
2) selected from mixture comprising 1,2-hexanediol and 1,2-octanediol,
3) aluminum chlorohydrate,
4) aluminum-zirconium tetrachlorohydrate, stabilized with glycine

TABLE 5
Particularly preferred cosmetic agents (information in wt.
%, based on the total weight of the propellant-free agent)

| | AF1 | | AF2 | | AF3 |
|---|---|---|---|---|---|
| Deodorant | 0.1-2.0 | Deodorant | 0.1-2.0 | Deodorant | 0.1-2.0 |
| active | | active | | active | |
| ingredient | | ingredient | | ingredient | |
| KM | 0.05-2.0 | KM | 0.05-2.0 | KM | 0.05-2.0 |
| ZK348 | 0.1-5.0 | ZK349 | 0.1-5.0 | ZK350 | 0.1-5.0 |
| Carrier 1) | to make | Carrier 1) | to make | Carrier 1) | to make |
| | 100 | | 100 | | 100 |
| | AF4 | | AF5 | | AF6 |
| Deodorant | 0.1-2.0 | Deodorant | 0.1-2.0 | Deodorant | 0.1-2.0 |
| active | | active | | active | |
| ingredient | | ingredient | | ingredient | |
| KM | 0.05-2.0 | KM | 0.05-2.0 | KM | 0.05-2.0 |
| ZK351 | 0.1-5.0 | ZK352 | 0.1-5.0 | ZK353 | 0.1-5.0 |
| Carrier 1) | to make | Carrier 1) | to make | Carrier 1) | to make |
| | 100 | | 100 | | 100 |
| | AF7 | | AF8 | | AF9 |
| Deodorant | 0.1-2.0 | Deodorant | 0.1-2.0 | Deodorant | 0.1-2.0 |
| active | | active | | active | |
| ingredient | | ingredient | | ingredient | |
| KM | 0.05-2.0 | KM | 0.05-2.0 | KM | 0.05-2.0 |
| ZK354 | 0.1-5.0 | ZK355 | 0.1-5.0 | ZK356 | 0.05-2.0 |
| Carrier 1) | to make | Carrier 1) | to make | Carrier 1) | to make |
| | 100 | | 100 | | 100 |
| | AF10 | | AF11 | | AF12 |
| Deodorant | 0.1-2.0 | Deodorant | 0.1-2.0 | Deodorant | 0.1-2.0 |
| active | | active | | active | |
| ingredient | | ingredient | | ingredient | |
| KM | 0.05-2.0 | KM | 0.05-2.0 | KM | 0.05-2.0 |
| ZK357 | 0.1-5.0 | ZK358 | 0.1-5.0 | ZK359 | 0.05-2.0 |
| Carrier 1) | to make | Carrier 1) | to make | Carrier 1) | to make |
| | 100 | | 100 | | 100 |
| | AF13 | | AF14 | | AF15 |
| Deodorant | 0.1-2.0 | Deodorant | 0.1-2.0 | Deodorant | 0.1-2.0 |
| active | | active | | active | |
| ingredient | | ingredient | | ingredient | |
| KM | 0.05-2.0 | KM | 0.05-2.0 | KM | 0.05-2.0 |
| ZK360 | 0.1-5.0 | ZK361 | 0.1-5.0 | ZK362 | 0.05-2.0 |
| Carrier 1) | to make | Carrier 1) | to make | Carrier 1) | to make |
| | 100 | | 100 | | 100 |
| | AF16 | | AF17 | | AF18 |
| Deodorant | 0.1-2.0 | Deodorant | 0.1-2.0 | Deodorant | 0.1-2.0 |
| active | | active | | active | |
| ingredient | | ingredient | | ingredient | |
| KM | 0.05-2.0 | KM | 0.05-2.0 | KM | 0.05-2.0 |
| ZK363 | 0.1-5.0 | ZK364 | 0.1-5.0 | ZK365 | 0.1-5.0 |
| Carrier 1) | to make | Carrier 1) | to make | Carrier 1) | to make |
| | 100 | | 100 | | 100 |
| | AF19 | | AF20 | | AF21 |
| Deodorant | 0.1-2.0 | Deodorant | 0.1-2.0 | Deodorant | 0.1-2.0 |
| active | | active | | active | |
| ingredient | | ingredient | | ingredient | |
| KM | 0.05-2.0 | KM | 0.05-2.0 | KM | 0.05-2.0 |
| ZK366 | 0.1-5.0 | ZK367 | 0.1-5.0 | ZK368 | 0.1-5.0 |
| Carrier 1) | to make | Carrier 1) | to make | Carrier 1) | to make |
| | 100 | | 100 | | 100 |
| | AF22 | | AF23 | | AF24 |
| Deodorant | 0.1-2.0 | Al salt | 2.0-8.0 | Al salt | 0.1-2.0 |
| active | | | | | |
| ingredient | | | | | |
| KM | 0.05-2.0 | KM | 0.05-2.0 | KM | 0.05-2.0 |
| ZK369 | 0.1-5.0 | ZK348 | 0.1-5.0 | ZK349 | 0.1-5.0 |
| Carrier 1) | to make | Carrier 1) | to make | Carrier 1) | to make |
| | 100 | | 100 | | 100 |
| | AF25 | | AF26 | | AF27 |
| Al salt | 2.0-8.0 | Al salt | 2.0-8.0 | Al salt | 2.0-8.0 |
| KM | 0.05-2.0 | KM | 0.05-2.0 | KM | 0.05-2.0 |
| ZK350 | 0.1-5.0 | ZK351 | 0.1-5.0 | ZK352 | 0.1-5.0 |
| Carrier 1) | to make | Carrier 1) | to make | Carrier 1) | to make |
| | 100 | | 100 | | 100 |
| | AF28 | | AF29 | | AF30 |
| Al salt | 2.0-8.0 | Al salt | 2.0-8.0 | Al salt | 2.0-8.0 |
| KM | 0.05-2.0 | KM | 0.05-2.0 | KM | 0.05-2.0 |
| ZK353 | 0.1-5.0 | ZK354 | 0.1-5.0 | ZK355 | 0.1-5.0 |
| Carrier 1) | to make | Carrier 1) | to make | Carrier 1) | to make |
| | 100 | | 100 | | 100 |
| | AF31 | | AF32 | | AF33 |
| Al salt | 2.0-8.0 | Al salt | 2.0-8.0 | Al salt | 2.0-8.0 |
| KM | 0.05-2.0 | KM | 0.05-2.0 | KM | 0.05-2.0 |
| ZK356 | 0.1-5.0 | ZK357 | 0.1-5.0 | ZK358 | 0.1-5.0 |
| Carrier 1) | to make | Carrier 1) | to make | Carrier 1) | to make |
| | 100 | | 100 | | 100 |
| | AF34 | | AF35 | | AF36 |
| Al salt | 2.0-8.0 | Al salt | 2.0-8.0 | Al salt | 2.0-8.0 |
| KM | 0.05-2.0 | KM | 0.05-2.0 | KM | 0.05-2.0 |
| ZK359 | 0.1-5.0 | ZK360 | 0.1-5.0 | ZK361 | 0.1-5.0 |
| Carrier 1) | to make | Carrier 1) | to make | Carrier 1) | to make |
| | 100 | | 100 | | 100 |
| | AF37 | | AF38 | | AF39 |
| Al salt | 2.0-8.0 | Al salt | 2.0-8.0 | Al salt | 2.0-8.0 |
| KM | 0.05-2.0 | KM | 0.05-2.0 | KM | 0.05-2.0 |
| ZK362 | 0.1-5.0 | ZK363 | 0.1-5.0 | ZK364 | 0.1-5.0 |
| Carrier 1) | to make | Carrier 1) | to make | Carrier 1) | to make |
| | 100 | | 100 | | 100 |
| | AF40 | | AF41 | | AF42 |
| Al salt | 2.0-8.0 | Al salt | 2.0-8.0 | Al salt | 2.0-8.0 |
| KM | 0.05-2.0 | KM | 0.05-2.0 | KM | 0.05-2.0 |
| ZK365 | 0.1-5.0 | ZK366 | 0.1-5.0 | ZK367 | 0.1-5.0 |
| Carrier 1) | to make | Carrier 1) | to make | Carrier 1) | to make |
| | 100 | | 100 | | 100 |
| | AF43 | | AF44 | | |
| Al salt | 2.0-8.0 | Al salt | 2.0-8.0 | | |
| KM | 0.05-2.0 | KM | 0.05-2.0 | | |
| ZK368 | 0.1-5.0 | ZK369 | 0.1-5.0 | | |
| Carrier 1) | to make | Carrier 1) | to make | | |
| | 100 | | 100 | | |
| | AF45 | | AF46 | | AF47 |
| Deodorant | 0.1-2.0 | Deodorant | 0.1-2.0 | Deodorant | 0.1-2.0 |
| active | | active | | active | |
| ingredient | | ingredient | | ingredient | |
| Al salt | 2.0-8.0 | Al salt | 2.0-8.0 | Al salt | 2.0-8.0 |
| KM | 0.05-2.0 | KM | 0.05-2.0 | KM | 0.05-2.0 |
| ZK348 | 0.1-5.0 | ZK349 | 0.1-5.0 | ZK350 | 0.1-5.0 |
| Carrier 1) | to make | Carrier 1) | to make | Carrier 1) | to make |
| | 100 | | 100 | | 100 |
| | AF48 | | AF49 | | AF50 |
| Deodorant | 0.1-2.0 | Deodorant | 0.1-2.0 | Deodorant | 0.1-2.0 |
| active | | active | | active | |
| ingredient | | ingredient | | ingredient | |
| Al salt | 2.0-8.0 | Al salt | 2.0-8.0 | Al salt | 2.0-8.0 |
| KM | 0.05-2.0 | KM | 0.05-2.0 | KM | 0.05-2.0 |
| ZK351 | 0.1-5.0 | ZK352 | 0.1-5.0 | ZK353 | 0.1-5.0 |
| Carrier 1) | to make | Carrier 1) | to make | Carrier 1) | to make |
| | 100 | | 100 | | 100 |
| | AF51 | | AF52 | | AF53 |
| Deodorant | 0.1-2.0 | Deodorant | 0.1-2.0 | Deodorant | 0.1-2.0 |
| active | | active | | active | |
| ingredient | | ingredient | | ingredient | |
| Al salt | 2.0-8.0 | Al salt | 2.0-8.0 | Al salt | 2.0-8.0 |
| KM | 0.05-2.0 | KM | 0.05-2.0 | KM | 0.05-2.0 |
| ZK354 | 0.1-5.0 | ZK355 | 0.1-5.0 | ZK356 | 0.1-5.0 |
| Carrier 1) | to make | Carrier 1) | to make | Carrier 1) | to make |
| | 100 | | 100 | | 100 |
| | AF54 | | AF55 | | AF56 |
| Deodorant | 0.1-2.0 | Deodorant | 0.1-2.0 | Deodorant | 0.1-2.0 |
| active | | active | | active | |
| ingredient | | ingredient | | ingredient | |
| Al salt | 2.0-8.0 | Al salt | 2.0-8.0 | Al salt | 2.0-8.0 |
| KM | 0.05-2.0 | KM | 0.05-2.0 | KM | 0.05-2.0 |
| ZK357 | 0.1-5.0 | ZK358 | 0.1-5.0 | ZK359 | 0.1-5.0 |
| Carrier 1) | to make | Carrier 1) | to make | Carrier 1) | to make |
| | 100 | | 100 | | 100 |
| | AF57 | | AF58 | | AF59 |
| Deodorant | 0.1-2.0 | Deodorant | 0.1-2.0 | Deodorant | 0.1-2.0 |
| active | | active | | active | |
| ingredient | | ingredient | | ingredient | |
| Al salt | 2.0-8.0 | Al salt | 2.0-8.0 | Al salt | 2.0-8.0 |
| KM | 0.05-2.0 | KM | 0.05-2.0 | KM | 0.05-2.0 |
| ZK360 | 0.1-5.0 | ZK361 | 0.1-5.0 | ZK362 | 0.1-5.0 |
| Carrier 1) | to make | Carrier 1) | to make | Carrier 1) | to make |
| | 100 | | 100 | | 100 |
| | AF60 | | AF61 | | AF62 |
| Deodorant | 0.1-2.0 | Deodorant | 0.1-2.0 | Deodorant | 0.1-2.0 |
| active | | active | | active | |
| ingredient | | ingredient | | ingredient | |
| Al salt | 2.0-8.0 | Al salt | 2.0-8.0 | Al salt | 2.0-8.0 |
| KM | 0.05-2.0 | KM | 0.05-2.0 | KM | 0.05-2.0 |
| ZK363 | 0.1-5.0 | ZK364 | 0.1-5.0 | ZK365 | 0.1-5.0 |
| Carrier 1) | to make | Carrier 1) | to make | Carrier 1) | to make |
| | 100 | | 100 | | 100 |
| | AF63 | | AF64 | | AF65 |
| Deodorant | 0.1-2.0 | Deodorant | 0.1-2.0 | Deodorant | 0.1-2.0 |
| active | | active | | active | |
| ingredient | | ingredient | | ingredient | |
| Al salt | 2.0-8.0 | Al salt | 2.0-8.0 | Al salt | 2.0-8.0 |
| KM | 0.05-2.0 | KM | 0.05-2.0 | KM | 0.05-2.0 |
| ZK366 | 0.1-5.0 | ZK367 | 0.1-5.0 | ZK368 | 0.1-5.0 |
| Carrier 1) | to make | Carrier 1) | to make | Carrier 1) | to make |
| | 100 | | 100 | | 100 |
| | AF66 | | AF67 | | AF68 |
| Deodorant | 0.1-2.0 | Deodorant | 0.1-2.0 | Deodorant | 0.1-2.0 |
| active | | active | | active | |
| ingredient 2) | | ingredient 2) | | ingredient 2) | |
| KM | 0.05-2.0 | KM | 0.05-2.0 | KM | 0.05-2.0 |
| ZK348 | 0.1-5.0 | ZK349 | 0.1-5.0 | ZK350 | 0.1-5.0 |
| Carrier 1) | to make | Carrier 1) | to make | Carrier 1) | to make |
| | 100 | | 100 | | 100 |
| | AF69 | | AF70 | | AF71 |
| Deodorant | 0.1-2.0 | Deodorant | 0.1-2.0 | Deodorant | 0.1-2.0 |
| active | | active | | active | |
| ingredient 2) | | ingredient 2) | | ingredient 2) | |
| KM | 0.05-2.0 | KM | 0.05-2.0 | KM | 0.05-2.0 |
| ZK351 | 0.1-5.0 | ZK352 | 0.1-5.0 | ZK353 | 0.1-5.0 |
| Carrier 1) | to make | Carrier 1) | to make | Carrier 1) | to make |
| | 100 | | 100 | | 100 |
| | AF72 | | AF73 | | AF74 |
| Deodorant | 0.1-2.0 | Deodorant | 0.1-2.0 | Deodorant | 0.1-2.0 |
| active | | active | | active | |
| ingredient 2) | | ingredient 2) | | ingredient 2) | |
| KM | 0.05-2.0 | KM | 0.05-2.0 | KM | 0.05-2.0 |
| ZK354 | 0.1-5.0 | ZK355 | 0.1-5.0 | ZK356 | 0.1-5.0 |
| Carrier 1) | to make | Carrier 1) | to make | Carrier 1) | to make |
| | 100 | | 100 | | 100 |
| | AF75 | | AF76 | | AF77 |
| Deodorant | 0.1-2.0 | Deodorant | 0.1-2.0 | Deodorant | 0.1-2.0 |
| active | | active | | active | |
| ingredient 2) | | ingredient 2) | | ingredient 2) | |
| KM | 0.05-2.0 | KM | 0.05-2.0 | KM | 0.05-2.0 |
| ZK357 | 0.1-5.0 | ZK358 | 0.1-5.0 | ZK359 | 0.1-5.0 |
| Carrier 1) | to make | Carrier 1) | to make | Carrier 1) | to make |
| | 100 | | 100 | | 100 |
| | AF78 | | AF79 | | AF80 |
| Deodorant | 0.1-2.0 | Deodorant | 0.1-2.0 | Deodorant | 0.1-2.0 |
| active | | active | | active | |
| ingredient 2) | | ingredient 2) | | ingredient 2) | |
| KM | 0.05-2.0 | KM | 0.05-2.0 | KM | 0.05-2.0 |
| ZK360 | 0.1-5.0 | ZK361 | 0.1-5.0 | ZK362 | 0.1-5.0 |
| Carrier 1) | to make | Carrier 1) | to make | Carrier 1) | to make |
| | 100 | | 100 | | 100 |
| | AF81 | | AF82 | | AF83 |
| Deodorant | 0.1-2.0 | Deodorant | 0.1-2.0 | Deodorant | 0.1-2.0 |
| active | | active | | active | |
| ingredient 2) | | ingredient 2) | | ingredient 2) | |
| KM | 0.05-2.0 | KM | 0.05-2.0 | KM | 0.05-2.0 |
| ZK363 | 0.1-5.0 | ZK364 | 0.1-5.0 | ZK365 | 0.1-5.0 |
| Carrier 1) | to make | Carrier 1) | to make | Carrier 1) | to make |
| | 100 | | 100 | | 100 |
| | AF84 | | AF85 | | AF86 |
| Deodorant | 0.1-2.0 | Deodorant | 0.1-2.0 | Deodorant | 0.1-2.0 |
| active | | active | | active | |
| ingredient 2) | | ingredient 2) | | ingredient 2) | |
| KM | 0.05-2.0 | KM | 0.05-2.0 | KM | 0.05-2.0 |
| ZK366 | 0.1-5.0 | ZK367 | 0.1-5.0 | ZK368 | 0.1-5.0 |
| Carrier 1) | to make | Carrier 1) | to make | Carrier 1) | to make |
| | 100 | | 100 | | 100 |
| | AF87 | | AF88 | | AF89 |
| Deodorant | 0.1-2.0 | Al salt 3) | 2.0-8.0 | Al salt 3) | 2.0-8.0 |
| active | | | | | |
| ingredient 2) | | | | | |
| KM | 0.05-2.0 | KM | 0.05-2.0 | KM | 0.05-2.0 |
| ZK369 | 0.1-5.0 | ZK348 | 0.1-5.0 | ZK349 | 0.1-5.0 |
| Carrier 1) | to make | Carrier 1) | to make | Carrier 1) | to make |
| | 100 | | 100 | | 100 |
| | AF90 | | AF91 | | AF92 |
| Al salt 3) | 2.0-8.0 | Al salt 3) | 2.0-8.0 | Al salt 3) | 2.0-8.0 |
| KM | 0.05-2.0 | KM | 0.05-2.0 | KM | 0.05-2.0 |
| ZK350 | 0.1-5.0 | ZK351 | 0.1-5.0 | ZK352 | 0.1-5.0 |
| Carrier 1) | to make | Carrier 1) | to make | Carrier 1) | to make |
| | 100 | | 100 | | 100 |
| | AF93 | | AF94 | | AF95 |
| Al salt 3) | 2.0-8.0 | Al salt 3) | 2.0-8.0 | Al salt 3) | 2.0-8.0 |
| KM | 0.05-2.0 | KM | 0.05-2.0 | KM | 0.05-2.0 |
| ZK353 | 0.1-5.0 | ZK354 | 0.1-5.0 | ZK355 | 0.1-5.0 |
| Carrier 1) | to make | Carrier 1) | to make | Carrier 1) | to make |
| | 100 | | 100 | | 100 |
| | AF96 | | AF97 | | AF98 |
| Al salt 3) | 2.0-8.0 | Al salt 3) | 2.0-8.0 | Al salt 3) | 2.0-8.0 |
| KM | 0.05-2.0 | KM | 0.05-2.0 | KM | 0.05-2.0 |
| ZK356 | 0.1-5.0 | ZK357 | 0.1-5.0 | ZK358 | 0.1-5.0 |
| Carrier 1) | to make | Carrier 1) | to make | Carrier 1) | to make |
| | 100 | | 100 | | 100 |
| | AF99 | | AF100 | | AF101 |
| Al salt 3) | 2.0-8.0 | Al salt 3) | 2.0-8.0 | Al salt 3) | 2.0-8.0 |
| KM | 0.05-2.0 | KM | 0.05-2.0 | KM | 0.05-2.0 |
| ZK359 | 0.1-5.0 | ZK360 | 0.1-5.0 | ZK361 | 0.1-5.0 |
| Carrier 1) | to make | Carrier 1) | to make | Carrier 1) | to make |
| | 100 | | 100 | | 100 |
| | AF102 | | AF103 | | AF104 |
| Al salt 3) | 2.0-8.0 | Al salt 3) | 2.0-8.0 | Al salt 3) | 2.0-8.0 |
| KM | 0.05-2.0 | KM | 0.05-2.0 | KM | 0.05-2.0 |
| ZK362 | 0.1-5.0 | ZK363 | 0.1-5.0 | ZK364 | 0.1-5.0 |
| Carrier 1) | to make | Carrier 1) | to make | Carrier 1) | to make |
| | 100 | | 100 | | 100 |
| | AF105 | | AF106 | | AF107 |
| Al salt 3) | 2.0-8.0 | Al salt 3) | 2.0-8.0 | Al salt 3) | 2.0-8.0 |
| KM | 0.05-2.0 | KM | 0.05-2.0 | KM | 0.05-2.0 |
| ZK365 | 0.1-5.0 | ZK366 | 0.1-5.0 | ZK367 | 0.1-5.0 |
| Carrier 1) | to make | Carrier 1) | to make | Carrier 1) | to make |
| | 100 | | 100 | | 100 |
| | AF108 | | AF109 | | |
| Al salt 3) | 2.0-8.0 | Al salt 3) | 2.0-8.0 | | |
| KM | 0.05-2.0 | KM | 0.05-2.0 | | |
| ZK368 | 0.1-5.0 | ZK369 | 0.1-5.0 | | |
| Carrier 1) | to make | Carrier 1) | to make | | |
| | 100 | | 100 | | |
| | AF110 | | AF111 | | AF112 |
| Deodorant | 0.1-2.0 | Deodorant | 0.1-2.0 | Deodorant | 0.1-2.0 |
| active | | active | | active | |
| ingredient 2) | | ingredient 2) | | ingredient 2) | |
| Al salt 3) | 2.0-8.0 | Al salt 3) | 2.0-8.0 | Al salt 3) | 2.0-8.0 |
| KM | 0.05-2.0 | KM | 0.05-2.0 | KM | 0.05-2.0 |
| ZK348 | 0.1-5.0 | ZK349 | 0.1-5.0 | ZK350 | 0.1-5.0 |
| Carrier 1) | to make | Carrier 1) | to make | Carrier 1) | to make |
| | 100 | | 100 | | 100 |
| | AF113 | | AF114 | | AF115 |
| Deodorant | 0.1-2.0 | Deodorant | 0.1-2.0 | Deodorant | 0.1-2.0 |
| active | | active | | active | |
| ingredient 2) | | ingredient 2) | | ingredient 2) | |
| Al salt 3) | 2.0-8.0 | Al salt 3) | 2.0-8.0 | Al salt 3) | 2.0-8.0 |
| KM | 0.05-2.0 | KM | 0.05-2.0 | KM | 0.05-2.0 |
| ZK351 | 0.1-5.0 | ZK352 | 0.1-5.0 | ZK353 | 0.1-5.0 |
| Carrier 1) | to make | Carrier 1) | to make | Carrier 1) | to make |
| | 100 | | 100 | | 100 |
| | AF116 | | AF117 | | AF118 |
| Deodorant | 0.1-2.0 | Deodorant | 0.1-2.0 | Deodorant | 0.1-2.0 |
| active | | active | | active | |
| ingredient 2) | | ingredient 2) | | ingredient 2) | |
| Al salt 3) | 2.0-8.0 | Al salt 3) | 2.0-8.0 | Al salt 3) | 2.0-8.0 |
| KM | 0.05-2.0 | KM | 0.05-2.0 | KM | 0.05-2.0 |
| ZK354 | 0.1-5.0 | ZK355 | 0.1-5.0 | ZK356 | 0.1-5.0 |
| Carrier 1) | to make | Carrier 1) | to make | Carrier 1) | to make |
| | 100 | | 100 | | 100 |
| | AF119 | | AF120 | | AF121 |
| Deodorant | 0.1-2.0 | Deodorant | 0.1-2.0 | Deodorant | 0.1-2.0 |
| active | | active | | active | |
| ingredient 2) | | ingredient 2) | | ingredient 2) | |
| Al salt 3) | 2.0-8.0 | Al salt 3) | 2.0-8.0 | Al salt 3) | 2.0-8.0 |
| KM | 0.05-2.0 | KM | 0.05-2.0 | KM | 0.05-2.0 |
| ZK357 | 0.1-5.0 | ZK358 | 0.1-5.0 | ZK359 | 0.1-5.0 |
| Carrier 1) | to make | Carrier 1) | to make | Carrier 1) | to make |
| | 100 | | 100 | | 100 |
| | AF122 | | AF123 | | AF124 |
| Deodorant | 0.1-2.0 | Deodorant | 0.1-2.0 | Deodorant | 0.1-2.0 |
| active | | active | | active | |
| ingredient 2) | | ingredient 2) | | ingredient 2) | |
| Al salt 3) | 2.0-8.0 | Al salt 3) | 2.0-8.0 | Al salt 3) | 2.0-8.0 |
| KM | 0.05-2.0 | KM | 0.05-2.0 | KM | 0.05-2.0 |
| ZK360 | 0.1-5.0 | ZK361 | 0.1-5.0 | ZK362 | 0.1-5.0 |
| Carrier 1) | to make | Carrier 1) | to make | Carrier 1) | to make |
| | 100 | | 100 | | 100 |
| | AF125 | | AF126 | | AF127 |
| Deodorant | 0.1-2.0 | Deodorant | 0.1-2.0 | Deodorant | 0.1-2.0 |
| active | | active | | active | |
| ingredient 2) | | ingredient 2) | | ingredient 2) | |
| Al salt 3) | 2.0-8.0 | Al salt 3) | 2.0-8.0 | Al salt 3) | 2.0-8.0 |
| KM | 0.05-2.0 | KM | 0.05-2.0 | KM | 0.05-2.0 |
| ZK363 | 0.1-5.0 | ZK364 | 0.1-5.0 | ZK365 | 0.1-5.0 |
| Carrier 1) | to make | Carrier 1) | to make | Carrier 1) | to make |
| | 100 | | 100 | | 100 |
| | AF128 | | AF129 | | AF130 |
| Deodorant | 0.1-2.0 | Deodorant | 0.1-2.0 | Deodorant | 0.1-2.0 |
| active | | active | | active | |
| ingredient 2) | | ingredient 2) | | ingredient 2) | |
| Al salt 3) | 2.0-8.0 | Al salt 3) | 2.0-8.0 | Al salt 3) | 2.0-8.0 |
| KM | 0.05-2.0 | KM | 0.05-2.0 | KM | 0.05-2.0 |
| ZK366 | 0.1-5.0 | ZK367 | 0.1-5.0 | ZK368 | 0.1-5.0 |
| Carrier 1) | to make | Carrier 1) | to make | Carrier 1) | to make |
| | 100 | | 100 | | 100 |
| | AF131 | | | | |
| Deodorant | 0.1-2.0 | | | | |
| active | | | | | |
| ingredient 2) | | | | | |
| Al salt 3) | 2.0-8.0 | | | | |
| KM | 0.05-2.0 | | | | |
| ZK369 | 0.1-5.0 | | | | |
| Carrier 1) | to make | | | | |
| | 100 | | | | |

1) aqueous, aqueous-alcoholic or anhydrous carrier,
2) selected from mixture comprising 1,2-hexanediol and 1,2-octanediol,
3) aluminum chlorohydrate,
4) aluminum-zirconium tetrachlorohydrate, stabilized with glycine

The preservatives (A) KM1 to KM1783 listed in Table 6 are used as preservatives KM in each of the above embodiments AF1 to AF504 of Table 2, Table 3 and Table 4, and in each of the embodiments AF1 to AF131 of Table 5. In these, s denotes sulfite, selected from the group consisting of sodium sulfite, ammonium bisulfite, ammonium sulfite, potassium sulfite, potassium hydrogen sulfite, sodium bisulfite, sodium metabisulfite and potassium metabisulfite and the mixtures thereof; h denotes hexetidine; b denotes benzyl alcohol; u denotes undecylenic acid; p denotes phenoxyisopropanol; pi denotes piroctone olamine; e denotes ethyl lauroyl arginate*HCl; a denotes formic acid; bz denotes benzethonium chloride; be denotes behentrimonium chloride; cb denotes cetrimonium bromide; cc denotes cetrimonium chloride; lb denotes laurtrimonium bromide; lc denotes laurtrimonium chloride; sb denotes steartrimonium bromide; sc denotes steartrimonium chloride; aq denotes quaternary ammonium compound; oc denotes o-cymen-5-ol; hx denotes hexamidine; hd denotes hexamidine diisethionate, and hp denotes hexamidine paraben. In the table, thus, for example, the information h+b denotes a mixture of hexetidine and benzyl alcohol. h+b+u accordingly is the combination of hexetidine, benzyl alcohol and undecylenic acid.

TABLE 6
First preservative (A) or mixtures of these preservatives

| KM1 | KM2 | KM3 | KM4 | KM5 | KM5 | KM6 | KM7 |
|---|---|---|---|---|---|---|---|
| s + h | h + b | b + u | u + p | p + pi | pi + e | e + a | a + bc |
| KM8 | KM9 | KM10 | KM11 | KM12 | KM13 | KM14 | KM15 |
| s + b | h + u | b + p | u + pi | p + e | pi + a | e + bc | a + cb |
| KM16 | KM17 | KM18 | KM19 | KM20 | KM21 | KM22 | KM23 |
| s + u | h + p | b + pi | u + e | p + a | pi + bc | e + cb | a + cc |
| KM24 | KM25 | KM26 | KM27 | KM28 | KM29 | KM30 | KM31 |
| s + p | h + pi | b + e | u + a | p + bc | pi + cb | e + cc | a + lb |
| KM32 | KM33 | KM34 | KM35 | KM36 | KM37 | KM38 | KM39 |
| s + pi | h + e | b + a | u + bc | p + cb | pi + cc | e + lb | a + lc |
| KM40 | KM41 | KM42 | KM43 | KM44 | KM45 | KM46 | KM47 |
| s + e | h + a | b + bc | u + cb | p + cc | pi + lb | e + lc | a + sb |
| KM48 | KM49 | KM50 | KM51 | KM52 | KM53 | KM54 | KM55 |
| s + a | h + bc | b + cb | u + cc | p + lb | pi + lc | e + sb | a + sc |
| KM56 | KM57 | KM58 | KM59 | KM60 | KM61 | KM62 | KM63 |
| s + bc | h + cb | b + cc | u + lb | p + lc | pi + sb | e + sc | a + bc |
| KM64 | KM65 | KM66 | KM67 | KM68 | KM69 | KM70 | KM71 |
| s + cb | h + cc | b + lb | u + lc | p + sb | pi + sc | e + bc | a + bz |
| KM72 | KM73 | KM74 | KM75 | KM76 | KM78 | KM79 | KM80 |
| s + cc | h + lb | b + lc | u + sb | p + sc | pi + bc | e + bz | s + lb |
| KM81 | KM82 | KM83 | KM84 | KM85 | KM86 | KM87 | KM88 |
| h + lc | b + sb | u + sc | p + bc | pi + bz | s + lc | h + sb | b + sc |
| KM89 | KM90 | KM91 | KM92 | KM93 | KM94 | KM95 | KM96 |
| u + bc | p + bz | s + sb | h + sc | b + bc | u + bz | s + sc | h + bc |
| KM97 | KM98 | KM99 | KM100 | KM101 | KM102 | KM103 | KM104 |
| b + bz | s + bc | h + bz | s + bz | bc + cb | cb + cc | cc + lb | lb + lc |
| KM105 | KM106 | KM107 | KM108 | KM109 | KM110 | KM111 | KM112 |
| lc + sb | sb + sc | sc + bc | bc + bz | bc + cc | cb + lb | cc + lc | lb + sb |
| KM113 | KM114 | KM115 | KM116 | KM117 | KM118 | KM119 | KM120 |
| lc + sc | sb + bc | sc + bz | bc + lb | cb + lc | cc + sb | lb + sc | lc + bc |
| KM121 | KM122 | KM123 | KM124 | KM125 | KM126 | KM127 | KM128 |
| sb + bz | bc + lc | cb + sb | cc + sc | lb + bc | lc + bz | bc + sb | cb + sc |
| KM129 | KM130 | KM131 | KM132 | KM133 | KM134 | KM135 | KM136 |
| cc + bc | lb + bz | bc + sc | cb + bc | cc + bz | bc + bc | cb + bz | bc + bz |
| KM137 | KM138 | KM139 | KM140 | KM141 | KM142 | KM143 | KM144 |
| s + h + b | s + b + u | s + u + p | s + p + pi | s + pi + e | s + e + a | s + a + bc | s + bc + cb |
| KM145 | KM146 | KM147 | KM148 | KM149 | KM150 | KM151 | KM152 |
| s + h + u | s + b + p | s + u + pi | s + p + e | s + pi + a | s + e + bc | s + a + cb | s + bc + cc |
| KM153 | KM154 | KM155 | KM156 | KM157 | KM158 | KM159 | KM160 |
| s + h + p | s + b + pi | s + u + e | s + p + a | s + pi + bc | s + e + cb | s + a + cc | s + bc + lb |
| KM161 | KM162 | KM163 | KM164 | KM165 | KM166 | KM167 | KM168 |
| s + h + pi | s + b + e | s + u + a | s + p + bc | s + pi + cb | s + e + cc | s + a + lb | s + bc + lc |
| KM169 | KM170 | KM171 | KM172 | KM173 | KM174 | KM175 | KM176 |
| s + h + e | s + b + a | s + u + bc | s + p + cb | s + pi + cc | s + e + lb | s + a + lc | s + bc + sb |
| KM177 | KM178 | KM179 | KM180 | KM181 | KM182 | KM183 | KM184 |
| s + h + a | s + b + bc | s + u + cb | s + p + cc | s + pi + lb | s + e + lc | s + a + sb | s + bc + sc |
| KM185 | KM186 | KM187 | KM188 | KM189 | KM190 | KM191 | KM192 |
| s + h + bc | s + b + cb | s + u + cc | s + p + lb | s + pi + lc | s + e + sb | s + a + sc | s + bc + bc |
| KM193 | KM194 | KM195 | KM196 | KM197 | KM198 | KM199 | KM200 |
| s + h + cb | s + b + cc | s + u + lb | s + p + lc | s + pi + sb | s + e + sc | s + a + bc | s + bc + bz |
| KM201 | KM202 | KM203 | KM204 | KM205 | KM206 | KM207 | KM208 |
| s + h + cc | s + b + lb | s + u + lc | s + p + sb | s + pi + sc | s + e + bc | s + a + bz | s + h + lb |
| KM209 | KM210 | KM211 | KM212 | KM213 | KM214 | KM215 | KM216 |
| s + b + lc | s + u + sb | s + p + sc | s + pi + bc | s + e + bz | s + h + lc | s + b + sb | s + u + sc |
| KM217 | KM218 | KM219 | KM220 | KM221 | KM222 | KM223 | KM224 |
| s + p + bc | s + pi + bz | s + h + sb | s + b + sc | s + u + bc | s + p + bz | s + h + sc | s + b + bc |
| KM225 | KM226 | KM227 | KM228 | KM229 | KM230 | KM231 | KM232 |
| s + u + bz | s + h + bc | s + b + bz | s + h + bz | s + cb + cc | s + cc + lb | s + lb + lc | s + lc + sb |
| KM233 | KM234 | KM235 | KM236 | KM237 | KM238 | KM239 | KM240 |
| s + sb + sc | s + sc + bc | s + bc + bz | s + cb + lb | s + cc + lc | s + lb + sb | s + lc + sc | s + sb + bc |
| KM241 | KM242 | KM243 | KM244 | KM245 | KM246 | KM247 | KM248 |
| s + sc + bz | s + cb + lc | s + cc + sb | s + lb + sc | s + lc + bc | s + sb + bz | s + cb + sb | s + cc + sc |
| KM249 | KM250 | KM251 | KM252 | KM253 | KM254 | KM255 | KM256 |
| s + lb + bc | s + lc + bz | s + cb + sc | s + cc + bc | s + lb + bz | s + cb + bc | s + cc + bz | s + cb + bz |
| KM257 | KM258 | KM259 | KM260 | KM261 | KM262 | KM263 | KM264 |
| h + b + u | h + u + p | h + p + pi | h + pi + e | h + e + a | h + a + bc | h + bc + cb | h + cb + cc |
| KM265 | KM266 | KM267 | KM268 | KM269 | KM270 | KM271 | KM272 |
| h + b + p | h + u + pi | h + p + e | h + pi + a | h + e + bc | h + a + cb | h + bc + cc | h + cb + lb |
| KM273 | KM274 | KM275 | KM276 | KM277 | KM278 | KM279 | KM280 |
| h + b + pi | h + u + e | h + p + a | h + pi + bc | h + e + cb | h + a + cc | h + bc + lb | h + cb + lc |
| KM281 | KM282 | KM283 | KM284 | KM285 | KM286 | KM287 | KM288 |
| h + b + e | h + u + a | h + p + bc | h + pi + cb | h + e + cc | h + a + lb | h + bc + lc | h + cb + sb |
| KM289 | KM290 | KM291 | KM292 | KM293 | KM294 | KM295 | KM296 |
| h + b + a | h + u + bc | h + p + cb | h + pi + cc | h + e + lb | h + a + lc | h + bc + sb | h + cb + sc |
| KM297 | KM298 | KM299 | KM300 | KM301 | KM302 | KM303 | KM304 |
| h + b + bc | h + u + cb | h + p + cc | h + pi + lb | h + e + lc | h + a + sb | h + bc + sc | h + cb + bc |
| KM305 | KM306 | KM307 | KM308 | KM309 | KM310 | KM311 | KM312 |
| h + b + cb | h + u + cc | h + p + lb | h + pi + lc | h + e + sb | h + a + sc | h + bc + bc | h + cb + bz |
| KM313 | KM314 | KM315 | KM316 | KM317 | KM318 | KM319 | KM320 |
| h + b + cc | h + u + lb | h + p + lc | h + pi + sb | h + e + sc | h + a + bc | h + bc + bz | h + b + lb |
| KM321 | KM322 | KM323 | KM324 | KM325 | KM326 | KM327 | KM328 |
| h + u + lc | h + p + sb | h + pi + sc | h + e + bc | h + a + bz | h + b + lc | h + u + sb | h + p + sc |
| KM329 | KM330 | KM331 | KM332 | KM333 | KM334 | KM335 | KM336 |
| h + pi + bc | h + e + bz | h + b + sb | h + u + sc | h + p + bc | h + pi + bz | h + b + sc | h + u + bc |
| KM337 | KM338 | KM339 | KM340 | KM341 | KM342 | KM343 | KM344 |
| h + p + bz | h + b + bc | h + u + bz | h + b + bz | h + cc + lb | h + lb + lc | h + lc + sb | h + sb + sc |
| KM345 | KM346 | KM347 | KM348 | KM349 | KM350 | KM351 | KM352 |
| h + sc + bc | h + bc + bz | h + cc + lc | h + lb + sb | h + lc + sc | h + sb + bc | h + sc + bz | h + cc + sb |
| KM353 | KM354 | KM355 | KM356 | KM357 | KM358 | KM359 | KM360 |
| h + lb + sc | h + lc + bc | h + sb + bz | h + cc + sc | h + lb + bc | h + lc + bz | h + cc + bc | h + lb + bz |
| KM361 | KM362 | KM363 | KM364 | KM365 | KM366 | KM367 | KM368 |
| h + cc + bz | b + u + p | b + p + pi | b + pi + e | b + e + a | b + a + bc | b + bc + cb | b + cb + cc |
| KM369 | KM370 | KM371 | KM372 | KM373 | KM374 | KM375 | KM376 |
| b + cc + lb | b + u + pi | b + p + e | b + pi + a | b + e + bc | b + a + cb | b + bc + cc | b + cb + lb |
| KM377 | KM378 | KM379 | KM380 | KM381 | KM382 | KM383 | KM384 |
| b + cc + lc | b + u + e | b + p + a | b + pi + bc | b + e + cb | b + a + cc | b + bc + lb | b + cb + lc |
| KM385 | KM386 | KM387 | KM388 | KM389 | KM390 | KM391 | KM392 |
| b + cc + sb | b + u + a | b + p + bc | b + pi + cb | b + e + cc | b + a + lb | b + bc + lc | b + cb + sb |
| KM393 | KM394 | KM395 | KM396 | KM397 | KM398 | KM399 | KM400 |
| b + cc + sc | b + u + bc | b + p + cb | b + pi + cc | b + e + lb | b + a + lc | b + bc + sb | b + cb + sc |
| KM401 | KM402 | KM403 | KM404 | KM405 | KM406 | KM407 | KM408 |
| b + cc + bc | b + u + cb | b + p + cc | b + pi + lb | b + e + lc | b + a + sb | b + bc + sc | b + cb + bc |
| KM409 | KM410 | KM411 | KM412 | KM413 | KM414 | KM415 | KM416 |
| b + cc + bz | b + u + cc | b + p + lb | b + pi + lc | b + e + sb | b + a + sc | b + bc + bc | b + cb + bz |
| KM417 | KM418 | KM419 | KM420 | KM421 | KM422 | KM423 | KM424 |
| b + u + lb | b + p + lc | b + pi + sb | b + e + sc | b + a + bc | b + bc + bz | b + u + lc | b + p + sb |
| KM425 | KM426 | KM427 | KM428 | KM429 | KM430 | KM431 | KM432 |
| b + pi + sc | b + e + bc | b + a + bz | b + u + sb | b + p + sc | b + pi + bc | b + e + bz | b + u + sc |
| KM433 | KM434 | KM435 | KM436 | KM437 | KM438 | KM439 | KM440 |
| b + p + bc | b + pi + bz | b + u + bc | b + p + bz | b + u + bz | b + lb + lc | b + lc + sb | b + sb + sc |
| KM441 | KM442 | KM443 | KM444 | KM445 | KM446 | KM447 | KM448 |
| b + sc + bc | b + bc + bz | b + lb + sb | b + lc + sc | b + sb + bc | b + sc + bz | b + lb + sc | b + lc + bc |
| KM449 | KM450 | KM451 | KM452 | KM453 | KM454 | KM455 | KM456 |
| b + sb + bz | b + lb + bc | b + lc + bz | b + lb + bz | u + p + pi | u + pi + e | u + e + a | u + a + bc |
| KM457 | KM458 | KM459 | KM460 | KM461 | KM462 | KM463 | KM464 |
| u + bc + cb | u + cb + cc | u + cc + lb | u + lb + lc | u + p + e | u + pi + a | u + e + bc | u + a + cb |
| KM465 | KM466 | KM467 | KM468 | KM469 | KM470 | KM471 | KM472 |
| u + bc + cc | u + cb + lb | u + cc + lc | u + lb + sb | u + p + a | u + pi + bc | u + e + cb | u + a + cc |
| KM473 | KM474 | KM475 | KM476 | KM477 | KM478 | KM479 | KM480 |
| u + bc + lb | u + cb + lc | u + cc + sb | u + lb + sc | u + p + bc | u + pi + cb | u + e + cc | u + a + lb |
| KM481 | KM482 | KM483 | KM484 | KM485 | KM486 | KM487 | KM488 |
| u + bc + lc | u + cb + sb | u + cc + sc | u + lb + bc | u + p + cb | u + pi + cc | u + e + lb | u + a + lc |
| KM489 | KM490 | KM491 | KM492 | KM493 | KM494 | KM495 | KM496 |
| u + bc + sb | u + cb + sc | u + cc + bc | u + lb + bz | u + p + cc | u + pi + lb | u + e + lc | u + a + sb |
| KM497 | KM498 | KM499 | KM500 | KM501 | KM502 | KM503 | KM504 |
| u + bc + sc | u + cb + bc | u + cc + bz | u + p + lb | u + pi + lc | u + e + sb | u + a + sc | u + bc + bc |
| KM505 | KM506 | KM507 | KM508 | KM509 | KM510 | KM511 | KM512 |
| u + cb + bz | u + p + lc | u + pi + sb | u + e + sc | u + a + bc | u + bc + bz | u + p + sb | u + pi + sc |
| KM513 | KM514 | KM515 | KM516 | KM517 | KM518 | KM519 | KM520 |
| u + e + bc | u + a + bz | u + p + sc | u + pi + bc | u + e + bz | u + p + bc | u + pi + bz | u + p + bz |
| KM521 | KM522 | KM523 | KM524 | KM525 | KM526 | KM527 | KM528 |
| p + pi + e | p + e + a | p + a + bc | p + bc + cb | p + cb + cc | p + cc + lb | p + lb + lc | p + lc + sb |
| KM529 | KM530 | KM531 | KM532 | KM533 | KM534 | KM535 | KM536 |
| p + pi + a | p + e + bc | p + a + cb | p + bc + cc | p + cb + lb | p + cc + lc | p + lb + sb | p + lc + sc |
| KM537 | KM538 | KM539 | KM540 | KM541 | KM542 | KM543 | KM544 |
| p + pi + bc | p + e + cb | p + a + cc | p + bc + lb | p + cb + lc | p + cc + sb | p + lb + sc | p + lc + bc |
| KM545 | KM546 | KM547 | KM548 | KM549 | KM550 | KM551 | KM552 |
| p + pi + cb | p + e + cc | p + a + lb | p + bc + lc | p + cb + sb | p + cc + sc | p + lb + bc | p + lc + bz |
| KM553 | KM554 | KM555 | KM556 | KM557 | KM558 | KM559 | KM560 |
| p + pi + cc | p + e + lb | p + a + lc | p + bc + sb | p + cb + sc | p + cc + bc | p + lb + bz | p + pi + lb |
| KM561 | KM562 | KM563 | KM564 | KM565 | KM566 | KM567 | KM568 |
| p + e + lc | p + a + sb | p + bc + sc | p + cb + bc | p + cc + bz | p + pi + lc | p + e + sb | p + a + sc |
| KM569 | KM570 | KM571 | KM572 | KM573 | KM574 | KM575 | KM576 |
| p + bc + bc | p + pi + bc | p + e + bz | p + e + sc | p + a + bc | p + bc + bz | p + pi + sc | p + e + bc |
| KM577 | KM578 | KM579 | KM580 | KM581 | KM582 | KM583 | KM584 |
| p + a + bz | p + pi + bz | u + lc + sb | u + sb + sc | u + sc + bc | u + bc + bz | u + lc + sc | u + sb + bc |
| KM585 | KM586 | KM587 | KM588 | KM589 | KM590 | KM591 | KM592 |
| u + sc + bz | u + lc + bc | u + sb + bz | u + lc + bz | p + sb + sc | p + sc + bc | p + bc + bz | p + sb + bc |
| KM593 | KM594 | KM595 | KM596 | KM597 | KM598 | KM599 | KM600 |
| p + sc + bz | p + sb + bz | pi + e + a | pi + a + bc | pi + bc + cb | pi + cb + cc | pi + cc + lb | pi + lb + lc |
| KM601 | KM602 | KM603 | KM604 | KM605 | KM606 | KM607 | KM608 |
| pi + lc + sb | pi + sb + sc | pi + e + bc | pi + a + cb | pi + bc + cc | pi + cb + lb | pi + cc + lc | pi + lb + sb |
| KM609 | KM610 | KM611 | KM612 | KM613 | KM614 | KM615 | KM616 |
| pi + lc + sc | pi + sb + bc | pi + e + cb | pi + a + cc | pi + bc + lb | pi + cb + lc | pi + cc + sb | pi + lb + sc |
| KM625 | KM626 | KM627 | KM628 | KM629 | KM630 | KM631 | KM632 |
| pi + lc + bc | pi + sb + bz | pi + e + cc | pi + a + lb | pi + bc + lc | pi + cb + sb | pi + cc + sc | pi + lb + bc |
| KM633 | KM634 | KM635 | KM636 | KM637 | KM638 | KM639 | KM640 |
| pi + lc + bz | pi + e + lb | pi + a + lc | pi + bc + sb | pi + cb + sc | pi + cc + bc | pi + lb + bz | pi + e + lc |
| KM641 | KM642 | KM643 | KM644 | KM645 | KM646 | KM647 | KM648 |
| pi + a + sb | pi + bc + sc | pi + cb + bc | pi + cc + bz | pi + e + sb | pi + a + sc | pi + bc + bc | pi + cb + bz |
| KM649 | KM650 | KM651 | KM652 | KM653 | KM654 | KM655 | KM656 |
| pi + e + sc | pi + a + bc | pi + bc + bz | pi + e + bc | pi + a + bz | pi + e + bz | pi + sc + bc | pi + bc + bz |
| KM657 | KM658 | KM659 | KM660 | KM661 | KM662 | KM663 | KM664 |
| pi + sc + bz | e + a + bc | e + bc + cb | e + cb + cc | e + cc + lb | e + lb + lc | e + lc + sb | e + sb + sc |
| KM665 | KM666 | KM667 | KM668 | KM669 | KM670 | KM671 | KM672 |
| e + sc + bc | e + a + cb | e + bc + cc | e + cb + lb | e + cc + lc | e + lb + sb | e + lc + sc | e + sb + bc |
| KM673 | KM674 | KM675 | KM676 | KM677 | KM678 | KM679 | KM680 |
| e + sc + bz | e + a + cc | e + bc + lb | e + cb + lc | e + cc + sb | e + lb + sc | e + lc + bc | e + sb + bz |
| KM681 | KM682 | KM683 | KM684 | KM685 | KM686 | KM687 | KM688 |
| e + a + lb | e + bc + lc | e + cb + sb | e + cc + sc | e + lb + bc | e + lc + bz | e + a + lc | e + bc + sb |
| KM689 | KM690 | KM691 | KM692 | KM693 | KM694 | KM695 | KM696 |
| e + cb + sc | e + cc + bc | e + lb + bz | e + a + sb | e + bc + sc | e + cb + bc | e + cc + bz | e + a + sc |
| KM697 | KM698 | KM699 | KM700 | KM701 | KM702 | KM703 | KM704 |
| e + bc + bc | e + cb + bz | e + a + bc | e + bc + bz | e + a + bz | e + bc + bz | a + bc + cb | a + cb + cc |
| KM705 | KM706 | KM707 | KM708 | KM709 | KM710 | KM711 | KM712 |
| a + cc + lb | a + lb + lc | a + lc + sb | a + sb + sc | a + sc + bc | a + bc + bz | a + bc + cc | a + cb + lb |
| KM713 | KM714 | KM715 | KM716 | KM717 | KM718 | KM719 | KM720 |
| a + cc + lc | a + lb + sb | a + lc + sc | a + sb + bc | a + sc + bz | a + bc + lb | a + cb + lc | a + cc + sb |
| KM721 | KM722 | KM723 | KM724 | KM725 | KM726 | KM727 | KM728 |
| a + lb + sc | a + lc + bc | a + sb + bz | a + bc + lc | a + cb + sb | a + cc + sc | a + lb + bc | a + lc + bz |
| KM729 | KM730 | KM731 | KM732 | KM733 | KM734 | KM735 | KM736 |
| a + bc + sb | a + cb + sc | a + cc + bc | a + lb + bz | a + bc + sc | a + cb + bc | a + cc + bz | a + bc + bc |
| KM737 | KM738 | KM739 | KM740 | KM741 | KM742 | KM743 | KM744 |
| a + cb + bz | a + bc + bz | bc + cb + | bc + cc + lb | bc + lb + lc | bc + lc + sb | bc + sb + | bc + sc + bc |
| | | cc | | | | sc | |
| KM745 | KM746 | KM747 | KM748 | KM749 | KM750 | KM751 | KM752 |
| bc + bc + | bc + cb + lb | bc + cc + lc | bc + lb + sb | bc + lc + sc | bc + sb + | bc + sc + | bc + cb + lc |
| bz | | | | | bc | bz | |
| KM753 | KM754 | KM755 | KM756 | KM757 | KM758 | KM759 | KM760 |
| bc + cc + sb | bc + lb + sc | bc + lc + bc | bc + sb + | bc + cb + | bc + cc + sc | bc + lb + bc | bc + lc + bz |
| | | | bz | sb | | | |
| KM761 | KM762 | KM763 | KM764 | KM765 | KM766 | KM767 | KM768 |
| bc + cb + sc | bc + cc + | bc + lb + bz | bc + cb + | bc + cc + bz | bc + cb + | cb + cc + lb | cb + lb + lc |
| | bc | | bc | | bz | | |
| KM769 | KM770 | KM771 | KM772 | KM773 | KM774 | KM775 | KM776 |
| cb + lc + sb | cb + sb + | cb + sc + | cb + bc + | cb + cc + lc | cb + lb + sb | cb + lc + sc | cb + sb + |
| | sc | bc | bz | | | | bc |
| KM777 | KM778 | KM779 | KM780 | KM781 | KM782 | KM783 | KM784 |
| cb + sc + bz | cb + cc + | cb + lb + sc | cb + lc + bc | cb + sb + | cb + cc + sc | cb + lb + bc | cb + lc + bz |
| | sb | | | bz | | | |
| KM785 | KM786 | KM787 | KM788 | KM789 | KM790 | KM791 | KM792 |
| cb + cc + bc | cb + lb + bz | cb + cc + | cc + lb + lc | cc + lc + sb | cc + sb + sc | cc + sc + bc | cc + bc + bz |
| | | bz | | | | | |
| KM793 | KM794 | KM795 | KM796 | KM797 | KM798 | KM799 | KM800 |
| cc + lb + sb | cc + lc + sc | cc + sb + | cc + sc + bz | cc + lb + sc | cc + lc + bc | cc + sb + | cc + lb + bc |
| | | bc | | | | bz | |
| KM801 | KM802 | KM803 | KM804 | KM805 | KM806 | KM807 | KM808 |
| cc + lc + bz | cc + lb + bz | lb + lc + sb | lb + sb + sc | lb + sc + bc | lb + bc + bz | lb + lc + sc | lb + sb + bc |
| KM809 | KM810 | KM811 | KM812 | KM813 | KM814 | KM815 | KM816 |
| lb + sc + bz | lb + lc + bc | lb + sb + bz | lb + lc + bz | lc + sb + sc | lc + sc + bc | lc + bc + bz | lc + sb + bc |
| KM817 | KM818 | KM819 | KM820 | KM821 | KM822 | KM823 | KM824 |
| lc + sc + bz | lc + sb + bz | sb + sc + | sb + bc + | sb + sc + | sc + bc + bz | sb + aq + oc | sc + aq + |
| | | bc | bz | bz | | | oc |
| KM825 | KM826 | KM827 | KM828 | KM829 | KM830 | KM831 | KM832 |
| s + h + b + u | s + h + u + p | s + h + p + pi | s + h + pi + e | s + h + a + | s + b + u + p | s + b + p + pi | s + b + pi + e |
| | | | | aq | | | |
| KM833 | KM834 | KM835 | KM836 | KM837 | KM838 | KM839 | KM840 |
| s + h + b + p | s + h + u + pi | s + h + p + e | s + h + pi + a | s + h + u + | s + b + u + pi | s + b + p + e | s + b + pi + a |
| | | | | aq | | | |
| KM841 | KM842 | KM843 | KM844 | KM845 | KM846 | KM847 | KM848 |
| s + h + b + pi | s + h + u + e | s + h + p + a | s + h + pi + | s + h + b + a | s + b + u + e | s + b + p + a | s + b + pi + |
| | | | aq | | | | aq |
| KM849 | KM850 | KM851 | KM852 | KM853 | KM854 | KM855 | KM856 |
| s + h + b + e | s + h + u + a | s + h + p + | s + b + u + a | s + b + p + | s + b + u + | s + h + b + | s + b + e + a |
| | | aq | | aq | aq | aq | |
| KM857 | KM858 | KM859 | KM860 | KM861 | KM862 | KM863 | KM864 |
| s + b + a + | s + u + p + pi | s + u + pi + e | s + u + e + a | s + u + a + | s + b + e + | s + u + p + e | s + u + pi + a |
| aq | | | | aq | aq | | |
| KM865 | KM866 | KM867 | KM868 | KM869 | KM870 | KM871 | KM872 |
| s + u + e + | s + u + p + a | s + u + pi + | s + u + p + | s + p + pi + e | s + p + e + a | s + p + a + aq | s + pi + e + a |
| aq | | aq | aq | | | | |
| KM873 | KM874 | KM875 | KM876 | KM877 | KM878 | KM879 | KM880 |
| s + pi + a + | s + e + a + | s + p + pi + a | s + p + e + | s + pi + e + | s + p + pi + | h + b + u + p | h + b + p + |
| aq | aq | | aq | aq | aq | | pi |
| KM881 | KM882 | KM883 | KM884 | KM885 | KM886 | KM887 | KM888 |
| h + b + pi + e | h + b + e + a | h + b + a + aq | h + u + p + | h + u + pi + e | h + u + e + a | h + b + u + | h + b + p + e |
| | | | pi | | | pi | |
| KM889 | KM890 | KM891 | KM892 | KM893 | KM894 | KM895 | KM896 |
| h + b + pi + a | h + b + e + | h + u + p + e | h + u + pi + a | h + u + e + | h + b + u + e | h + b + p + a | h + b + pi + |
| | aq | | | aq | | | aq |
| KM897 | KM898 | KM899 | KM900 | KM901 | KM902 | KM903 | KM904 |
| h + u + p + a | h + u + pi + | h + b + u + a | h + b + p + | h + u + p + | h + b + u + | h + u + a + | h + p + pi + e |
| | aq | | aq | aq | aq | aq | |
| KM905 | KM906 | KM907 | KM908 | KM909 | KM910 | KM911 | KM912 |
| h + p + e + a | h + p + a + aq | h + pi + e + a | h + pi + e + | h + e + a + aq | h + p + pi + a | h + p + e + aq | h + pi + e + |
| | | | aq | | | | aq |
| KM913 | KM914 | KM915 | KM916 | KM917 | KM918 | KM919 | KM920 |
| h + p + pi + | b + u + p + | b + u + pi + e | b + u + e + a | b + u + a + | b + p + pi + e | b + p + e + a | b + p + a + |
| aq | pi | | | aq | | | aq |
| KM921 | KM922 | KM923 | KM924 | KM925 | KM926 | KM927 | KM928 |
| b + pi + e + a | b + pi + a + | b + e + a + | b + u + p + e | b + u + pi + a | b + u + e + | b + p + pi + a | b + p + e + |
| | aq | aq | | | aq | | aq |
| KM929 | KM930 | KM931 | KM932 | KM933 | KM934 | KM935 | KM936 |
| b + pi + e + | b + u + p + a | b + u + pi + | b + p + pi + | b + u + p + | u + p + pi + e | u + p + e + a | u + p + a + |
| aq | | aq | aq | aq | | | aq |
| KM937 | KM938 | KM939 | KM940 | KM941 | KM942 | KM943 | KM944 |
| u + pi + e + a | u + pi + a + | u + e + a + | u + p + pi + a | u + p + e + | u + pi + e + | u + p + pi + | p + pi + e + a |
| | aq | aq | | aq | aq | aq | |
| KM945 | KM946 | KM947 | KM948 | KM949 | KM950 | KM951 | KM952 |
| p + pi + a + | p + pi + e + | pi + e + a + | s + h + b + u + | s + h + b + u + | s + oc | h + oc | b + oc |
| aq | aq | aq | p + pi + a + | p + pi + a + | | | |
| | | | e + aq | e + bc + cb + | | | |
| | | | | cc + | | | |
| | | | | lb + lc + sb + | | | |
| | | | | sc + bc + bz | | | |
| KM953 | KM954 | KM955 | KM956 | KM957 | KM958 | KM959 | KM960 |
| u + oc | p + oc | pi + oc | e + oc | a + oc | bz + oc | bc + oc | cb + oc |
| KM961 | KM962 | KM963 | KM964 | KM965 | KM966 | KM967 | KM968 |
| cc + oc | lb + oc | lc + oc | sb + oc | sc + oc | aq + oc | s + h + oc | s + b + oc |
| KM969 | KM970 | KM971 | KM972 | KM973 | KM974 | KM975 | KM976 |
| s + u + oc | s + p + oc | s + pi + oc | s + e + oc | s + a + oc | s + bz + oc | s + bc + oc | s + bc + oc |
| KM977 | KM978 | KM979 | KM980 | KM981 | KM982 | KM983 | KM984 |
| s + cb + oc | s + cc + oc | s + lb + oc | s + lc + oc | s + sb + oc | s + sc + oc | s + aq + oc | h + b + oc |
| KM985 | KM986 | KM987 | KM988 | KM989 | KM990 | KM991 | KM992 |
| h + u + oc | h + p + oc | h + pi + oc | h + e + oc | h + a + oc | h + bz + oc | h + bc + oc | h + bc + oc |
| KM993 | KM994 | KM995 | KM996 | KM997 | KM998 | KM999 | KM1000 |
| h + cb + oc | h + cc + oc | h + lb + oc | h + lc + oc | h + sb + oc | h + sc + oc | h + aq + oc | u + p + oc |
| KM1001 | KM1002 | KM1003 | KM1004 | KM1005 | KM1006 | KM1007 | KM1008 |
| u + pi + oc | u + e + oc | u + a + oc | u + bz + oc | u + bc + oc | u + bc + oc | u + cb + oc | u + cc + oc |
| KM1009 | KM1010 | KM1011 | KM1012 | KM1013 | KM1014 | KM1015 | KM1016 |
| u + lb + oc | u + lc + oc | u + sb + oc | u + sc + oc | u + aq + oc | p + pi + oc | p + e + oc | p + a + oc |
| KM1017 | KM1018 | KM1019 | KM1020 | KM1021 | KM1022 | KM1023 | KM1024 |
| p + bz + oc | p + bc + oc | p + cb + oc | p + cc + oc | p + lb + oc | p + lc + oc | p + sb + oc | p + sc + oc |
| KM1025 | KM1026 | KM1027 | KM1028 | KM1029 | KM1030 | KM1031 | KM1032 |
| pi + e + oc | pi + a + oc | pi + bz + oc | pi + bc + oc | pi + bc + oc | pi + cb + oc | pi + cc + oc | pi + lb + oc |
| KM1033 | KM1034 | KM1035 | KM1036 | KM1037 | KM1038 | KM1039 | KM1040 |
| pi + lc + oc | pi + sb + | pi + sc + oc | pi + aq + oc | e + a + oc | e + bz + oc | e + bc + oc | e + cb + oc |
| | oc | | | | | | |
| KM1041 | KM1042 | KM1043 | KM1044 | KM1045 | KM1046 | KM1047 | KM1048 |
| e + cc + oc | e + lb + oc | e + lc + oc | e + sb + oc | e + sc + oc | e + aq + oc | a + bz + oc | a + bc + oc |
| KM1049 | KM1050 | KM1051 | KM1052 | KM1053 | KM1054 | KM1055 | KM1056 |
| a + bc + oc | a + cb + oc | a + cc + oc | a + lb + oc | a + lc + oc | a + sb + oc | a + sc + oc | p + aq + oc |
| KM1057 | KM1058 | KM1059 | KM1060 | KM1061 | KM1062 | KM1063 | KM1064 |
| a + aq + | bz + bc + | bz + cb + oc | bz + cc + oc | bz + lb + oc | bz + lc + oc | bz + sb + oc | bz + sc + |
| oc | oc | | | | | | oc |
| KM1065 | KM1066 | KM1067 | KM1068 | KM1069 | KM1070 | KM1071 | KM1072 |
| bz + aq + oc | bc + cb + | bc + cc + oc | bc + lb + oc | bc + lc + oc | bc + sb + oc | bc + sc + oc | bc + aq + |
| | oc | | | | | | oc |
| KM1073 | KM1074 | KM1075 | KM1076 | KM1077 | KM1078 | KM1079 | KM1080 |
| cb + cc + | cb + lb + | cb + lc + oc | cb + sb + oc | cb + sc + oc | cb + aq + oc | cc + lb + oc | cc + lc + oc |
| oc | oc | | | | | | |
| KM1081 | KM1082 | KM1083 | KM1084 | KM1085 | KM1086 | KM1087 | KM1088 |
| cc + sb + | cc + sc + | cc + aq + oc | lb + lc + oc | lb + sb + oc | lb + sc + oc | lb + aq + oc | lc + sb + oc |
| oc | oc | | | | | | |
| KM1089 | KM1090 | KM1091 | KM1092 | KM1093 | KM1094 | KM1095 | KM1096 |
| lc + sc + | lc + aq + | sb + sc + oc | s + h + b + oc | s + b + u + oc | s + u + p + oc | s + p + pi + | s + pi + e + |
| oc | oc | | | | | oc | oc |
| KM1097 | KM1098 | KM1099 | KM1100 | KM1101 | KM1102 | KM1103 | KM1104 |
| s + e + a + oc | s + a + aq + | s + h + a + | s + h + u + | s + h + oc + | s + h + pi + | s + h + p + | s + b + p + |
| | oc | oc | oc | oc | oc | oc | oc |
| KM1105 | KM1106 | KM1107 | KM1108 | KM1109 | KM1110 | KM1111 | KM1112 |
| s + b + u + | s + h + b + | s + b + a + | s + u + a + | s + b + e + | s + u + e + | s + u + pi + | s + u + p + |
| oc | oc | oc | oc | oc | oc | oc | oc |
| KM1113 | KM1114 | KM1115 | KM1116 | KM1117 | KM1118 | KM1119 | KM1120 |
| s + p + a + oc | s + pi + a + | s + e + a + | s + p + e + | s + pi + e + | s + p + pi + | h + b + a + | h + b + e + |
| | oc | oc | oc | oc | oc | oc | oc |
| KM1121 | KM1122 | KM1123 | KM1124 | KM1125 | KM1126 | KM1127 | KM1128 |
| h + u + e + | h + b + pi + | h + u + pi + | h + b + p + | h + u + p + | h + b + u + | h + u + a + | h + p + a + |
| oc | oc | oc | oc | oc | oc | oc | oc |
| KM1129 | KM1130 | KM1131 | KM1132 | KM1133 | KM1134 | KM1135 | KM1136 |
| h + pi + e + | h + e + a + oc | h + p + e + | h + pi + e + | h + p + pi + | b + u + a + | b + p + a + | b + pi + e + |
| oc | | oc | oc | oc | oc | oc | oc |
| KM1137 | KM1138 | KM1139 | KM1140 | KM1141 | KM1142 | KM1143 | KM1144 |
| b + u + pi + | b + p + pi + | b + u + p + | u + p + a + | u + pi + a + | u + e + a + | u + p + e + | u + pi + e + |
| oc | oc | oc | oc | oc | oc | oc | oc |
| KM1145 | KM1146 | KM1147 | KM1148 | KM1149 | KM1150 | KM1151 | KM1152 |
| u + p + pi + | p + pi + a + | p + pi + e + | pi + e + a + | e + a + aq + | s + h + b + u + | s + h + b + u + | s + h + b + u + |
| oc | oc | oc | oc | oc | p + pi + a + | p + pi + a + | p + pi + a + |
| | | | | | e + aq + oc + | e + aq + oc + | e + bc + cb + |
| | | | | | hd | hp | cc + lb + |
| | | | | | | | lc + sb + |
| | | | | | | | sc + bc + bz + |
| | | | | | | | oc + hd |
| KM1153 | KM1154 | KM1155 | KM1156 | KM1157 | KM1158 | KM1159 | KM1160 |
| s + h + b + u + | s + hx | h + hx | b + hx | s + hd | h + hd | b + hd | s + hp |
| p + pi + a + | | | | | | | |
| e + bc + cb + | | | | | | | |
| cc + lb + | | | | | | | |
| lc + sb + | | | | | | | |
| sc + bc + bz + | | | | | | | |
| oc + | | | | | | | |
| hp | | | | | | | |
| KM1161 | KM1162 | KM1163 | KM1164 | KM1165 | KM1166 | KM1167 | KM1168 |
| u + hx | p + hx | pi + hx | e + hx | a + hx | bz + hx | bc + hx | cb + hx |
| KM1169 | KM1170 | KM1171 | KM1172 | KM1173 | KM1174 | KM1175 | KM1176 |
| cc + hx | lb + hx | lc + hx | sb + hx | sc + hx | aq + hx | s + h + hx | s + b + hx |
| KM1177 | KM1178 | KM1179 | KM1180 | KM1181 | KM1182 | KM1183 | KM1184 |
| s + u + hx | s + p + hx | s + pi + hx | s + e + hx | s + a + hx | s + bz + hx | s + bc + hx | s + bc + hx |
| KM1185 | KM1186 | KM1187 | KM1188 | KM1189 | KM1190 | KM1191 | KM1192 |
| s + cb + hx | s + cc + hx | s + lb + hx | s + lc + hx | s + sb + hx | s + sc + hx | s + aq + hx | h + b + hx |
| KM1193 | KM1194 | KM1195 | KM1196 | KM1197 | KM1198 | KM1199 | KM1200 |
| h + u + hx | h + p + hx | h + pi + hx | h + e + hx | h + a + hx | h + bz + hx | h + bc + hx | h + bc + hx |
| KM1201 | KM1202 | KM1203 | KM1204 | KM1205 | KM1206 | KM1207 | KM1208 |
| h + cb + hx | h + cc + hx | h + lb + hx | h + lc + hx | h + sb + hx | h + sc + hx | h + aq + hx | u + p + hx |
| KM1209 | KM1210 | KM1211 | KM1212 | KM1213 | KM1214 | KM1215 | KM1216 |
| u + pi + hx | u + e + hx | u + a + hx | u + bz + hx | u + bc + hx | u + bc + hx | u + cb + hx | u + cc + hx |
| KM1217 | KM1218 | KM1219 | KM1220 | KM1221 | KM1222 | KM1223 | KM1224 |
| u + lb + hx | u + lc + hx | u + sb + hx | u + sc + hx | u + aq + hx | p + pi + hx | p + e + hx | p + a + hx |
| KM1225 | KM1226 | KM1227 | KM1228 | KM1229 | KM1230 | KM1231 | KM1232 |
| p + bz + hx | p + bc + hx | p + cb + hx | p + cc + hx | p + lb + hx | p + lc + hx | p + sb + hx | p + sc + hx |
| KM1233 | KM1234 | KM1235 | KM1236 | KM1237 | KM1238 | KM1239 | KM1240 |
| pi + e + hx | pi + a + hx | pi + bz + hx | pi + bc + hx | pi + bc + hx | pi + cb + hx | pi + cc + hx | pi + lb + hx |
| KM1241 | KM1242 | KM1243 | KM1244 | KM1245 | KM1246 | KM1247 | KM1248 |
| pi + lc + hx | pi + sb + | pi + sc + hx | pi + aq + hx | e + a + hx | e + bz + hx | e + bc + hx | e + cb + hx |
| | hx | | | | | | |
| KM1249 | KM1250 | KM1251 | KM1252 | KM1253 | KM1254 | KM1255 | KM1256 |
| e + cc + hx | e + lb + hx | e + lc + hx | e + sb + hx | e + sc + hx | e + aq + hx | a + bz + hx | a + bc + hx |
| KM1257 | KM1258 | KM1259 | KM1260 | KM1261 | KM1262 | KM1263 | KM1264 |
| a + bc + hx | a + cb + hx | a + cc + hx | a + lb + hx | a + lc + hx | a + sb + hx | a + sc + hx | p + aq + hx |
| KM1265 | KM1266 | KM1267 | KM1268 | KM1269 | KM1270 | KM1271 | KM1272 |
| a + aq + | bz + bc + | bz + cb + hx | bz + cc + hx | bz + lb + hx | bz + lc + hx | bz + sb + hx | bz + sc + |
| hx | hx | | | | | | hx |
| KM1273 | KM1274 | KM1275 | KM1276 | KM1277 | KM1278 | KM1279 | KM1280 |
| bz + aq + hx | bc + cb + | bc + cc + hx | bc + lb + hx | bc + lc + hx | bc + sb + hx | bc + sc + hx | bc + aq + |
| | hx | | | | | | hx |
| KM1281 | KM1282 | KM1283 | KM1284 | KM1285 | KM1286 | KM1287 | KM1288 |
| cb + cc + | cb + lb + | cb + lc + hx | cb + sb + hx | cb + sc + hx | cb + aq + hx | cc + lb + hx | cc + lc + hx |
| hx | hx | | | | | | |
| KM1289 | KM1290 | KM1291 | KM1292 | KM1293 | KM1294 | KM1295 | KM1296 |
| cc + sb + | cc + sc + | cc + aq + hx | lb + lc + hx | lb + sb + hx | lb + sc + hx | lb + aq + hx | lc + sb + hx |
| hx | hx | | | | | | |
| KM1297 | KM1298 | KM1299 | KM1300 | KM1301 | KM1302 | KM1303 | KM1304 |
| lc + sc + | lc + aq + | sb + sc + hx | s + h + b + hx | s + b + u + hx | s + u + p + hx | s + p + pi + | s + pi + e + |
| hx | hx | | | | | hx | hx |
| KM1305 | KM1306 | KM1307 | KM1308 | KM1309 | KM1310 | KM1311 | KM1312 |
| s + e + a + hx | s + a + aq + | s + h + a + | s + h + u + | s + h + hx + | s + h + pi + | s + h + p + | s + b + p + |
| | hx | hx | hx | hx | hx | hx | hx |
| KM1313 | KM1314 | KM1315 | KM1316 | KM1317 | KM1318 | KM1319 | KM1320 |
| s + b + u + | s + h + b + | s + b + a + | s + u + a + | s + b + e + | s + u + e + | s + u + pi + | s + u + p + |
| hx | hx | hx | hx | hx | hx | hx | hx |
| KM1321 | KM1322 | KM1323 | KM1324 | KM1325 | KM1326 | KM1327 | KM1328 |
| s + p + a + hx | s + pi + a + | s + e + a + | s + p + e + | s + pi + e + | s + p + pi + | h + b + a + | h + b + e + |
| | hx | hx | hx | hx | hx | hx | hx |
| KM1329 | KM1330 | KM1331 | KM1332 | KM1333 | KM1334 | KM1335 | KM1336 |
| h + u + e + | h + b + pi + | h + u + pi + | h + b + p + | h + u + p + | h + b + u + | h + u + a + | h + p + a + |
| hx | hx | hx | hx | hx | hx | hx | hx |
| KM1337 | KM1338 | KM1339 | KM1340 | KM1341 | KM1342 | KM1343 | KM1344 |
| h + pi + e + | h + e + a + hx | h + p + e + | h + pi + e + | h + p + pi + | b + u + a + | b + p + a + | b + pi + e + |
| hx | | hx | hx | hx | hx | hx | hx |
| KM1345 | KM1346 | KM1347 | KM1348 | KM1349 | KM1350 | KM1351 | KM1352 |
| b + u + pi + | b + p + pi + | b + u + p + | u + p + a + | u + pi + a + | u + e + a + | u + p + e + | u + pi + e + |
| hx | hx | hx | hx | hx | hx | hx | hx |
| KM1353 | KM1354 | KM1355 | KM1356 | KM1357 | KM1358 | KM1359 | KM1360 |
| u + p + pi + | p + pi + a + | p + pi + e + | pi + e + a + | e + a + aq + | sb + aq + hx | sc + aq + | aq + oc + |
| hx | hx | hx | hx | hx | | hx | hx |
| KM1361 | KM1362 | KM1363 | KM1364 | KM1365 | KM1366 | KM1367 | KM1368 |
| u + hd | p + hd | pi + hd | e + hd | a + hd | bz + hd | bc + hd | cb + hd |
| KM1369 | KM1370 | KM1371 | KM1372 | KM1373 | KM1374 | KM1375 | KM1376 |
| cc + hd | lb + hd | lc + hd | sb + hd | sc + hd | aq + hd | s + h + hd | s + b + hd |
| KM1377 | KM1378 | KM1379 | KM1380 | KM1381 | KM1382 | KM1383 | KM1384 |
| s + u + hd | s + p + hd | s + pi + hd | s + e + hd | s + a + hd | s + bz + hd | s + bc + hd | s + bc + hd |
| KM1385 | KM1386 | KM1387 | KM1388 | KM1389 | KM1390 | KM1391 | KM1392 |
| s + cb + hd | s + cc + hd | s + lb + hd | s + lc + hd | s + sb + hd | s + sc + hd | s + aq + hd | h + b + hd |
| KM1393 | KM1394 | KM1395 | KM1396 | KM1397 | KM1398 | KM1399 | KM1400 |
| h + u + hd | h + p + hd | h + pi + hd | h + e + hd | h + a + hd | h + bz + hd | h + bc + hd | h + bc + hd |
| KM1401 | KM1402 | KM1403 | KM1404 | KM1405 | KM1406 | KM1407 | KM1408 |
| h + cb + | h + cc + hd | h + lb + hd | h + lc + hd | h + sb + hd | h + sc + hd | h + aq + hd | u + p + hd |
| hd | | | | | | | |
| KM1409 | KM1410 | KM1411 | KM1412 | KM1413 | KM1414 | KM1415 | KM1416 |
| u + pi + hd | u + e + hd | u + a + hd | u + bz + hd | u + bc + hd | u + bc + hd | u + cb + hd | u + cc + hd |
| KM1417 | KM1418 | KM1419 | KM1420 | KM1421 | KM1422 | KM1423 | KM1424 |
| u + lb + hd | u + lc + hd | u + sb + hd | u + sc + hd | u + aq + hd | p + pi + hd | p + e + hd | p + a + hd |
| KM1425 | KM1426 | KM1427 | KM1428 | KM1429 | KM1430 | KM1431 | KM1432 |
| p + bz + hd | p + bc + hd | p + cb + hd | p + cc + hd | p + lb + hd | p + lc + hd | p + sb + hd | p + sc + hd |
| KM1433 | KM1434 | KM1435 | KM1436 | KM1437 | KM1438 | KM1439 | KM1440 |
| pi + e + hd | pi + a + hd | pi + bz + hd | pi + bc + hd | pi + bc + hd | pi + cb + hd | pi + cc + hd | pi + lb + hd |
| KM1441 | KM1442 | KM1443 | KM1444 | KM1445 | KM1446 | KM1447 | KM1448 |
| pi + lc + hd | pi + sb + | pi + sc + hd | pi + aq + hd | e + a + hd | e + bz + hd | e + bc + hd | e + cb + hd |
| | hd | | | | | | |
| KM1449 | KM1450 | KM1451 | KM1452 | KM1453 | KM1454 | KM1455 | KM1456 |
| e + cc + hd | e + lb + hd | e + lc + hd | e + sb + hd | e + sc + hd | e + aq + hd | a + bz + hd | a + bc + hd |
| KM1457 | KM1458 | KM1459 | KM1460 | KM1461 | KM1462 | KM1463 | KM1464 |
| a + bc + | a + cb + | a + cc + hd | a + lb + hd | a + lc + hd | a + sb + hd | a + sc + hd | p + aq + hd |
| hd | hd | | | | | | |
| KM1465 | KM1466 | KM1467 | KM1468 | KM1469 | KM1470 | KM1471 | KM1472 |
| a + aq + | bz + bc + | bz + cb + hd | bz + cc + hd | bz + lb + hd | bz + lc + hd | bz + sb + hd | bz + sc + |
| hd | hd | | | | | | hd |
| KM1473 | KM1474 | KM1475 | KM1476 | KM1477 | KM1478 | KM1479 | KM1480 |
| bz + aq + hd | bc + cb + | bc + cc + hd | bc + lb + hd | bc + lc + hd | bc + sb + hd | bc + sc + hd | bc + aq + |
| | hd | | | | | | hd |
| KM1481 | KM1482 | KM1483 | KM1484 | KM1485 | KM1486 | KM1487 | KM1488 |
| cb + cc + | cb + lb + | cb + lc + hd | cb + sb + hd | cb + sc + hd | cb + aq + hd | cc + lb + hd | cc + lc + hd |
| hd | hd | | | | | | |
| KM1489 | KM1490 | KM1491 | KM1492 | KM1493 | KM1494 | KM1495 | KM1496 |
| cc + sb + | cc + sc + | cc + aq + hd | lb + lc + hd | lb + sb + hd | lb + sc + hd | lb + aq + hd | lc + sb + hd |
| hd | hd | | | | | | |
| KM1497 | KM1498 | KM1499 | KM1500 | KM1501 | KM1502 | KM1503 | KM1504 |
| lc + sc + | lc + aq + | sb + sc + hd | s + h + b + hd | s + b + u + hd | s + u + p + hd | s + p + pi + | s + pi + e + |
| hd | hd | | | | | hd | hd |
| KM1505 | KM1506 | KM1507 | KM1508 | KM1509 | KM1510 | KM1511 | KM1512 |
| s + e + a + hd | s + a + aq + | s + h + a + | s + h + u + | s + h + hd + | s + h + pi + | s + h + p + | s + b + p + |
| | hd | hd | hd | hd | hd | hd | hd |
| KM1513 | KM1514 | KM1515 | KM1516 | KM1517 | KM1518 | KM1519 | KM1520 |
| s + b + u + | s + h + b + | s + b + a + | s + u + a + | s + b + e + | s + u + e + | s + u + pi + | s + u + p + |
| hd | hd | hd | hd | hd | hd | hd | hd |
| KM1521 | KM1522 | KM1523 | KM1524 | KM1525 | KM1526 | KM1527 | KM1528 |
| s + p + a + hd | s + pi + a + | s + e + a + | s + p + e + | s + pi + e + | s + p + pi + | h + b + a + | h + b + e + |
| | hd | hd | hd | hd | hd | hd | hd |
| KM1529 | KM1530 | KM1531 | KM1532 | KM1533 | KM1534 | KM1535 | KM1536 |
| h + u + e + | h + b + pi + | h + u + pi + | h + b + p + | h + u + p + | h + b + u + | h + u + a + | h + p + a + |
| hd | hd | hd | hd | hd | hd | hd | hd |
| KM1537 | KM1538 | KM1539 | KM1540 | KM1541 | KM1542 | KM1543 | KM1544 |
| h + pi + e + | h + e + a + hd | h + p + e + | h + pi + e + | h + p + pi + | b + u + a + | b + p + a + | b + pi + e + |
| hd | | hd | hd | hd | hd | hd | hd |
| KM1545 | KM1546 | KM1547 | KM1548 | KM1549 | KM1550 | KM1551 | KM1552 |
| b + u + pi + | b + p + pi + | b + u + p + | u + p + a + | u + pi + a + | u + e + a + | u + p + e + | u + pi + e + |
| hd | hd | hd | hd | hd | hd | hd | hd |
| KM1553 | KM1554 | KM1555 | KM1556 | KM1557 | KM1558 | KM1559 | KM1560 |
| u + p + pi + | p + pi + a + | p + pi + e + | pi + e + a + | e + a + aq + | sb + aq + hd | sc + aq + | aq + oc + |
| hd | hd | hd | hd | hd | | hd | hd |
| KM1561 | KM1562 | KM1563 | KM1564 | KM1565 | KM1566 | KM1567 | KM1568 |
| u + hp | p + hp | pi + hp | e + hp | a + hp | bz + hp | bc + hp | cb + hp |
| KM1569 | KM1570 | KM1571 | KM1572 | KM1573 | KM1574 | KM1575 | KM1576 |
| cc + hp | lb + hp | lc + hp | sb + hp | sc + hp | aq + hp | s + h + hp | s + b + hp |
| KM1577 | KM1578 | KM1579 | KM1580 | KM1581 | KM1582 | KM1583 | KM1584 |
| s + u + hp | s + p + hp | s + pi + hp | s + e + hp | s + a + hp | s + bz + hp | s + bc + hp | s + bc + hp |
| KM1585 | KM1586 | KM1587 | KM1588 | KM1589 | KM1590 | KM1591 | KM1592 |
| s + cb + hp | s + cc + hp | s + lb + hp | s + lc + hp | s + sb + hp | s + sc + hp | s + aq + hp | h + b + hp |
| KM1593 | KM1594 | KM1595 | KM1596 | KM1597 | KM1598 | KM1599 | KM1600 |
| h + u + hp | h + p + hp | h + pi + hp | h + e + hp | h + a + hp | h + bz + hp | h + bc + hp | h + bc + hp |
| KM1601 | KM1602 | KM1603 | KM1604 | KM1605 | KM1606 | KM1607 | KM1608 |
| h + cb + | h + cc + hp | h + lb + hp | h + lc + hp | h + sb + hp | h + sc + hp | h + aq + hp | u + p + hp |
| hp | | | | | | | |
| KM1609 | KM1610 | KM1611 | KM1612 | KM1613 | KM1614 | KM1615 | KM1616 |
| u + pi + hp | u + e + hp | u + a + hp | u + bz + hp | u + bc + hp | u + bc + hp | u + cb + hp | u + cc + hp |
| KM1617 | KM1618 | KM1619 | KM1620 | KM1621 | KM1622 | KM1623 | KM1624 |
| u + lb + hp | u + lc + hp | u + sb + hp | u + sc + hp | u + aq + hp | p + pi + hp | p + e + hp | p + a + hp |
| KM1625 | KM1626 | KM1627 | KM1628 | KM1629 | KM1630 | KM1631 | KM1632 |
| p + bz + | p + bc + hp | p + cb + hp | p + cc + hp | p + lb + hp | p + lc + hp | p + sb + hp | p + sc + hp |
| hp | | | | | | | |
| KM1633 | KM1634 | KM1635 | KM1636 | KM1637 | KM1638 | KM1639 | KM1640 |
| pi + e + hp | pi + a + hp | pi + bz + hp | pi + bc + hp | pi + bc + hp | pi + cb + hp | pi + cc + hp | pi + lb + hp |
| KM1641 | KM1642 | KM1643 | KM1644 | KM1645 | KM1646 | KM1647 | KM1648 |
| pi + lc + hp | pi + sb + | pi + sc + hp | pi + aq + hp | e + a + hp | e + bz + hp | e + bc + hp | e + cb + hp |
| | hp | | | | | | |
| KM1649 | KM1650 | KM1651 | KM1652 | KM1653 | KM1654 | KM1655 | KM1656 |
| e + cc + hp | e + lb + hp | e + lc + hp | e + sb + hp | e + sc + hp | e + aq + hp | a + bz + hp | a + bc + hp |
| KM1657 | KM1658 | KM1659 | KM1660 | KM1661 | KM1662 | KM1663 | KM1664 |
| a + bc + | a + cb + | a + cc + hp | a + lb + hp | a + lc + hp | a + sb + hp | a + sc + hp | p + aq + hp |
| hp | hp | | | | | | |
| KM1665 | KM1666 | KM1667 | KM1668 | KM1669 | KM1670 | KM1671 | KM1672 |
| a + aq + | bz + bc + | bz + cb + hp | bz + cc + hp | bz + lb + hp | bz + lc + hp | bz + sb + hp | bz + sc + |
| hp | hp | | | | | | hp |
| KM1673 | KM1674 | KM1675 | KM1676 | KM1677 | KM1678 | KM1679 | KM1680 |
| bz + aq + hp | bc + cb + | bc + cc + hp | bc + lb + hp | bc + lc + hp | bc + sb + hp | bc + sc + hp | bc + aq + |
| | hp | | | | | | hp |
| KM1681 | KM1682 | KM1683 | KM1684 | KM1685 | KM1686 | KM1687 | KM1688 |
| cb + cc + | cb + lb + | cb + lc + hp | cb + sb + hp | cb + sc + hp | cb + aq + hp | cc + lb + hp | cc + lc + hp |
| hp | hp | | | | | | |
| KM1689 | KM1690 | KM1691 | KM1692 | KM1693 | KM1694 | KM1695 | KM1696 |
| cc + sb + | cc + sc + | cc + aq + hp | lb + lc + hp | lb + sb + hp | lb + sc + hp | lb + aq + hp | lc + sb + hp |
| hp | hp | | | | | | |
| KM1697 | KM1698 | KM1699 | KM1700 | KM1701 | KM1702 | KM1703 | KM1704 |
| lc + sc + | lc + aq + | sb + sc + hp | s + h + b + hp | s + b + u + hp | s + u + p + hp | s + p + pi + | s + pi + e + |
| hp | hp | | | | | hp | hp |
| KM1705 | KM1706 | KM1707 | KM1708 | KM1709 | KM1710 | KM1711 | KM1712 |
| s + e + a + hp | s + a + aq + | s + h + a + | s + h + u + | s + h + hp + | s + h + pi + | s + h + p + | s + b + p + |
| | hp | hp | hp | hp | hp | hp | hp |
| KM1713 | KM1714 | KM1715 | KM1716 | KM1717 | KM1718 | KM1719 | KM1720 |
| s + b + u + | s + h + b + | s + b + a + | s + u + a + | s + b + e + | s + u + e + | s + u + pi + | s + u + p + |
| hp | hp | hp | hp | hp | hp | hp | hp |
| KM1721 | KM1722 | KM1723 | KM1724 | KM1725 | KM1726 | KM1727 | KM1728 |
| s + p + a + hp | s + pi + a + | s + e + a + | s + p + e + | s + pi + e + | s + p + pi + | h + b + a + | h + b + e + |
| | hp | hp | hp | hp | hp | hp | hp |
| KM1729 | KM1730 | KM1731 | KM1732 | KM1733 | KM1734 | KM1735 | KM1736 |
| h + u + e + | h + b + pi + | h + u + pi + | h + b + p + | h + u + p + | h + b + u + | h + u + a + | h + p + a + |
| hp | hp | hp | hp | hp | hp | hp | hp |
| KM1737 | KM1738 | KM1739 | KM1740 | KM1741 | KM1742 | KM1743 | KM1744 |
| h + pi + e + | h + e + a + hp | h + p + e + | h + pi + e + | h + p + pi + | b + u + a + | b + p + a + | b + pi + e + |
| hp | | hp | hp | hp | hp | hp | hp |
| KM1745 | KM1746 | KM1747 | KM1748 | KM1749 | KM1750 | KM1751 | KM1752 |
| b + u + pi + | b + p + pi + | b + u + p + | u + p + a + | u + pi + a + | u + e + a + | u + p + e + | u + pi + e + |
| hp | hp | hp | hp | hp | hp | hp | hp |
| KM1753 | KM1754 | KM1755 | KM1756 | KM1757 | KM1758 | KM1759 | KM1760 |
| u + p + pi + | p + pi + a + | p + pi + e + | pi + e + a + | e + a + aq + | sb + aq + hp | sc + aq + | aq + oc + |
| hp | hp | hp | hp | hp | | hp | hp |
| KM1761 | KM1762 | KM1763 | KM1764 | KM1765 | KM1766 | KM1767 | KM1768 |
| h + hp | b + hp | s | h | b | u | p | pi |
| KM1769 | KM1770 | KM1771 | KM1772 | KM1773 | KM1774 | KM1775 | KM1776 |
| e | a | bz | bc | cb | cc | lb | lc |

| KM1777 | KM1778 | KM1779 | KM1780 | KM1781 | KM1782 | KM1783 |
|---|---|---|---|---|---|---|
| sb | sc | aq | oc | hx | hd | hp |

The above-described embodiments of the cosmetic agents exhibit a good deodorant and/or antiperspirant performance and offer outstanding preservation. The synergistic effect of the preservatives used allows the amounts thereof to be reduced, without adversely affecting the preserving action. The cosmetic agents according to the invention thus cause particularly little irritation and sensitivity.

The cosmetic agents according to the invention can additionally include further auxiliary substances. The antiperspirant cosmetic agents according to the invention preferably comprise at least one further auxiliary substance, selected from the group consisting of (i) emulsifiers and/or surfactants; (ii) hydrogel-forming agents; (iii) chelating agents; (iv) waxes; (v) odorants; (vi) skin-cooling active ingredients; (vii) thickeners; and (viii) the mixtures thereof.

Preferably suited emulsifiers and surfactants according to the invention are selected from anionic, cationic, non-ionic, amphoteric, in particular ampholytic and zwitterionic, emulsifiers and surfactants. Surfactants are amphiphilic (bifunctional) compounds, which are composed of at least one hydrophobic molecule part and at least one hydrophilic molecule part. The hydrophobic group is preferably a hydrocarbon chain having 8 to 28 carbon atoms, which can be saturated or unsaturated, linear or branched. This C₈ to C₂₈ alkyl chain is particularly preferably linear.

Anionic surfactants shall be understood to mean surfactants carrying exclusively anionic charges; they include carboxyl groups, sulfonic acid groups, or sulfate groups, for example. Particularly preferred anionic surfactants are alkyl sulfates, alkyl ether sulfates, acyl glutamates, and C₈₋₂₄ carboxylic acids, and the salts thereof, known as soaps.

Cationic surfactants shall be understood to mean surfactants carrying exclusively cationic charges; they include quaternary ammonium groups, for example. Cationic surfactants of the quaternary ammonium compound, esterquat and amidoamine types are preferred. Preferred quaternary ammonium compounds are ammonium halides and the imidazolium compounds known under the INCI names Quaternium-27 and Quaternium-83. Other cationic surfactants that can be used according to the invention are the quaternized protein hydrolysates. Preferred esterquats are quaternized ester salts of fatty acids with triethanolamine, quaternized ester salts of fatty acids with diethanol alkylamines, and quaternized ester salts of fatty acids with 1,2-dihydroxypropyl dialkylamines.

Amphoteric surfactants are divided into ampholytic surfactants and zwitterionic surfactants. Ampholytic surfactants are understood to include those surface active compounds that carry both acidic (—COOH or —SO₃H groups, for example) and basic hydrophilic groups (amino groups, for example) and can exhibit acidic or basic behavior, depending on the conditions. A person skilled in the art considers zwitterionic surfactants to be surfactants that carry both a negative charge and a positive charge in the same molecule. Examples of preferred zwitterionic surfactants are betaines, N-alkyl-N,N-dimethylammonium glycinates, N-acyl-aminopropyl-N,N-dimethylammonium glycinates, and 2-alkyl-3-carboxymethyl-3-hydroxyethyl imidazolines, each having 8 to 24 carbon atoms in the alkyl group. Examples of preferred ampholytic surfactants are N-alkyl glycines, N-alkylaminopropionic acids, N-alkylaminobutytic acids, N-alkyliminodipropionic acids, N-hydroxyethyl-N-alkylamidopropyl glycines, N-alkyl taurines, N-alkyl sarcosines, 2-alkylaminopropionic acids, and alkylaminoacetic acids, each having 8 to 24 carbon atoms in the alkyl group.

The compositions according to the invention formulated as emulsions, in particular as oil-in-water emulsions, preferably include at least one non-ionic oil-in-water emulsifier having an HLB value of more than 7 to 20. These are emulsifiers that are generally known to a person skilled in the art, as they are listed in Kirk-Othmer, “Encyclopedia of Chemical Technology”, 3rd edition, 1979, Volume 8, pages 9133, for example. For ethoxylated products, the HLB value is calculated according to the formula HLB=(100−L):5, where L is the weight proportion of the lipophilic groups, which is to say of the fatty alkyl or fatty acyl groups, in the ethylene oxide adducts, expressed in percent by weight. In this connection, it may be preferred according to the invention if furthermore a water-in-oil emulsifier having an HLB value of greater than 1.0 and less than/equal to 7.0 is used. Suitable non-ionic oil-in-water emulsifiers and suitable non-ionic water-in-oil emulsifiers within the scope of the present invention are described in the German unexamined patent application DE 102006004957 A1, for example. Particularly well-suited non-ionic surfactants are silicone copolyols comprising ethylene oxide units or ethylene oxide and propylene oxide units, which are likewise disclosed in the unexamined patent application DE 102006004957 A1.

For thickening the antiperspirant cosmetic agents according to the invention preferably hydrogel-forming substances are used that are selected from cellulose ethers, especially hydroxyalkyl celluloses, in particular hydroxypropyl cellulose, hydroxypropyl methyl cellulose, hydroxyethyl cellulose, carboxymethyl cellulose, cetyl hydroxyethylcellulose, hydroxybutyl methyl cellulose, methyl hydroxyethyl cellulose, furthermore xanthan gum, sclerotium gum, succinoglycans, polygalactomannan gums, in particular guar gums and locust bean gum, in particular guar gum and locust bean gum itself and the non-ionic hydroxyalkyl guar derivatives and locust bean gum derivatives, such as hydroxypropyl guar, carboxymethyl hydroxypropyl guar, hydroxypropyl methyl guar, hydroxyethyl guar and carboxymethyl guar, furthermore pectins, agar, caragheen (carrageenan), tragacanth, gum arabic, karaya gum, tara gum, gellan gum, gelatin, casein, propylene glycol alginate, alginic acids and the salts thereof, in particular sodium alginate, potassium alginate and calcium alginate, furthermore polyvinylpyrrolidones, polyvinyl alcohols, polyacrylamides, furthermore—albeit less preferred—physically (such as by way of pre-gelatinization) and/or chemically modified starches, in particular hydroxypropylated starch phosphates and starch octenyl succinates and the aluminum, calcium or sodium salts thereof, furthermore—likewise less preferred—acrylic acid/acrylate copolymers, acrylic acid/acrylamide copolymers, acrylic acid/vinylpyrrolidone copolymers, acrylic acid/vinylformamide copolymers, and polyacrylates. Particularly preferred hydrogel-forming agents are selected from cellulose ethers, especially from hydroxyalkyl celluloses, in particular from hydroxypropyl cellulose, hydroxypropyl methyl cellulose, hydroxyethyl cellulose, carboxymethyl cellulose, cetyl hydroxyethylcellulose, hydroxybutyl methyl cellulose and methyl hydroxyethyl cellulose, and mixtures thereof. The hydrogel-forming agent used is preferably hydroxyethyl cellulose.

Furthermore, the cosmetic agents can comprise at least one chelating agent, which is preferably selected from ethylenediaminetetraacetic acid (EDTA) and the salts thereof, and from nitrilotriacetic acid (NTA), and mixtures of these substances, in a total amount of 0.01 to 0.5 wt. %, especially 0.02 to 0.3 wt. %, in particular 0.05 to 0.1 wt. %, based on the total weight of the propellant-free cosmetic agent.

In addition, the cosmetic agents according to the invention can comprise an odorant. The term “odorants” within the meaning of the present invention shall be understood to mean substances having a molar mass of 74 to 300 g/mol, which include at least one osmophoric group in the molecule and have an odor and/or a flavor, which is to say they are capable of stimulating the receptors of the hair cells of the olfactory system. Osmophoric groups are groups that are covalently bound to the molecular skeleton in the form of hydroxy groups, formyl groups, oxo groups, alkoxy carbonyl groups, nitrile groups, nitro groups, azide groups and the like. In this connection, the term “odorants” within the meaning of the present invention also covers perfume oils, perfumes, or perfume oil components that are liquid at 20° C. and 1,013 hPa. The odorant is preferably selected from the group consisting of
(i) esters, in particular benzyl acetate, phenoxyethyl isobutyrate, p-tert.-butylcyclohexyl acetate, linalyl acetate, dimethyl benzyl carbinyl acetate (DMBCA), phenylethyl acetate, benzyl acetate, ethyl methylphenyl glycinate, allyl cyclohexyl propionate, styrallyl propionate, benzyl salicylate, cyclohexyl salicylate, floramate, melusate, and jasmecyclate;
(ii) ethers, in particular benzyl ethyl ethers and Ambroxan;
(iii) aldehydes, in particular linear alkanals having 8 to 18 carbon atoms, citral, citronellal, citronellyl oxy acetaldehyde, cyclamen aldehyde, lilial and bourgeonal;
(iv) ketones, in particular jonone, alpha-isomethyl ionone and methyl cedryl ketone;
(v) alcohols, in particular anethol, citronellol, eugenol, geraniol, linalool, phenylethyl alcohol and terpineol;
(vi) hydrocarbons, in particular terpenes such as limonene and pinene; and
(vii) the mixtures thereof.
   Preferably mixtures of different odorants are used, which together produce an appealing odorous note.

Cosmetic agents according to the invention that have a particularly appealing odor are obtained when the odorant is present in a total amount of 0.00001 to 10 wt. %, especially 0.001 to 9 wt. %, preferably 0.01 to 8 wt. %, more preferably 0.5 to 7 wt. %, and in particular 1 to 6 wt. %, based on the total weight of the propellant-free cosmetic agent.

In addition, the cosmetic agents according to the invention can comprise at least one wax. “Waxes” within the scope of the present invention shall be understood to mean substances that are kneadable or solid to brittle-hard at 20° C., have a coarse to microcrystalline structure, and are translucent to colors to opaque, but not vitreous. These substances furthermore melt above 25° C. without decomposing, are easily liquid (low viscosity) just above the melting point, have a highly temperature-dependent consistency and solubility, and can be polished under light pressure. The wax can preferably be selected from the group consisting of
(i) coconut fatty acid glycerol monoesters, diesters and triesters;
(ii) Butyrospermum Parkii (shea butter);
(iii) esters of saturated, monohydric C₈₋₁₈ alcohols with saturated C₁₂₋₁₈ monocarboxylic acids;
(iv) linear, primary C₁₂-C₂₄ alkanols;
(v) esters of a saturated, monohydric C₁₆-C₆₀ alkanol and a saturated C₈-C₃₆ monocarboxylic acid, in particular cetyl behenate, stearyl behenate and C₂₀-C₄₀ alkyl stearate;
(vi) glycerol triesters of saturated linear C₁₂ to C₃₀ carboxylic acids, which may be hydroxylated, in particular hydrogenated palm oil, hydrogenated coconut oil, hydrogenated castor oil, glyceryl tribehenate, and glyceryltri-12-hydroxy stearate;
(vii) natural plant-based waxes, in particular candelilla wax, carnauba wax, Japan wax, sugar cane wax, ouricoury wax, cork wax, sunflower wax, fruit waxes;
(viii) animal waxes, in particular beeswax, shellac wax, and cetaceum;
(ix) synthetic waxes, in particular montan ester waxes, hydrogenated jojoba waxes and sasol waxes, polyalkylene waxes and polyethylene glycol waxes, C₂₀ to C₄₀ dialkyl esters of dimer acids, C₃₀₋₅₀ alkyl beeswax, and alkyl and alkyl aryl esters of dimeric fatty acids, paraffin waxes; and
(x) the mixtures thereof.

Commercial products bearing the INCI name Cocoglycerides, in particular the commercial products Novata® (from BASF), particularly preferably Novata® AB, a mixture of C₁₂₋₁₈ mono-, di-, and triglycerides that melts in the range from 30 to 32° C., and the products of the Softisan series (Sasol Germany GmbH) bearing the INCI name Hydrogenated Cocoglycerides, in particular Softisan 100, 133, 134, 138, 142, are particularly preferred. Further preferred esters of saturated, monohydric C₁₂₋₁₈ alcohols with saturated C₁₂₋₁₈ monocarboxylic acids are stearyl laurate, cetearyl stearate (such as Crodamol® CSS), cetyl palmitate (such as Cutina® CP), and myristyl myristate (such as Cetiol® MM). Furthermore a Cd₂₀ to C₄₀ alkyl stearate is preferably used as the wax component. This ester is known under the name Kester Wax® K82H or Kester Wax® K80H and is sold by Koster Keunen Inc.

It is preferred within the scope of the present invention if the wax is present in a total amount of 0.01 to 60 wt. %, especially 3.0 to 30 wt. %, preferably 5.0 to 18 wt. %, and in particular 6 to 15 wt. %, based on the total weight of the propellant-free cosmetic agent.

According to a further embodiment of the present invention, the antiperspirant cosmetic agents furthermore include at least one skin-cooling active ingredient. Suitable skin-cooling active ingredients according to the invention are, for example, menthol, isopulegol and menthol derivatives, such as menthyl lactate, menthyl glycolate, menthyl ethyl oxamate, menthyl pyrrolidone carboxylic acid, menthyl methyl ether, menthoxypropanediol, menthone glycerin acetal (9-methyl-6-(1-methylethyl)-1,4-dioxaspiro(4.5)decane-2-methanol), monomenthyl succinate, 2-hydroxymethyl-3,5,5-trimethylcyclohexanol, and 5-methyl-2-(1-methylethyl)cyclohexyl-N-ethyloxamate. Menthol, isopulegol, menthyl lactate, menthoxypropanediol, menthylpyrrolidone carboxylic acid and 5-methyl-2-(1-methylethyl)cyclohexyl-N-ethyloxamate and mixtures of these substances, in particular mixtures of menthol and menthyl lactate, menthol, menthol glycolate and menthyl lactate, menthol and menthoxypropanediol, or menthol and isopulegol, are preferred as skin-cooling active ingredients.

Furthermore, lipophilic thickeners can be used according to the invention as auxiliary substances. The at least one antiperspirant aluminum salt is preferably suspended undissolved in at least one cosmetic oil that is liquid at 20° C. and 1,013 hPa. To improve applicability, at least one lipophilic thickener can also be added to this suspension as a suspending aid. Preferred lipophilic thickeners according to the invention are selected from hydrophobized clay minerals and fumed silica.

Moreover, in principle all further components that are known to a person skilled in the art for such cosmetic compositions may be used in the cosmetic agents according to the invention. Further active ingredients, auxiliary substances and additives are, for example:
structurants such as maleic acid and lactic acid;
fiber structure-improving active ingredients, in particular monosaccharides, disaccharides and oligosaccharides such as glucose, galactose, fructose, fructose and lactose;
dyes for coloring the agent;
substances for setting the pH value, for example α- and β-hydroxycarboxylic acids;
active ingredients such as allantoin and bisabolol;
ceramides. Ceramides shall be understood to mean N-acylsphingosine (fatty acid amides of sphingosine) or synthetic analogs of such lipids (known as pseudoceramides);
pigments;
viscosity regulators such as salts (NaCl);
cationic, non-ionic and amphoteric polymers;
vitamins, in particular from groups A, B, C, E, F and H;
protein hydrolysates and cationized protein hydrolysates;
humectants or auxiliary penetrating agents and/or swelling agents, in particular urea and urea derivatives, guanidine and the derivatives thereof, arginine and the derivatives thereof, waterglass, imidazole and the derivatives thereof, histidine and the derivatives thereof;
plants extracts, such as of green tea, white tea, oak bark, stinging nettle, witch hazel, hops, chamomile, burdock root, horsetail, hawthorn, lime blossom, lychee, almond, aloe vera, pine needle, horse chestnut, sandalwood, juniper, coconut, mango, apricot, lime, wheat, kiwi, melon, orange, grapefruit, sage, rosemary, birch, mallow, lady's smock, creeping thyme, yarrow, thyme, balm, restharrow, coltsfoot, marshmallow, ginseng, ginger root, Echinacea purpurea, Olea europea, Foeniculum vulgare, and Apium graveolens.

The above-described further ingredients may be present in a total amount of 0.001 to 50 wt. %, especially 0.01 to 40 wt. %, preferably 0.1 to 30 wt. %, and in particular 0.5 to 20 wt. %, based on the total weight of the propellant-free cosmetic agent.

A second subject matter of the present invention is a non-therapeutic cosmetic method for preventing and/or reducing perspiration of the body and/or for preventing and/or reducing body odor, in which a cosmetic agent according to the invention is applied to the skin, and in particular to the skin of the axilla region, and remains on the skin for at least 1 hour, especially for at least 2 hours, preferably for at least 4 hours, and in particular for at least 6 hours.

What was said above with respect to the cosmetic agents according to the invention applies, mutatis mutandis, with respect to further preferred embodiments of the method according to the invention, and in particular with respect to the cosmetic agents used therein.

What was said with respect to the cosmetic agents according to the invention and with respect to the method according to the invention applies, mutatis mutandis, with respect to further embodiments of the use according to the invention.

In summary, the present invention is in particular characterized by the following items:
1. A cosmetic agent, comprising in at least one cosmetically compatible carrier
   a) 0.0005 to 35 wt. %, based on the total weight of the propellant-free cosmetic agent, of at least one active ingredient, selected from the group consisting of deodorant active ingredients, antiperspirant aluminum salts and the mixtures thereof;
   b) 2.0 to 95 wt. %, based on the total weight of the propellant-containing cosmetic agent, of at least one propellant;
   c) at least one first preservative (A), selected from the group consisting of sulfite(s), hexetidine, benzyl alcohol(s), undecylenic acid, phenoxyisopropanol, piroctone olamine, ethyl lauroyl arginate, formic acid, quaternary ammonium compounds, o-cymen-5-ol and hexamidine and the salts thereof; and
   d) at least one second preservative (B), selected from the group consisting of benzoic acid and the salts thereof, propionic acid and the salts thereof, salicylic acid and the salts thereof, sorbic acid and the salts thereof, zinc salts, paraben(s), polyaminopropyl biguanide, phenoxyethanol, climbazole, chlorhexidine and the salts thereof, glutaral, chlorphenesin, dimethyl oxazolidine, diazolidinyl urea, and mixtures of these preservatives.
2. The cosmetic agent according to item 1, characterized by comprising at least one deodorant active ingredient, selected from the group consisting of (i) silver salts; (ii) alkanediols having 5 to 12 carbon atoms, in particular 3-(2-ethylhexyloxy)-1,2-propanediol, 1,2-hexanediol and 1,2-octanediol; (iii) triethyl citrates; (iv) active ingredients against exoesterases, in particular against arylsulfatase, lipase, beta-glucuronidase and cystathionine β-lyase; (v) cationic phospholipids; (vii) odor absorbers, in particular silicates, such as montmorillonite, kaolinite, illite, beidellite, nontronite, saponite, hectorite, bentonite, smectite, and talcum, zeolites, zinc ricinoleate, cyclodextrins; (vii) deodorizingly acting ion exchangers; (viii) antimicrobial substances; (ix) prebiotically active components; and (x) the mixtures thereof.
3. The cosmetic agent according to either item 1 or 2, characterized by comprising, based on the total weight of the propellant-free cosmetic agent, 0.0005 to 20 wt. %, especially 0.1 to 12 wt. %, preferably 0.1 to 10 wt. %, and in particular 0.1 to 2.0 wt. % of at least one deodorant active ingredient.
4. The cosmetic agent according to any one of the preceding items, characterized by comprising at least one antiperspirant aluminum salt, selected from the group consisting of (i) water-soluble astringent inorganic salts of aluminum, in particular aluminum chlorohydrate, aluminum sesquichlorohydrate, aluminum dichlorohydrate, aluminum hydroxide, potassium aluminum sulfate, aluminum bromohydrate, aluminum chloride, aluminum sulfate; (ii) water-soluble astringent organic salts of aluminum, in particular aluminum chlorohydrex propylene glycol, aluminum chlorohydrex polyethylene glycol, aluminum propylene glycol complexes, aluminum sesquichlorohydrex propylene glycol, aluminum sesquichlorohydrex polyethylene glycol, aluminum propylene glycol dichlorohydrex, aluminum polyethylene glycol dichlorohydrex, aluminum undecylenoyl collagen amino acid, sodium aluminum lactate, sodium aluminum chlorohydroxy lactate, aluminum lipoamino acids, aluminum lactate, aluminum chlorohydroxy allantoinate, sodium aluminum chlorohydroxy lactate; and (iii) the mixtures thereof.
5. The cosmetic agent according to any one of the preceding items, characterized by comprising, based on the total weight of the propellant-free cosmetic agent, 0.1 to 35 wt. %, especially 0.5 to 25 wt. %, preferably 1 to 15 wt. %, more preferably 1.5 to 10 wt. %, and in particular 2.0 to 8.0 wt. % of at least one antiperspirant aluminum salt.
6. The cosmetic agent according to any one of the preceding items, characterized by comprising at least one propellant, selected from the group consisting of propane, propene, n-butane, iso-butane, iso-butene, n-pentane, pentene, iso-pentane, iso-pentene, methane, ethane, dimethyl ether, nitrogen, air, oxygen, nitrous oxide, 1,1,1,3-tetrafluoroethane, heptafluoro-n-propane, perfluoroethane, monochlorodifluoromethane, 1,1-difluoroethane, tetrafluoropropene, carbon dioxide, and the mixtures thereof.
7. The cosmetic agent according to any one of the preceding items, characterized by comprising, based on the total weight of the propellant-containing cosmetic agent, 2.0 to 90 wt. %, especially 30 to 85 wt. %, preferably 35 to 80 wt. %, and in particular 40 to 75 wt. % of at least one deodorant active ingredient.
8. The cosmetic agent according to any one of the preceding items, characterized by comprising at least two preservatives (A), selected from the group consisting of sulfite(s), hexetidine, benzyl alcohol(s), undecylenic acid, phenoxyisopropanol, piroctone olamine, ethyl lauroyl arginate, formic acid, quaternary ammonium compounds, o-cymen-5-ol and hexamidine and the salts thereof.
9. The cosmetic agent according to any one of the preceding items, characterized by comprising at least three preservatives (A), selected from the group consisting of sulfite(s), hexetidine, benzyl alcohol(s), undecylenic acid, phenoxyisopropanol, piroctone olamine, ethyl lauroyl arginate, formic acid, quaternary ammonium compounds, o-cymen-5-ol and hexamidine and the salts thereof.
10. The cosmetic agent according to any one of the preceding items, characterized by comprising at least four preservatives (A), selected from the group consisting of sulfite(s), hexetidine, benzyl alcohol(s), undecylenic acid, phenoxyisopropanol, piroctone olamine, ethyl lauroyl arginate, formic acid, quaternary ammonium compounds, o-cymen-5-ol and hexamidine and the salts thereof.
11. The cosmetic agent according to any one of the preceding items, characterized by comprising at least five preservatives (A), selected from the group consisting of sulfite(s), hexetidine, benzyl alcohol(s), undecylenic acid, phenoxyisopropanol, piroctone olamine, ethyl lauroyl arginate, formic acid, quaternary ammonium compounds, o-cymen-5-ol and hexamidine and the salts thereof.
12. The cosmetic agent according to any one of the preceding items, characterized by comprising a mixture of sulfite(s), hexetidine, benzyl alcohol(s), undecylenic acid, phenoxyisopropanol, piroctone olamine, ethyl lauroyl arginate, formic acid, quaternary ammonium compounds, o-cymen-5-ol and hexamidine and the salts thereof, as preservative (A).
13. The cosmetic agent according to one of the preceding items, characterized by comprising at least one compound, selected from the group consisting of behentrimonium chloride, cetrimonium bromide, cetrimonium chloride, laurtrimonium bromide, laurtrimonium chloride, steartrimonium bromide, steartrimonium chloride, benzethonium chloride, benzalkonium chloride, and the mixtures thereof, as quaternary ammonium compounds.
14. The cosmetic agent according to any one of the preceding items, characterized by comprising, based on the total weight of the propellant-free cosmetic agent, 0.001 to 10 wt. %, especially 0.005 to 7.0 wt. %, preferably 0.01 to 4.0 wt. %, and in particular 0.05 to 2.0 wt. % of at least one first preservative (A).
15. The cosmetic agent according to any one of the preceding items, characterized by comprising, based on the total weight of the propellant-free cosmetic agent, 0.001 to 12 wt. %, especially 0.005 to 7.0 wt. %, preferably 0.05 to 6.0 wt. %, and in particular 0.1 to 5.0 wt. % of at least one second preservative (B).
16. A non-therapeutic cosmetic method for preventing and/or reducing perspiration of the body and/or for preventing and/or reducing body odor, in which a propellant-free cosmetic agent according to any one of items 1 to 15 is applied to the skin, and in particular to the skin of the axilla region, and remains on the skin for at least 1 hour, especially for at least 2 hours, preferably for at least 4 hours, and in particular for at least 6 hours.

The following examples describe the present invention in more detail, without limiting it to these examples.

### Examples

Sprayable, translucent antiperspirant microemulsions (information in wt. %, based on the microemulsion without propellant)

| | 1.1 | 1.2 | 1.3 | 1.4 | 1.5 |
|---|---|---|---|---|---|
| Plantaren ® 1200 | 1.7 | 1.7 | — | 1.7 | 1.7 |
| Plantaren ® 2000 | 1.15 | 1.4 | 2.40 | 1.15 | 1.4 |
| Glycerol monooleate | 0.7 | 0.7 | — | 0.7 | 0.7 |
| Dioctyl ether | 4 | 4 | 0.1 | 4 | 4 |
| Octyldodecanol | 1 | 1 | 0.02 | 1 | 1 |
| Perfume oil | 1 | 1 | 1 | 1 | 1 |
| Aluminum chlorohydrate | 8 | 5 | 5 | 10 | 9 |
| 1,2-propylene glycol | 5 | 5 | — | 5 | 5 |
| Glycerol | — | — | 5 | — | — |
| 2-benzylheptane-1-ol | 0.5 | — | — | 0.5 | 0.5 |
| Triethyl citrate | — | 0.5 | 0.5 | 0.2 | — |
| Triclosan | — | — | — | — | — |
| Zinc lactate | — | 0.2 | — | — | — |
| Benzoic acid | — | — | — | — | 2.5 |
| Sodium benzoate | 0.2 | — | — | — | 0.5 |
| Propionic acid | — | — | 1.0 | — | 2.0 |
| Salicylic acid | — | — | — | — | 0.5 |
| Sodium salicylate | 0.3 | — | — | — | 0.5 |
| Zinc pyrithione | — | 0.5 | — | — | 0.5 |
| Methyl paraben | — | — | 0.1 | | 0.2 |
| Ethylparaben | 0.1 | — | — | — | 0.1 |
| Polyaminopropyl | — | — | — | 0.2 | 0.3 |
| biguanide | | | | | |
| Phenoxyethanol | — | — | 0.8 | — | 1.0 |
| Climbazole | — | 0.3 | — | — | 0.5 |
| Chlorhexidine | — | — | — | — | 0.3 |
| Glutaral | — | — | — | 0.1 | 0.1 |
| Chlorphenesin | — | — | — | 0.2 | 0.3 |
| Dimethyloxazolidine | — | 0.1 | — | — | 0.1 |
| Diazolidinyl urea | — | — | — | — | 0.5 |
| KM¹⁾ | 0.1 | 0.2 | 0.5 | 0.05 | 1.0 |
| Water, deionized | to make | to make | to make | to make | to make |
| | 100.0 | 100.0 | 100.0 | 100.0 | 100.0 |

¹⁾preservative (A), selected from at least one of the preservatives listed in Table 6, or mixtures of these agents KM1 to KM1783

Microemulsion is applied from a pressurized container (aerosol) using a suitable spray system. Propellants that may be used include air, nitrous oxide, nitrogen, CO₂ and other compressible gases.

Antiperspirant emulsion (O/W) (information in wt. %, based on the O/W emulsion without propellant)

| | 2.1 | 2.2 | 2.3 | 2.4 | 2.5 |
|---|---|---|---|---|---|
| Aluminum zirconium | 23.7 | — | 23.7 | — | 23.7 |
| tetrachlorohydrex gly | | | | | |
| Locron L | — | 30.0 | — | 30.0 | — |
| Steareth-2 | 2.4 | 2.2 | 2.4 | 2.2 | 2.4 |
| Steareth-21 | 1.6 | 1.4 | 1.6 | 1.4 | 1.6 |
| Perfume | 1.2 | 1.0 | 1.2 | 1.0 | 1.2 |
| PPG-15 stearyl ether | 0.5 | 1.5 | 0.5 | 1.5 | 0.5 |
| Aluminum starch | 0.1 | — | 0.1 | — | 0.1 |
| octenylsuccinate | | | | | |
| Benzoic acid | — | — | — | 1.5 | 2.5 |
| Sodium benzoate | — | — | — | — | 0.5 |
| Propionic acid | — | — | — | — | 2.0 |
| Salicylic acid | 0.3 | — | — | — | 0.5 |
| Sodium salicylate | | — | — | — | 0.5 |
| Zinc pyrithione | 0.4 | — | — | — | 0.5 |
| Methyl paraben | — | — | 0.1 | — | 0.2 |
| Ethylparaben | — | — | 0.1 | — | 0.1 |
| Polyaminopropyl | — | 0.2 | — | — | 0.3 |
| biguanide | | | | | |
| Phenoxyethanol | — | — | — | 0.8 | 1.0 |
| Climbazole | — | — | — | — | 0.5 |
| Chlorhexidine | — | — | — | — | 0.3 |
| Glutaral | — | 0.1 | — | — | 0.1 |
| Chlorphenesin | — | — | — | — | 0.3 |
| Dimethyloxazolidine | — | — | 0.1 | — | 0.1 |
| Diazolidinyl urea | — | — | — | 0.4 | 0.5 |
| KM¹⁾ | 0.5 | 0.4 | 0.5 | 0.4 | 0.5 |
| Water | to make | to make | to make | to make | to make |
| | 100.0 | 100.0 | 100.0 | 100.0 | 100.0 |

¹⁾preservative (A), selected from at least one of the preservatives listed in Table 6, or mixtures of these agents KM1 to KM1783

Emulsions 2.1 to 2.5 are each applied from a pressurized container (aerosol) using a suitable spray system. Propellants that may be used include air, nitrous oxide, nitrogen, CO₂ and other compressible gases.

Deodorant (information in wt. %, based on the deodorant without propellant)

| | 3.1 | 3.2 | 3.3 | 3.4 | 3.5 |
|---|---|---|---|---|---|
| Ethanol 96% (cosmetic; | 55.0 | 55.0 | 55.0 | 55.0 | 55.0 |
| denatured) | | | | | |
| Triethyl citrate | 3.0 | 3.0 | 3.0 | 3.0 | 3.0 |
| Cremophor ® RH 455 | 0.5 | 0.5 | 0.5 | 0.5 | 0.5 |
| Eucarol ® AGE/EC/UP | 1.0 | 1.0 | 1.0 | 1.0 | 1.0 |
| Cocamidopropyl PG | 0.4 | 0.4 | 0.4 | 0.4 | 0.4 |
| dimonium chloride | | | | | |
| phosphate | | | | | |
| Perfume oil | 1.0 | 1.0 | 1.0 | 1.0 | 1.0 |
| Benzoic acid | — | — | 2.0 | 1.8 | 2.5 |
| Sodium benzoate | — | — | — | — | 0.5 |
| Propionic acid | — | — | 1.5 | — | 2.0 |
| Salicylic acid | — | 0.3 | — | — | 0.5 |
| Sodium salicylate | — | — | — | — | 0.5 |
| Zinc pyrithione | 0.5 | — | — | — | 0.5 |
| Methyl paraben | — | — | — | — | 0.2 |
| Ethylparaben | — | — | — | — | 0.1 |
| Polyaminopropyl | 0.2 | — | — | — | 0.3 |
| biguanide | | | | | |
| Phenoxyethanol | — | — | — | 0.8 | 1.0 |
| Climbazole | — | 0.4 | — | | 0.5 |
| Chlorhexidine | — | — | — | — | 0.3 |
| Glutaral | — | — | — | — | 0.1 |
| Chlorphenesin | — | — | — | — | 0.3 |
| Dimethyloxazolidine | — | — | — | — | 0.1 |
| Diazolidinyl urea | — | 0.4 | — | — | 0.5 |
| KM¹⁾ | 0.5 | 1.0 | 0.2 | 0.2 | 0.7 |
| Water | to make | to make | to make | to make | to make |
| | 100 | 100 | 100 | 100 | 100 |

¹⁾preservative (A), selected from at least one of the preservatives listed in Table 6, or mixtures of these agents KM1 to KM1783

Deodorants 3.1 to 3.5 are each applied from a pressurized container (aerosol) using a suitable spray system. Propellants that may be used include air, nitrous oxide, nitrogen, CO₂ and other compressible gases.

Water-in-oil emulsions according to the invention (all quantity information in wt. %, based on the total weight of the water-in-oil emulsion without propellant)

| | 4.1 | 4.2 | 4.3 | 4.4 | 4.5 |
|---|---|---|---|---|---|
| Aluminum chlorohydrate | 33 | 33 | 33 | 33 | 33 |
| C₁₀-C₁₃ isoalkane | 8.9 | 8.9 | — | 8.9 | 8.9 |
| Cyclopentasiloxane | — | — | 8.9 | — | — |
| PEG/PPG-18/18 | 1.4 | 1.4 | 1.4 | 1.4 | 1.4 |
| Dimethicone | | | | | |
| Isoceteth-20 | 0.5 | 0.5 | 0.5 | 0.5 | 0.5 |
| Dimethicone | 4.2 | 4.2 | 4.2 | 4.2 | 4.2 |
| Isopropyl myristate | 9.0 | 9.0 | 9.0 | 9.0 | 9.0 |
| 1,2-propanediol | 7.0 | 25 | 25 | 7.0 | 25 |
| Phenoxyethanol | 0.5 | 0.5 | 0.5 | 0.5 | 0.5 |
| Perfume | 2.5 | 2.5 | 2.5 | 2.5 | 2.5 |
| L-menthol | 0.4 | — | — | 0.4 | — |
| Benzoic acid | — | — | 1.2 | — | 2.5 |
| Sodium benzoate | 0.4 | | — | 0.4 | 0.5 |
| Propionic acid | — | — | — | — | 2.0 |
| Salicylic acid | — | — | 0.4 | — | 0.5 |
| Sodium salicylate | — | — | — | — | 0.5 |
| Zinc pyrithione | 0.4 | — | — | — | 0.5 |
| Methyl paraben | — | — | 0.1 | — | 0.2 |
| Ethylparaben | — | — | — | 0.1 | 0.1 |
| Polyaminopropyl | — | — | — | — | 0.3 |
| biguanide | | | | | |
| Phenoxyethanol | — | 0.8 | — | — | 1.0 |
| Climbazole | — | — | — | — | 0.5 |
| Chlorhexidine | 0.2 | — | — | — | 0.3 |
| Glutaral | — | 0.1 | — | — | 0.1 |
| Chlorphenesin | — | 0.2 | — | — | 0.3 |
| Dimethyloxazolidine | — | — | — | 0.1 | 0.1 |
| Diazolidinyl urea | — | — | — | — | 0.5 |
| KM¹⁾ | 0.2 | 0.25 | 1.0 | 0.7 | 0.5 |
| Water | to make | to make | to make | to make | to make |
| | 100 | 100 | 100 | 100 | 100 |

¹⁾preservative (A), selected from at least one of the preservatives listed in Table 6, or mixtures of these agents KM1 to KM11783

Emulsions 4.1 to 4.5 are each applied from a pressurized container (aerosol) using a suitable spray system. The aerosol container is filled with 15, 20, 25 or 30% of the formulation together with a suitable propellant gas, such as a propane/butane/isobutane mixture (pressure 1.6 to 3.8 bar) (to make 100).

Emulsion according to PIT method (all information in wt. %, based on the propellant-free emulsion):

| | 5.1 | 5.2 | 5.3 | 5.4 | 5.5 |
|---|---|---|---|---|---|
| Emulgade ® SE-PF | 5 | 35.6 | 5 | 35.6 | 5 |
| Eumulgin ® B1 | 3 | 13 | 3 | 13 | 3 |
| Cetiol ® SN | 5 | 6 | 5 | 6 | 5 |
| Cetiol ® CC | 5 | 8 | 5 | 8 | 5 |
| Glycerol 86% | 0.1 | 0.2 | 0.1 | 0.2 | 0.1 |
| Perfume | 1 | 1 | 1 | 1 | 1 |
| Benzoic acid | 0.8 | — | — | — | 2.5 |
| Sodium benzoate | — | — | — | — | 0.5 |
| Propionic acid | — | — | — | 1.5 | 2.0 |
| Salicylic acid | 0.5 | — | — | — | 0.5 |
| Sodium salicylate | — | — | — | — | 0.5 |
| Zinc pyrithione | — | — | 0.4 | | 0.5 |
| Methyl paraben | — | — | — | 0.2 | 0.2 |
| Ethylparaben | 0.1 | — | — | | 0.1 |
| Polyaminopropyl | — | 0.2 | — | | 0.3 |
| biguanide | | | | | |
| Phenoxyethanol | — | | — | 0.8 | 1.0 |
| Climbazole | — | | — | — | 0.5 |
| Chlorhexidine | — | 0.2 | — | — | 0.3 |
| Glutaral | — | — | 0.1 | — | 0.1 |
| Chlorphenesin | — | — | — | — | 0.3 |
| Dimethyloxazolidine | — | — | 0.1 | — | 0.1 |
| Diazolidinyl urea | — | 0.4 | — | — | 0.5 |
| KM¹⁾ | 0.1 | 0.5 | 0.1 | 0.2 | 1.0 |
| Water | to make | to make | to make | to make | to make |
| | 100 | 100 | 100 | 100 | 100 |

¹⁾preservative (A), selected from at least one of the preservatives listed in Table 6, or mixtures of these agents KM1 to KM1783

Emulsions 5.1 to 5.5 are each applied from a pressurized container (aerosol) using a suitable spray system. Propellants that may be used include air, nitrous oxide, nitrogen, CO₂ and other compressible gases.

The following commercial products were used:

| Commercial | | Supplier/ |
|---|---|---|
| product | INCI | manufacturer |
| Cetiol ® SN | CETEARYL ISONONANOATE | BASF |
| Cetiol ® CC | DICAPRYLYL CARBONATE | BASF |
| Cremophor ® | PEG-60 HYDROGENATED CASTOR | BASF |
| RH 455 | OIL | |
| Eucarol ® | SODIUM COCO-GLUCOSIDE | Cesalpinia |
| AGE/EC/UP | TARTRATE | |
| Dow Corning | Dimethicone, PEG/PPG-18/18 | Dow |
| ES-5227 DM | dimethicone at a weight ratio of 3:1 | Corning |
| Emulgade SE-PF | GLYCERYL STEARATE, | BASF |
| | CETEARETH-20, CETEARETH-12, | |
| | CETEARYL ALCOHOL, CETYL | |
| | PALMITATE | |
| Eumulgin ® B1 | Ceteareth-12 | BASF |
| Finsolv TN | C12-15 Alkyl benzoate | Innospec |
| Locron L | Aluminum chlorohydrate | Clariant |
| (AS = 50%) | | |
| Plantaren ® 1200 | LAURYL GLUCOSIDE, approx. | BASF |
| | 50% AS | |
| Plantaren ® 2000 | DECYL GLUCOSIDE, approx.. | BASF |
| | 50% AS | |

While at least one exemplary embodiment has been presented in the foregoing detailed description of the invention, it should be appreciated that a vast number of variations exist. It should also be appreciated that the exemplary embodiment or exemplary embodiments are only examples, and are not intended to limit the scope, applicability, or configuration of the invention in any way. Rather, the foregoing detailed description will provide those skilled in the art with a convenient road map for implementing an exemplary embodiment of the invention, it being understood that various changes may be made in the function and arrangement of elements described in an exemplary embodiment without departing from the scope of the invention as set forth in the appended claims and their legal equivalents.

What is claimed is:

1. A cosmetic agent, comprising in at least one cosmetically compatible carrier
a) 0.0005 to 35 wt. %, based on the total weight of the propellant-free cosmetic agent, of at least one active ingredient, selected from the group consisting of deodorant active ingredients, antiperspirant aluminum salts and the mixtures thereof;
b) 2.0 to 95 wt. %, based on the total weight of the propellant-containing cosmetic agent, of at least one propellant;
c) at least one first preservative (A), selected from the group consisting of sulfite(s), hexetidine, benzyl alcohol(s), undecylenic acid, phenoxyisopropanol, piroctone olamine, ethyl lauroyl arginate, formic acid, quaternary ammonium compounds, o-cymen-5-ol and hexamidine and the salts thereof; and
d) at least one second preservative (B), selected from the group consisting of benzoic acid and the salts thereof, propionic acid and the salts thereof, salicylic acid and the salts thereof, sorbic acid and the salts thereof, zinc salts, paraben(s), polyaminopropyl biguanide, phenoxyethanol, climbazole, chlorhexidine and the salts thereof, glutaral, chlorphenesin, dimethyl oxazolidine, diazolidinyl urea, and mixtures of these preservatives.

2. The cosmetic agent according to claim 1, wherein the cosmetic agent includes at least one deodorant active ingredient comprising 0.0005 to 20 wt. % based on the total weight of the propellant-free cosmetic agent.

3. The cosmetic agent according to claim 1, wherein the cosmetic agent includes at least one deodorant active ingredient comprising 0.1 to 12 wt. % based on the total weight of the propellant-free cosmetic agent.

4. The cosmetic agent according to claim 1, wherein the cosmetic agent includes at least one deodorant active ingredient comprising 0.1 to 10 wt. % based on the total weight of the propellant-free cosmetic agent.

5. The cosmetic agent according to claim 1, wherein the cosmetic agent includes at least one deodorant active ingredient comprising 0.1 to 2.0 wt. % based on the total weight of the propellant-free cosmetic agent.

6. The cosmetic agent according to claim 1, wherein the cosmetic agent includes at least one antiperspirant aluminum salt which comprises 0.1 to 35 wt. % based on the total weight of the propellant-free cosmetic agent.

7. The cosmetic agent according to claim 1, wherein the cosmetic agent includes at least one antiperspirant aluminum salt which comprises 0.5 to 25 wt. % based on the total weight of the propellant-free cosmetic agent.

8. The cosmetic agent according to claim 1, wherein the cosmetic agent includes at least one antiperspirant aluminum salt which comprises 1 to 15 wt. % based on the total weight of the propellant-free cosmetic agent.

9. The cosmetic agent according to claim 1, wherein the cosmetic agent includes at least one antiperspirant aluminum salt which comprises 1.5 to 10 wt. % based on the total weight of the propellant-free cosmetic agent.

10. The cosmetic agent according to claim 1, wherein the cosmetic agent includes at least one antiperspirant aluminum salt which comprises 2.0 to 8.0 wt. % based on the total weight of the propellant-free cosmetic agent.

11. The cosmetic agent according to claim 1, wherein the cosmetic agent includes at least two preservatives (A) selected from the group consisting of sulfite(s), hexetidine, benzyl alcohol(s), undecylenic acid, phenoxyisopropanol, piroctone olamine, ethyl lauroyl arginate, formic acid, quaternary ammonium compounds, o-cymen-5-ol and hexamidine and the salts thereof.

12. The cosmetic agent according to claim 1, wherein the cosmetic agent includes at least three preservatives (A), selected from the group consisting of sulfite(s), hexetidine, benzyl alcohol(s), undecylenic acid, phenoxyisopropanol, piroctone olamine, ethyl lauroyl arginate, formic acid, quaternary ammonium compounds, o-cymen-5-ol and hexamidine and the salts thereof.

13. The cosmetic agent according to claim 1, wherein the cosmetic agent includes at least four preservatives (A), selected from the group consisting of sulfite(s), hexetidine, benzyl alcohol(s), undecylenic acid, phenoxyisopropanol, piroctone olamine, ethyl lauroyl arginate, formic acid, quaternary ammonium compounds, o-cymen-5-ol and hexamidine and the salts thereof.

14. The cosmetic agent according to claim 1, wherein the at least one preservative A is a mixture of sulfite(s), hexetidine, benzyl alcohol(s), undecylenic acid, phenoxyisopropanol, piroctone olamine, ethyl lauroyl arginate, formic acid, quaternary ammonium compounds, o-cymen-5-ol and hexamidine and the salts thereof, as preservative (A).

15. The cosmetic agent according to claim 1, wherein the at least one first preservative (A) comprises 0.001 to 10 wt. % based on the total weight of the propellant-free cosmetic agent.

16. The cosmetic agent according to claim 1, wherein the at least one first preservative (A) comprises 0.01 to 4.0 wt. % based on the total weight of the propellant-free cosmetic agent.

17. The cosmetic agent according to claim 1, wherein the at least one first preservative (A) comprises 0.05 to 2.0 wt. % based on the total weight of the propellant-free cosmetic agent.

18. The cosmetic agent according to claim 1, wherein the at least one second preservative (B) comprises 0.001 to 12 wt. % based on the total weight of the propellant-free cosmetic agent.

19. The cosmetic agent according to claim 1, wherein the at least one second preservative (B) comprises 0.1 to 5.0 wt. % based on the total weight of the propellant-free cosmetic agent.

20. A non-therapeutic cosmetic method for preventing and/or reducing perspiration of the body and/or for preventing and/or reducing body odor, in which a propellant-free cosmetic agent according to claim 1 is applied to the skin, and is left on the skin for at least 1 hour.
